(19) United States
(12) Patent Application Publication
(10) Pub. No.: US 2024/0318172 A1
(43) Pub. Date: Sep. 26, 2024

(54) PRODUCTS AND METHODS FOR INHIBITION OF EXPRESSION OF PERIPHERAL MYELIN PROTEIN-22

(71) Applicants: RESEARCH INSTITUTE AT NATIONWIDE CHILDREN'S HOSPITAL, Columbus, OH (US); The Cyprus Foundation for Muscular Dystrophy Research D/B/A The Cyprus Institute of Neurology..., Agios Dometios (CY)

(72) Inventors: Scott Quenton Harper, Powell, OH (US); Kleopas Kleopa, Agios Dometios (CY); Marina Stavrou, Agios Dometios (CY)

(21) Appl. No.: 18/038,900

(22) PCT Filed: Nov. 30, 2021
(86) PCT No.: PCT/US21/61177

§ 371 Date: May 25, 2023

Related U.S. Application Data
(60) Provisional application No. 63/120,190, filed on Dec. 1, 2020.

(51) Int. Cl. C12N 15/113; A61K 31/7088; A61K 48/00; A61P 21/00; C12N 15/86

(52) U.S. Cl. CPC C12N 15/113; A61K 31/7088; A61K 48/005; A61P 21/00; C12N 15/86; C12N 2310/11; C12N 2310/141; C12N 2750/14143

(57) ABSTRACT

RNA interference-based methods and products for inhibiting the expression of a peripheral myelin protein-22 gene are provided. RNAs that inhibit the peripheral myelin protein-22 gene are provided as well as DMAs encoding the RNAs. Delivery vehicles such as recombinant adeno-associated viruses deliver DMAs encoding RNAs that inhibit the peripheral myelin protein-22 gene. The methods treat Charcot-Marie-Tooth Disease such as Charcot-Marie-Tooth Disease Type 1 A (CMT1A).

### FIELD

RNA interference-based methods and products for inhibiting the expression of a peripheral myelin protein-22 gene are provided. RNAs that inhibit the peripheral myelin protein-22 gene are provided as well as DNAs encoding the RNAs. Delivery vehicles such as recombinant adeno-associated viruses deliver DNAs encoding RNAs that inhibit the peripheral myelin protein-22 gene. The methods treat Charcot-Marie-Tooth Disease such as Charcot-Marie-Tooth Disease Type 1A (CMT1A).

### INCORPORATION BY REFERENCE OF THE SEQUENCE LISTING

This application contains, as a separate part of disclosure, a Sequence Listing in computer-readable form (Filename: 56204_SeqListing.txt; 6,159,677 bytes-ASCII text file dated Nov. 30, 2021) which is incorporated by reference herein in its entirety.

### BACKGROUND

Charcot-Marie-Tooth disease (CMT) refers to a heterogeneous group of hereditary peripheral neuropathies that affect 1 in 2500 people. The most common type, CMT Type 1, is a demyelinating peripheral neuropathy. The CMT Type 1 subtype that affects more than 50% of all CMT cases and about 70-80% of CMT Type 1 cases is autosomal dominant demyelinating CMT neuropathy type 1A [CMT1A (MIM 118220)].

CMT1A is most frequently caused by a dominantly inherited 1.4 Mb tandem intra-chromosomal duplication on chromosome 17p11.2-p12. The duplication results in three copies of the peripheral myelin protein-22 (PMP22) gene which are translated into PMP22 protein [Timmerman et al., Nature Genetics, 1(3): 171-175 (1992) and Valentijn et al., Nature Genetics, 1(3): 166-170 (1992)]. In some cases, point mutations in PMP22 may also lead to dominant CMT1A and generally present with the most severe phenotype [Matsunami et al., Nature Genetics, 1(3):176-179 (1992), Patel et al., Nature Genetics, 1(3): 159-165 (1992), Timmerman et al., supra]. Patients with CMT1A develop slowly progressive distal muscle weakness and atrophy, sensory loss, and absent reflexes with a typical onset at adolescence. CMT1A shows high variability in disease severity even within the same family. Sensory responses are usually absent while motor nerve conduction velocities (MNCVs) are slowed, ranging from 5 to 35 m/s in the forearm, but most average around 20 m/s, with uniform and symmetric findings in different nerves. Although MNCVs do not change significantly over decades, motor amplitudes and the number of motor units decrease slowly, reflecting axonal loss, which correlates with clinical disability.

The PMP22 protein is a 22-kDa intrinsic tetraspan glycoprotein primarily produced by myelinating Schwann cells (SCs) during development and makes 2-5% of peripheral nervous system (PNS) compact myelin. This protein is crucial for SC growth and differentiation, myelogenesis, myelin thickness and in the maintenance of PNS axons and myelin. PMP22 is also involved in the linkage of cytoskeletal actin with the plasma membrane, serving as a regulator of cholesterol content in lipid rafts. Despite the fact that PMP22 mRNA is expressed in almost every tissue, PMP22 protein is only found in myelinating SCs, suggesting a tissue specific post-transcriptional regulation of PMP22 mRNA [Maier et al., Mol. Cell. Neuroscience, 24(3): 803-817 (2003) and Roux et al., J. Comp. Neurol., 474(4): 578-588 (2004)].

The 5′-UTR of the PMP22 gene includes two known promoters, P1 and P2. Their respective transcripts differ only at their 5′ non-coding region [Bosse et al., J. Neuroscience Res., 37(4): 529-537 (1994) and Suter et al., J. Biol. Chem., 269(41): 25795-25808 (1994)] but result in six splice variants which exhibit a tissue-specific expression pattern [Visigalli et al., Hum. Mut., 37(1): 98-109 (2015)]. PMP22 regulation is achieved through its intronic regions and enhancer elements within them [Jones et al., Hum. Mol. Genet., 21(7): 1581-1591 (2012); Srinivasan et al., Nuc. Acids Res., 40(14): 6449-6460 (2012); and Pantera et al., Hum. Mol. Genet., 27(16): 2830-2839 (2018)]. P1 promoter transcripts are SC-specific, while the P2 promoter transcripts are expressed in non-PNS tissues. Therefore, duplication of the PMP22 gene or of its key transcriptional binding sites shifts the ratio of splicing isoforms and alters methylation, microRNA binding and post-translational modification sites [Verrier et al., Glia, 57(12): 1265-1279 (2009) and Lee et al., Exp. Neurobiology, 28(2): 279-288 (2019)]. In normal myelinating and non-myelinating SCs, approximately 20% of the newly synthesized PMP22 is glycosylated while the remaining ˜80% is targeted for proteasomal endoplasmic reticulum (ER)-associated degradation (ERAD).

CMT1A is thought to depend on gene dosage effects of PMP22 because CMT1A patients have increased PMP22 mRNA [Yoshikawa et al., Ann. Neurol., 35(4): 445-450 (1994)] and protein [Gabriel et al., Neurology, 49(6): 1635-1640 (2015)] levels in their sural nerve biopsies. Individuals who carry a combination of one deleted and one duplicated PMP22 allele do not present CMT1A-like phenotype as they have a balanced gene dosage. Some CMT1A phenotypes may also result from a different size or type of duplication on chromosome 17p that affects PMP22 expression [Pantera, supra]. CMT1A patients with 1.4 Mb duplication may have variable PMP22 levels in skin biopsies not necessarily correlating with disease severity [Nobbio et al., Brain, 137(Pt 6): 1614-1620 (2014) and Katona et al., Brain, 132(Pt 7): 1734-1740 (2014)]. Nevertheless, supporting the PMP22 gene dosage effect as the driving mechanism of CMT1A, rodent models overexpressing PMP22 reproduced a CMT1A-like phenotype [Sereda et al., Neuron, 16(5): 1049-60 (1996); Huxley et al., Hum. Mol. Genet., 5(5): 563-569 (1996); Huxley et al., Hum. Mol. Genet., 7(3): 449-458 (1998); Magyar et al., J. Neuroscience, 16(17): 5351-5360 (1996); Perea et al., Hum. Mol. Genet., 10(10): p. 1007-1018 (2001); Robaglia-Schlupp et al., Brain, 125(Pt 10): 2213-2221 (2002); and Robertson et al., J. Anat., 200(4): 377-390 (2002)], which was ameliorated after interrupting PMP22 overexpression [Fledrich et al., Nat. Med., 20(9): 1055-1061 (2014); Lee, supra; Perea, supra; Sereda et al., Nat. Med., 9(12): 1533-1537 (2003); Passage et al., Nat. Med., 10(4): 396-401 (2004); Meyer et al., Ann. Neurology, 61(1): 61-72 (2007); Chumakov et al., Orphanet Journal of Rare Diseases, 9(1): 201 (2014); Lee et al., Neurobiology of Disease, 100: 99-107 (2017); Zhao et al., J. Clin. Invest., 128(1): 359-368 (2018); Prukop et al., PLoS One, 14(1): e0209752 (2019); and Lee et al., Nuc. Acids Res., 48(1): 130-140 (2020)].

Overexpressed PMP22 has been shown to saturate the proteasomal capacity for degradation, leading to perinuclear or cytoplasmic PMP22 accumulation, decreased overall proteasomal activity, and ER stress. PMP22 is also involved in early steps of myelinogenesis, in the determination of myelin thickness and maintenance. PMP22 duplication destabilizes the architecture, protein stoichiometry and function of the myelin sheath and SC, leading to demyelination, remyelination, the characteristic onion bulb formation and SC apoptosis. As a consequence, impairment in SC-axon interactions and dysfunctional neurofilament structure result in higher packing density and less phosphorylation of neurofilaments accompanied by slower axonal transport and myelination rates.

Current treatments for CMT1A remain geared toward general symptom management in the form of physical therapy or corrective surgery.

There thus exists a need in the art for products and methods for treatment of CMT1A.

### SUMMARY

The disclosure herein provides methods to specifically induce silencing of overexpressed PMP22 by RNA interference (RNAi) using vectors expressing artificial inhibitory RNAs targeting the PMP22 mRNA. The artificial inhibitory RNAs contemplated include, but are not limited to, small interfering RNAs (siRNAs) (also referred to as short interfering RNAs, small inhibitory RNAs or short inhibitory RNAs), short hairpin RNAs (shRNAs) and miRNA shuttles (artificial miRNAs) that inhibit expression of the PMP22 gene. The artificial inhibitory RNAs are referred to as miPMP22s herein. The miPMP22s are small regulatory sequences that act post-transcriptionally by targeting, for example, the 3′UTR of PMP22 mRNA in a reverse complementary manner resulting in reduced PMP22 mRNA and protein levels. Use of the methods and products is indicated, for example, in preventing or treating CMT1A.

PMP22 inhibitory RNAs are provided as well as polynucleotides encoding one or more of the miPMP22s. The disclosure provides nucleic acids comprising RNA-encoding template nucleotide sequences comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity to the sequence set forth in any one of SEQ ID NOs: 1-8.

Exemplary miPMP22s comprise the full length sequences set out in any one of SEQ ID NOs: 9-16 or variants thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identity to the sequence set forth in any one of SEQ ID NOs: 9-16. Corresponding final processed antisense guide strand sequences are respectively set out in SEQ ID NOs: 17-24, or are variants thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identity to the sequence set forth in any one of SEQ ID NOs: 17-24. The processed antisense guide strand is the strand of the mature miRNA duplex that becomes the RNA component of the RNA induced silencing complex ultimately responsible for sequence-specific gene silencing. The disclosure additionally provides the antisense guide strands set out in FIG. 48 and contemplates variants of each of those antisense guide strands that are at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identical. The disclosure additionally provides the antisense guide strands set out in FIG. 50 and contemplates variants of each of those antisense guide strands that are at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identical.

miPMP22s can specifically bind to a segment of a messenger RNA (mRNA) encoded by a human PMP22 gene (represented by SEQ ID NO: 25 which is a human PMP22 cDNA), wherein the segment conserved relative to mRNA encoded by the wild-type mouse PMP22 gene (represented by SEQ ID NO: 27 which is a mouse PMP22 cDNA). For example, a miPMP22 can specifically bind a mRNA segment that is complementary to a sequence within nucleotides 1-2423 of SEQ ID NO: 25. More particularly, a miPMP22 can specifically bind a mRNA segment that is complementary to a sequence within nucleotides 1412-1433 of SEQ ID NO: 25 (the nucleotides bound by, for example, miPMP22-868) or 1415-1436 of SEQ ID NO: 25 (the nucleotides bound by, for example, miPMP22-871).

Delivery of DNA encoding miPMP22s can be achieved using a delivery vehicle that delivers the DNA(s) to a Schwann cell. For example, recombinant AAV (rAAV) vectors can be used to deliver DNA encoding miPMP22s. Other vectors (for example, other viral vectors such as lentivirus, adenovirus, retrovirus, equine-associated virus, alphavirus, pox viruses, herpes virus, polio virus, sindbis virus and vaccinia viruses) can also be used to deliver polynucleotides encoding miPMP22s. Thus, also provided are viral vectors encoding one or more miPMP22s. When the viral vector is a rAAV, the rAAV lack AAV rep and cap genes. The rAAV can be self-complementary (sc) AAV. As another example, non-viral vectors such as lipid nanoparticles can be used for delivery.

Provided herein are rAAV, each encoding a miPMP22. Also provided are rAAV encoding one or more miPMP22s. A rAAV (with a single-stranded genome, scAAV) encoding one or more miPMP22s can encode one, two, three, four, five, six, seven or eight miPMP22s. A scAAV encoding one or more miPMP22s can encode one, two, three or four miPMP22s.

Compositions are provided comprising the nucleic acids or viral vectors described herein.

Further provided are methods of preventing or inhibiting expression of the PMP22 gene in a cell comprising contacting the cell with a delivery vehicle (such as rAAV) encoding a miPMP22 wherein, if the delivery vehicle is rAAV, the rAAV lacks rep and cap genes. In the methods, expression of the duplicated and/or mutant PMP22 allele is inhibited by at least 10, at least 20, at least 30, at least 40, at least 50, at least 60, at least 70, at least 80, at least 90, at least 95, at least 98 percent, at least 99 percent, or 100 percent. In the methods, expression of the wild-type PMP22 allele is inhibited by at least 10, at least 20, at least 30, at least 40, at least 50, at least 60, at least 70, at least 80, at least 90, at least 95, at least 98 percent, at least 99 percent, or 100 percent.

Still further provided are methods of delivering DNA encoding a miPMP22 to an subject in need thereof, comprising administering to the subject a delivery vehicle (such as rAAV) comprising DNA encoding the miPMP22 wherein, if the delivery vehicle is rAAV, the rAAV lacks rep and cap genes. Other methods of delivering DNA encoding the miPMP22 to an subject in need thereof, comprise administering to the subject a delivery vehicle (such as rAAV) comprising DNA encoding one or more miPMP22 wherein, if the delivery vehicle is rAAV, the rAAV lacks rep and cap genes.

Methods are also provided of preventing or treating CMT1A comprising administering a delivery vehicle (such as rAAV) comprising DNA encoding a miPMP22 wherein, if the delivery vehicle is rAAV, the rAAV lacks rep and cap genes. Other methods of preventing or treating CMT1A comprise administering a delivery vehicle (such as rAAV) comprising DNA encoding one or more miPMP22 wherein, if the delivery vehicle is rAAV, the rAAV lacks rep and cap genes. The methods result in restoration of PMP22 expression to at least 25 percent, at least 30, at least 40, at least 50, at least 60, at least 70, at least 80, at least 90, at least 95, at least 98 percent, at least 99 percent, or 100 percent or more, of normal PMP22 expression in an unaffected subject.

The disclosure provides a delivery vehicle that is a viral vector comprising the nucleic acids described herein and/or a combination of any one or more thereof. Viral vectors provided include, but are not limited to an adeno-associated virus (AAV), adenovirus, lentivirus, retrovirus, poxvirus, baculovirus, herpes simplex virus, vaccinia virus, or a synthetic virus. The viral vector can be an AAV. The AAV lacks rep and cap genes. The AAV can be a recombinant AAV (rAAV) or a self-complementary recombinant AAV (scAAV). The AAV is, for example, AAV-1, AAV-2, AAV-3, AAV-4, AAV-5, AAV-6, AAV-7, AAV-8, AAV-9, AAV-10, AAV-11, AAV-12, AAV-13, AAV-anc80, or AAV rh.74. The AAV can be AAV-9. The AAV can be a pseudotyped AAV, for example, an AAV2/8 or AAV2/9.

The disclosure provides a composition comprising the nucleic acids described herein and a pharmaceutically acceptable carrier. The disclosure provides a composition comprising a viral vector comprising the nucleic acids described herein, and/or a combination of any one or more thereof and a pharmaceutically acceptable carrier.

The disclosure provides a composition comprising a delivery vehicle capable of delivering agents to a Schwann cell a nucleic acid encoding a miPMP22, wherein the miPMP22 binds a segment of a mRNA encoded by a human PMP22 gene (wherein the segment either does or does not encode sequence comprising a mutation associated with CMT1A); wherein the segment is conserved relative to the wild-type mouse PMP22 gene, and, optionally, a pharmaceutically acceptable carrier. The human PMP22 gene can comprise the sequence of SEQ ID NO: 25, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, identity. The mouse PMP22 gene can comprise the sequence of SEQ ID NO: 27, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99%, identity. A miPMP22 specifically binds, for example, a mRNA segment that is complementary to a sequence within nucleotides 1412-1433 of SEQ ID NO: 25 (the nucleotides bound by, for example, miPMP22-868) or 1415-1436 of SEQ ID NO: 25 (the nucleotides bound by, for example, miPMP22-871).

The disclosure provides a delivery vehicle in the compositions that is a viral vector. The viral vector in the compositions can be, for example, an adeno-associated virus (AAV), adenovirus, lentivirus, retrovirus, poxvirus, baculovirus, herpes simplex virus, vaccinia virus, or a synthetic virus. The viral vector can be an AAV. The AAV lacks rep and cap genes. The AAV can be a recombinant AAV (rAAV) or a self-complementary recombinant AAV (scAAV). The AAV is or has a capsid serotype selected from, for example, AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74. The AAV can be or can have a capsid serotype of AAV-9. The AAV can be a pseudotyped AAV, such as AAV2/8 or AAV2/9.

The disclosure provides methods of delivering to a Schwann cell comprising a duplicated and/or mutant PMP22 gene: (a) a nucleic acid comprising a template nucleic acid encoding a miPMP22 comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-8; a nucleic acid encoding the full length miPMP22 sequences set out in any one of SEQ ID NOs: 9-16 or variants thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identity to the sequence set forth in any one of SEQ ID NOs: 9-16; a nucleic acid encoding a miPMP22 processed antisense guide strand comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 17-24; a nucleic acid encoding one or more antisense guide strands set out in FIG. 48, or a variant of an antisense guide strand in FIG. 48 that is at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identical; or a nucleic acid encoding one or more antisense guide strands set out in FIG. 50, or a variant of an antisense guide strand in FIG. 50 that is at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identical; (b) a vector comprising any one or more of the nucleic acids described herein; or (c) a composition comprising any one or more of the nucleic acids or vectors described herein.

The disclosure provides a method of treating a subject suffering from a duplicated and/or mutant PMP22 gene, the method comprising administering to the subject(a) a nucleic acid comprising a template nucleic acid encoding a miPMP22 comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-8; a nucleic acid encoding the full length miPMP22 sequences set out in any one of SEQ ID NOs: 9-16 or variants thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identity to the sequence set forth in any one of SEQ ID NOs: 9-16; a nucleic acid encoding a miPMP22 processed antisense guide strand comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 17-24; a nucleic acid encoding one or more antisense guide strands set out in FIG. 48, or a variant of an antisense guide strand in FIG. 48 that is at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identical; or a nucleic acid encoding one or more antisense guide strands set out in FIG. 50, or a variant of an antisense guide strand in FIG. 50 that is at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identical; (b) a vector comprising any one or more of the nucleic acids described herein; or (c) a composition comprising any one or more of the nucleic acids or vectors described herein.

The disclosure contemplates the subject treated by methods herein suffers from CMT1A. The disclosure also contemplates treatment of a subject that is at risk for CMT1A due to a duplication or mutation of the PMP22 gene. The subject can be a mammalian animal. The subject can be a human subject.

The disclosure also provides uses of at least one nucleic acid as described herein, at least one viral vector as described herein, or a composition as described herein in making a medicament for, or in treating a subject suffering from, a duplicated and/or mutant PMP22 gene.

The disclosure also provides uses of at least one nucleic acid as described herein, at least one viral vector as described herein, or a composition as described herein in making a medicament for or in treating CMT1A in a subject in need thereof.

Other features and advantages of the disclosure will be apparent from the following description of the drawing and the detailed description. It should be understood, however, that the drawing, detailed description, and the examples, while indicating embodiments of the disclosed subject matter, are given by way of illustration only, because various changes and modifications within the spirit and scope of the disclosure will become apparent from the drawing, detailed description, and the examples.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1 shows an example of an artificial miRNA shuttle sequence to demonstrate folding and processing sites. The mature guide strand is underlined. Grey arrowheads indicate Drosha cut sites; black arrowheads indicate Dicer cut sites. Shaded sequences at extreme 5′ and 3′ ends are restriction sites in the template DNA used to clone the miRNA shuttles in front of the U6 promoter.

FIG. 2 shows a human PMP22 full length cDNA sequence (SEQ ID NO: 25) in which alternating shading shows exon boundaries (5 exons) and the underlined sequence is the longest human PMP22 protein coding open reading frame (ORF).

FIG. 3 shows the human PMP22 full-length ORF sequence with a translated PMP22 protein sequence (SEQ ID NO: 26).

FIG. 4 shows a mouse PMP22 cDNA sequence (SEQ ID NO: 27) in which alternating shading shows exon boundaries (5 exons).

FIG. 5A-B shows the human PMP22 cDNA with miPMP22 binding sites. All miPMP22 target sequences are located in Exon 5 (in the 3′ UTR region). Underlined sequence is the PMP22 full-length open reading frame.

FIG. 6 shows the full-length miPMP22-868 sequence (SEQ ID NO: 9).

FIG. 7 shows binding interactions of miPMP22-868 and miPMP22-871 with mouse and human PMP22.

FIG. 8 shows the full-length miPMP22-871 sequence (SEQ ID NO: 10).

FIG. 9 shows the full-length miPMP22-869 sequence (SEQ ID NO: 11).

FIG. 10 shows the full-length miPMP22-872 sequence (SEQ ID NO: 12).

FIG. 11 shows the full-length miPMP22-1706 sequence (SEQ ID NO: 13).

FIG. 12 shows the full-length miPMP22-1740 sequence (SEQ ID NO: 14).

FIG. 13 shows the full-length miPMP22-1741 sequence (SEQ ID NO: 15).

FIG. 14 shows the full-length miPMP22-1834 sequence (SEQ ID NO: 16).

FIG. 15A-D shows qPCR results of in vitro testing of PMP22 knockdown by miPMP22s.

FIG. 16A-D shows in vivo expression of AAV9 in lumbar roots of adult mice. Representative images of lumbar root sections of a non-injected mouse (A) and of mice 4 and 8 weeks after lumbar intrathecal injection of AAV9-U6-miRLacZ-CMV-EGFP (B-C). Sections were immunostained for eGFP (red) indicating cells expressing the reporter gene along with miRLacZ. eGFP was also auto-fluorescent (green). Cell nuclei were stained with DAPI (blue). Arrow heads reveal examples of perinuclear eGFP immunoreactivity in SCs. Quantification of the percentage of eGFP positive cells is shown in D (Mean, SD). Data were compared using the Student t-test, p<0.0001. Averages: 4 weeks 45.95, 8 weeks 56.82.

FIG. 17A-D shows in vivo expression of AAV9 in sciatic nerves of adult mice. Representative images of sciatic nerve sections (A-C) and sciatic nerve teased fibers (E-G) of a non-injected mouse (A,E) and of mice 4 (B,F) and 8 (C,G) weeks after lumbar intrathecal injection with AAV9-U6-miRLacZ-CMV-EGFP (B-C). Sections were immunostained for eGFP (red) indicating cell expressing the reporter gene along with miRLacZ. eGFP was also auto-fluorescent (green). Cell nuclei were stained with DAPI (blue). Arrow heads reveal examples of perinuclear eGFP immunoreactivity in SCs. Quantification of the percentage of eGFP positive cells is shown in D (Mean, SD). Data were compared using the Student t-test, p=0.0460. Averages: 4 weeks 42.07, 8 weeks 45.74.

FIG. 18A-D shows in vivo expression of AAV9 in femoral nerves of adult mice. Representative images of teased femoral nerve fibers of a non-injected mouse (A) and of mice 4 and 8 weeks after lumbar intrathecal injection with AAV9 expressing AAV9-U6-miRLacZ-CMV-EGFP (B-C). Fibers were immunostained for eGFP (red) indicating cell expressing the reporter gene along with miRLacZ. eGFP was also auto-fluorescent (green). Cell nuclei were stained with DAPI (blue). Arrow heads reveal examples of perinuclear eGFP immunoreactivity in SCs. Quantification of the percentage of eGFP positive cells is shown in D (Mean, SD). Data were compared using the Student t-test, p=0.0336. Averages: 4 weeks 31.17, 8 weeks 41.09.

FIG. 19A-F shows immunoblot and VGCN analysis of AAV9-miLacZ eGFP reporter gene expression. Representative images of immunoblot analysis of eGFP expression levels 4 and 8 weeks post intrathecal lumbar injection in lumbar root (A) and sciatic nerve (D) lysates with AAV9 expressing miRLacZ along with the EGFP reporter gene. Tissue samples from C61-Het non-injected mice were used as a negative control. Tubulin blot was used as loading control. For the quantification, eGFP to tubulin optic density ratio was calculated (Mean, SD). Data were compared using the Student t-test, p=0.0272 (B, E). AAV9-miLacZ VGCN in lumbar roots (C) and sciatic nerves (F) were calculated at 4 and 8 weeks after intrathecal lumbar injection. VGCN data were compared using the Student t-test and no statistical significance was found between the two time points (Mean, SD). Averages: Western blot roots: 4 weeks 0.92, 8 weeks 0.99; Western blot sciatic nerve: 4 weeks 0.48, 8 weeks 0.81; VCN roots: 4 weeks 3.24, 8 weeks 1.09; VCN sciatic nerve: 4 weeks 0.41, 8 weeks 0.38.

FIG. 20A-F shows results of in vivo testing of AAV9-miR871 effects in hu/mu PMP22 and other myelin-related genes in the lumbar spinal roots of CMT1A mouse model. Real Time PCR analysis of hu/mu PMP22 and other myelin-related genes expression in C61 Het mice six weeks after injection with AAV9-miR871 relative to AAV9-miRLacZ (non-targeting control) vector injected littermates (presented as baseline). For all above transcripts expression analysis muGAPDH was used as a housekeeping gene to normalize for loading and relative change was determined using the 2-ΔΔCT method (Mean, SD).

FIG. 21A-F shows results of in vivo testing of AAV9-miR871 effects in hu/mu PMP22 and other myelin-related genes in the sciatic nerves of CMT1A mouse model. Real Time PCR analysis of hu/mu PMP22 and other myelin-related genes expression in C61 Het mice six weeks after injection with AAV9-miR871 relative to AAV9-miRLacZ (non-targeting control) vector injected littermates (presented as baseline). For all above transcripts expression analysis muGAPDH was used as a housekeeping gene to normalize for loading and relative change was determined using the 2-ΔΔCT method (Mean, SD).

FIG. 22A-F shows results of in vivo testing of AAV9-miR871 effects in hu/mu PMP22 and other myelin-related genes in the femoral nerves of CMT1A mouse model. Real Time PCR analysis of hu/mu PMP22 and other myelin-related genes expression in C61 Het mice six weeks after injection with AAV9-miR871 relative to AAV9-miRLacZ (non-targeting control) vector injected littermates (presented as baseline). For all above transcripts expression analysis muGAPDH was used as a housekeeping gene to normalize for loading and relative change was determined using the 2-ΔΔCT method (Mean, SD).

FIG. 23A-F shows results of in vivo testing of AAV9-miR868 effects in hu/mu PMP22 and other myelin-related genes in the lumbar spinal roots of CMT1A mouse model. Real Time PCR analysis of hu/mu PMP22 and other myelin-related genes expression in C61 Het mice six weeks after injection with AAV9-miR871 relative to AAV9-miRLacZ (non-targeting control) vector injected littermates (presented as baseline). For all above transcripts expression analysis muGAPDH was used as a housekeeping gene to normalize for loading and relative change was determined using the 2-ΔΔCT method (Mean, SD).

FIG. 24A-F shows results of in vivo testing of AAV9-miR868 effects in hu/mu PMP22 and other myelin-related genes in the sciatic nerves of CMT1A mouse model. Real Time PCR analysis of hu/mu PMP22 and other myelin-related genes expression in C61 Het mice six weeks after injection with AAV9-miR871 relative to AAV9-miRLacZ (non-targeting control) vector injected littermates (presented as baseline). For all above transcripts expression analysis muGAPDH was used as a housekeeping gene to normalize for loading and relative change was determined using the 2-ΔΔCT method (Mean, SD).

FIG. 25A-F shows results of in vivo testing of AAV9-miR868 effects in hu/mu PMP22 and other myelin-related genes in the femoral nerves of CMT1A mouse model. Real Time PCR analysis of hu/mu PMP22 and other myelin-related genes expression in C61 Het mice six weeks after injection with AAV9-miR871 relative to AAV9-miRLacZ (non-targeting control) vector injected littermates (presented as baseline). For all above transcripts expression analysis muGAPDH was used as a housekeeping gene to normalize for loading and relative change was determined using the 2-ΔΔCT method (Mean, SD).

FIG. 26A-D shows results of in vivo testing of AAV9-miR871 effects on HuPMP22 and MPZ proteins in the lumbar roots of a CMT1A mouse model. Immunoblot analysis of HuPMP22 (A) expression levels in lumbar root lysates 6 weeks post lumbar intrathecal injection with AAV9 expressing miR871 or miRLacZ. Tissue samples from non-injected mice were used as a negative control. Tubulin blot and MPZ gel band were used as loading controls (B, C). MPZ expression was altered after treatment (D). For the quantification, HuPMP22 to tubulin, HuPMP22 to MPZ gel band and MPZ gel band to tubulin, optic density ratios were calculated (Mean, SD). Data were compared using the Student t-test.

FIG. 27A-D shows results of in vivo testing of AAV9-miR871 effects on HuPMP22 and MPZ proteins in the sciatic nerves of a CMT1A mouse model. Immunoblot analysis of HuPMP22 (A) expression levels in sciatic nerve lysates 6 weeks post lumbar intrathecal injection with AAV9 expressing miR871 or miRLacZ. Tissue samples from non-injected mice were used as a negative control. Tubulin blot and MPZ gel band were used as loading controls (B, C). MPZ expression was altered after treatment (D). For the quantification, HuPMP22 to tubulin, HuPMP22 to MPZ gel band and MPZ gel band to tubulin, optic density ratios were calculated (Mean, SD). Data were compared using the Student t-test.

FIG. 28A-D shows results of in vivo testing of AAV9-miR871 effects on HuPMP22 and MPZ proteins in the femoral nerves of a CMT1A mouse model. Immunoblot analysis of HuPMP22 (A) expression levels in femoral nerve lysates 6 weeks post lumbar intrathecal injection with AAV9 expressing miR871 or miRLacZ. Tissue samples from non-injected mice were used as a negative control. Tubulin blot and MPZ gel band were used as loading controls (B, C). MPZ expression was altered after treatment (D). For the quantification, HuPMP22 to tubulin, HuPMP22 to MPZ gel band and MPZ gel band to tubulin, optic density ratios were calculated (Mean, SD). Data were compared using the Student t-test.

FIG. 29 shows Early and Late Treatment trial design in the C61 Het mouse model of CMT1A. The early treatment trial design was also employed for WT mice, expressing only normal levels of murine PMP22.

FIG. 30A-B shows rotarod results at baseline and following early treatment at 5 rpm. A: AAV9-miR871 treated compared to AAV9-miRLacZ (mock) treated control mice, as indicated. Two months old non-injected WT and non-injected C61 Het mice showed no significant difference, while 4 and 6 months old WT mice performed better than their age-matched AAV9-miRLacZ treated C61 Het mice. At the age of 4 and 6 months (2 and 4 months post-injection) AAV9-miR871-treated C61 Het mice showed improved motor performance compared to the mock group, and did not differ significantly from WT mice of the same age (AAV9-miR871: n=16, AAV9-miRLacZ: n=16, 2 m C61 Het: n=32, 2-6 months old WT: n=10). B: Time course analysis demonstrates the improvement of AAV9-miR871 treated compared to mock treated C61 Het mice in rotarod 2 and 4 months post-injection (at 4 and 6 months of age). Values represent mean±SD. Data were compared using Mann-Whitney Test: 4 m WT vs. 4 m Het-miRLacZ: p=0.0017, 4 m Het-miRLacZ vs. 4 m Het-miR871: p=0.0003, 6 m WT vs. 6 m Het-miRLacZ: p=0.0003, 6 m Het-miRLacZ vs. 6 m Het-miR871: p<0.0001. Averages: 2 mWT: 594.12, 2 m Het: 549, 4 m WT: 596.1, 4 m Het-miRLacZ: 512.96, 4 m Het-miR871: 597.59, 6 m WT: 585.42, 6 m Het-miRLacZ: 390.43, 6 m Het-miR871: 584.96.

FIG. 31A-B shows rotarod results at baseline and following early treatment at 17.5 rpm: A: At baseline at the age of 2 months, before treatment, all C61 Het mice performed worse than WT mice. Likewise WT mice performed better than mock treated C61 Het mice at 4 and 6 months of age. At the age of 4 and 6 months AAV9-miR871-treated C61 Het mice showed improved motor performance compared to the control vector treated and reached the performance of WT mice (AAV9-miR871: n=16, AAV9-miRLacZ: n=16, 2 m C61 Het: n=32, 2-6 months old WT: n=10). B: Time course analysis demonstrates improved motor performance of AAV9-miR871 treated C61 Het mice in rotarod 2 and 4 months post-injection (at 4 and 6 months of age). Values represent mean±SD. Data were compared using Mann-Whitney Test: 2 m WT vs. 2 m Het-miRLacZ: p<0.0001, 4 m WT vs. 4 m Het-miRLacZ: p<0.0001, 4 m Het-miRLacZ vs. 4 m Het-miR871: p<0.0001, 4 m WT vs. 4 m Het-miR871: p<0.0001, 6 m WT vs. 6 m Het-miRLacZ: p<0.0001, 6 m Het-miRLacZ vs. 6 m Het-miR871: p<0.0001, 6 m WT vs. 6 m Het-miR871: p=0.0106. Averages: 2 mWT: 582.21, 2 m Het: 235.79, 4 m WT: 497.8, 4 m Het-miRLacZ: 172.93, 4 m Het-miR871: 504.09, 6 m WT: 416.16, 6 m Het-miRLacZ: 69.54, 6 m Het-miR871: 470.48.

FIG. 32A-B shows foot grip analysis at baseline and following early treatment: A: At baseline at the age of 2 months, before treatment, all C61 Het mice performed worse than non-injected WT mice. Likewise WT mice performed better than mock treated C61 Het mice at 4 and 6 months of age. At the age of 4 and 6 months AAV9-miR871-treated C61 Het mice showed improved grip strength performance compared to AAV9-miRLacZ treated control mice (AAV9-miR871: n=16, AAV9-miRLacZ: n=16, 2 m C61 Het: n=32, 2-6 months old WT: n=10). B:Time course analysis showed improved performance of AAV9-miR871 treated C61 Het mice in foot grip analysis 2 and 4 months post-injection (4 and 6 months of age). Values represent mean±SD. Data were compared using Mann-Whitney Test: 2 m WT vs. 2 m Het-miRLacZ: p<0.0001 4 m WT vs. 4 m Het-miRLacZ: p<0.0001, 4 m Het-miRLacZ vs. 4 m Het-miR871: p<0.0001, 4 m WT vs. 4 m Het-miR871: p<0.0001, 6 m WT vs. 6 m Het-miRLacZ: p<0.0001, 6 m Het-miRLacZ vs. 6 m Het-miR871: p<0.0001, 6 m WT vs. 6 m Het-miR871: p=0.0106. Averages: 2 mWT: 51.28, 2 m Het: 22.69, 4 m WT: 42.73, 4 m Het-miRLacZ: 17.80, 4 m Het-miR871: 29.53, 6 m WT: 36.81, 6 m Het-miRLacZ: 15.92, 6 m Het-miR871: 31.70.

FIG. 33A-B shows wire hang test analysis at baseline and following early treatment: A: At baseline at the age of 2 months, before treatment, all C61 Het mice performed worse than non-injected WT mice. Likewise WT mice performed better than mock treated C61 Het mice at 4 and 6 months of age. At the age of 4 and 6 months AAV9-miR871-treated C61 Het mice showed improved hang test performance compared to AAV9-miRLacZ treated control mice (AAV9-miR871: n=16, AAV9-miRLacZ: n=16, 2 m C61 Het: n=32, 2-6 months old WT: n=10). AAV9-miR871-treated mice at 4 months did not manage to reach WT performances, this is in contrast to 6-months old AAV9-miR871 treated mice that did not differ significantly to their age matched AAV9-miRLacZ treated B: Time course analysis showed improved performance of AAV9-miR871 treated C61 Het mice in hang test analysis 2 and 4 months post-injection (4 and 6 months of age). Values represent mean±SD. Data were compared using Mann-Whitney Test: 2 m WT vs. 2 m Het-miRLacZ: p<0.0001, 4 m WT vs. 4 m Het-miRLacZ: p<0.0001, 4 m Het-miRLacZ vs. 4 m Het-miR871: p<0.0001, 4 m WT vs. 4 m Het-miR871: p<0.0001, 6 m WT vs. 6 m Het-miRLacZ: p<0.0001, 6 m Het-miRLacZ vs. 6 m Het-miR871: p<0.0001, 6 m WT vs. 6 m Het-miR871: p=0.0106. Averages: 2 mWT: 593.70, 2 m Het: 306.96, 4 m WT: 527.28, 4 m Het-miRLacZ: 155.64, 4 m Het-miR871: 381.53, 6 m WT: 351.30, 6 m Het-miRLacZ: 130.81, 6 m Het-miR871: 371.65.

FIG. 34A-B shows rotarod results at baseline and following late treatment at 5 rpm. A: AAV9-miR871 treated compared to AAV9-miRLacZ (mock) treated control mice, as indicated. At all examined ages WT mice performed better than age-matched C61-Het or C61-Het AAV9-miRLacZ treated mice. At the age of 8 and 10 months (2 and 4 months post-injection) AAV9-miR871-treated C61 Het mice showed improved motor performance compared to the mock group, and did not differ significantly from WT mice of the same age (8 m AAV9-miR871: n=16, 8 m AAV9-miRLacZ: n=16, 10 m AAV9-miR871: n=10, 10 m AAV9-miRLacZ: n=8, 6 m C61 Het: n=32, 6-8 months old WT: n=10). B: Time course analysis demonstrates the improvement of AAV9-miR871 treated compared to mock treated C61 Het mice. Values represent mean±SD. Data were compared using Mann-Whitney Test: 6 m WT vs. 6 m Het-miRLacZ: p<0.0001, 8 m WT vs. 8 m Het-miRLacZ: p<0.0001, 8 m Het-miRLacZ vs. 8 m Het-miR871: p<0.0001, 10 m WT vs. 10 m Het-miRLacZ: p<0.0001, 10 m Het-miRLacZ vs. 10 m Het-miR871: p<0.0001. Averages: 6 mWT: 583.80, 6 m Het: 345.04, 8 m WT: 578.49, 8 m Het-miRLacZ: 221.93, 8 m Het-miR871: 566.01, 10 m WT: 584.20, 10 m Het-miRLacZ: 148.57, 10 m Het-miR871: 558.40.

FIG. 35A-B shows rotarod results at baseline and following late treatment at 17.5 rpm. A: AAV9-miR871 treated compared to AAV9-miRLacZ (mock) treated control mice, as indicated. At all examined ages WT mice performed better than age-matched C61-Het or C61-Het AAV9-miRLacZ treated mice. At the age of 8 and 10 months (2 and 4 months post-injection) AAV9-miR871-treated C61 Het mice showed improved motor performance compared to the mock group, and did not differ significantly from WT mice of the same age, with only exception being 8 m WT vs. 8 m Het-miR871 (8 m AAV9-miR871: n=16, 8 m AAV9-miRLacZ: n=16, 10 m AAV9-miR871: n=10, 10 m AAV9-miRLacZ: n=8, 6 m C61 Het: n=32, 6-8 months old WT: n=10). B: Time course analysis demonstrates the improvement of AAV9-miR871 treated compared to mock treated C61 Het mice. Values represent mean±SD. Data were compared using Mann-Whitney Test: 6 m WT vs. 6 m Het-miRLacZ: p<0.0001, 8 m WT vs. 8 m Het-miRLacZ: p<0.0001, 8 m Het-miRLacZ vs. 8 m Het-miR871: p<0.0001, 8 m WT vs. 8 m Het-miR871: p=0.0015, 10 m WT vs. 10 m Het-miRLacZ: p<0.0001, 10 m Het-miRLacZ vs. 10 m Het-miR871: p<0.0001. Averages: 6 mWT: 417.96, 6 m Het: 66.12, 8 m WT: 453.38, 8 m Het-miRLacZ: 19.24, 8 m Het-miR871: 293.95, 10 m WT: 328.96, 10 m Het-miRLacZ: 16.89, 10 m Het-miR871: 304.76.

FIG. 36A-B shows grip strength results at baseline and following late treatment. A: AAV9-miR871 treated compared to AAV9-miRLacZ (mock) treated control mice, as indicated. At all examined ages WT mice performed better than age-matched C61-Het or C61-Het AAV9-miRLacZ treated mice. At the age of 8 and 10 months (2 and 4 months post-injection) AAV9-miR871-treated C61 Het mice showed improved motor performance compared to the mock group, and did not differ significantly from WT mice of the same age (8 m AAV9-miR871: n=16, 8 m AAV9-miRLacZ: n=16, 10 m AAV9-miR871: n=10, 10 m AAV9-miRLacZ: n=8, 6 m C61 Het: n=32, 6-8 months old WT: n=10). B: Time course analysis demonstrates the improvement of AAV9-miR871 treated compared to mock treated C61 Het mice. Values represent mean±SD. Data were compared using Mann-Whitney Test: 6 m WT vs. 6 m Het-miRLacZ: p<0.0001, 8 m WT vs. 8 m Het-miRLacZ: p<0.0001, 8 m Het-miRLacZ vs. 8 m Het-miR871: p<0.0001, 10 m WT vs. 10 m Het-miRLacZ: p<0.0001, 10 m Het-miRLacZ vs. 10 m Het-miR871: p<0.0001. Averages: 6 mWT: 36.81, 6 m Het: 16.59, 8 m WT: 27.29, 8 m Het-miRLacZ: 16.20, 8 m Het-miR871: 26.82, 10 m WT: 25.62, 10 m Het-miRLacZ: 14.00, 10 m Het-miR871: 22.23.

FIG. 37A-B hang test results at baseline and following late treatment. A: AAV9-miR871 treated compared to AAV9-miRLacZ (mock) treated control mice, as indicated. At all examined ages WT mice performed better than age-matched C61-Het or C61-Het AAV9-miRLacZ treated mice. At the age of 8 and 10 months (2 and 4 months post-injection) AAV9-miR871-treated C61 Het mice showed improved motor performance compared to the mock group, and did not differ significantly from WT mice of the same age (8 m AAV9-miR871: n=16, 8 m AAV9-miRLacZ: n=16, 10 m AAV9-miR871: n=10, 10 m AAV9-miRLacZ: n=8, 6 m C61 Het: n=32, 6-8 months old WT: n=10). B: Time course analysis demonstrates the improvement of AAV9-miR871 treated compared to mock treated C61 Het mice. Values represent mean±SD. Data were compared using Mann-Whitney Test: 6 m WT vs. 6 m Het-miRLacZ: p<0.0001, 8 m WT vs. 8 m Het-miRLacZ: p<0.0001, 8 m Het-miRLacZ vs. 8 m Het-miR871: p=0.0004, 10 m WT vs. 10 m Het-miRLacZ: p=0.0062, 10 m WT vs. 10 m Het-miRLacZ: p=0.0338. Averages: 6 mWT: 351.30, 6 m Het: 55.23, 8 m WT: 238.40, 8 m Het-miRLacZ: 39.79, 8 m Het-miR871: 162.99, 10 m WT: 165.20, 10 m Het-miRLacZ: 70.90, 10 m Het-miR871: 127.54.

FIG. 38A-B shows physiological and phenotypical improvement in AAV9-miR871 early treated C61 Het mice. A: Motor nerve conduction velocity (MNCV) was improved in the 6-month old AAV9-miR871 treated C61 Het mice (n=8) compared to the AAV9-miRLacZ vector injected littermates (n=8) and approached the values of WT mice (n=6). Values represent mean±SD. Data were compared using the Mann-Whitney: 6 m WT vs. 6 m Het-miRLacZ: p=0.0003, 6 m Het-miRLacZ vs. 6 m Het-miR871: p<0.0001, 6 m WT vs. 6 m Het-miR871: p=0.1412. Averages: 6 m WT: 41.61, 6 m Het-miRLacZ: 25.90, 6 m Het-miR871: 36.88. B: Representative images of peripheral neuropathy phenotype evaluation of C61 Het mice early treatment group with either AAV9-miR871 or AAV9-miRLacZ. Six-month-old C61 Het AAV9-miRLacZ treated mice presented abnormal clenching of toes and clasping of hind limb phenotype upon suspension by the tail, suggestive of the presence of a peripheral nervous system defect. This phenotype is completely rescued in C61 Het AAV9-miR871 treated mice that present normal clenching without clasping of hind limbs.

FIG. 39A-B shows physiological and phenotypical improvement in AAV9-miR871 late treated C61 Het mice. A: Motor nerve conduction velocity (MNCV) was improved in the 6-month old AAV9-miR871 treated C61 Het mice (n=6) compared to the AAV9-miRLacZ vector injected littermates (n=5) and without though reaching the values of WT mice (n=4). Values represent mean±SD. Data were compared using the Mann-Whitney: 10 m WT vs. 10 m Het-miRLacZ: p=0.0079, 10 m Het-miRLacZ vs. 10 m Het-miR871: p=0.0040, 10 m WT vs. 10 m Het-miR871: p=0.0333. Averages: 6 m WT: 43.38, 6 m Het-miRLacZ: 24.12, 6 m Het-miR871: 37.69. B: Representative images of peripheral neuropathy phenotype evaluation of C61 Het mice late treatment group with either AAV9-miR871 or AAV9-miRLacZ. Ten-month-old C61 Het AAV9-miRLacZ treated mice presented abnormal clenching of toes and clasping of hind limb phenotype upon suspension by the tail, suggestive of the presence of a PNS defect. This phenotype is completely rescued in C61 Het AAV9-miR871 treated mice that present normal clenching without clasping of hind limbs.

FIG. 40A-D shows roots semithin sections of early treated CMT1A mouse model. Toluidine blue stained longitudinal (A,B) and transverse (C,D) semithin sections of lumbar motor spinal roots of C61 Het mice following early-treatment with either AAV9-miR871 or AAV9-miRLacZ vector. Representative images of semithin sections of anterior lumbar motor spinal roots attached to the spinal cord at low and higher (A-D) magnification. Thinly myelinated (t), demyelinated (*) fibers and onion bulb formations (o).

FIG. 41A-C shows quantification of the percentages of abnormally myelinated fibers in multiple early-treated roots (n=16 mice per group) confirms significant improvement in the numbers of abnormally myelinated fibers (A-B), as well as significant reduction in the numbers of onion bulb formations (C) in the fully treated compared with mock vector treated littermates. Values represent mean±SD. Data were compared using the Mann-Whitney. Averages: A: miRLacZ: 15.35, miR871: 11.74, B: miRLacZ: 49.74, miR871: 25.36, C: miRLacZ: 8.06, miR871: 0.69.

FIG. 42A-B shows femoral nerve semithin sections of early treated CMT1A mouse model. Toluidine blue stained semithin sections of femoral nerves of C61 Het mice following early-treatment with either AAV9-miR871 or AAV9-miRLacZ vector. Representative images of semithin sections of femoral nerve at low and higher (A-B) magnification. Thinly myelinated (t), demyelinated (*) fibers.

FIG. 43A-C shows quantification of the percentages of abnormally myelinated fibers in multiple early-treated femoral nerves (n=16 mice per group) confirms significant improvement in the numbers of abnormally myelinated fibers (A-B), onion bulb formations were limited and did not differ significantly when comparing fully and mock vector treated littermates. Values represent mean±SD. Data were compared using the Mann-Whitney. Averages: A: miRLacZ: 19.36, miR871: 6.83, B: miRLacZ: 2.33, miR871: 1.09, C: miRLacZ: 0.69, miR871: 0.25.

FIG. 44A-B shows roots semithin sections of late treated CMT1A mouse model. Toluidine blue stained semithin sections of lumbar motor spinal roots of C61 Het mice following intrathecal delivery of the either AAV9-miR871 or AAV9-miRLacZ vector. Representative images of semithin sections of anterior lumbar motor spinal roots attached to the spinal cord at low and higher (A-B) magnification. Thinly myelinated (t), demyelinated (*) fibers and onion bulb formations (o).

FIG. 45A-C shows quantification of the percentages of abnormally myelinated fibers in multiple late-treated roots (n=7 mice per group) confirms significant improvement in the numbers of abnormally myelinated fibers (A-B), as well as significant reduction in the numbers of onion bulb formations (C) in the fully treated compared with mock vector treated littermates. Values represent mean±SD. Data were compared using the Mann-Whitney. Averages: A: miRLacZ: 19.39, miR871: 14.62, B: miRLacZ: 52.25, miR871: 32.65, C: miRLacZ: 38.86, miR871: 2.71.

FIG. 46A-B shows femoral nerve semithin sections of late treated CMT1A mouse model. Toluidine blue stained semithin sections of femoral nerves of C61 Het mice following late treatment with either AAV9-miR871 or AAV9-miRLacZ vector. Representative images of semithin sections of anterior lumbar motor spinal roots attached to the spinal cord at low and higher (A-B) magnification. Thinly myelinated (t), demyelinated (*) fibers.

FIG. 47A-C shows quantification of the percentages of abnormally myelinated fibers in multiple late-treated femoral nerves (miRLacZ n=7, miR871 n=10) confirms significant improvement in the numbers of abnormally myelinated fibers (A-B), onion bulb formations were limited and did not differ significantly when comparing fully and mock vector treated littermates. Values represent mean±SD. Data were compared using the Mann-Whitney. Averages: A: miRLacZ: 21.95, miR871: 11.31, B: miRLacZ: 2.08, miR871: 1.37, C: miRLacZ: 0.57, miR871: 0.20.

FIG. 48 shows 19-23 nucleotide miPMP22 antisense guide strands capable of targeting the PMP22-215 full length cDNA sequence. Each column in the figure shows antisense guide strand sequences of one of the lengths (e.g., the 19-nucleotide guide strands) in the 5′ to 3′ orientation and each column continues onto (spans) the next pages of the figure.

FIG. 49 shows the PMP22-204 cDNA sequence.

FIG. 50 shows 19-23 nucleotide miPMP22 antisense guide strands capable of targeting the PMP22-204 cDNA sequence. Each column in the figure shows antisense guide strand sequences of one of the lengths (e.g., the 19-nucleotide guide strands) where each sequence is shown in the 5′ to 3′ orientation and each column continues onto (spans) the next pages of the figure.

FIG. 51 shows results of in vivo testing of AAV9-miR871 effects in mu PMP22 in the lumbar spinal roots, sciatic nerve and femoral nerve of wild type (WT) mice, expressing only normal levels of murine PMP22, 6 weeks post-injection. Real Time PCR analysis of mu PMP22 gene expression in WT mice 6 weeks after injection with AAV9-miR871 relative to AAV9-miRLacZ (non-targeting control) vector injected littermates (presented as baseline). For the above transcripts expression analysis, muGAPDH was used as a housekeeping gene to normalize for loading and relative change was determined using the 2^{-ΔΔCT} method (Mean, SD). miR871: lumbar roots n=4, sciatic nerve n=4, femoral nerve n=2. miRLacZ: lumbar roots n=4, sciatic nerve n=4, femoral nerve n=1. Averages: mu PMP22: roots: −0.356856675, sciatic nerve: −0.521269475, femoral nerve: −0.8747207.

FIG. 52 shows results of in vivo testing of AAV9-miR871 effects in myelin-related genes in the lumbar spinal roots, sciatic nerve and femoral nerve of wild type (WT) mice, expressing only normal levels of murine PMP22, 6 weeks post-injection. Real Time PCR analysis of myelin-related genes expression in WT mice 6 weeks after injection with AAV9-miR871 relative to AAV9-miRLacZ (non-targeting control) vector injected littermates (presented as baseline). For the above transcripts expression analysis, muGAPDH was used as a housekeeping gene to normalize for loading and relative change was determined using the 2^{-ΔΔCT} method (Mean, SD). miR871: lumbar roots n=4, sciatic nerve n=4, femoral nerve n=2. miRLacZ: lumbar roots n=4, sciatic nerve n=4, femoral nerve n=1. Averages: mu MPZ: roots: 1.307563825, sciatic nerve: 1.475276575, femoral nerve: 2.22841445, mu CNP: roots: 0.856114775, sciatic nerve: 0.79534705, femoral nerve: 5.025213, mu Gldn: roots: 1.31614995, sciatic nerve: 1.011103935, femoral nerve: 2.85548965, mu GJB1: roots: 1.0224301, sciatic nerve: 2.091169, femoral nerve: 4.06045285.

FIG. 53A-C shows results of in vivo testing of AAV9-miR871 effects on muPMP22 and MPZ proteins in the lumbar roots of WT mice, expressing only normal levels of murine PMP22 at 6 weeks post injection. Immunoblot analysis of muPMP22 (A) expression levels in lumbar roots lysates 6 weeks post lumbar intrathecal injection with AAV9 expressing miR871 or miRLacZ. Tissue samples (lumbar roots and spinal cord) from non-injected mice were used as a control. Tubulin blot was used as a loading control (A-C). MuPMP22 expression was reduced while MPZ expression was not significantly altered after treatment (A-C). Immunoblot of eGFP reporter gene was used in order to confirm the success of the injection (A). For the quantification, muPMP22 to tubulin and MPZ to tubulin, optic density ratios were calculated (Mean, SD). Data were compared using the Student t-test: muPMP22: p=0.0004, muMPZ: p=0.4465. Normalized averages: miR871-muPMP22: 0.34, miR871-MPZ: 0.98.

FIG. 54A-C shows results of in vivo testing of AAV9-miR871 effects on muPMP22 and MPZ proteins in the sciatic nerves of WT mice, expressing only normal levels of murine PMP22 at 6 weeks post injection. Immunoblot analysis of muPMP22 (A) expression levels in sciatic nerves lysates 6 weeks post lumbar intrathecal injection with AAV9 expressing miR871 or miRLacZ. Tissue samples (sciatic nerve and spinal cord) from non-injected mice were used as a control. Tubulin blot was used as a loading control (A-C). MuPMP22 expression was reduced while MPZ expression was increased after treatment (A-C). Immunoblot of eGFP reporter gene was used in order to confirm the success of the injection (A). For the quantification, muPMP22 to tubulin and MPZ to tubulin, optic density ratios were calculated (Mean, SD). Data were compared using the Student t-test: muPMP22: p=0.0024, muMPZ: p=0.0287. Normalized averages: miR871-muPMP22: 0.31, miR871-MPZ: 1.54.

FIG. 55A-C shows results of in vivo testing of AAV9-miR871 effects on muPMP22 and MPZ proteins in the femoral nerves of WT mice, expressing only normal levels of murine PMP22 at 6 weeks post injection. Immunoblot analysis of muPMP22 (A) expression levels in femoral nerves lysates 6 weeks post lumbar intrathecal injection with AAV9 expressing miR871 or miRLacZ. Tissue samples (femoral nerve and spinal cord) from non-injected mice were used as a control. Tubulin blot was used as a loading control (A-C). MuPMP22 expression was reduced while MPZ expression was not significantly altered after treatment (A-C). Immunoblot of eGFP reporter gene was used in order to confirm the success of the injection (A). For the quantification, muPMP22 to tubulin and MPZ to tubulin, optic density ratios were calculated (Mean, SD). Data were compared using the Student t-test: muPMP22: p=0.0099, muMPZ: p=0.2515. Normalized averages: miR871-muPMP22: 0.15, miR871-MPZ: 1.17.

FIG. 56A-B shows rotarod results at 5 rpm of WT injected mice, expressing only normal levels of murine PMP22, at baseline and following injection with AAV9-miR871 at 2 months of age. A: AAV9-miR871 injected compared to AAV9-miRLacZ (mock) injected control mice, as indicated. Prior injection, two months old WT mice did not differ from non-injected WT mice. At the age of 4 months (2 months post-injection) AAV9-miR871-injected WT mice showed impaired motor performance compared to the WT mock and non-injected groups. At the age of 6 months AAV9-miR871-injected WT mice did not differ from age-matched non-injected WT or mock mice groups. At all examined ages, mock injected mice did not differ from WT non-injected mice (AAV9-miR871: n=10, AAV9-miRLacZ: n=10, 2 m WT to be injected: n=20, 2-6 months old WT: n=10). B: Time course analysis demonstrates the performance of AAV9-miR871 injected compared to mock injected WT mice. Values represent mean±SD. Data were compared using One way ANOVA with Tukey's Multiple Comparison Test. Significance level of all comparisons, P<0.05. Averages: 2 mWT: 594.12, 2 m WT prior injection: 599.51, 4 m WT: 596.1, 4 m WT-miRLacZ: 586.78, 4 m WT-miR871: 496.72, 6 m WT: 585.42, 6 m WT-miRLacZ: 565.78, 6 m WT-miR871: 570.76.

FIG. 57A-B shows rotarod at 17.5 rpm results of WT injected mice, expressing only normal levels of murine PMP22, at baseline and following injection with AAV9-miR871 at 2 months of age. A: AAV9-miR871 injected compared to AAV9-miRLacZ (mock) injected control mice, as indicated. Prior injection, two months old WT mice did not differ from non-injected WT mice. At the age of 4 months (2 months post-injection) AAV9-miR871-injected WT mice showed impaired motor performance compared to the WT mock and non-injected groups. At the age of 6 months AAV9-miR871-injected WT mice did not differ from age-matched non-injected WT or mock mice groups. At all examined ages, mock injected mice did not differ from WT non-injected mice (AAV9-miR871: n=10, AAV9-miRLacZ: n=10, 2 m WT to be injected: n=20, 2-6 months old WT: n=10). B: Time course analysis demonstrates the performance of AAV9-miR871 injected compared to mock injected WT mice. Values represent mean±SD. Data were compared using One way ANOVA with Tukey's Multiple Comparison Test. Significance level of all comparisons, P<0.05. Averages: 2 mWT: 582.22, 2 m WT prior injection: 565.06, 4 m WT: 497.8, 4 m WT-miRLacZ: 461.44, 4 m WT-miR871: 237, 6 m WT: 416.16, 6 m WT-miRLacZ: 474.32, 6 m WT-miR871: 377.38.

FIG. 58A-B shows grip strength results of WT injected mice, expressing only normal levels of murine PMP22, at baseline and following injection with AAV9-miR871 at 2 months of age. A: AAV9-miR871 injected compared to AAV9-miRLacZ (mock) injected control mice, as indicated. Prior injection, two months old WT mice did not differ from non-injected WT mice. At all examined ages, WT non-injected and mock injected mice performed better than their age-matched WT-AAV9-miR871 mice. At all examined ages, mock injected mice did not differ from WT non-injected mice (AAV9-miR871: n=10, AAV9-miRLacZ: n=10, 2 m WT to be injected: n=20, 2-6 months old WT: n=10). B: Time course analysis demonstrates the performance of WT AAV9-miR871 injected compared to mock injected WT mice. Values represent mean±SD. Data were compared using One way ANOVA with Tukey's Multiple Comparison Test. Significance level of all comparisons, P<0.05. Averages: 2 mWT: 51.28, 2 m WT prior injection: 46.11, 4 m WT: 42.73, 4 m WT-miRLacZ: 40.09, 4 m WT-miR871: 23.34, 6 m WT: 36.81, 6 m WT-miRLacZ: 32.98, 6 m WT-miR871: 24.48.

FIG. 59A-B shows hang test results of WT injected mice, expressing only normal levels of murine PMP22, at baseline and following injection with AAV9-miR871 at 2 months of age. A: AAV9-miR871 injected compared to AAV9-miRLacZ (mock) injected control mice, as indicated. Prior injection, two months old WT mice did not differ from non-injected WT mice. At the age of 4 months (2 months post injection), non-injected WT, mock and AAV9-miR871 injected mice performed similarly. At the age of 6 months (4 months post-injection) AAV9-miR871-injected WT mice showed impaired performance compared to the WT mock and non-injected groups. At all examined ages, mock injected mice did not differ from WT non-injected mice (AAV9-miR871: n=10, AAV9-miRLacZ: n=10, 2 m WT to be injected: n=20, 2-6 months old WT: n=10). B: Time course analysis demonstrates the performance of WT AAV9-miR871 injected compared to mock injected WT mice. Values represent mean±SD. Data were compared using One way ANOVA with Tukey's Multiple Comparison Test. Significance level of all comparisons, P<0.05. Averages: 2 mWT: 593.7, 2 m WT prior injection: 585.26, 4 m WT: 527.28, 4 m WT-miRLacZ: 503.92, 4 m WT-miR871: 410.2, 6 m WT: 351.3, 6 m WT-miRLacZ: 449.06, 6 m WT-miR871: 161.94.

FIG. 60 shows physiological improvement in AAV9-miR871 early treated C61 Het mice. Amplitude of the compound muscle action potential (CMAP) was improved in the 6-month old AAV9-miR871 treated C61 Het mice (n=8) compared to the AAV9-miRLacZ vector injected littermates (n=8) but did not reach the values of WT mice (n=6). Values represent mean±SD. Data were compared using One way ANOVA with Tukey's Multiple Comparison Test. Averages (mV): 6 m WT: 6.90, 6 m Het-miRLacZ: 1.44, 6 m Het-miR871: 3.51.

FIG. 61 shows physiological performance of AAV9-miR871 late treated C61 Het mice. Amplitude of the compound muscle action potential (CMAP) was not improved in the 10-month old AAV9-miR871 treated C61 Het mice (n=8) compared to the AAV9-miRLacZ vector injected littermates (n=8) and did not reach the values of WT mice (n=6). Values represent mean±SD. Data were compared using One way ANOVA with Tukey's Multiple Comparison Test. Averages (mV): 10 m WT: 5.33, 10 m Het-miRLacZ: 2.40, 10 m Het-miR871: 2.98.

FIG. 62 shows physiological performance of AAV9-miR871 WT-injected mice. Amplitude of the compound muscle action potential (CMAP) was reduced in 6-month old AAV9-miR871 treated WT mice (n=5) compared to the AAV9-miRLacZ vector injected littermates (n=4) (B) while motor nerve conduction velocities (MNCV) were not affected (A), compared to the values of WT non-injected mice (n=6). Values represent mean±SD. Data were compared using One way ANOVA with Tukey's Multiple Comparison Test. Averages: 6 m WT: 6.89, 6 m WT-miRLacZ: 7.03, 6 m WT-miR871: 4.62. Averages for MNCVs: 6 m WT: 41.61, 6 m WT-miRLacZ: 42.09, 6 m WT-miR871: 40.07.

FIG. 63 shows hind limb clasping phenotype of WT injected mice with either AAV9-miRLacZ or AAV9-miR871 vectors at four months post injection (6 months of age). Quantification of hind limb clasping angle and representative images of peripheral neuropathy phenotype evaluation of WT mice group injected with either AAV9-miR871 or AAV9-miRLacZ. There was no difference among 6-months old WT, WT injected with AAV9-miRLacZ or WT injected with AAV9-miR871 mice. Averages for CMAPs (mV): 6 m WT: 73.19, 6 m WT-miRLacZ: 70.54, 6 m WT-miR871: 59.09.

FIG. 64 shows the immune response analysis 6 weeks and 4 months post injection of anterior lumbar roots sections of baseline WT and C61-Het mice as well as of C61-Het mice injected with AAV9-miRLacZ. The percentage of B-cell marker CD20, leukocyte marker CD45, macrophage marker CD68 and T-cell marker CD3 was calculated in relation to total cell number (Mean, SD). Data were compared using One way ANOVA with Tukey's Multiple Comparison Test. Significance level of all comparisons, P<0.05 (WT: n=4, C61 Het: n=4, C61 Het-miRLacZ: n=4). Averages: CD20: 6 weeks: WT: 0.06, Het: 0.55, Het-miRLacZ: 0.69, 4 months: WT: 0.18, Het: 1.94, Het-miRLacZ: 1.37, CD45: WT: 0.23, Het: 7.82, Het-miRLacZ: 7.43, 4 months: WT: 1.49, Het: 8.46, Het-miRLacZ: 8.13, CD68: WT: 2.38, Het: 3.59, Het-miRLacZ: 3.34, 4 months: WT: 1.00, Het: 6.88, Het-miRLacZ: 7.62, CD3: WT: 0.07, Het: 0.76, Het-miRLacZ: 0.84, 4 months: WT: 0.23, Het: 1.43, Het-miRLacZ: 1.20.

FIG. 65 shows the immune response analysis 6 weeks and 4 months post injection of sciatic nerve sections of baseline WT and C61-Het mice as well as of C61-Het mice injected with AAV9-miRLacZ. The percentage of B-cell marker CD20, leukocyte marker CD45, macrophage marker CD68 and T-cell marker CD3 was calculated in relation to total cell number (Mean, SD). Data were compared using One way ANOVA with Tukey's Multiple Comparison Test. Significance level of all comparisons, P<0.05 (WT: n=4, C61 Het: n=4, C61 Het-miRLacZ: n=4). Averages: CD20: 6 weeks: WT: 2.38, Het: 3.59, Het-miRLacZ: 3.34, 4 months: WT: 1.00, Het: 6.88, Het-miRLacZ: 7.62, CD45: WT: 2.76, Het: 7.03, Het-miRLacZ: 6.64, 4 months: WT: 1.94, Het: 6.36, Het-miRLacZ: 6.62, CD68: WT: 2.76, Het: 7.03, Het-miRLacZ: 6.64, 4 months: WT: 1.94, Het: 6.36, Het-miRLacZ: 6.62, CD3: WT: 0.85, Het: 2.27, Het-miRLacZ: 1.86, 4 months: WT: 0.66, Het: 4.57, Het-miRLacZ: 4.46.

FIG. 66 shows the immune response analysis 6 weeks and 4 months post injection of liver sections of baseline WT and C61-Het mice as well as of C61-Het mice injected with AAV9-miRLacZ. The percentage of B-cell marker CD20, leukocyte marker CD45, macrophage marker CD68 and T-cell marker CD3 was calculated in relation to total cell number (Mean, SD). Data were compared using One way ANOVA with Tukey's Multiple Comparison Test. Significance level of all comparisons, P<0.05 (WT: n=4, C61 Het: n=4, C61 Het-miRLacZ: n=4). Averages: CD20: 6 weeks: WT: 0.09, Het: 0.62, Het-miRLacZ: 0.64, 4 months: WT: 0.16, Het: 0.59, Het-miRLacZ: 0.62, CD45: WT: 0.77, Het: 0.66, Het-miRLacZ: 2.50, 4 months: WT: 1.36, Het: 1.00, Het-miRLacZ: 1.24, CD68: WT: 1.28, Het: 1.15, Het-miRLacZ: 1.17, 4 months: WT: 0.66, Het: 1.03, Het-miRLacZ: 1.23, CD3: WT: 0.72, Het: 0.77, Het-miRLacZ: 1.69, 4 months: WT: 1.01, Het: 1.10, Het-miRLacZ: 0.92.

FIG. 67 shows plasma neurofilament light (NfL) concentration (pg/ml) in 6-months old baseline WT (n=4), C61 Het (n=4), C61 Het AAV9-miRLacZ-treated (n=6) and C61 Het AAV9-miR871-treated (n=6) mice. Nfl concentrations are a dynamic measure of axonal damage and serve as a biomarker for CMT disease severity. Six-months old baseline C61 Het mice presented higher concentrations of Nfl in their plasma compared to aged matched baseline WT mice. AAV9-miRLacZ injection to C61 Het mice did not affected Nfl levels when compared to aged matched non-injected C61 Het mice. Early treated C61 Het mice with AAV9-miR871 presented reduced concentrations of Nfl in their plasma when compared to C61 Het mice injected with AAV9-miRLacZ. AAV9-miR871 scores approached WT levels. Averages: 6 m WT: 131.10, 6 m C61 Het: 418.07, C61 Het AAV9-miRLacZ: 540.65, C61 Het AAV9-miR871: 321.37.

FIG. 68 shows plasma neurofilament light (NfL) concentration (pg/ml) in 10-months old baseline WT (n=4), C61 Het (n=4), C61 Het AAV9-miRLacZ-treated (n=6) and C61 Het AAV9-miR871-treated (n=6) mice. Nfl concentrations are a dynamic measure of axonal damage and serve as a biomarker for CMT disease severity. Ten-months old baseline C61 Het presented higher concentrations of Nfl in their plasma compared to aged matched baseline WT mice. AAV9-miRLacZ injection to C61 Het mice did not affected Nfl levels when compared to aged matched non-injected C61 Het mice. Late treatment with AAV9-miR871 was not sufficient to improve Nfl levels at 10 months old C61 Het mice. Averages: 10 m WT: 88.07, 10 m C61 Het: 539.66, C61 Het AAV9-miRLacZ: 471.99, C61 Het AAV9-miR871: 559.28.

FIG. 69 shows plasma neurofilament light (NfL) concentration (pg/ml) in 6-months old baseline WT (n=4), WT AAV9-miRLacZ-treated (n=5) and WT AAV9-miR871-treated (n=5) mice. Nfl concentrations are a dynamic measure of axonal damage. There was no difference among non-injected and injected WT mice in terms of plasma Nfl concertation. Averages: 6 m WT: 131.10, WT AAV9-miRLacZ: 128.93, WT AAV9-miR871: 104.92.

FIG. 70 shows the immune response analysis of early treatment anterior lumbar roots immunohistochemistry sections of baseline WT and C61-Het mice as well as of C61-Het mice injected with AAV9-miR871 at 4 months post-injection (6 months old mice). The percentage of B-cell marker CD20, leukocyte marker CD45, macrophage marker CD68 and T-cell marker CD3 was calculated in relation to total cell number (Mean, SD). Non-injected 6 months old C61 Het lumbar roots presented elevated scores of all immune response markers that were decreased down to WT levels after AAV9-miR871 injection. Data were compared using One way ANOVA with Tukey's Multiple Comparison Test. Significance level of all comparisons, P<0.05. Averages: CD20: WT: 0.18, C61 Het: 1.94, C61 Het-AAV9-miR871: 0.25, CD45: WT: 1.49, C61 Het: 8.46, C61 Het-AAV9-miR871: 2.39, CD68: WT: 1.00, C61 Het: 6.88, C61 Het-AAV9-miR871: 0.98, CD3: WT: 0.23, C61 Het: 1.43, C61 Het-AAV9-miR871: 0.60.

FIG. 71 shows the immune response analysis of early treatment sciatic nerve immunohistochemistry sections of baseline WT and C61-Het mice as well as of C61-Het mice injected with AAV9-miR871 at 4 months post-injection (6 months old mice). The percentage of B-cell marker CD20, leukocyte marker CD45, macrophage marker CD68 and T-cell marker CD3 was calculated in relation to total cell number (Mean, SD). Non-injected 6 months old C61 Het sciatic nerves presented elevated scores of all immune response markers that were decreased down to WT levels after AAV9-miR871 injection. Data were compared using One way ANOVA with Tukey's Multiple Comparison Test. Significance level of all comparisons, P<0.05. Averages: CD20: WT: 0.05, C61 Het: 1.25, C61 Het-AAV9-miR871: 0.51, CD45: WT: 1.93, C61 Het: 6.36, C61 Het-AAV9-miR871: 2.68, CD68: WT: 0.66, C61 Het: 4.58, C61 Het-AAV9-miR871: 1.22, CD3: WT: 0.16, C61 Het: 0.59, C61 Het-AAV9-miR871: 0.25.

FIG. 72 shows the immune response analysis of early treatment liver immunohistochemistry sections of baseline WT and C61-Het mice as well as of C61-Het mice injected with AAV9-miR871 at 4 months post-injection (6 months old mice). The percentage of B-cell marker CD20, leukocyte marker CD45, macrophage marker CD68 and T-cell marker CD3 was calculated in relation to total cell number (Mean, SD). Non-injected 6 months old WT, C61 Het and C61 Het injected with AAV9-miR871 livers presented similar scores of immune response markers. Data were compared using One way ANOVA with Tukey's Multiple Comparison Test. Significance level of all comparisons, P<0.05. Averages: CD20: WT: 1.36, C61 Het: 1.00, C61 Het-AAV9-miR871: 1.19, CD45: WT: 0.66, C61 Het: 1.03, C61 Het-AAV9-miR871: 0.91, CD68: WT: 1.99, C61 Het: 2.42, C61 Het-AAV9-miR871: 2.52, CD3: WT: 1.01, C61 Het: 1.09, C61 Het-AAV9-miR871: 1.11.

FIG. 73 shows the immune response analysis of late treatment anterior lumbar roots sections of baseline WT and C61-Het mice as well as of C61-Het mice injected with AAV9-miR871 at 4 months post-injection (10 months old mice). The percentage of B-cell marker CD20, leukocyte marker CD45, macrophage marker CD68 and T-cell marker CD3 was calculated in relation to total cell number (Mean, SD). Non-injected 10 months old C61 Het lumbar roots presented elevated scores of all immune response markers that were decreased down to WT levels after AAV9-miR871 injection, with only exception being CD45 positive cells that despite their significant decrease they did not reach WT levels. Data were compared using One way ANOVA with Tukey's Multiple Comparison Test. Significance level of all comparisons, P<0.05. Averages: CD20: WT: 0.39, C61 Het: 1.80, C61 Het-AAV9-miR871: 0.45, CD45: WT: 4.07, C61 Het: 13.45, C61 Het-AAV9-miR871: 7.32, CD68: WT: 2.32, C61 Het: 7.81, C61 Het-AAV9-miR871: 1.25, CD3: WT: 0.45, C61 Het: 2.19, C61 Het-AAV9-miR871: 0.55.

FIG. 74 shows the immune response analysis of late treatment sciatic nerve sections of baseline WT and C61-Het mice as well as of C61-Het mice injected with AAV9-miR871 at 4 months post-injection (10 months old mice). The percentage of B-cell marker CD20, leukocyte marker CD45, macrophage marker CD68 and T-cell marker CD3 was calculated in relation to total cell number (Mean, SD). Non-injected 10 months old C61 Het sciatic nerves presented elevated scores of all immune response markers that were decreased down to WT levels after AAV9-miR871 injection. Data were compared using One way ANOVA with Tukey's Multiple Comparison Test. Significance level of all comparisons, P<0.05. Averages: CD20: WT: 0.14, C61 Het: 1.29, C61 Het-AAV9-miR871: 0.43, CD45: WT: 6.21, C61 Het: 12.34, C61 Het-AAV9-miR871: 8.52, CD68: WT: 2.17, C61 Het: 5.25, C61 Het-AAV9-miR871: 2.51, CD3: WT: 0.37, C61 Het: 1.65, C61 Het-AAV9-miR871: 0.57.

FIG. 75 shows the immune response analysis of late treatment liver sections of baseline WT and C61-Het mice as well as of C61-Het mice injected with AAV9-miR871 at 4 months post-injection (10 months old mice). The percentage of B-cell marker CD20, leukocyte marker CD45, macrophage marker CD68 and T-cell marker CD3 was calculated in relation to total cell number (Mean, SD). Non-injected 10 months old WT, C61 Het and C61 Het injected with AAV9-miR871 livers presented similar scores of immune response markers. Data were compared using One way ANOVA with Tukey's Multiple Comparison Test. Significance level of all comparisons, P<0.05. Averages: CD20: WT: 4.04, C61 Het: 4.69, C61 Het-AAV9-miR871: 4.89, CD45: WT: 3.84, C61 Het: 3.03, C61 Het-AAV9-miR871: 3.98, CD68: WT: 12.63, C61 Het: 11.86, C61 Het-AAV9-miR871: 10.68, CD3: WT: 1.48, C61 Het: 2.74, C61 Het-AAV9-miR871: 2.84.

FIG. 76 shows VGCN of PNS and non-PNS tissues of early treated mice at 4 months post injection (mice 6 months old). Averages: Roots: 3.57, sciatic nerve: 3.57, femoral nerve: 0.55, brain: 0.19, liver: 21.29, kidney: 0.17, lung: 0.17, quadriceps: 0.09, heart: 0.48, stomach: 0.05, eye: 0.43.

FIG. 77 shows VGCN of PNS and non-PNS tissues of late treated mice at 4 months post injection (mice 10 months old). Averages: Roots: 1.84, sciatic nerve: 2.73, femoral nerve: 1.41, brain: 0.34, liver: 20.74, kidney: 0.63, lung: 0.85, quadriceps: 0.33, heart: 3.86, stomach: 0.17, eye: 0.17.

### DETAILED DESCRIPTION

The products and methods described herein are used in the treatment of diseases associated with a duplicated and/or mutant PMP22 gene. Diseases associated with PMP22 include, for example, CMT1A.

A nucleic acid encoding human PMP22 is set forth in SEQ ID NO: 25. Various products and methods of the disclosure can target variants of the human PMP22 nucleotide sequence set forth in SEQ ID NO: 25. The variants can exhibit 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 25.

A nucleic acid encoding mouse PMP22 is set forth in SEQ ID NO: 27. Various products and methods of the disclosure can target variants of the nucleotide sequence set forth in SEQ ID NO: 27. The variants can exhibit 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 27.

The disclosure includes the use of RNA interference to inhibit or interfere with the expression of PMP22 to ameliorate and/or treat subjects with diseases or disorders resulting from overexpression of PMP22. RNA interference (RNAi) is a mechanism of gene regulation in eukaryotic cells that has been considered for the treatment of various diseases. RNAi refers to post-transcriptional control of gene expression mediated by inhibitory RNAs. The inhibitory RNAs are small (21-25 nucleotides in length), noncoding RNAs that share sequence homology and base-pair with cognate messenger RNAs (mRNAs). The interaction between the inhibitory RNAs and mRNAs directs cellular gene silencing machinery to prevent the translation of the mRNAs. The RNAi pathway is summarized in Duan (Ed.), Section 7.3 of Chapter 7 in Muscle Gene Therapy, Springer Science+Business Media, LLC (2010).

As an understanding of natural RNAi pathways has developed, researchers have designed artificial inhibitory RNAs for use in regulating expression of target genes for treating disease. Several classes of small RNAs are known to trigger RNAi processes in mammalian cells [Davidson et al., Nat. Rev. Genet., 12:329-40 (2011); Harper, Arch. Neurol., 66:933-938 (2009)]. Artificial inhibitory RNAs expressed in vivo from plasmid- or virus-based vectors and may achieve long term gene silencing with a single administration, for as long as the vector is present within target cell nuclei and the driving promoter is active [Davidson et al., Methods Enzymol., 392:145-73, (2005)]. Importantly, this vector-expressed approach leverages the decades-long advancements already made in the muscle gene therapy field, but instead of expressing protein coding genes, the vector cargo in RNAi therapy strategies are artificial inhibitory RNAs targeting disease genes-of-interest.

Products and methods are provided herein that comprise shRNA to affect PMP22 expression (e.g., knockdown or inhibit expression). An shRNA is an artificial RNA molecule with a tight hairpin turn that can be used to silence target gene expression via RNA interference (RNAi). shRNA is an advantageous mediator of RNAi in that it has a relatively low rate of degradation and turnover, but it requires use of an expression vector. Once the vector has transduced the host genome, the shRNA is then transcribed in the nucleus by polymerase II or polymerase III, depending on the promoter choice. The product mimics pri-microRNA (pri-miRNA) and is processed by Drosha. The resulting pre-shRNA is exported from the nucleus by Exportin 5. This product is then processed by Dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both of these cases, the shRNA leads to target gene silencing. The disclosure includes the production and administration of a viral vector expressing PMP22 antisense sequences via shRNA. The expression of shRNAs is regulated by the use of various promoters. The disclosure contemplates use of polymerase III promoters, such as U6 and H1 promoters, or polymerase II promoters. U6 shRNAs are exemplified.

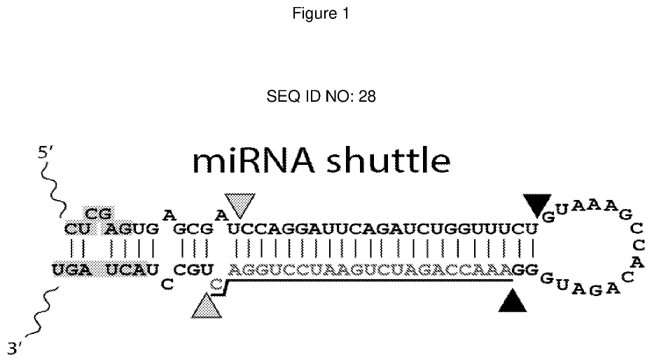

The products and methods provided herein can comprise miRNA shuttles to modify PMP22 expression (e.g., knockdown or inhibit expression). Like shRNAs, miRNA shuttles are expressed intracellularly from DNA transgenes. miRNA shuttles typically contain natural miRNA sequences required to direct correct processing, but the natural, mature miRNA duplex in the stem is replaced by the sequences specific for the intended target transcript (e.g., as shown in FIG. 1). Following expression, the artificial miRNA is cleaved by Drosha and Dicer to release the embedded siRNA-like region. Polymerase Ill promoters, such as U6 and H1 promoters, and polymerase II promoters are also used to drive expression of the miRNA shuttles.

The disclosure provides nucleic acids encoding miPMP22s to inhibit the expression of the PMP22 gene. The disclosure provides a nucleic acid encoding a miPMP22 wherein the nucleic acid comprises at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the polynucleotide sequence set forth in any one of SEQ ID NOs: 1-8. The disclosure provides a nucleic acid encoding a miPMP22 processed antisense guide strand comprising at least about 70%, 75, 80%, 81%, 82%, 83%, 84%, 85%, 86%, 87%, 88%, 89%, 90%, 91%, 92%, 93%, 94%, 95%, 96%, 97%, 98%, 99%, or 100% identity to the miPMP22 processed antisense guide strand sequence set forth in any one of SEQ ID NOs: 17-24.

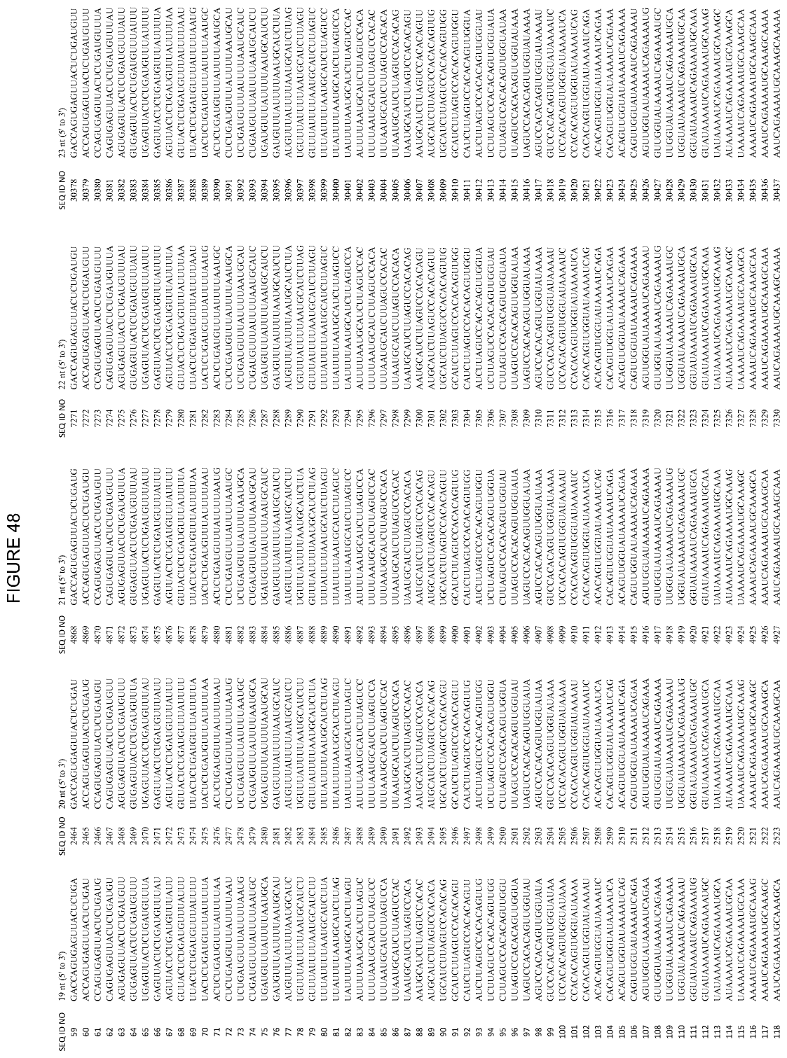

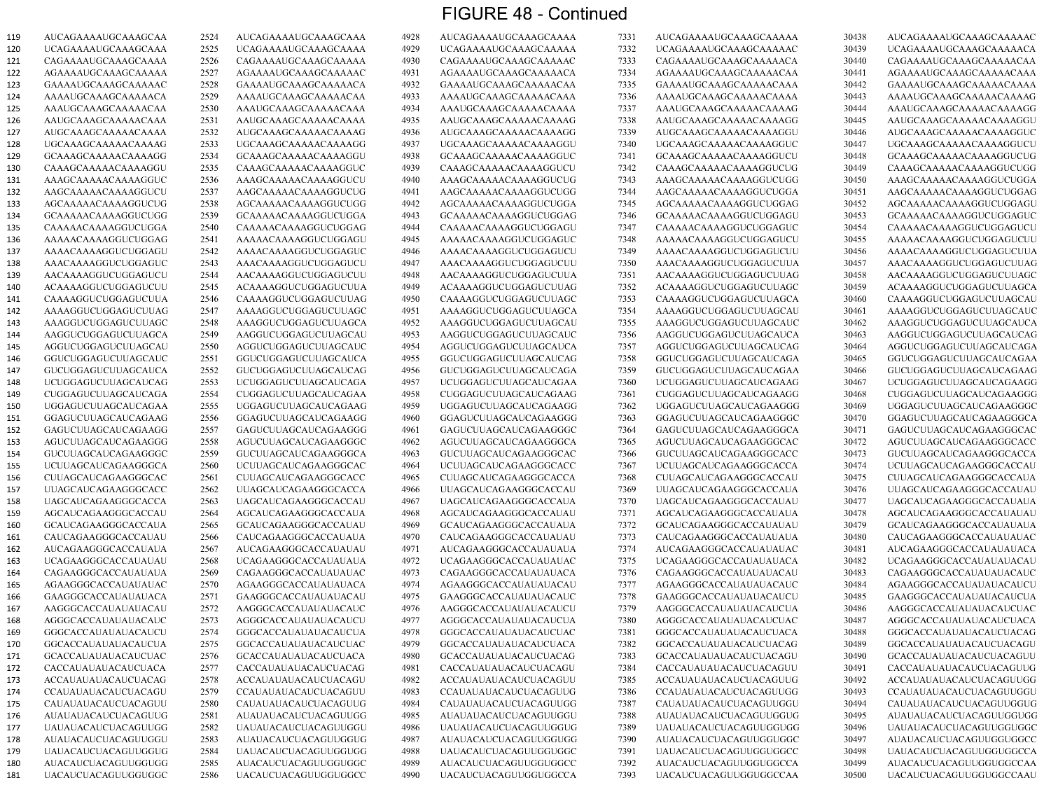

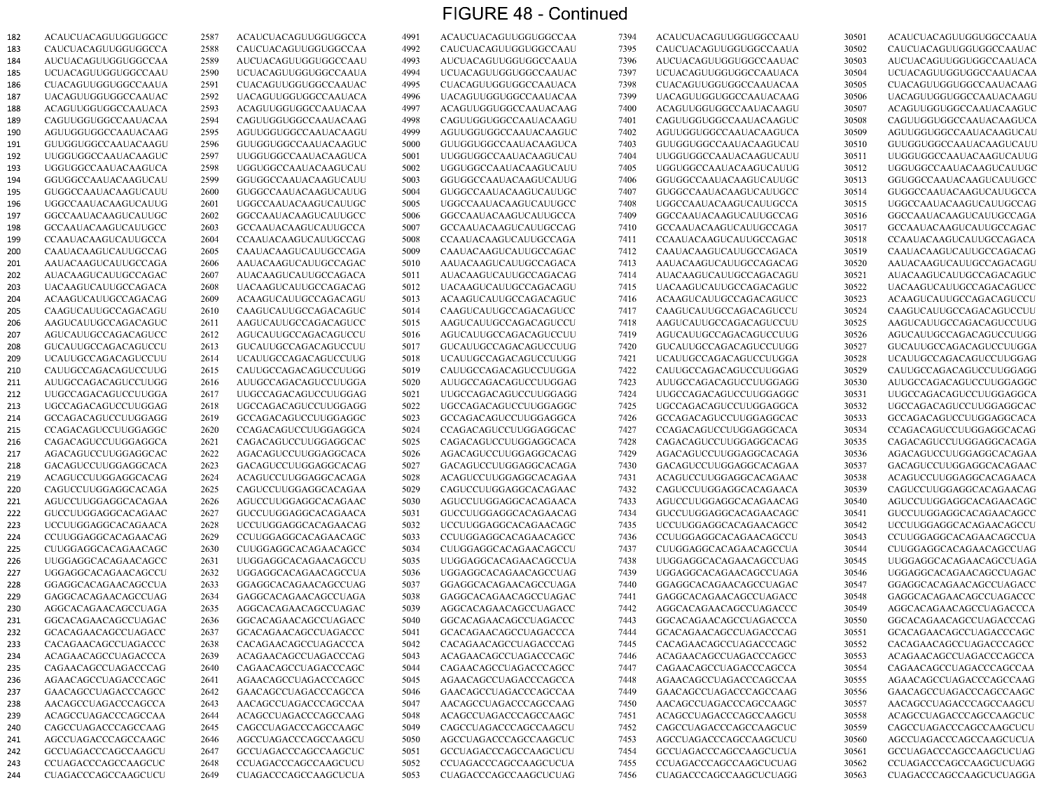

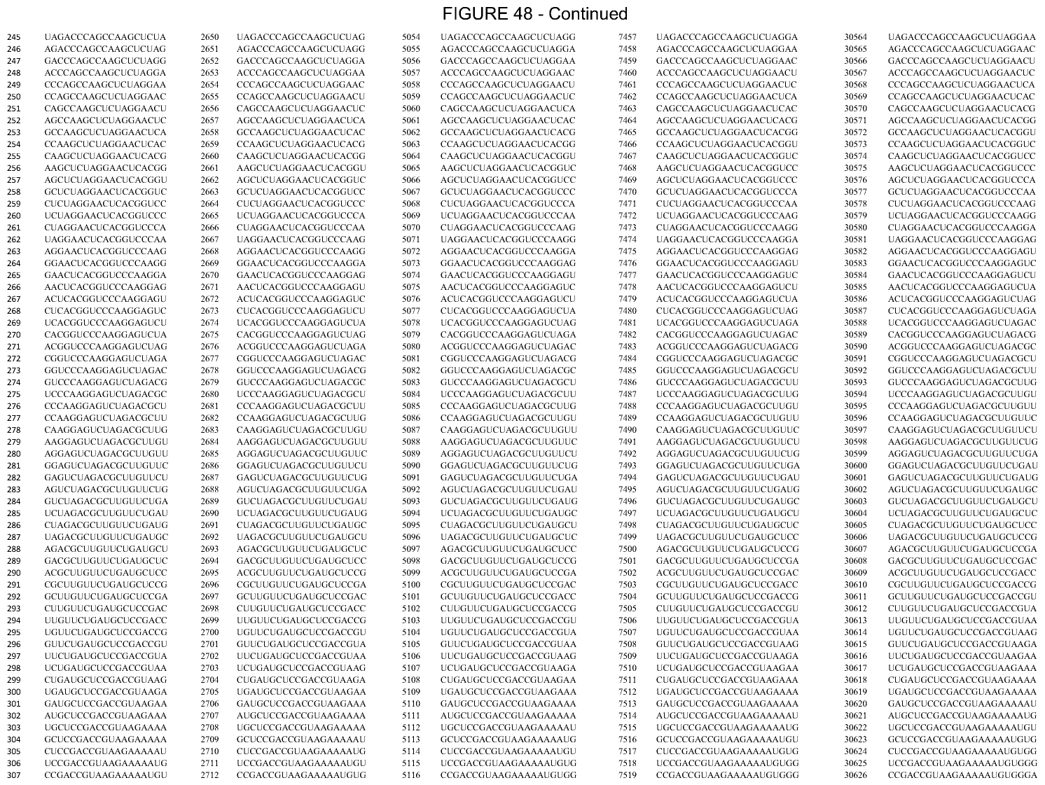

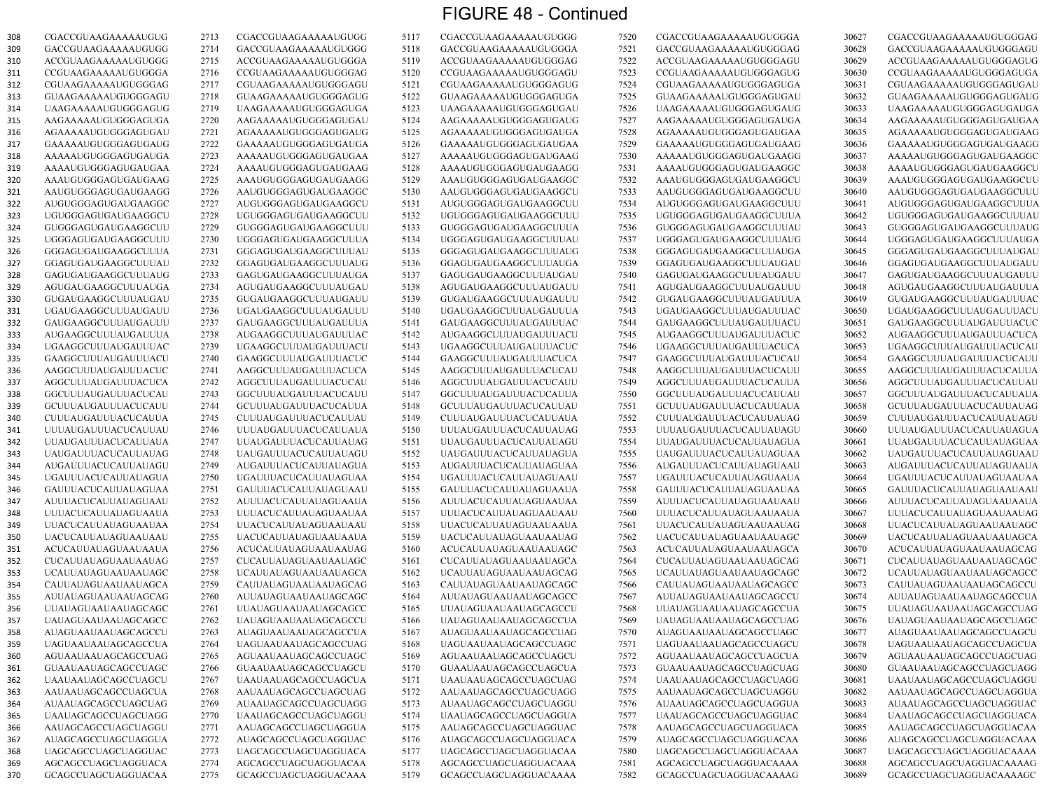

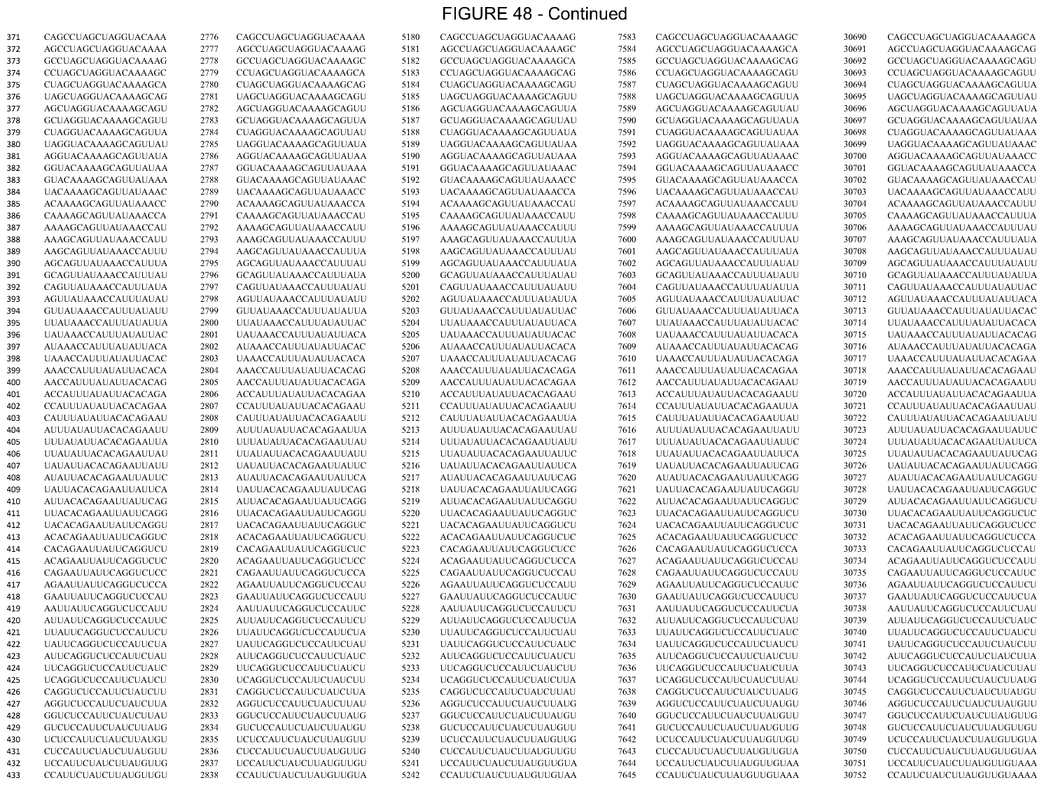

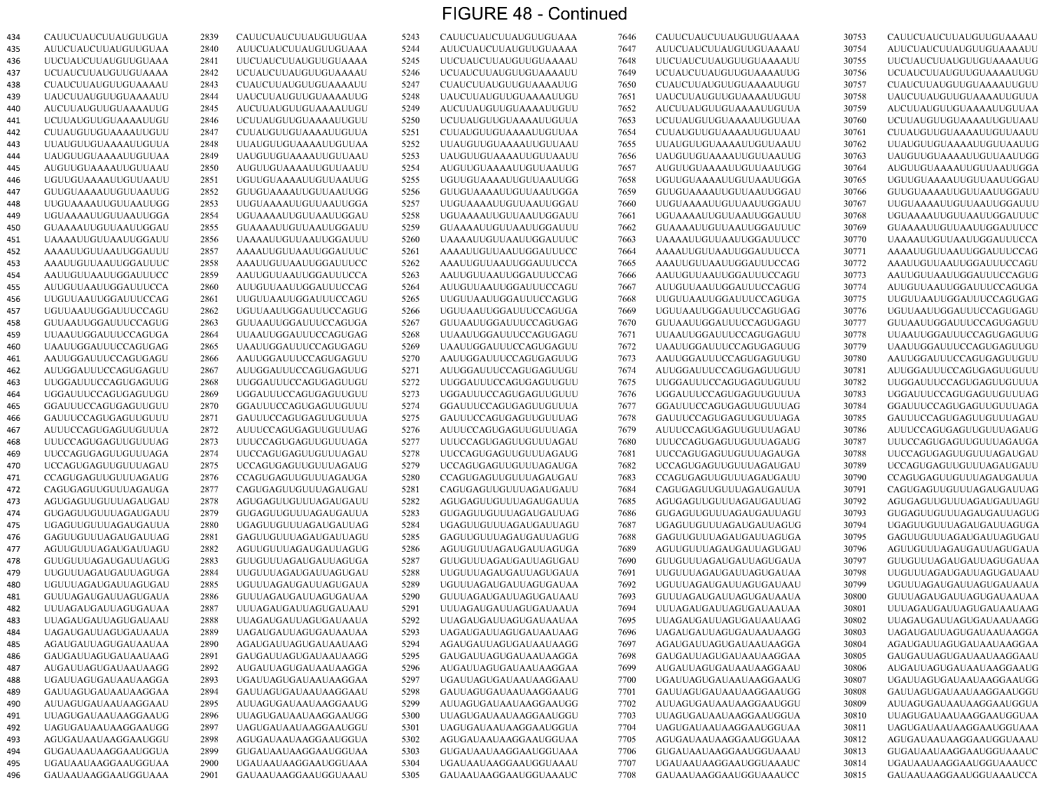

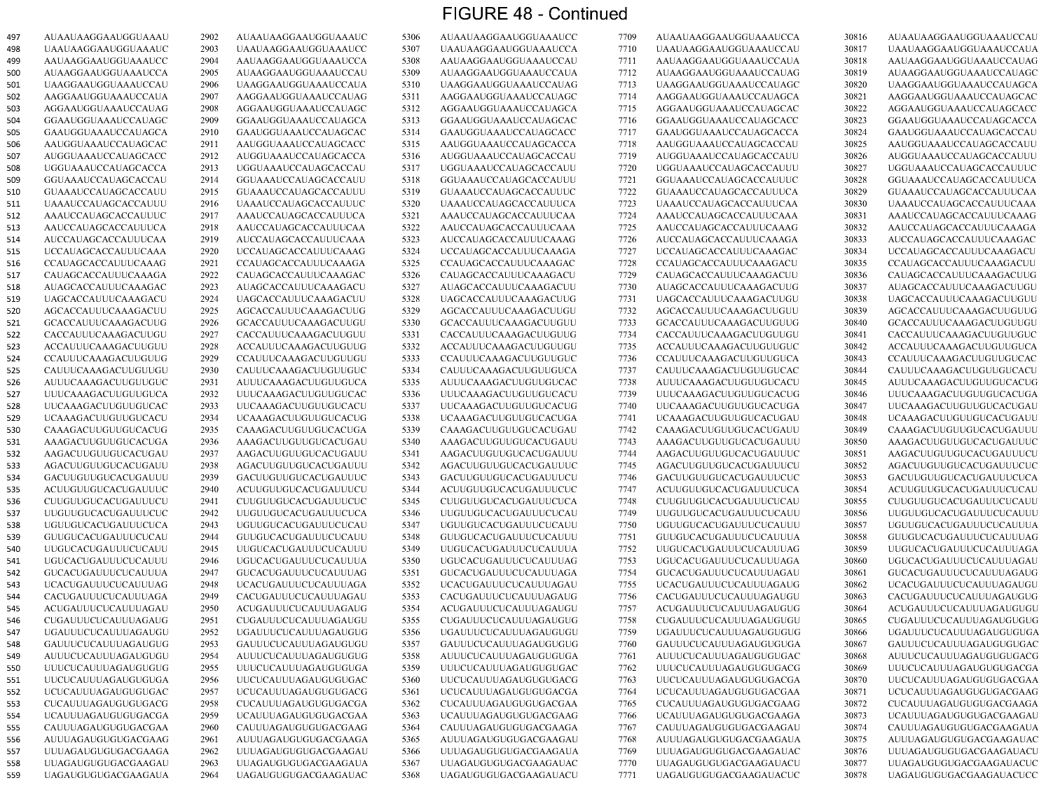

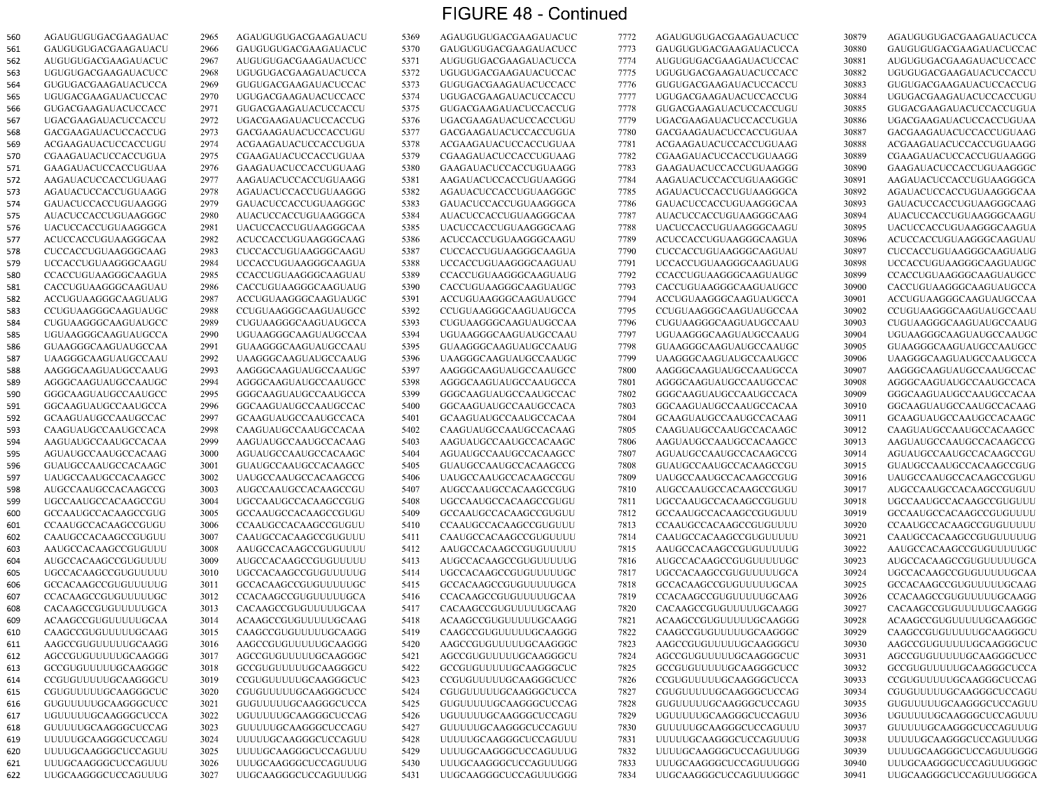

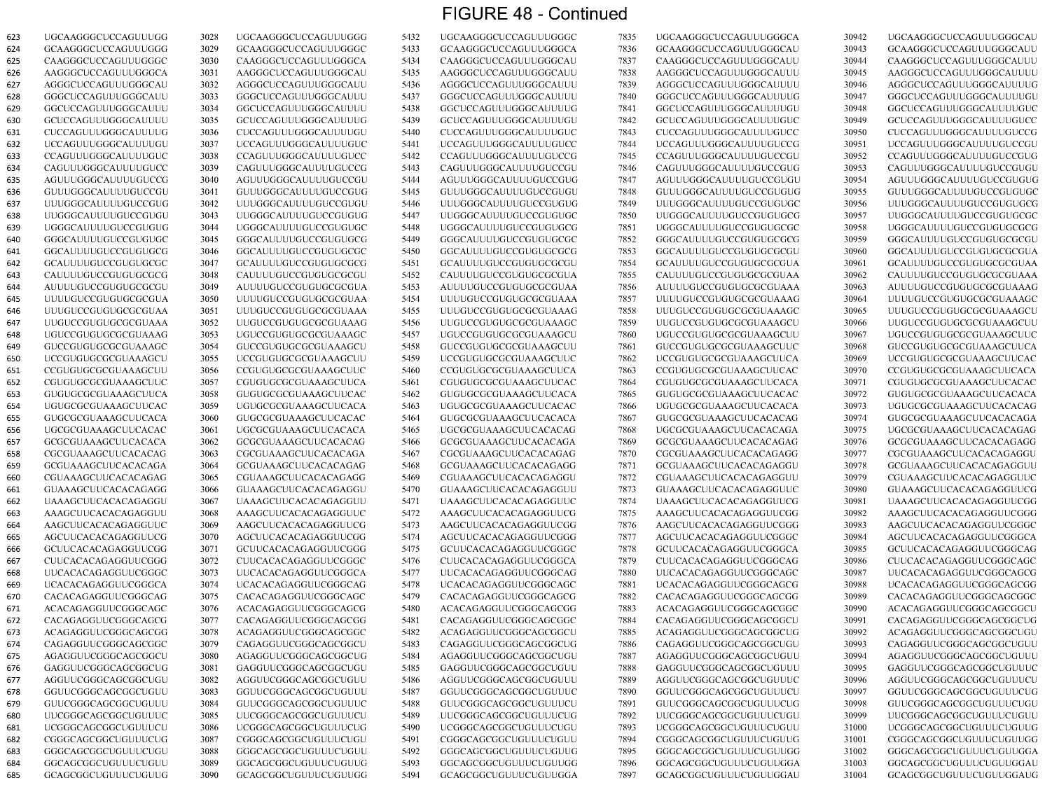

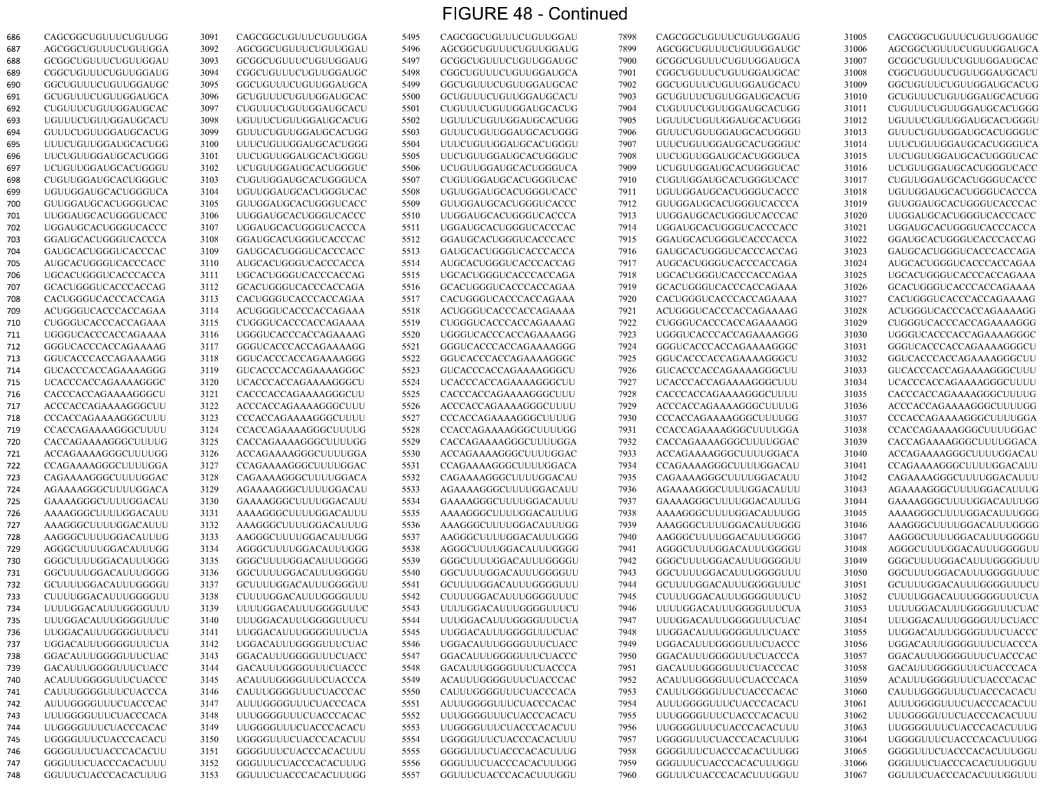

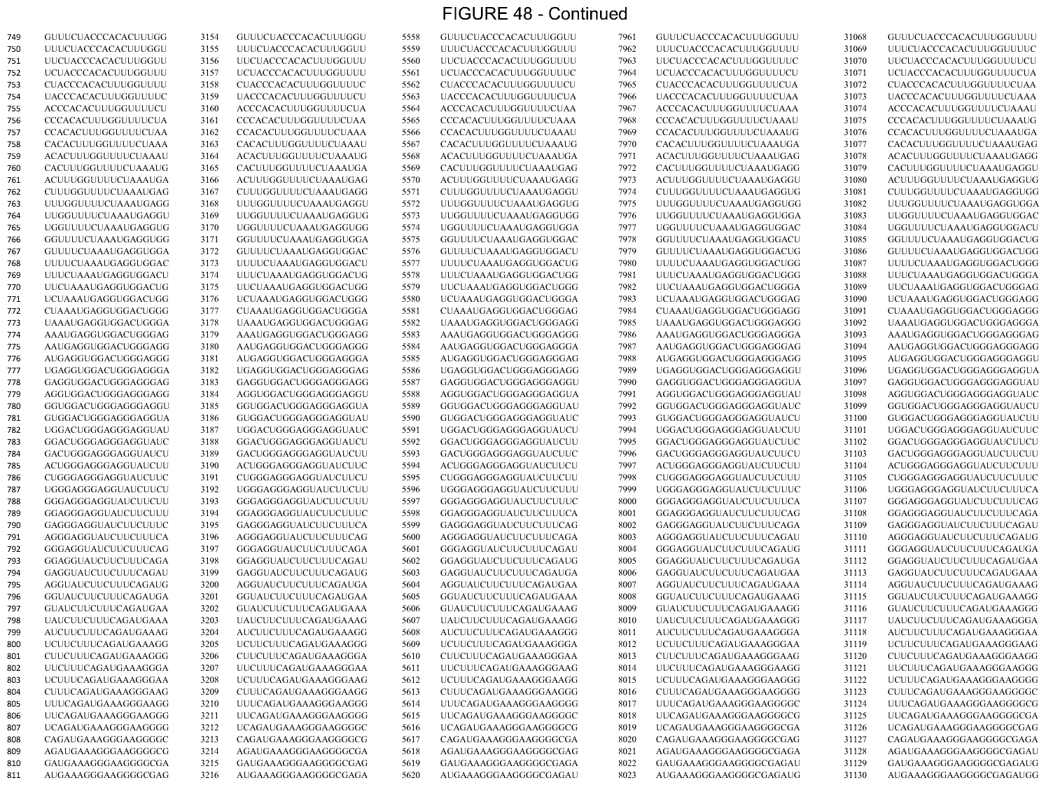

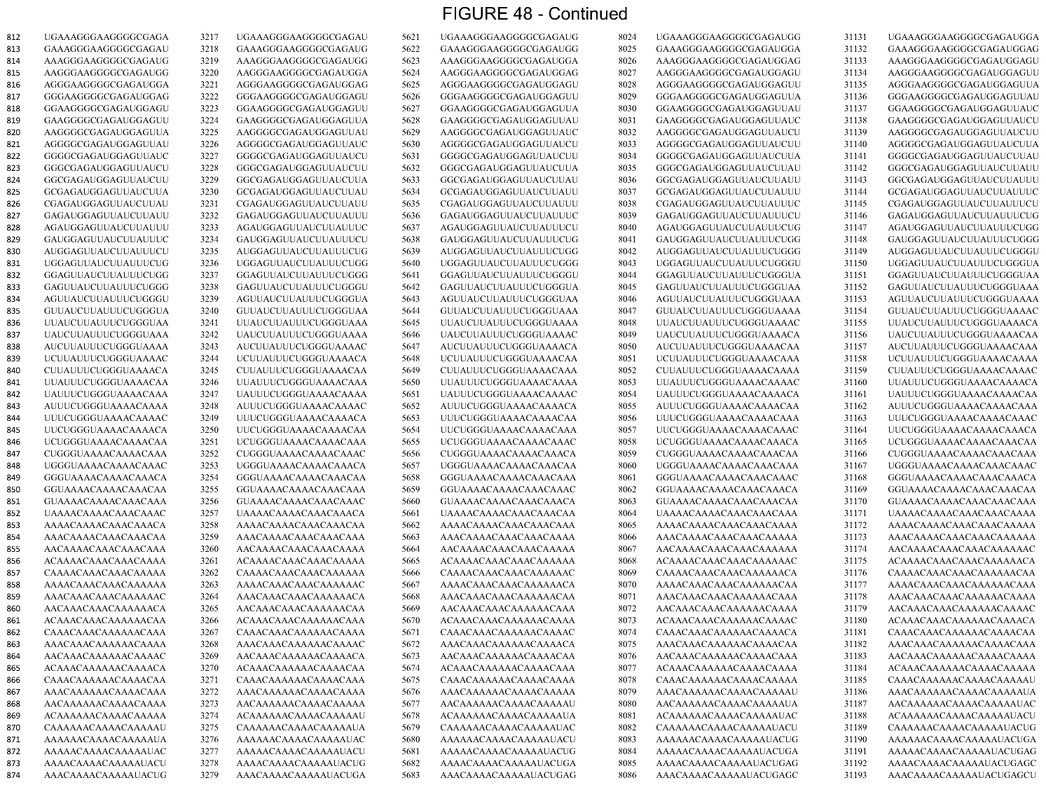

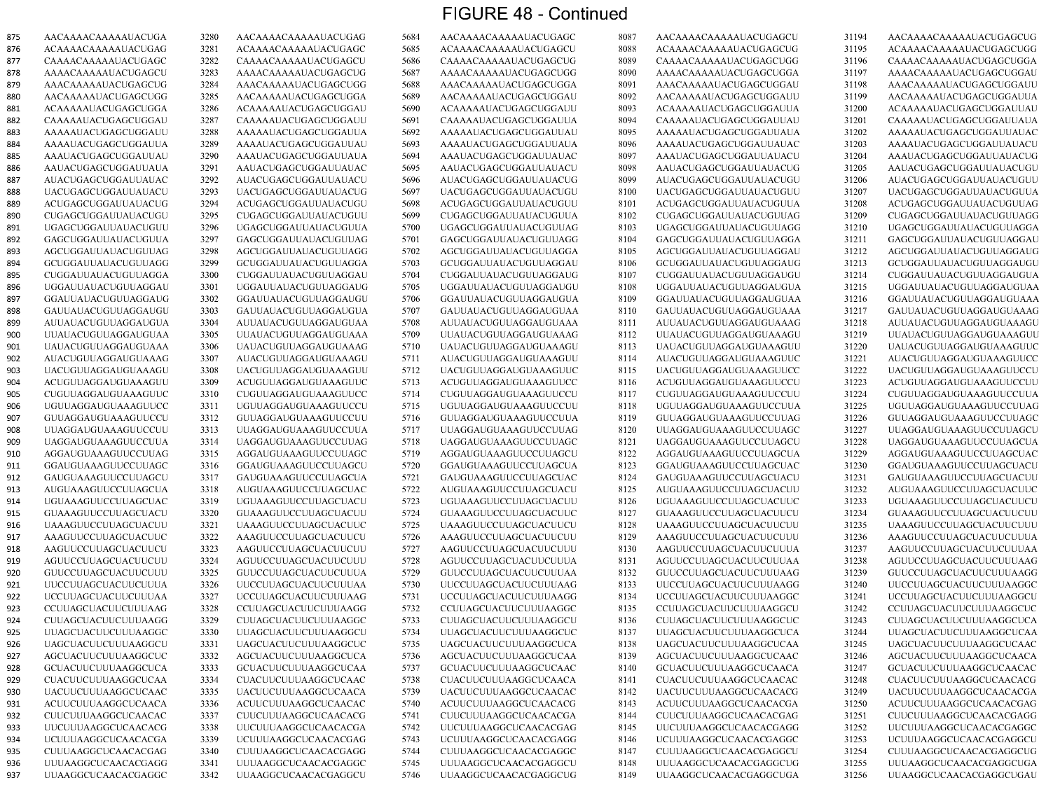

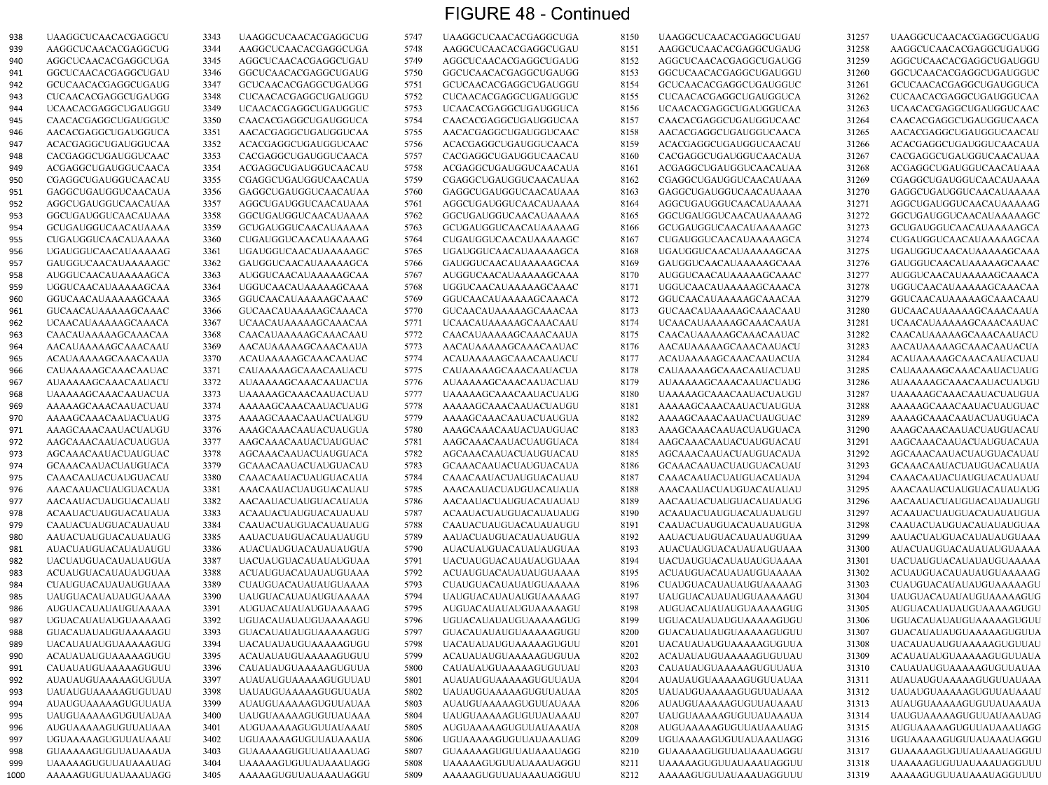

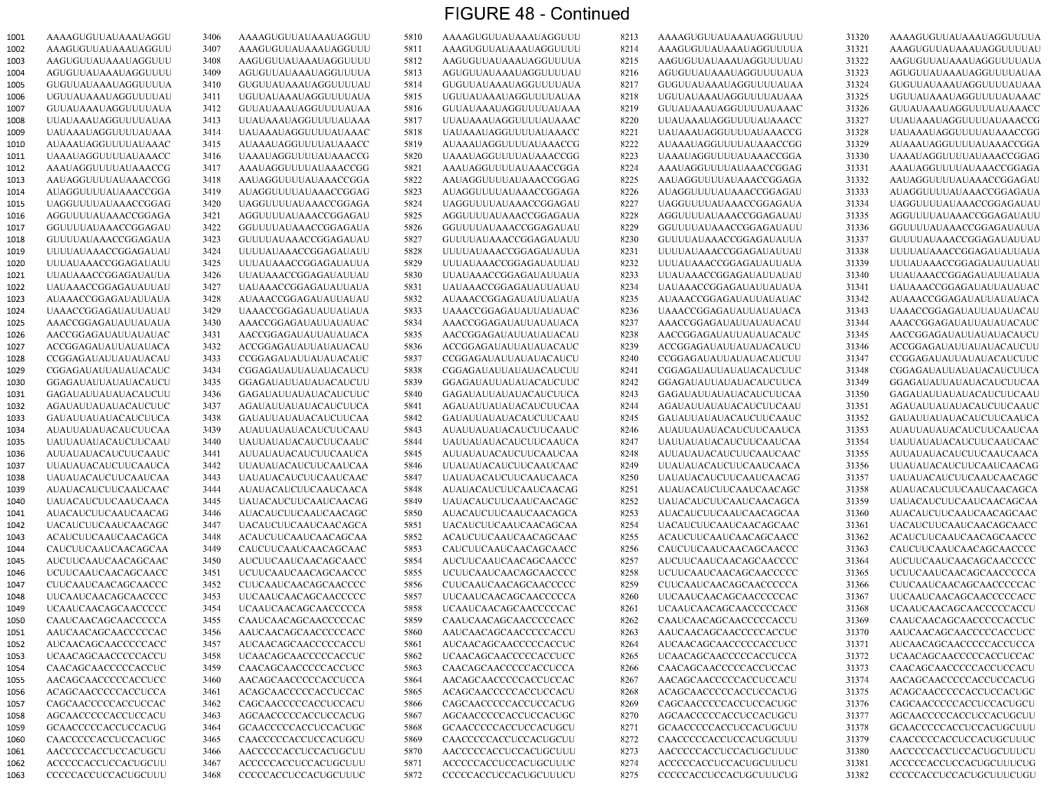

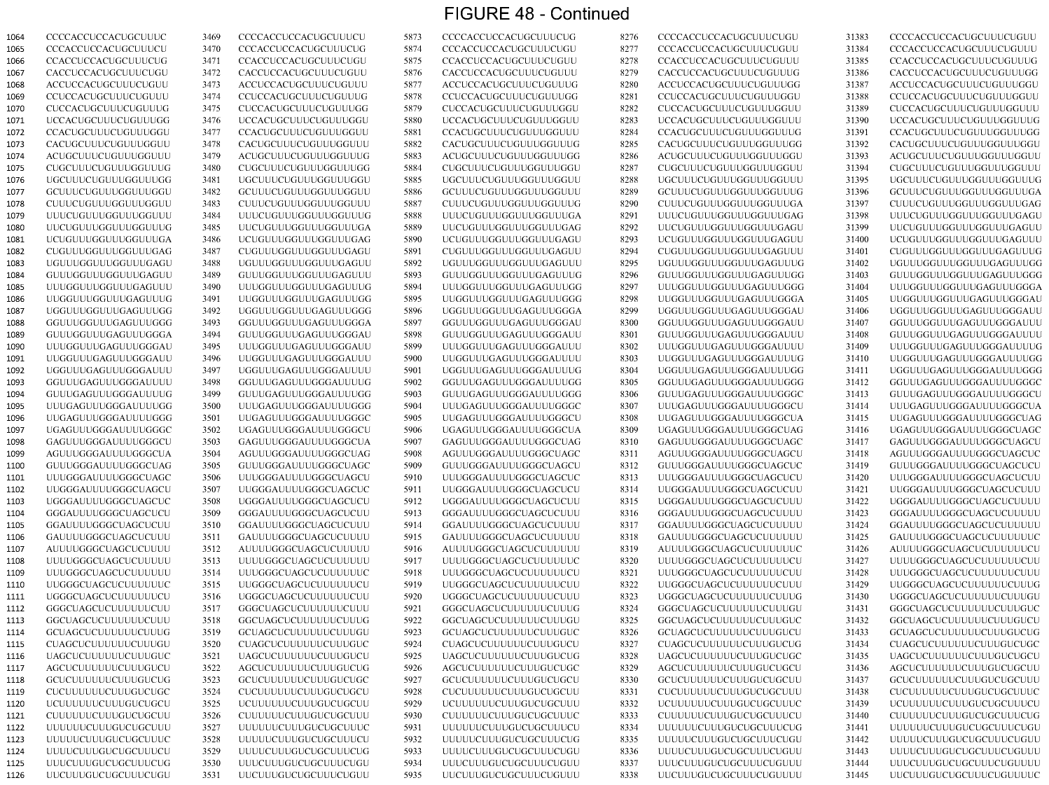

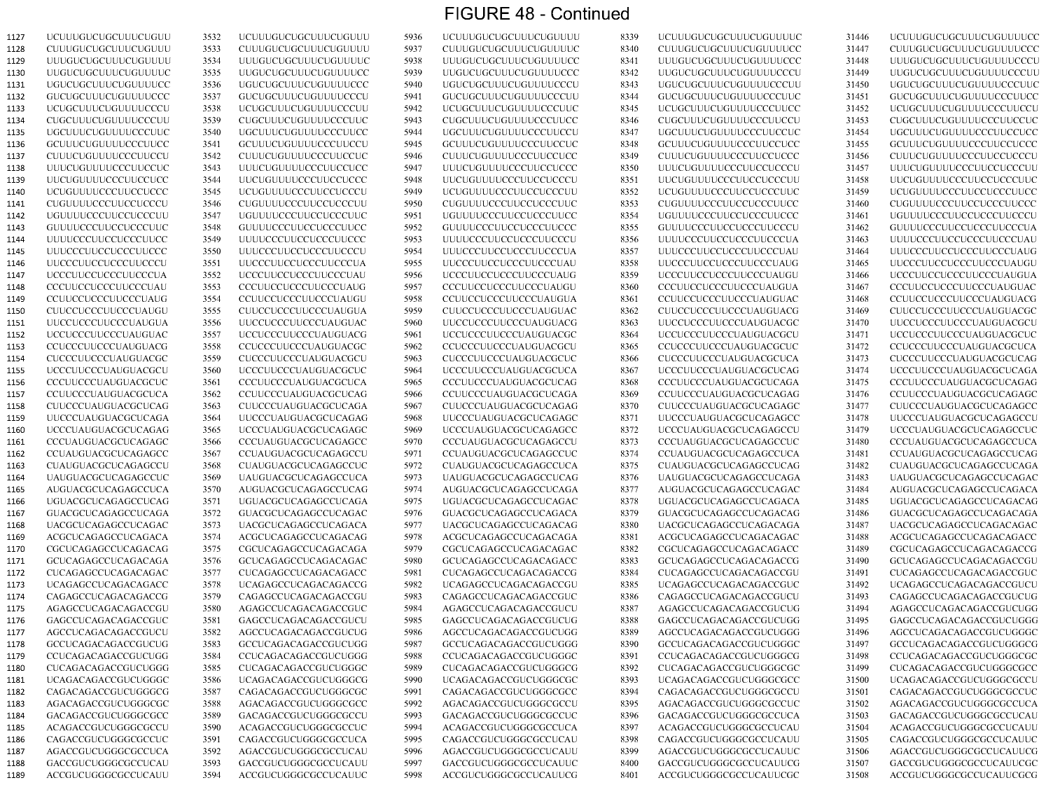

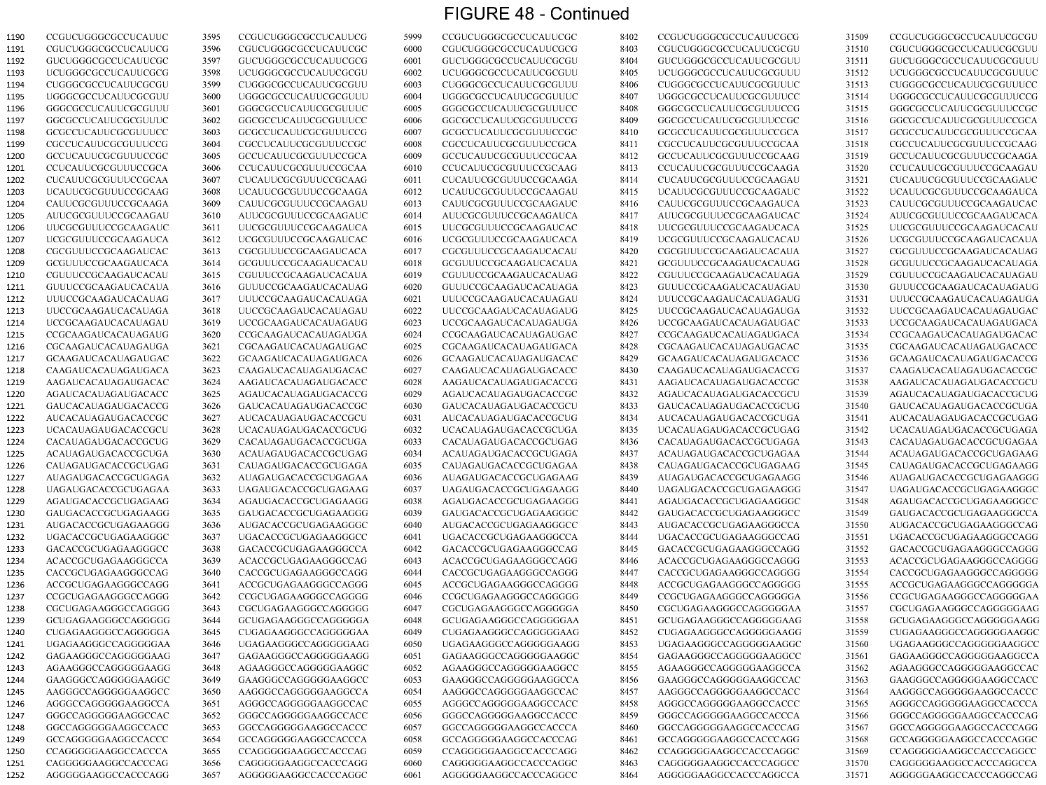

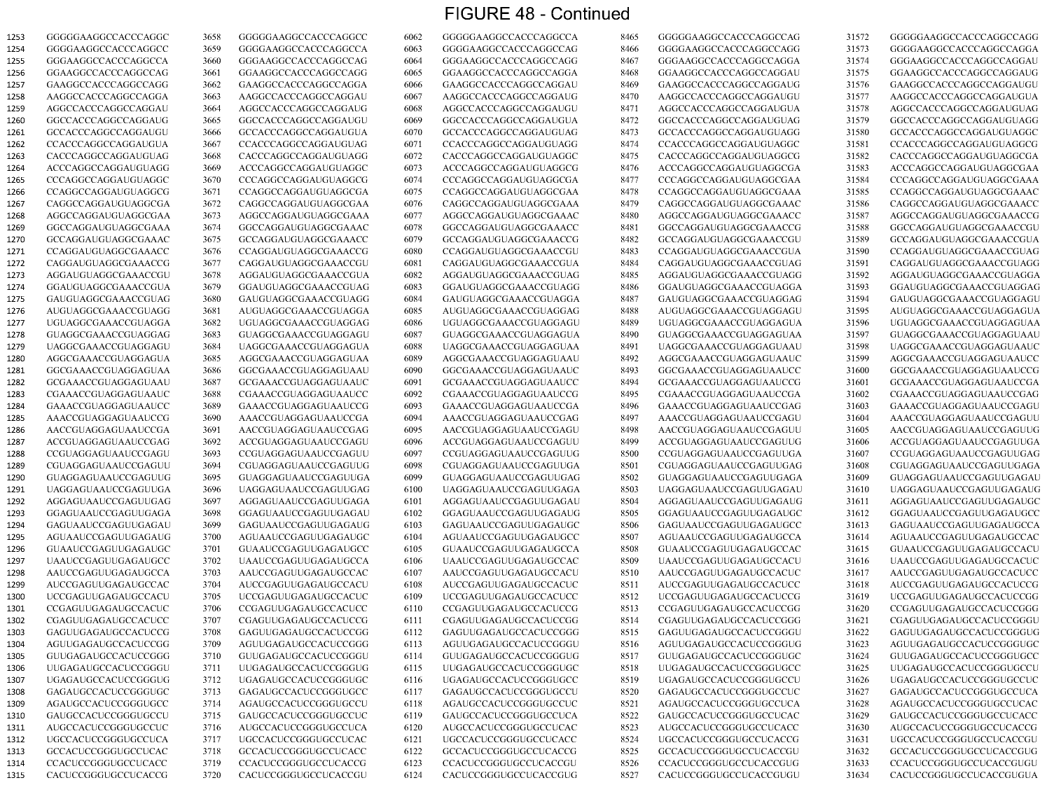

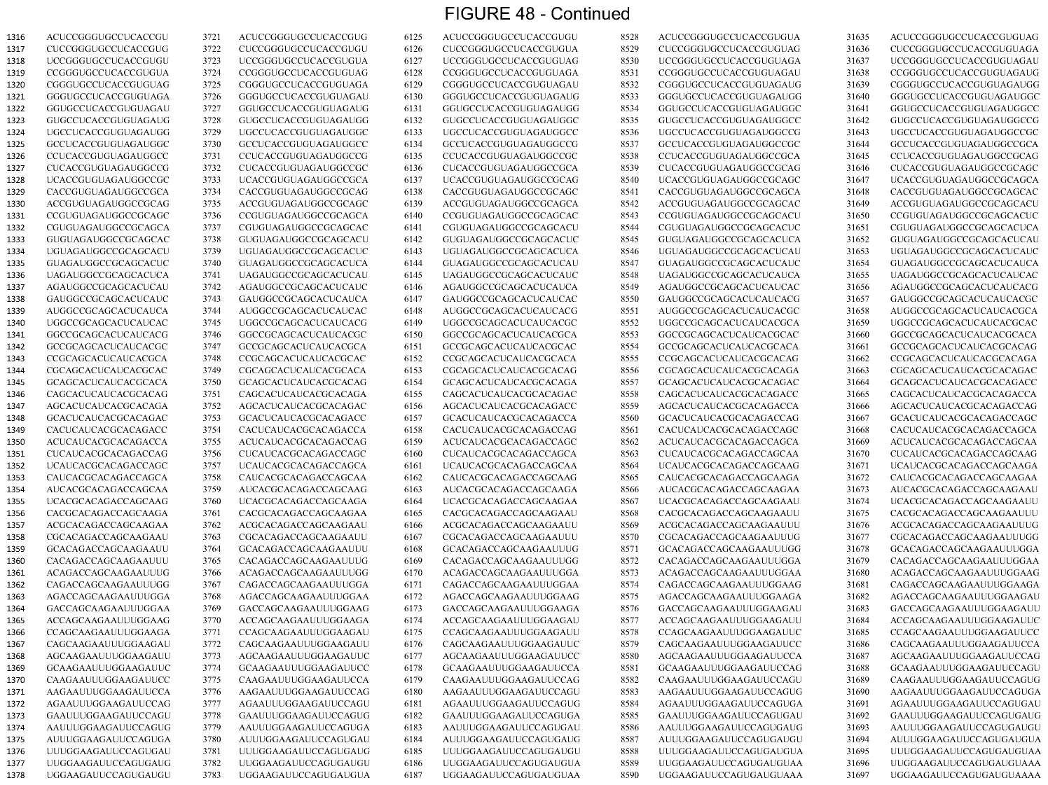

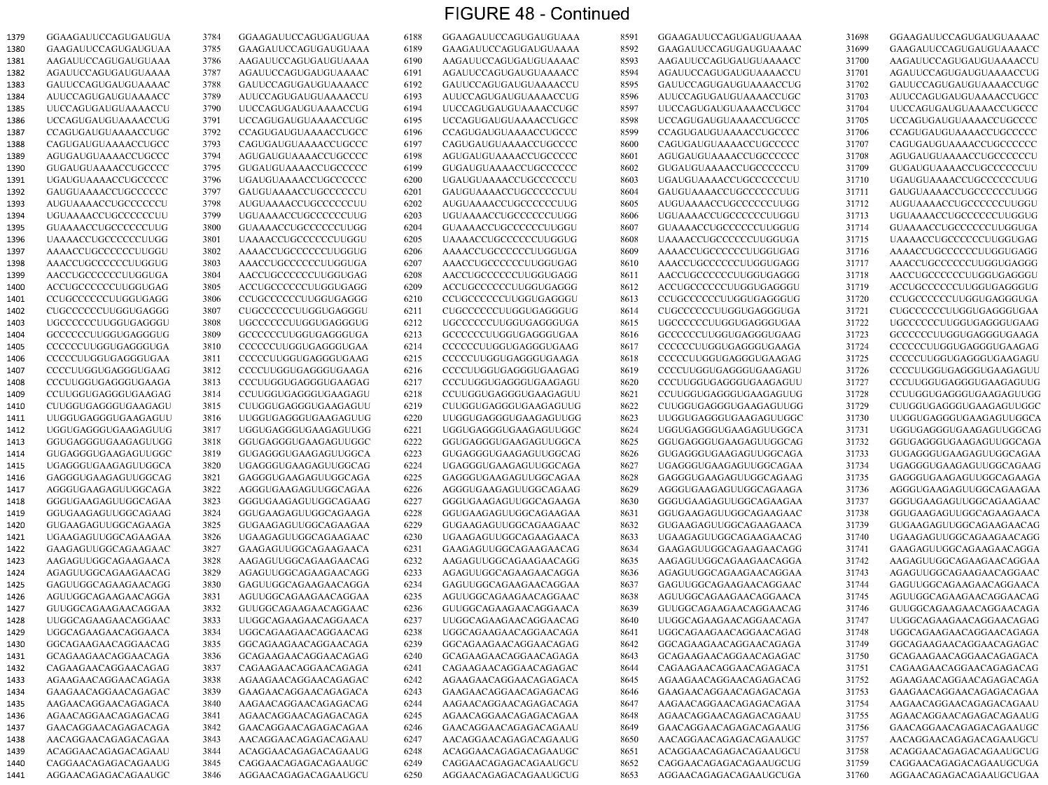

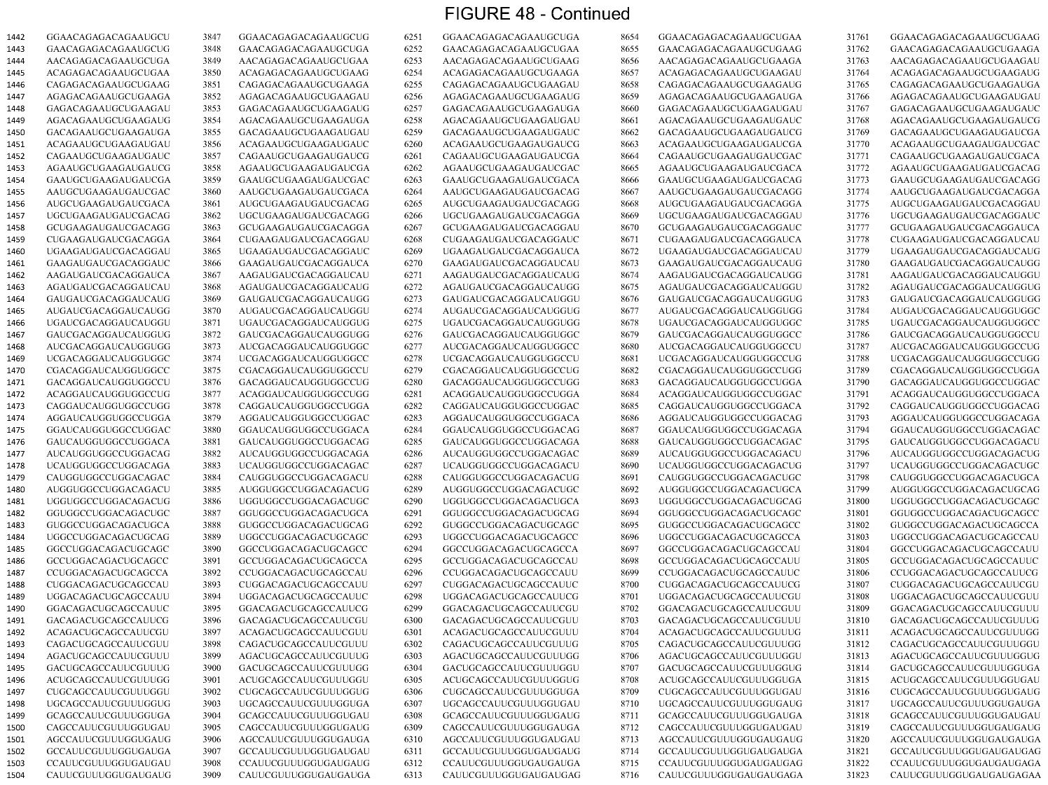

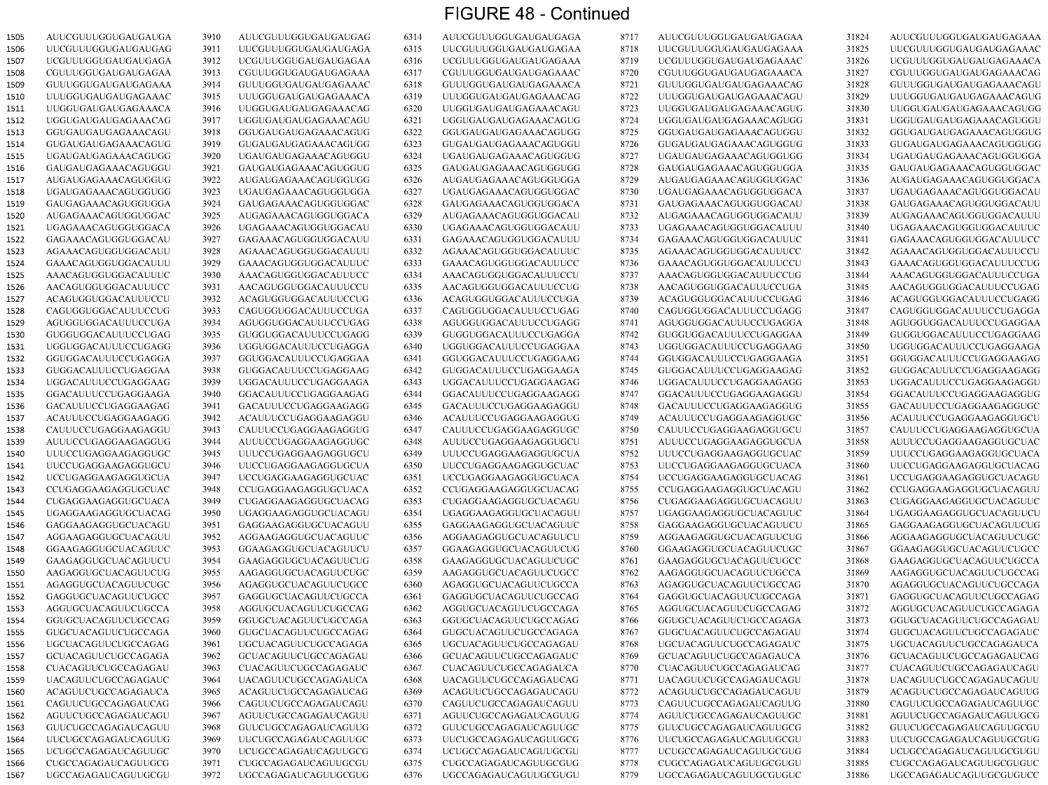

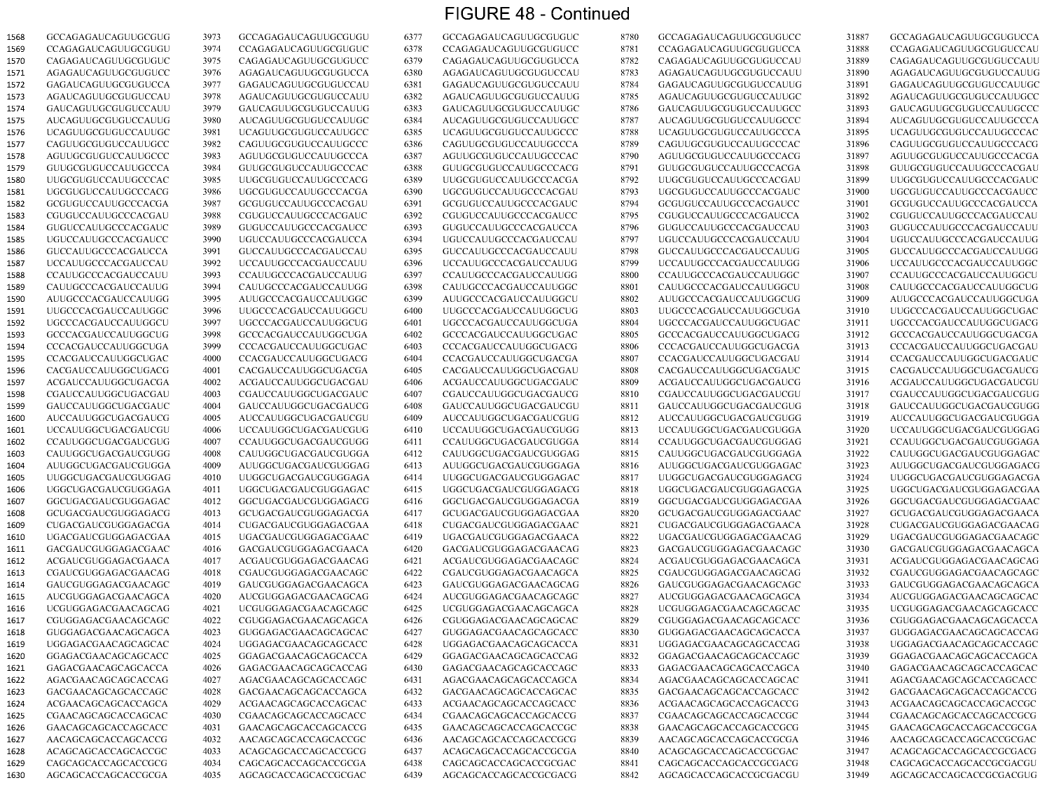

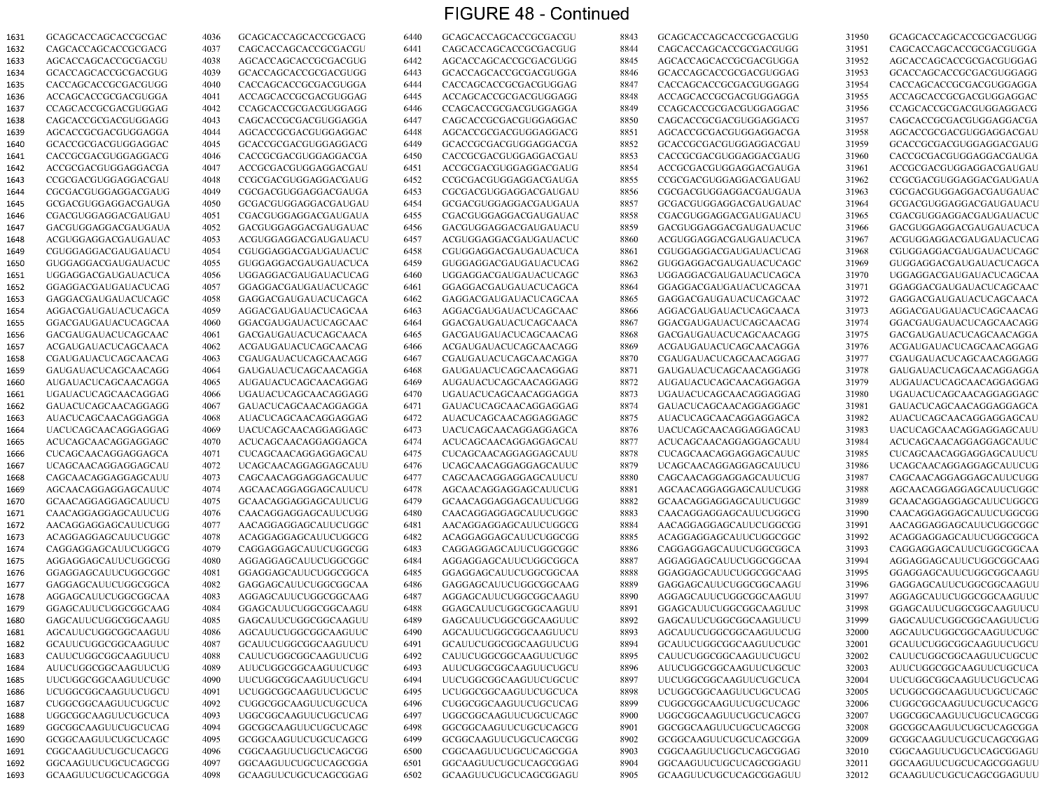

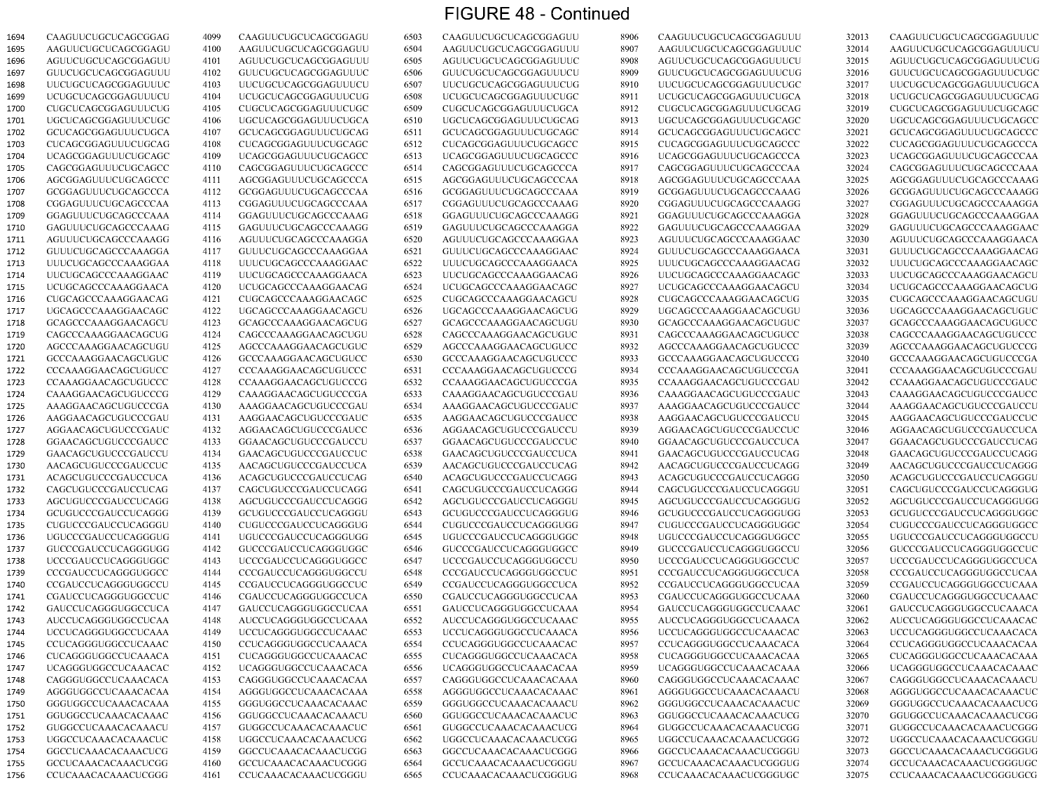

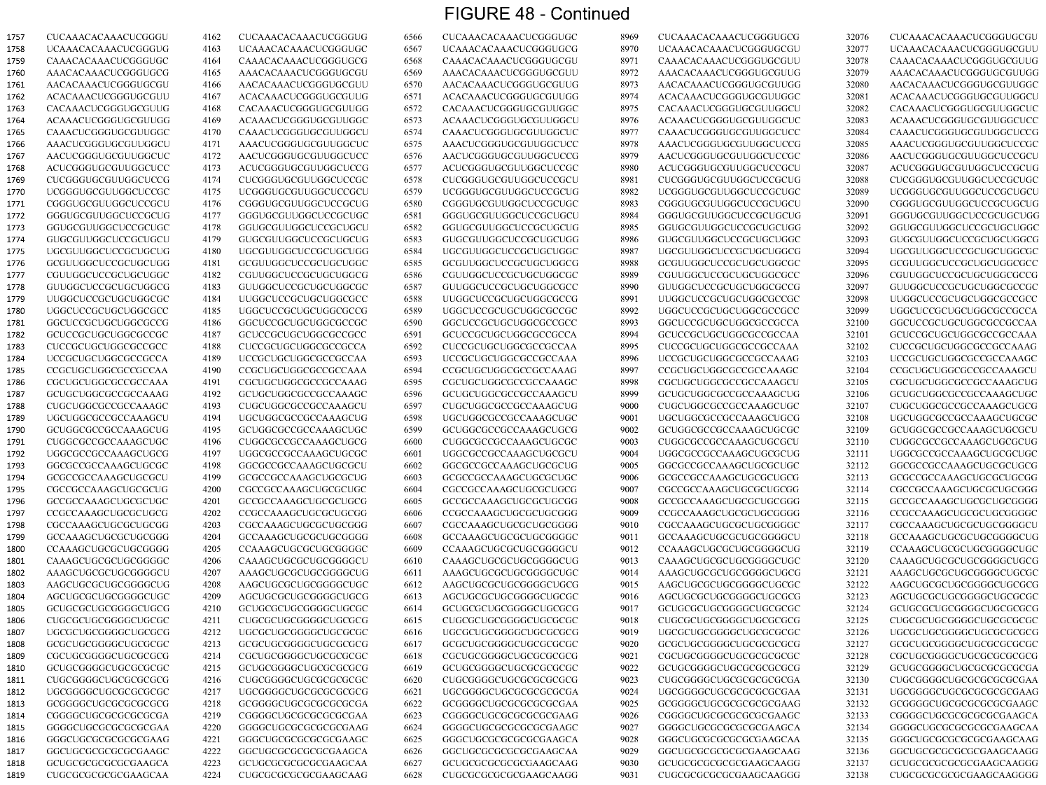

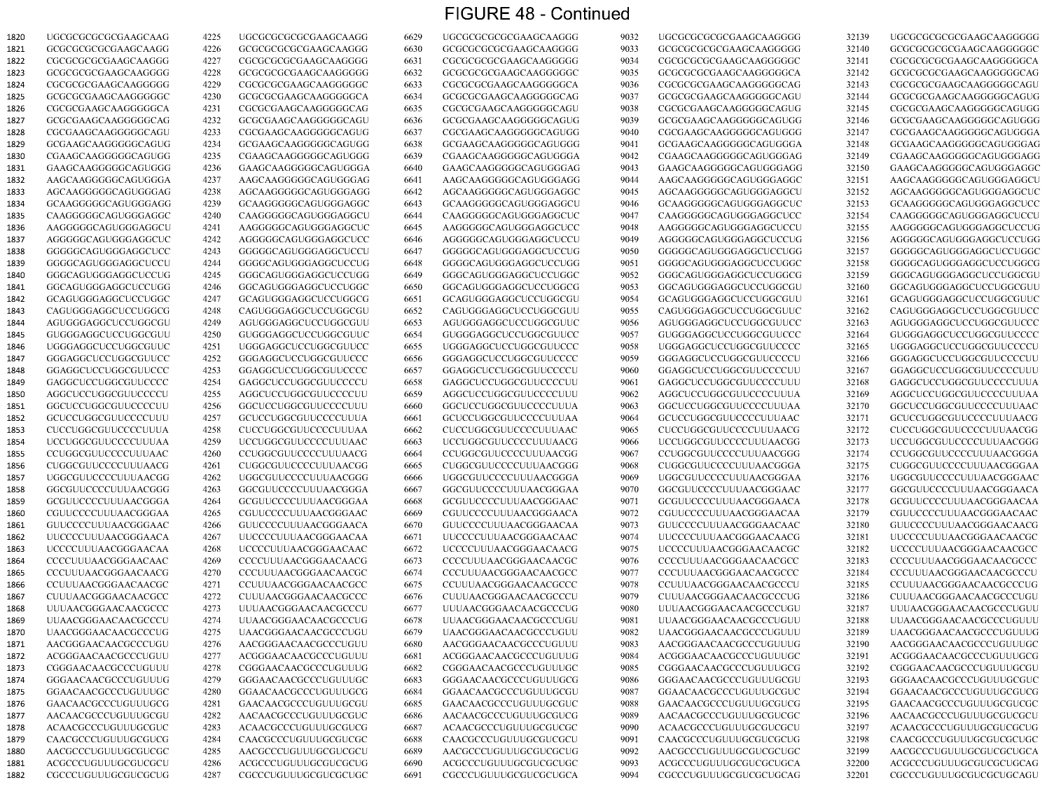

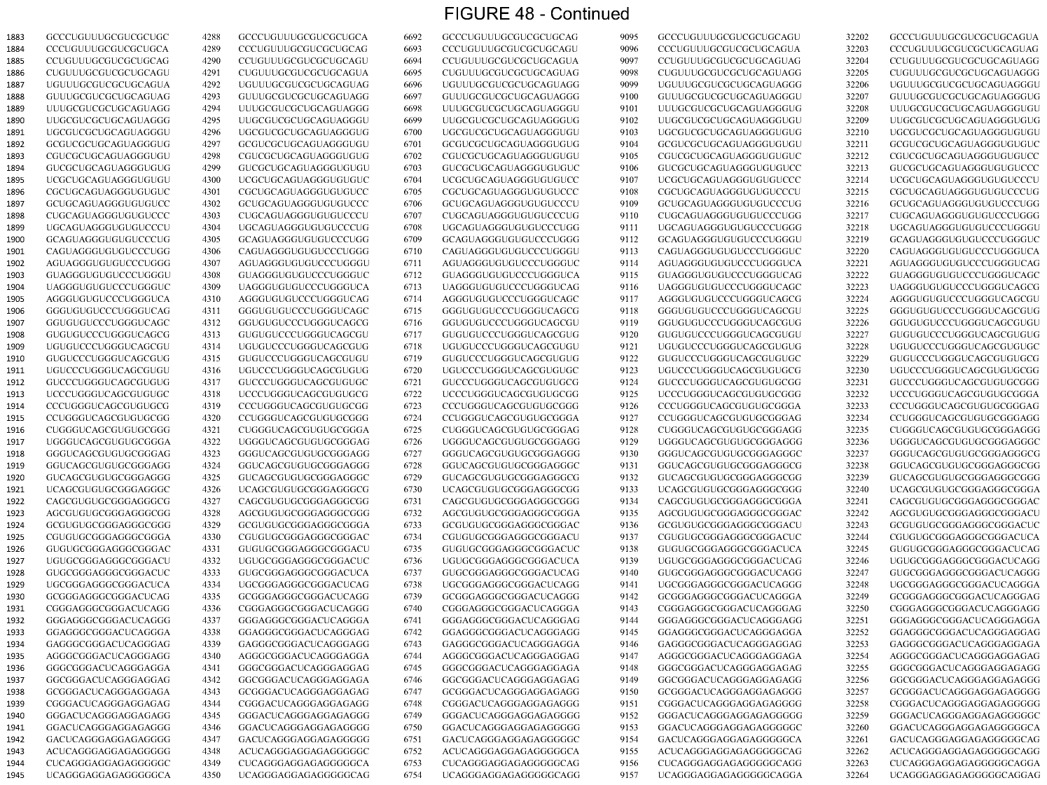

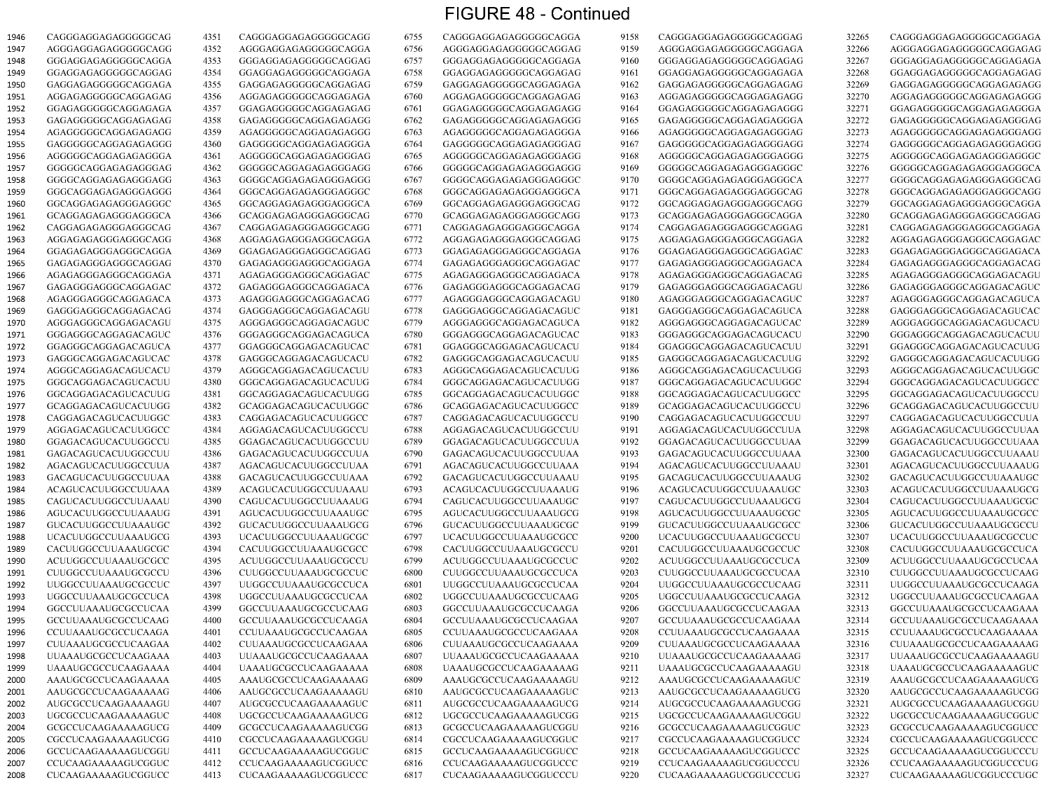

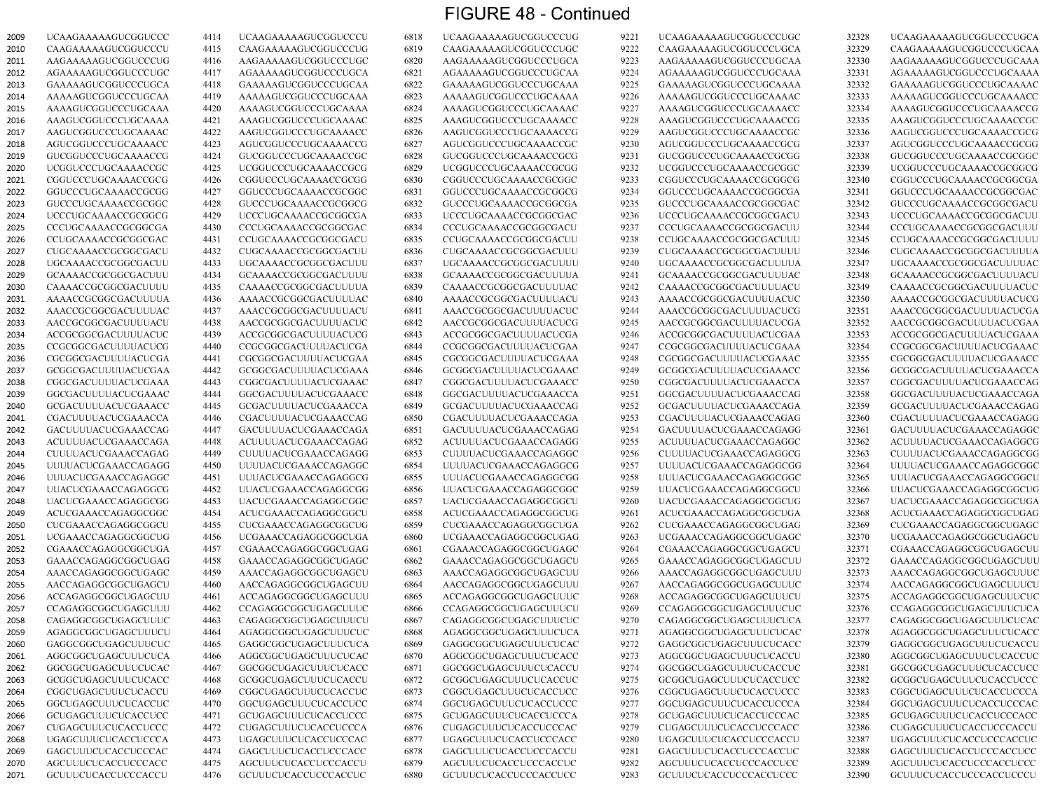

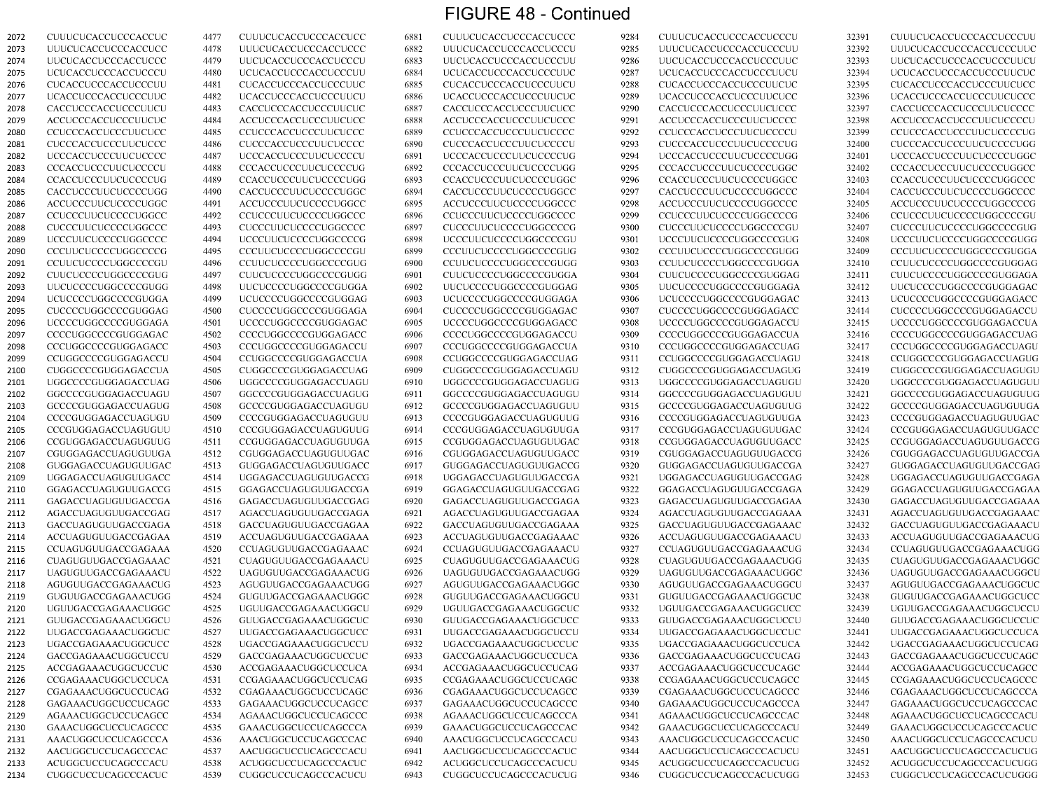

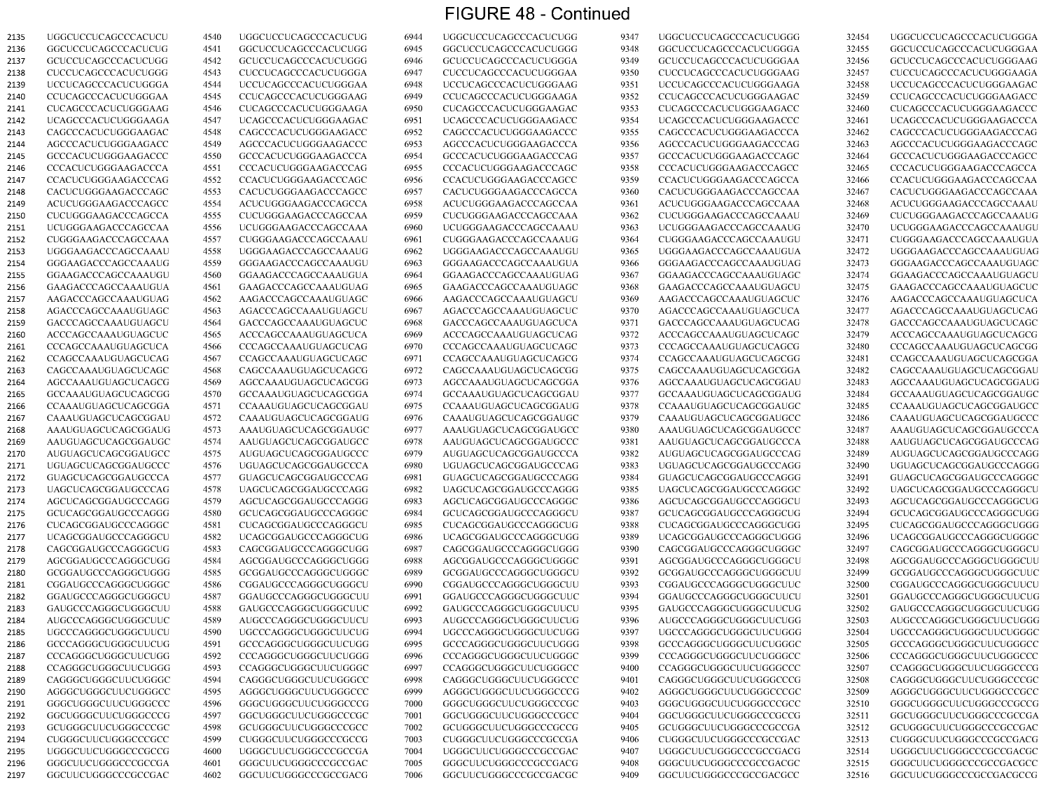

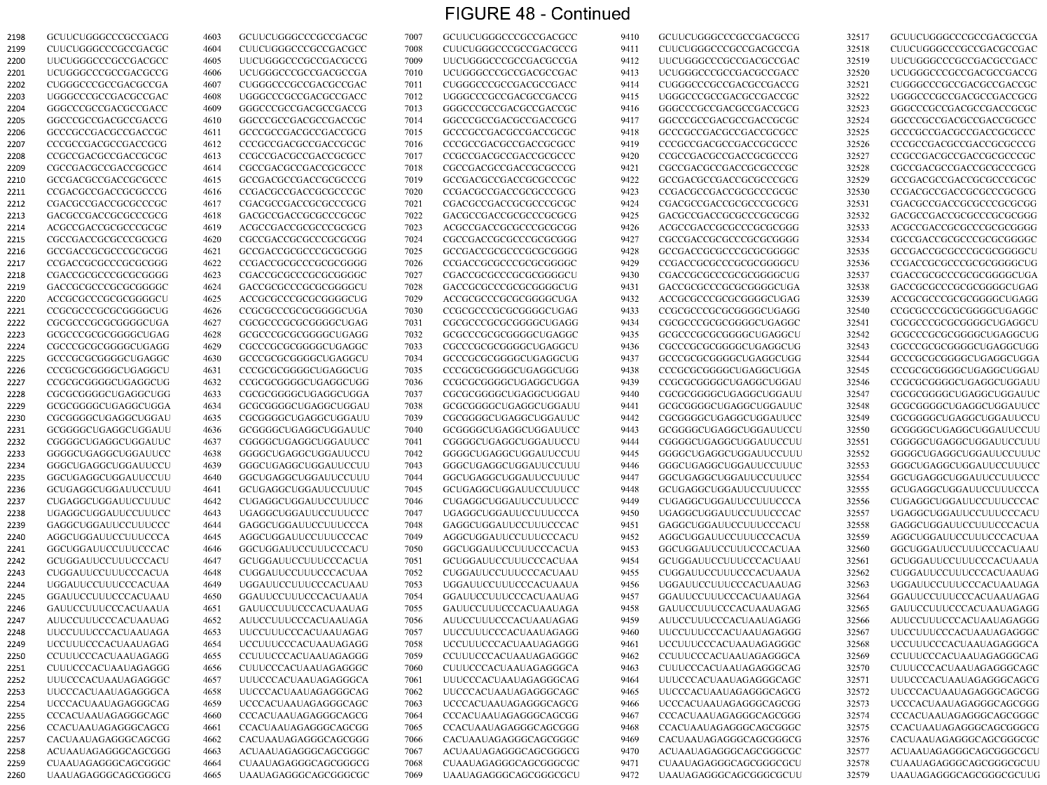

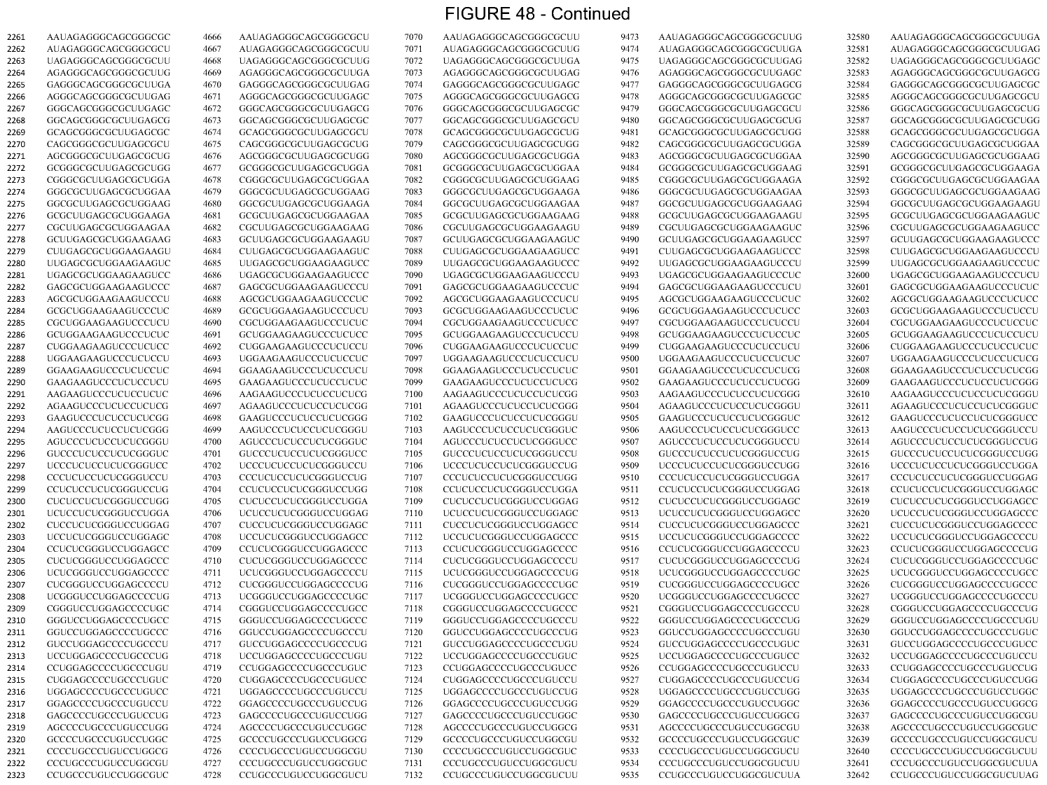

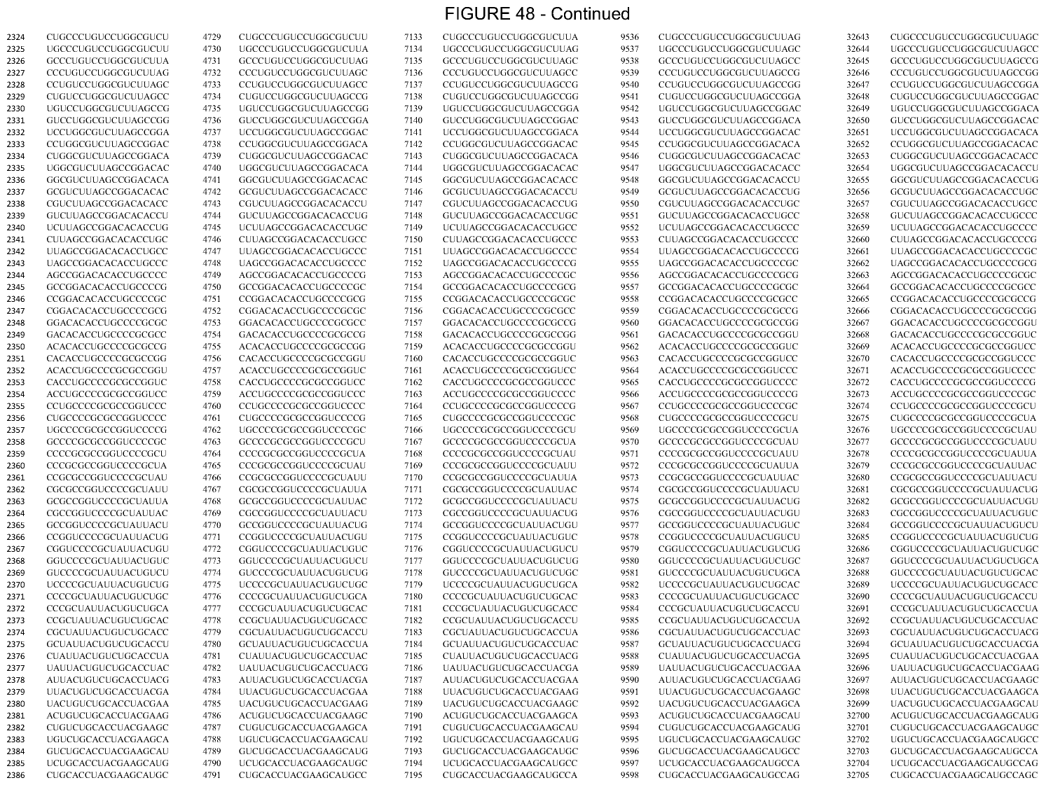

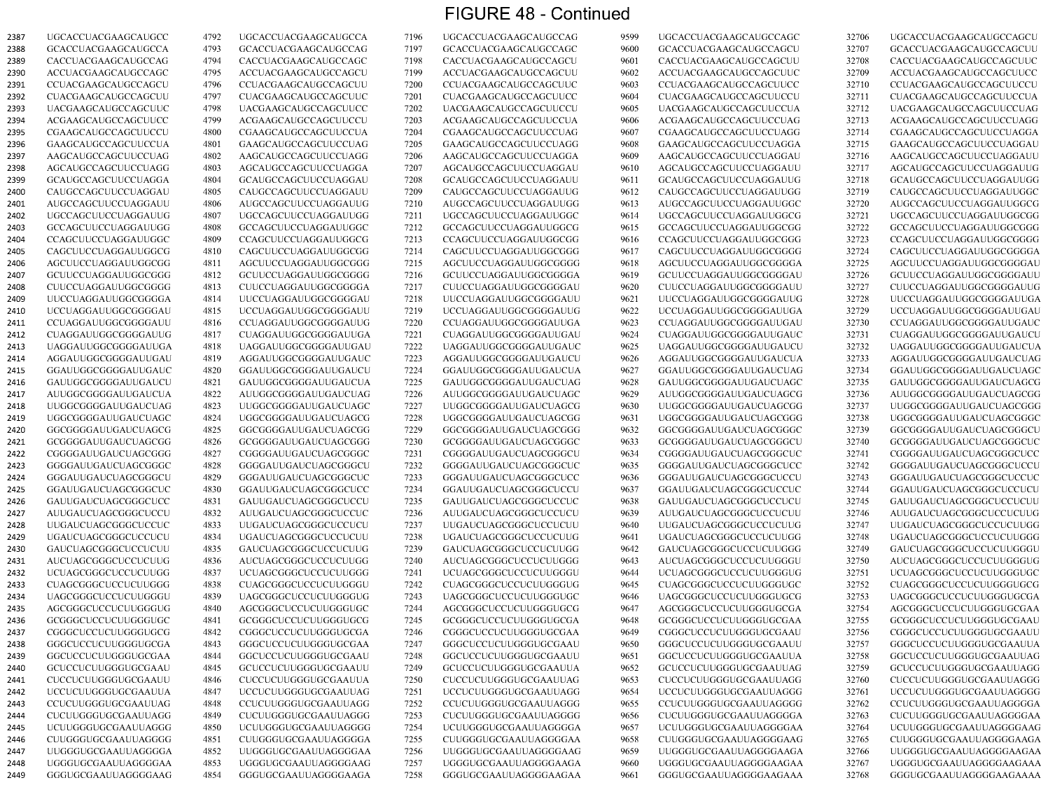

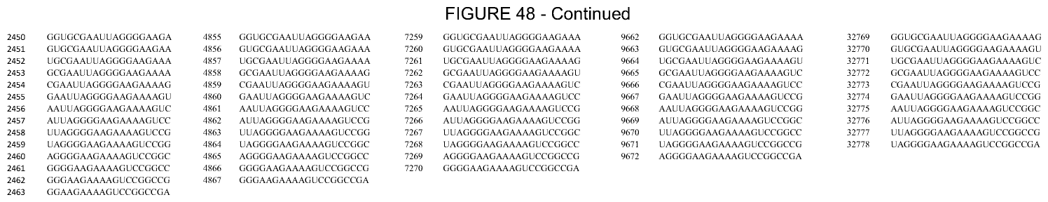

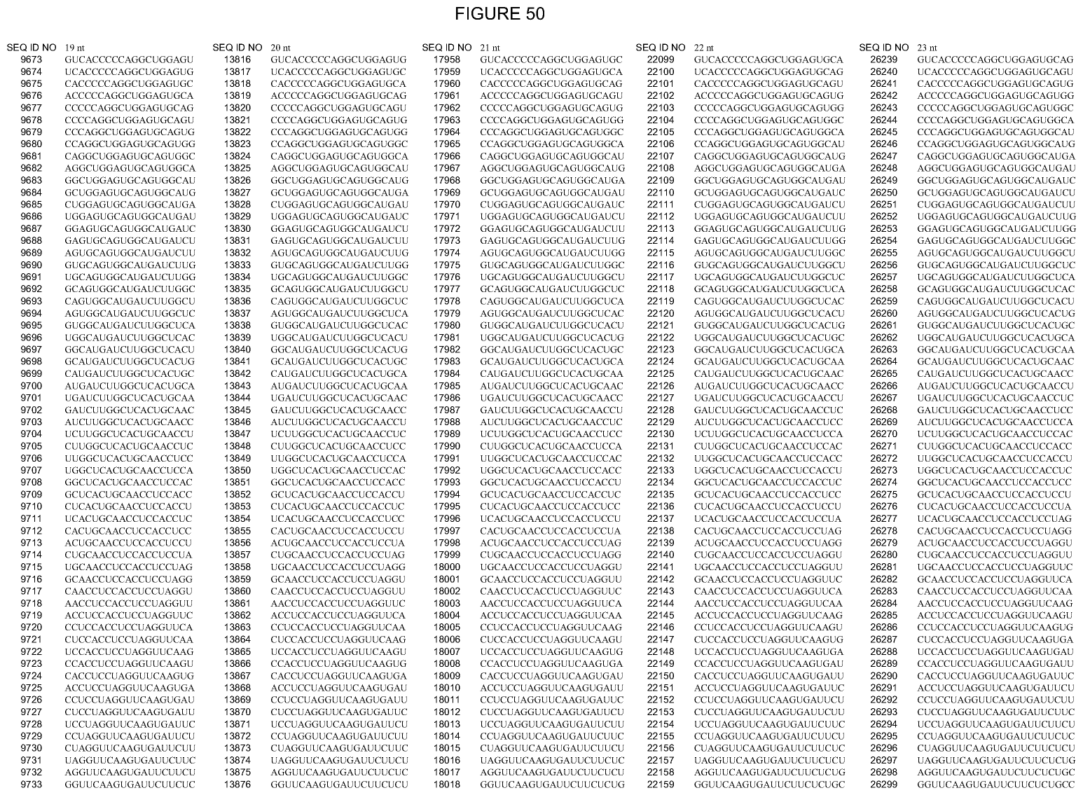

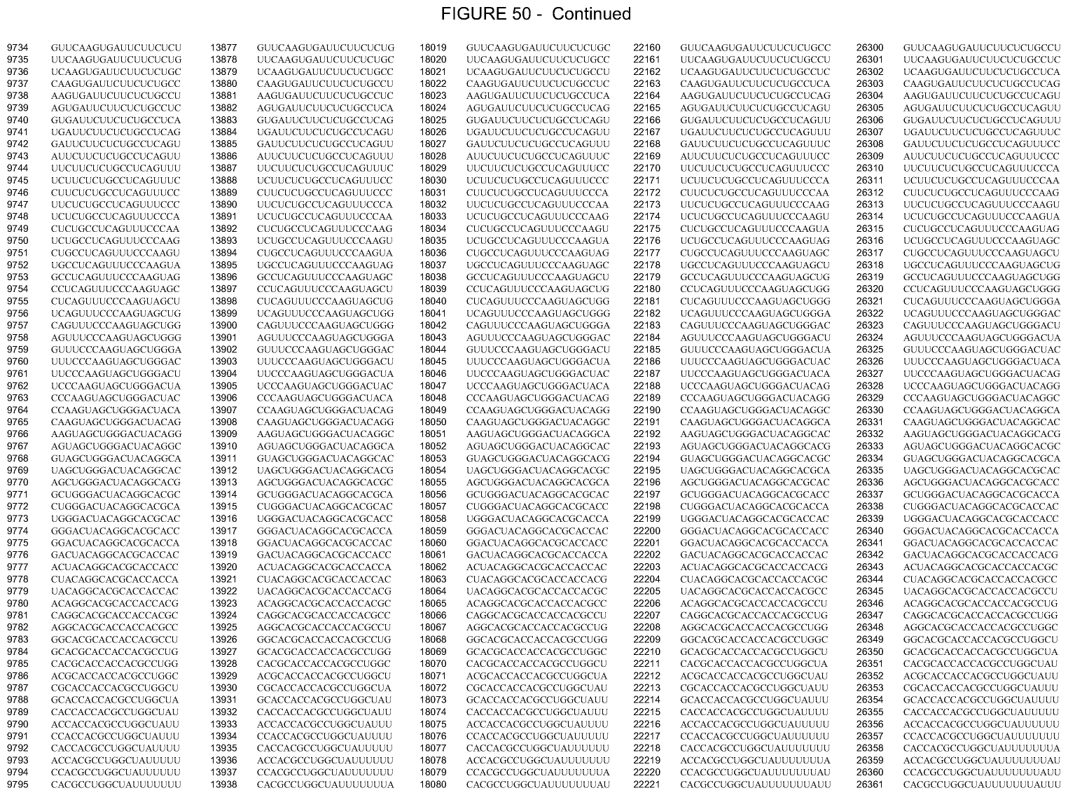

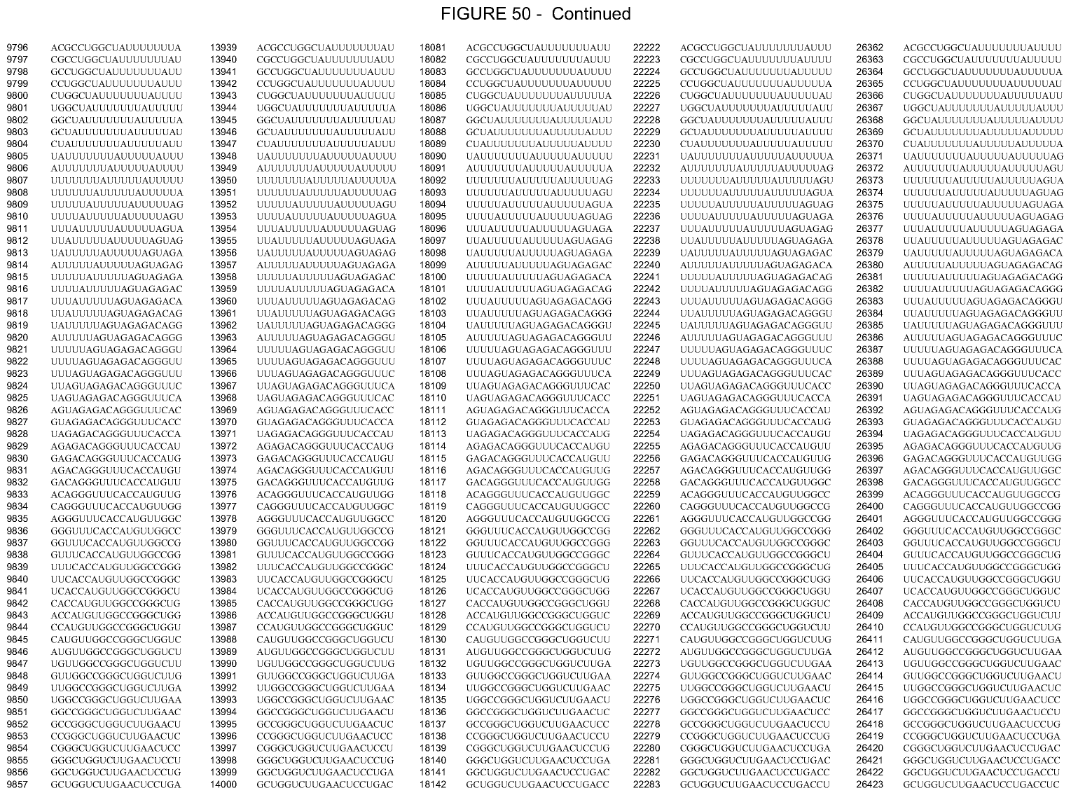

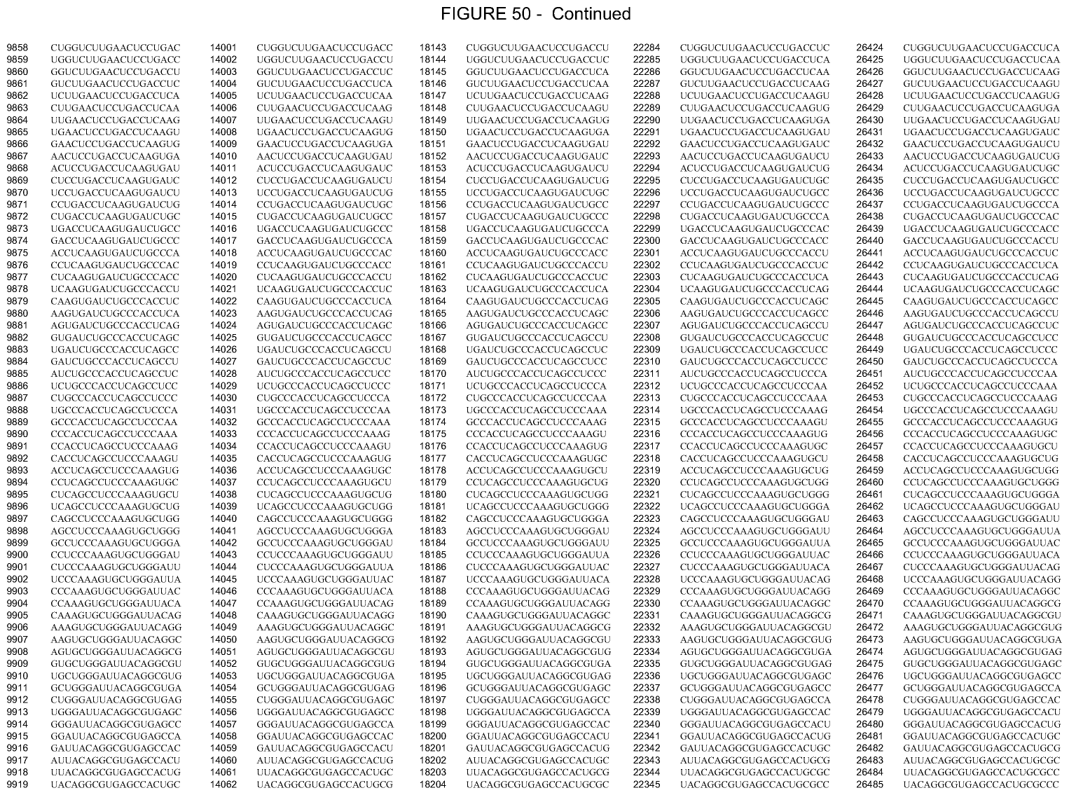

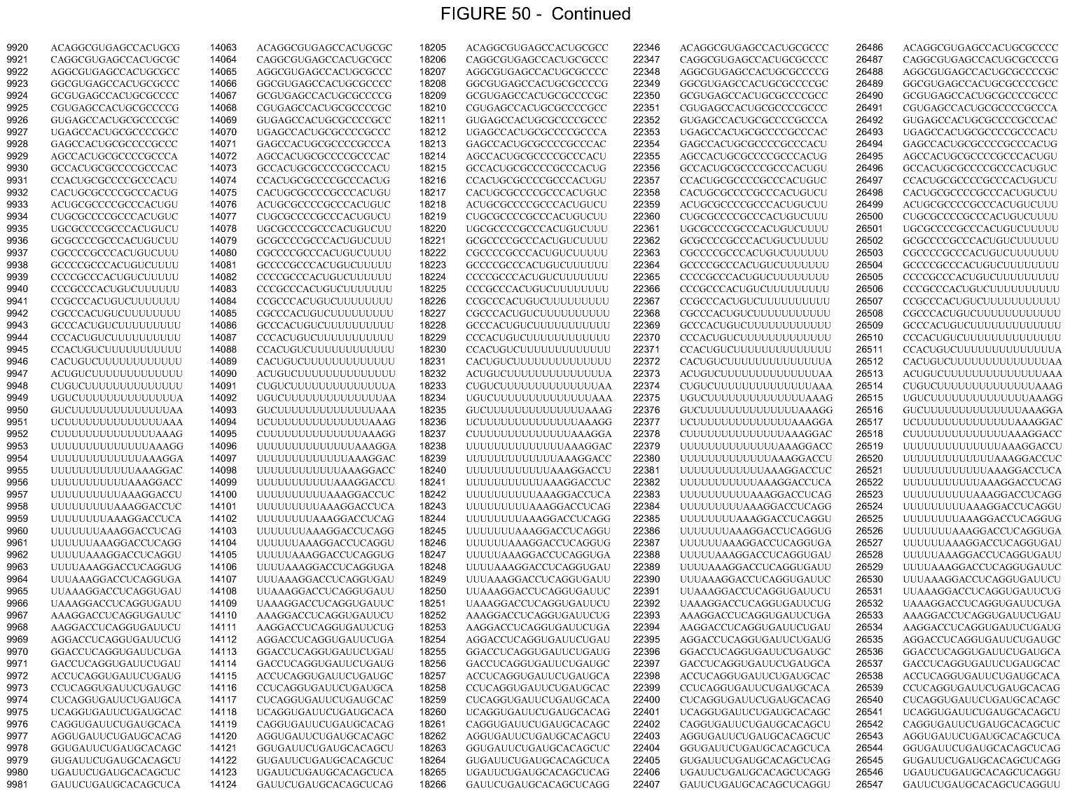

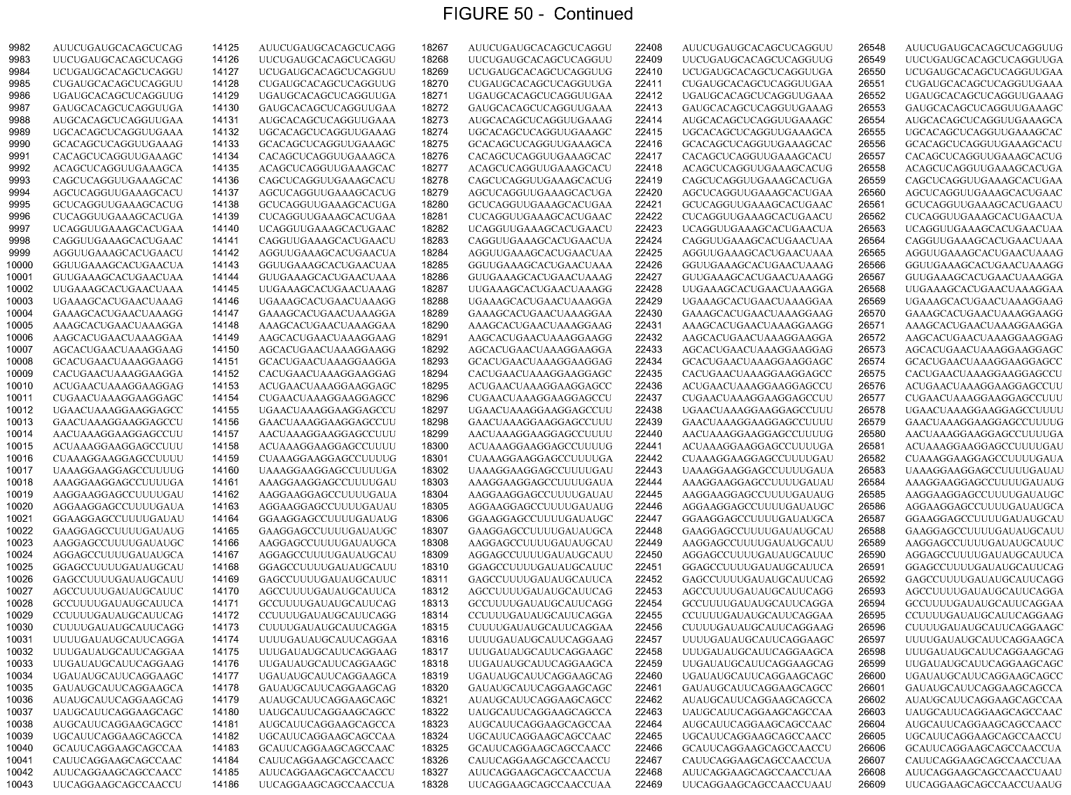

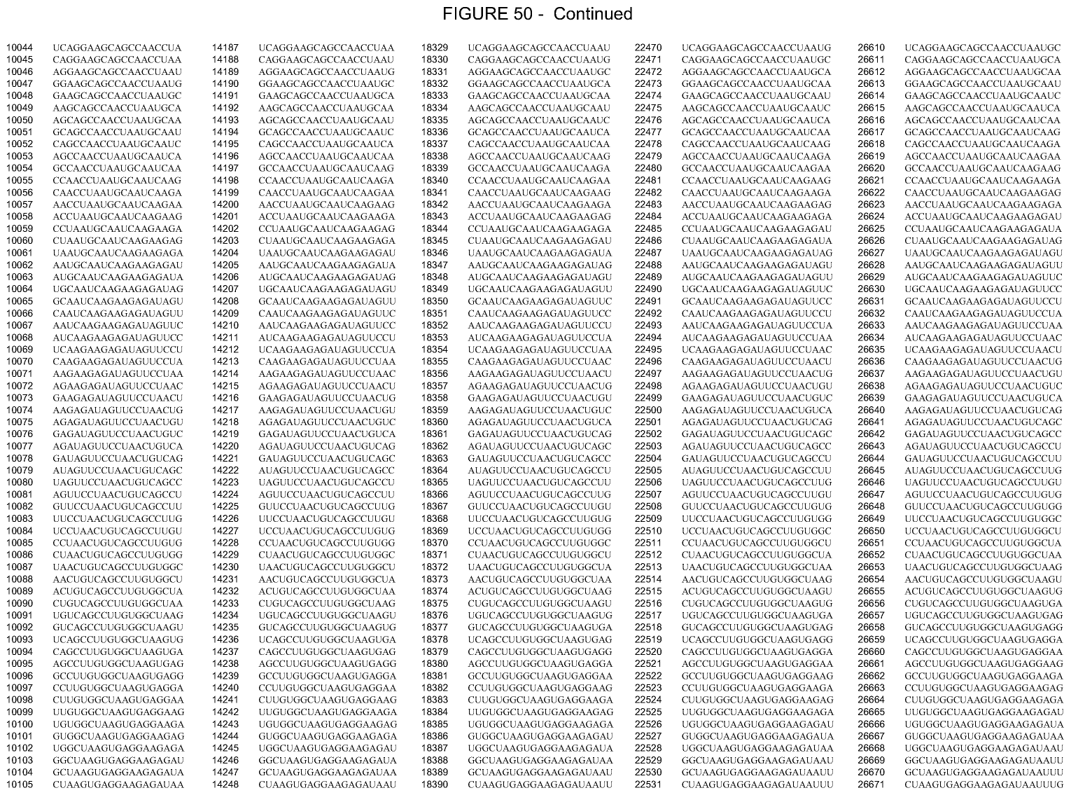

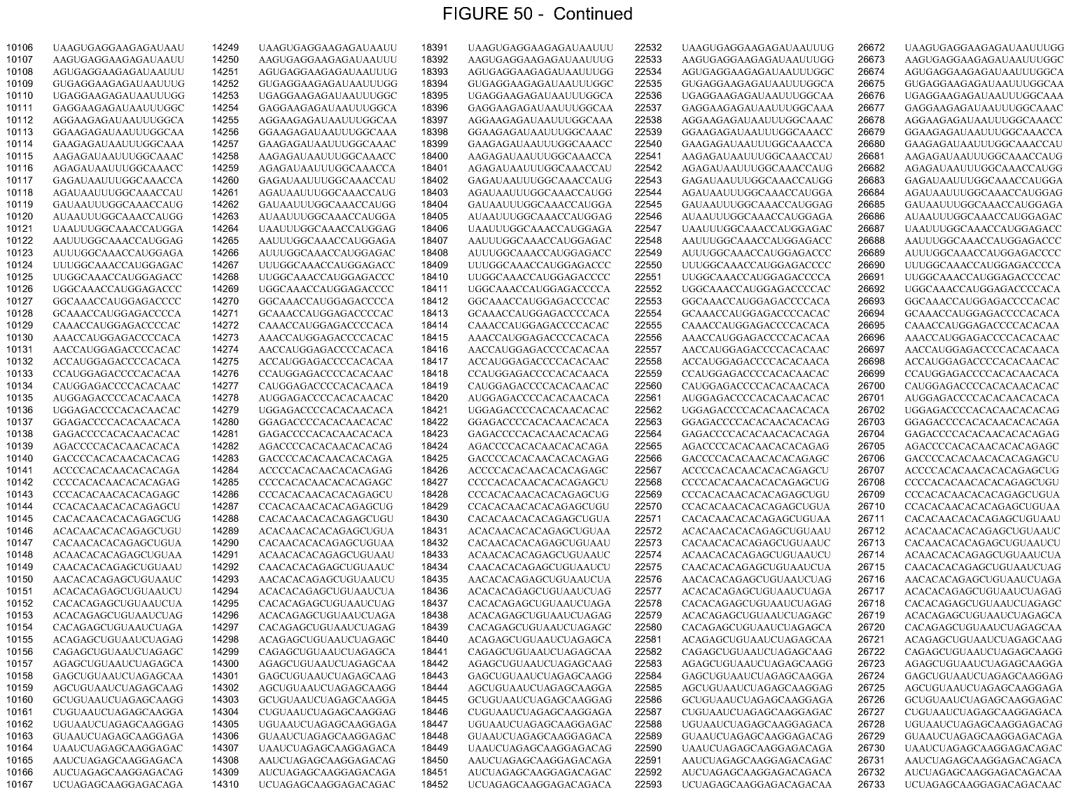

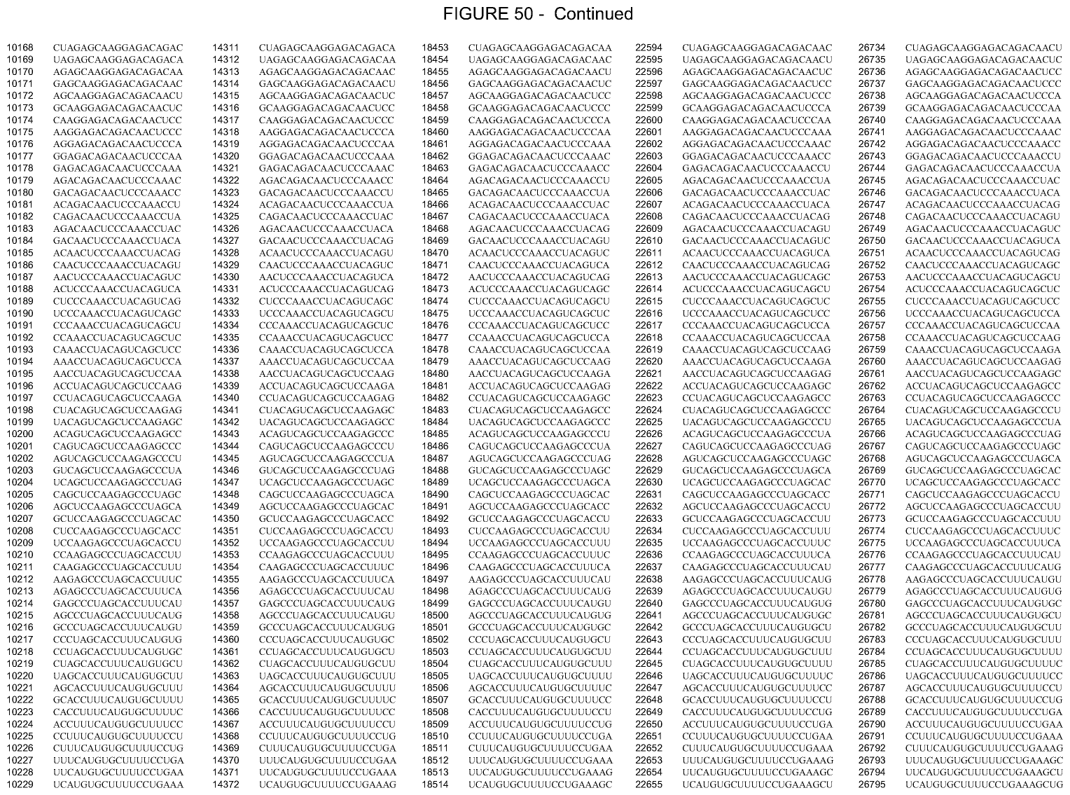

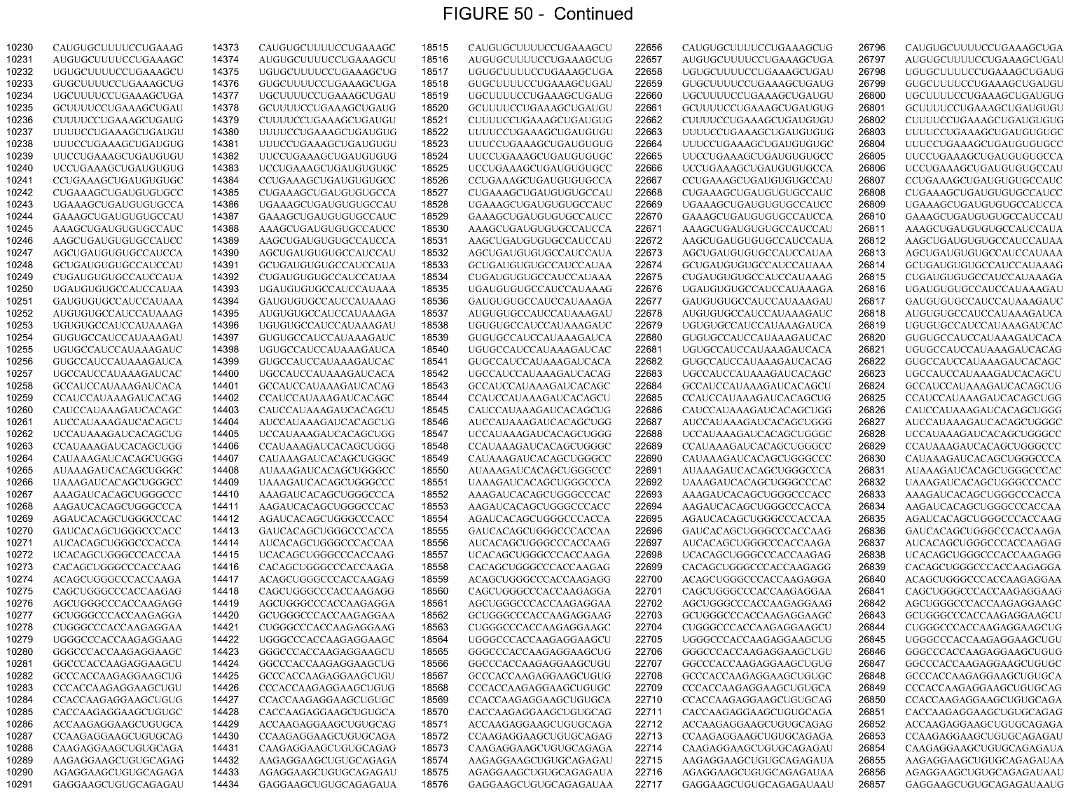

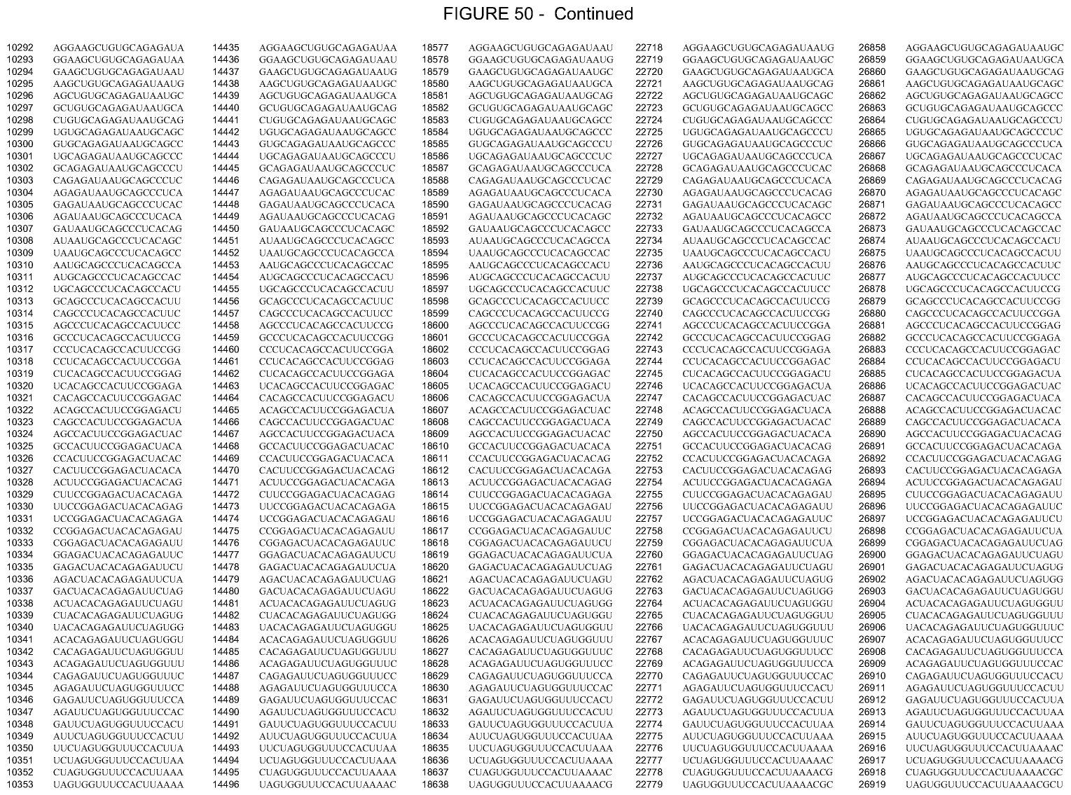

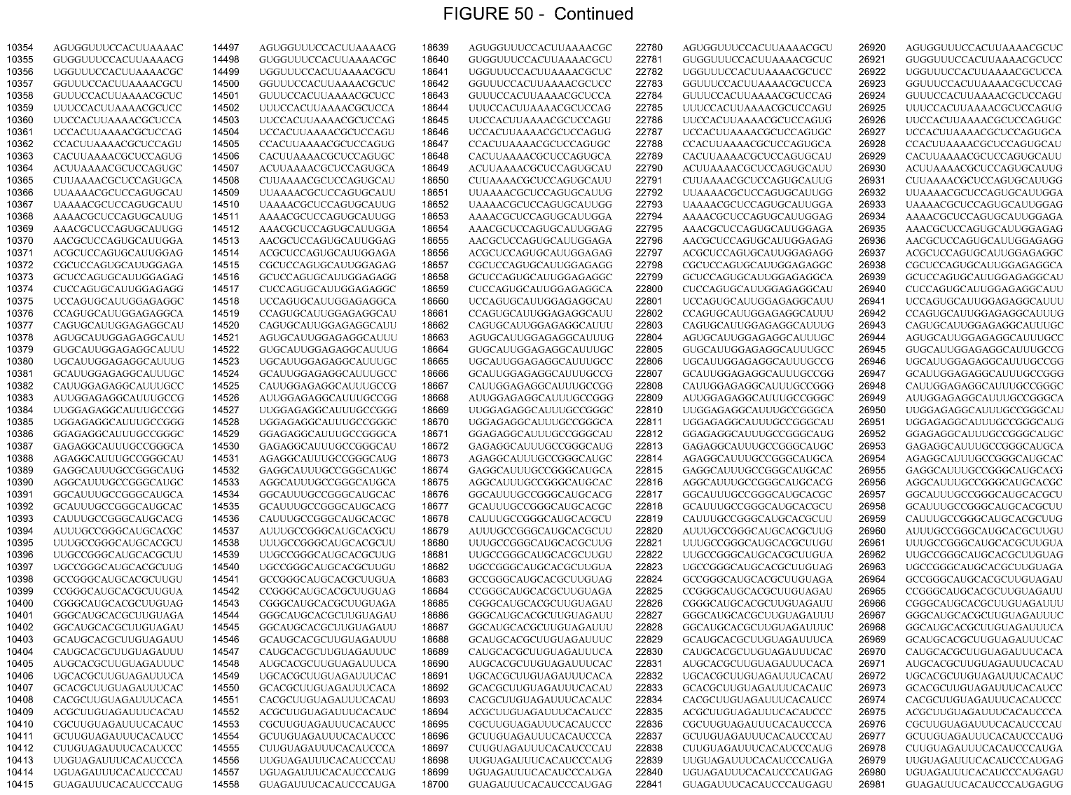

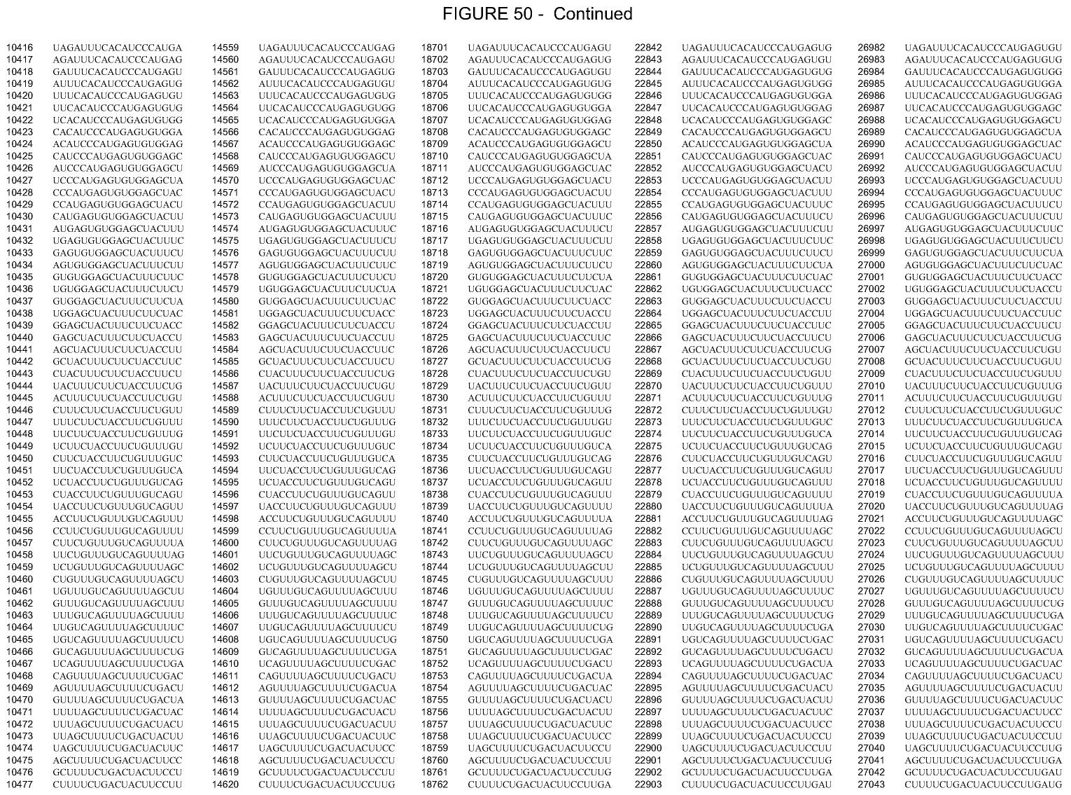

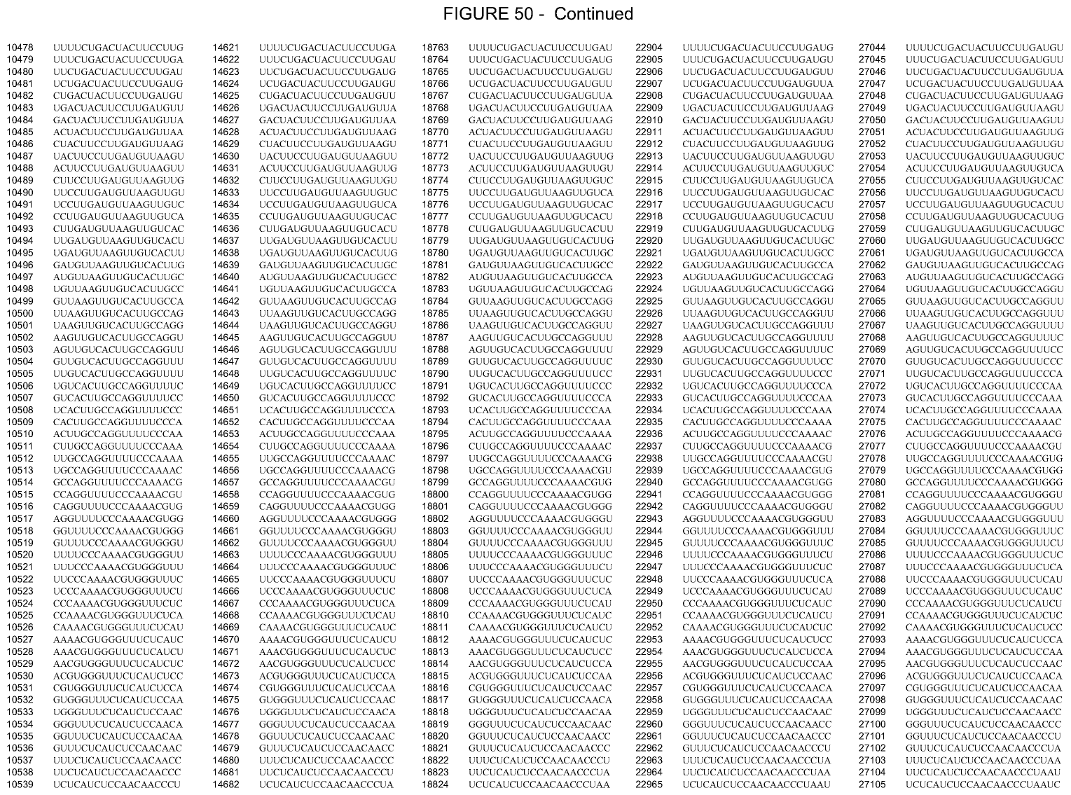

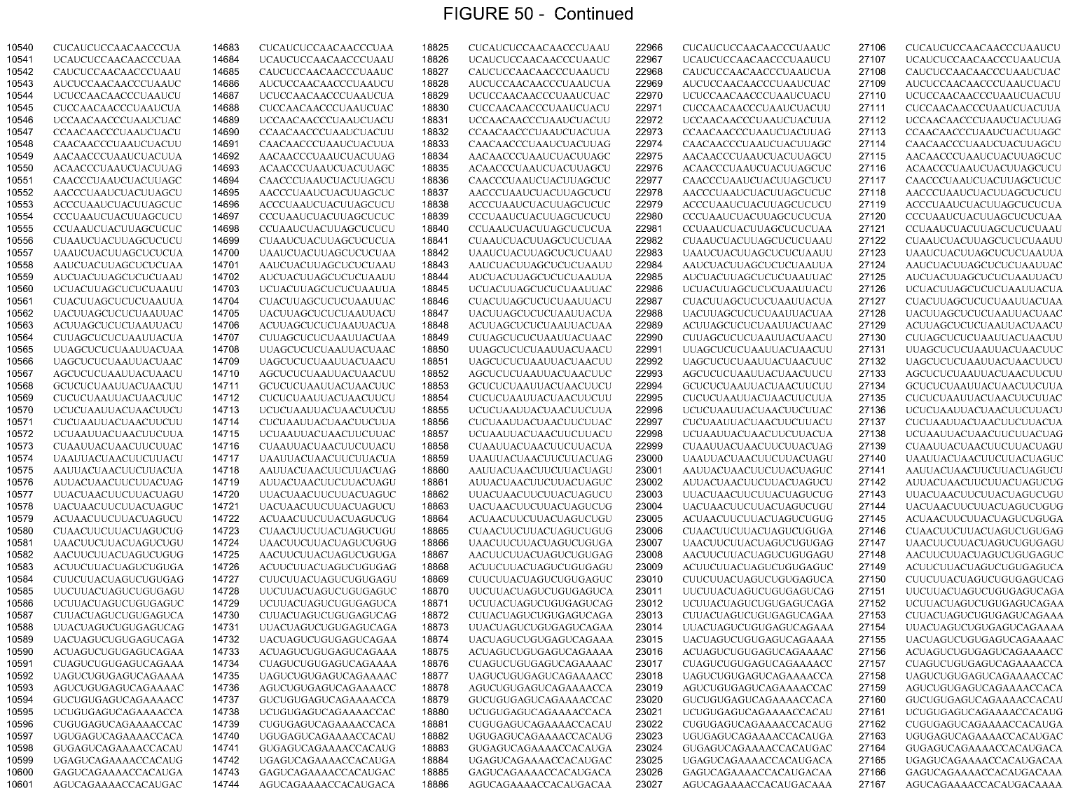

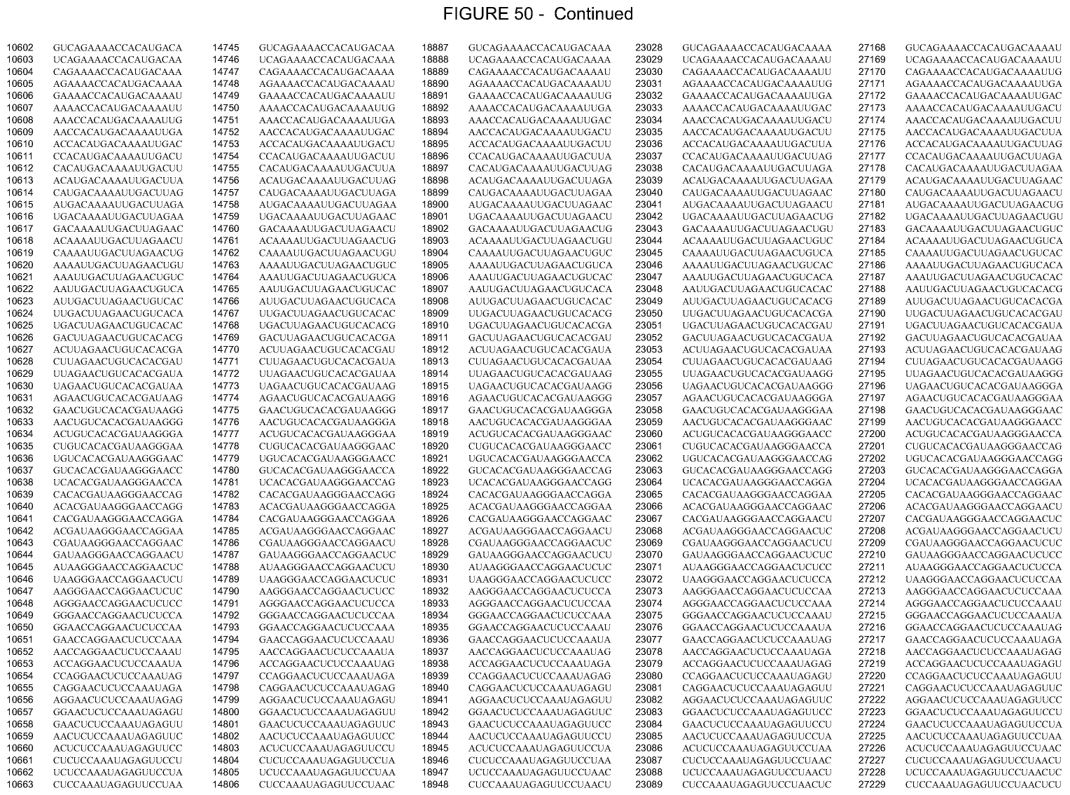

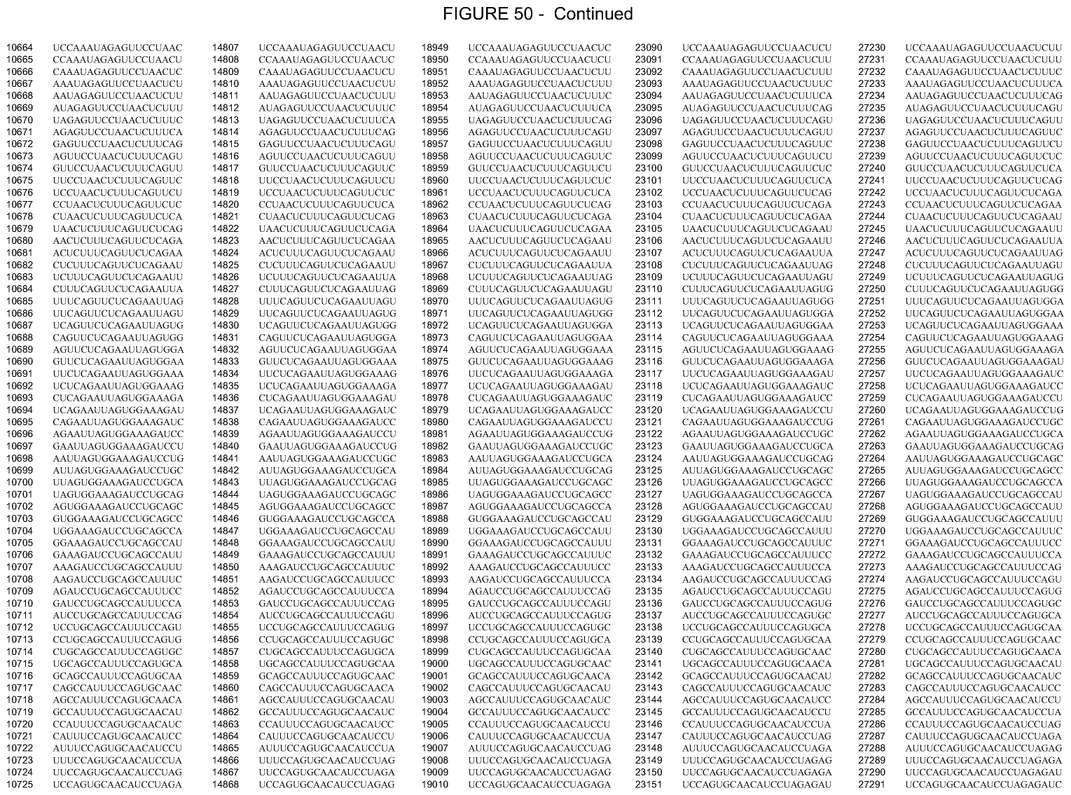

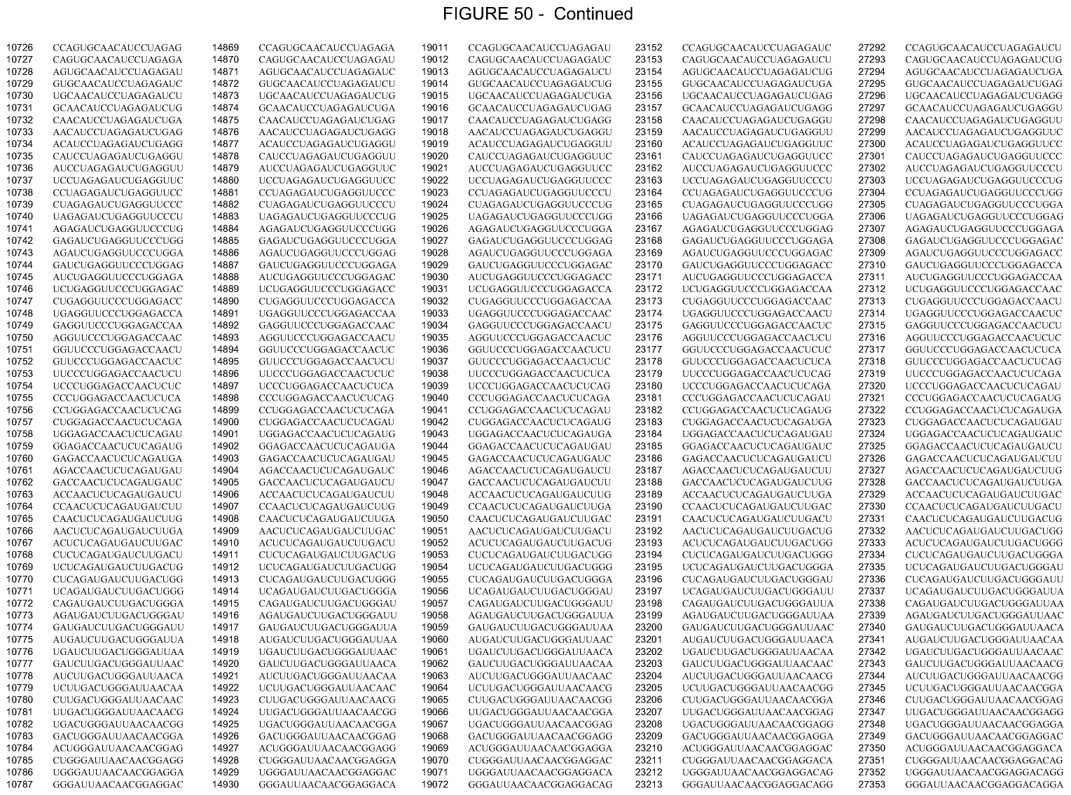

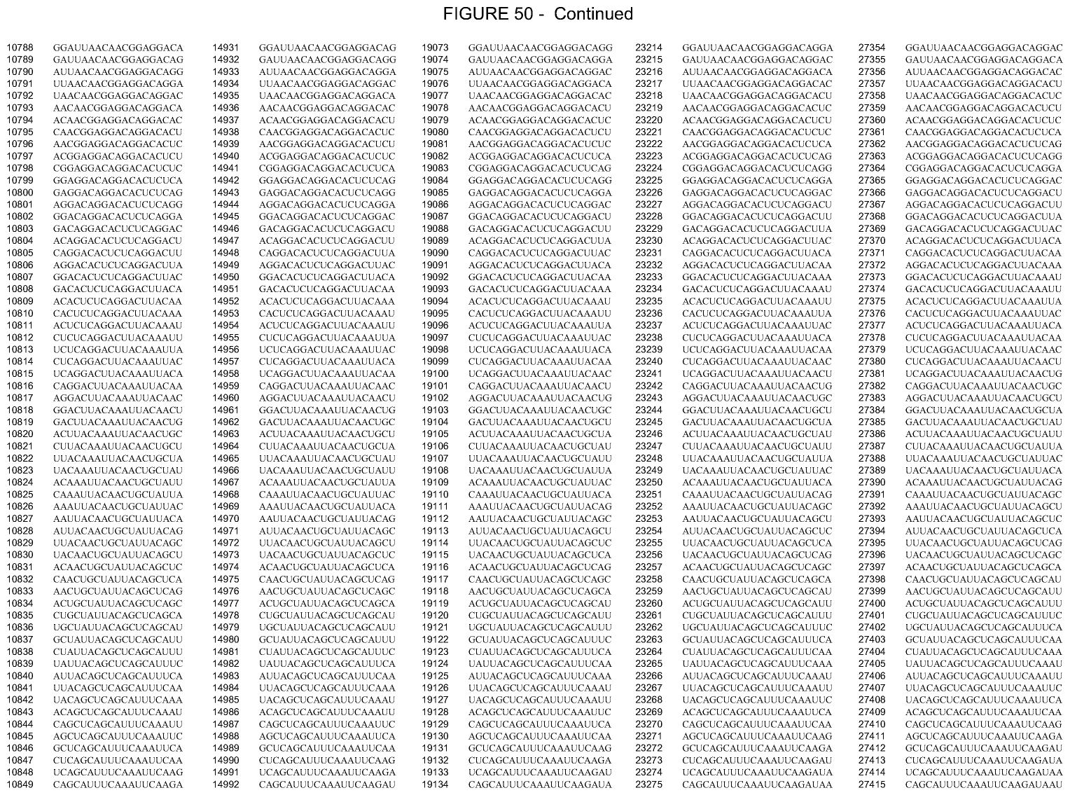

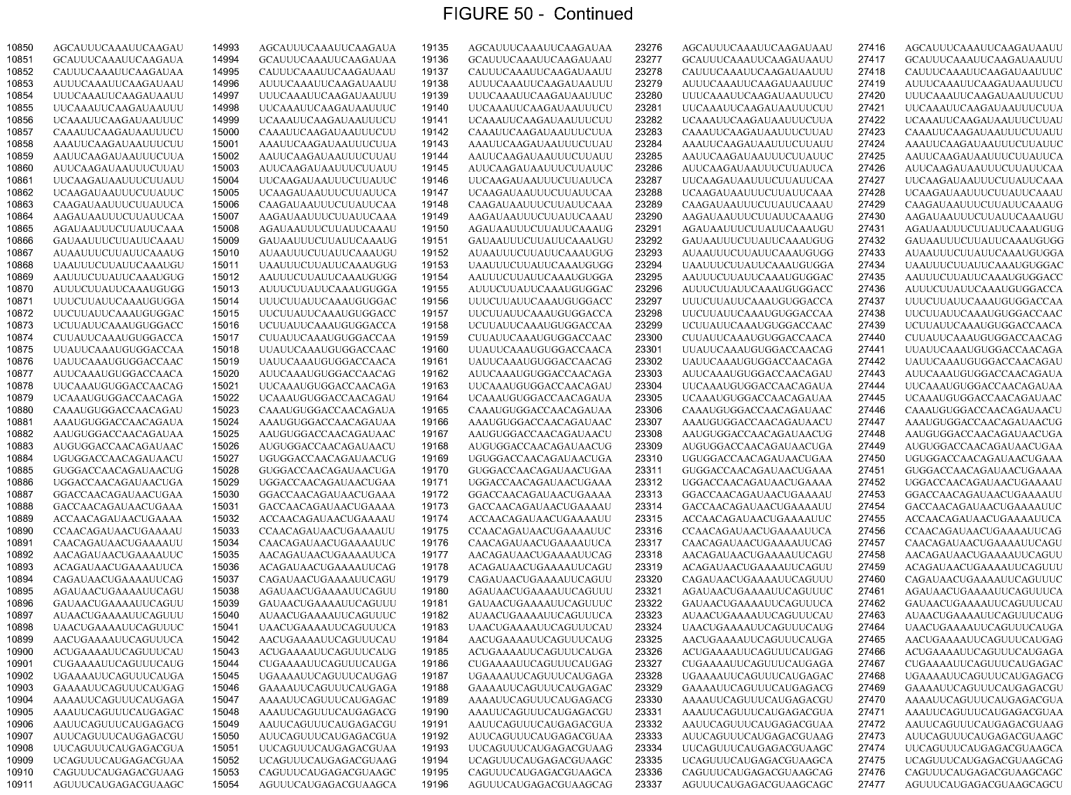

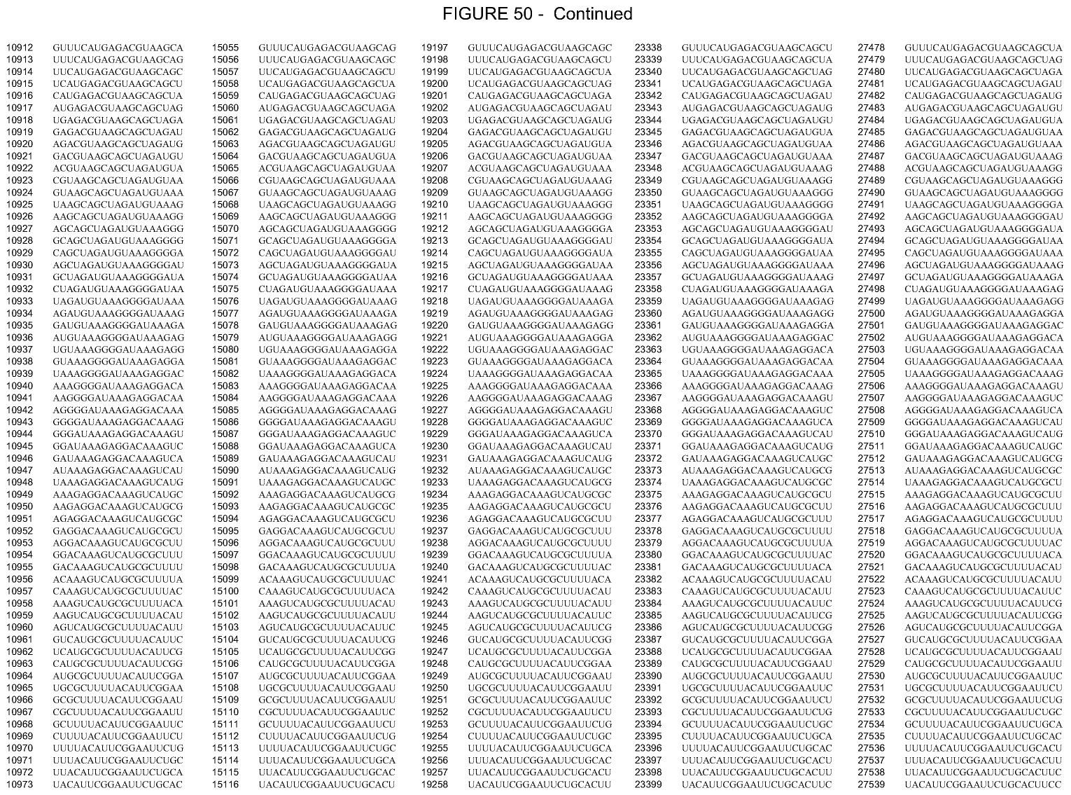

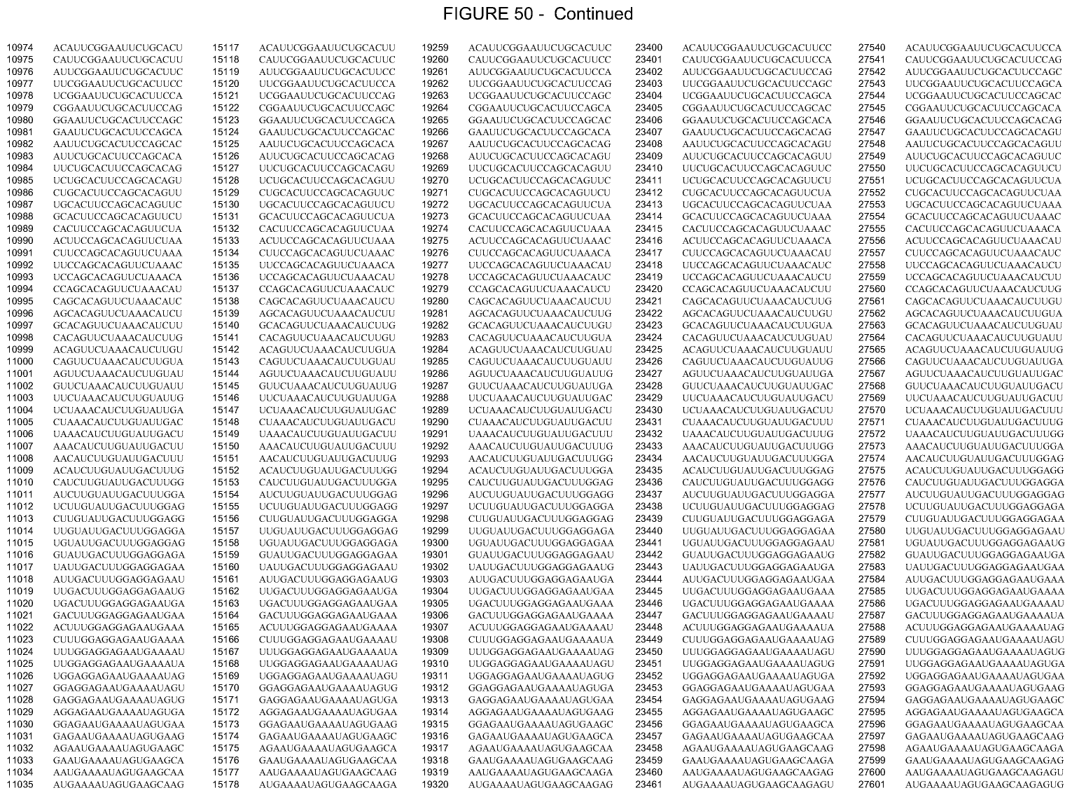

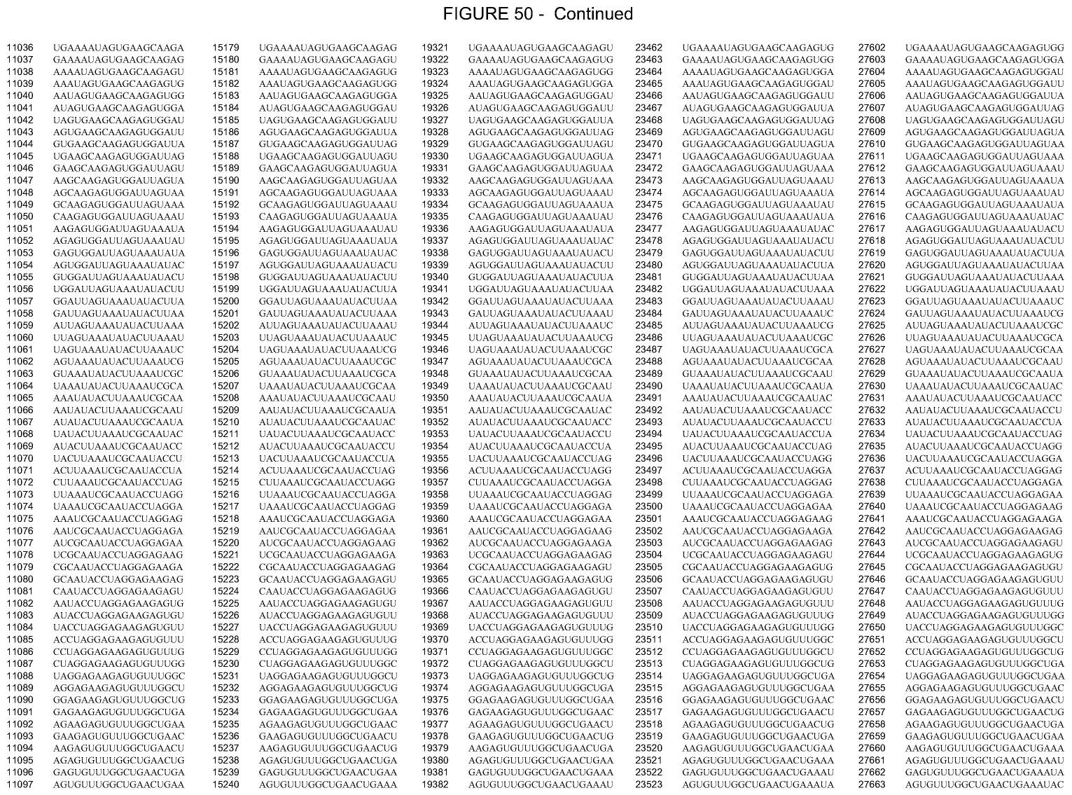

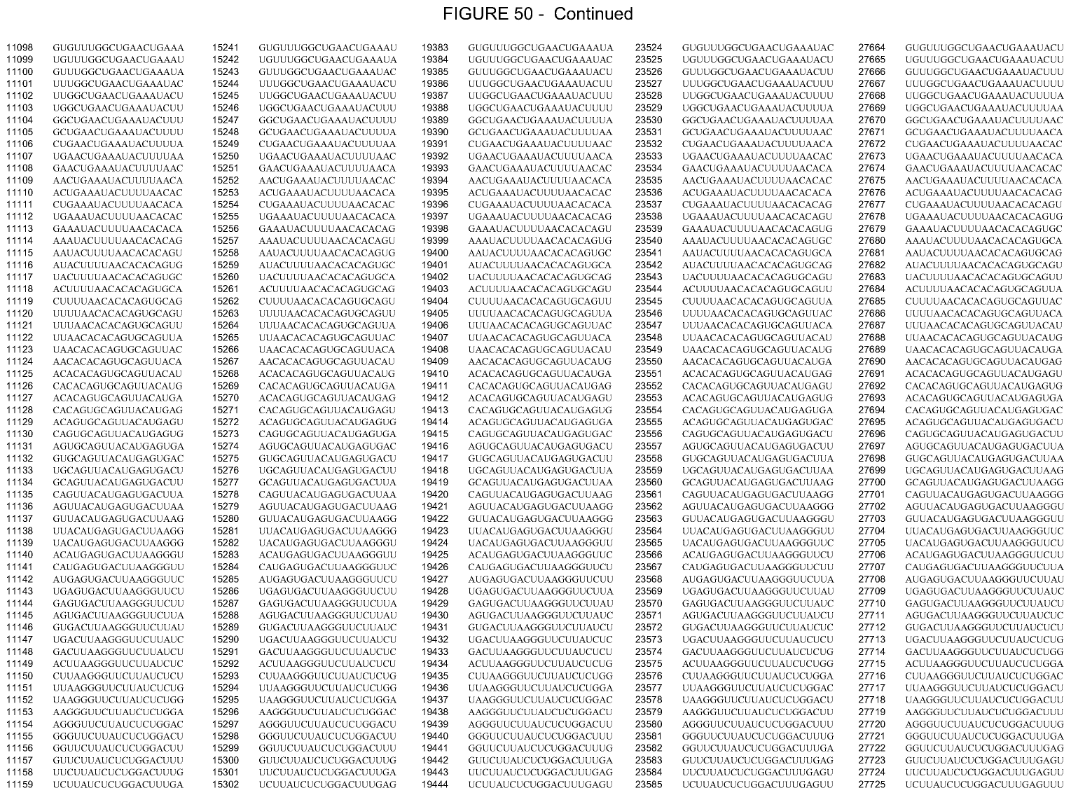

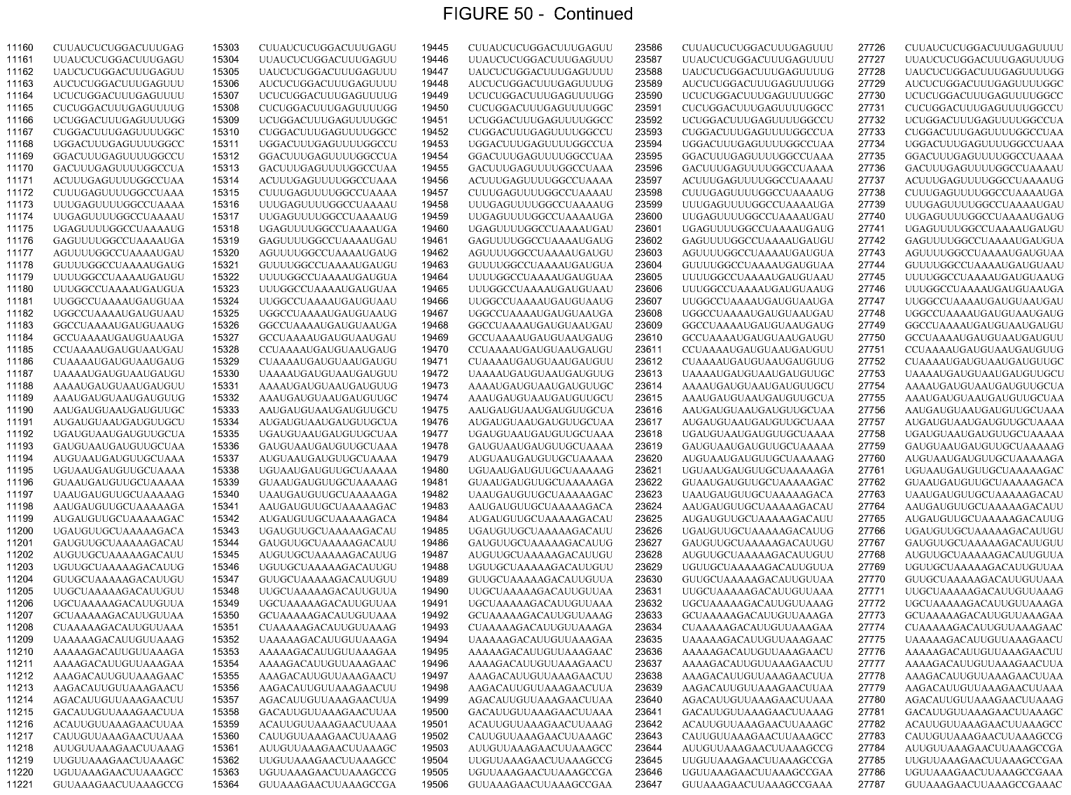

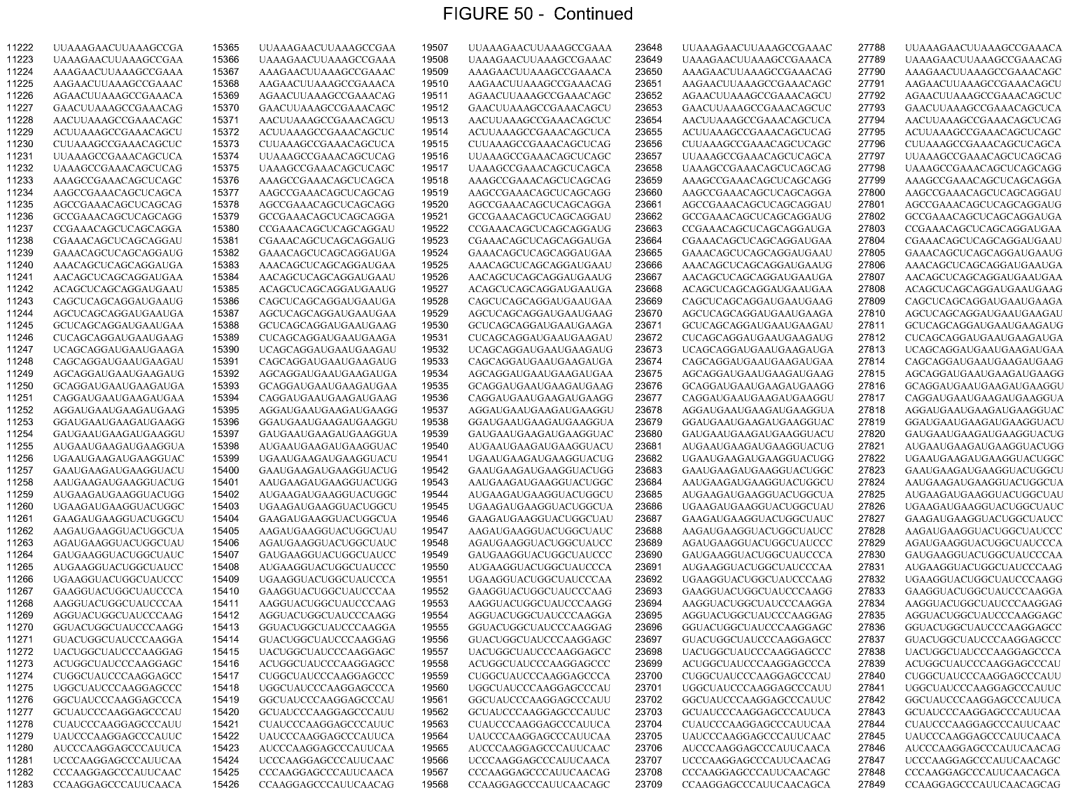

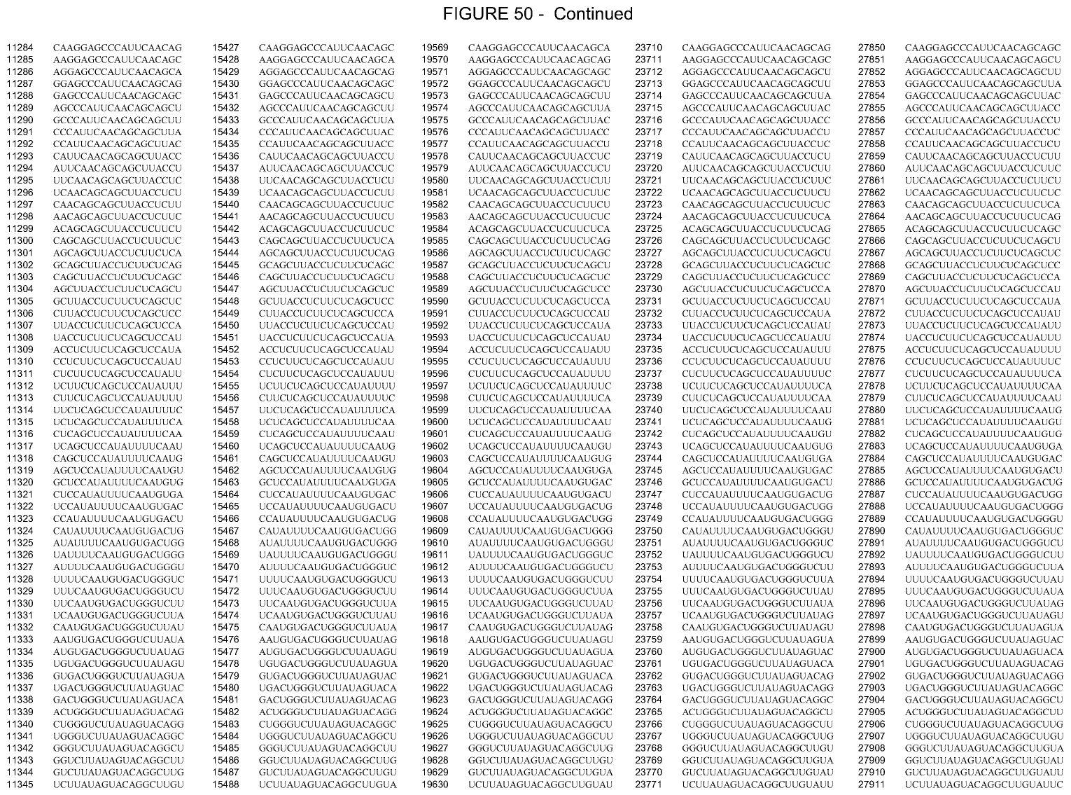

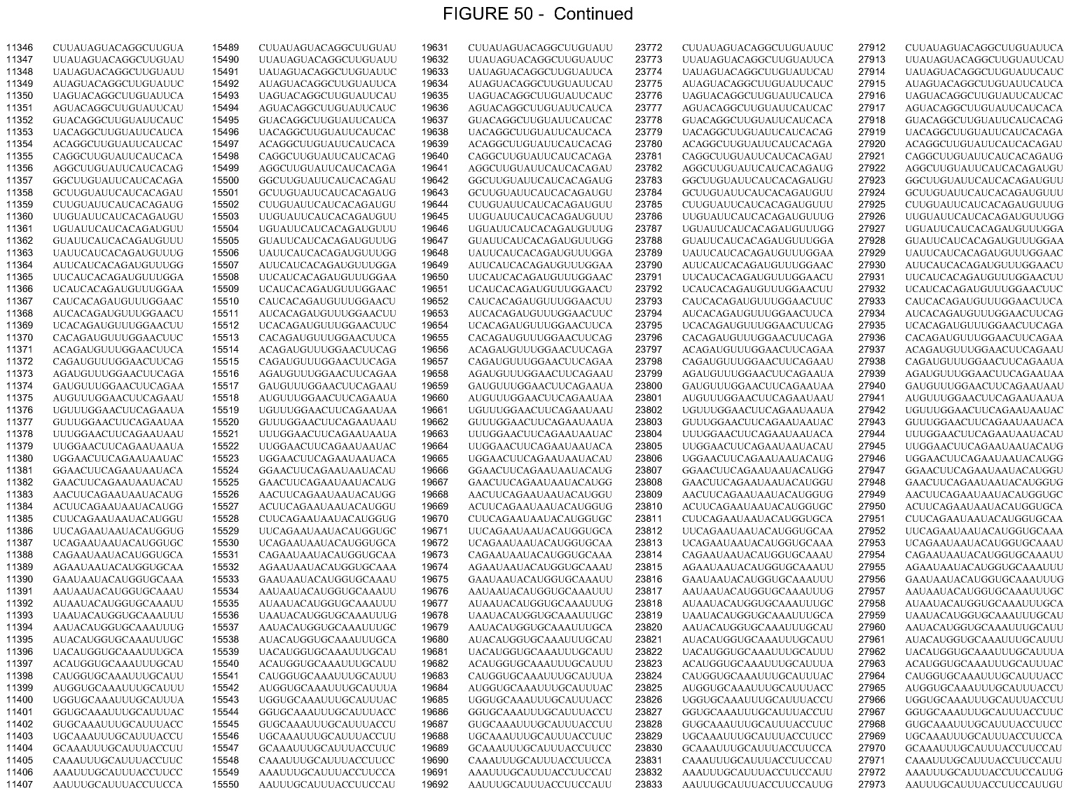

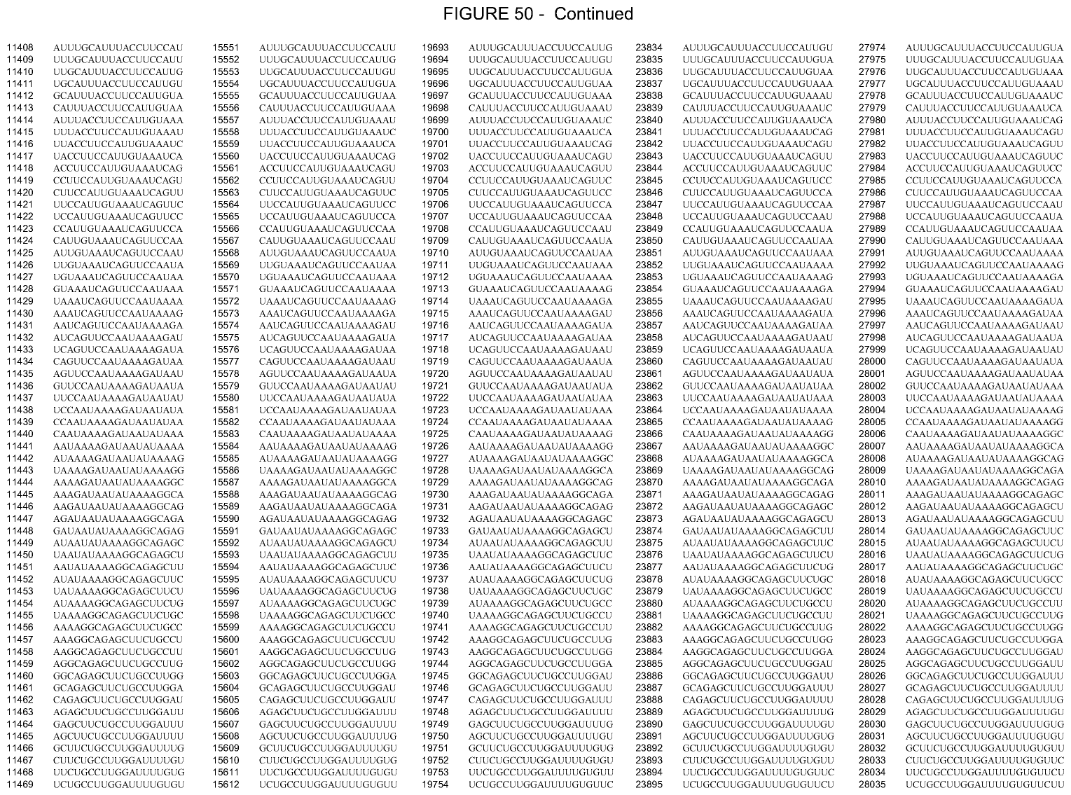

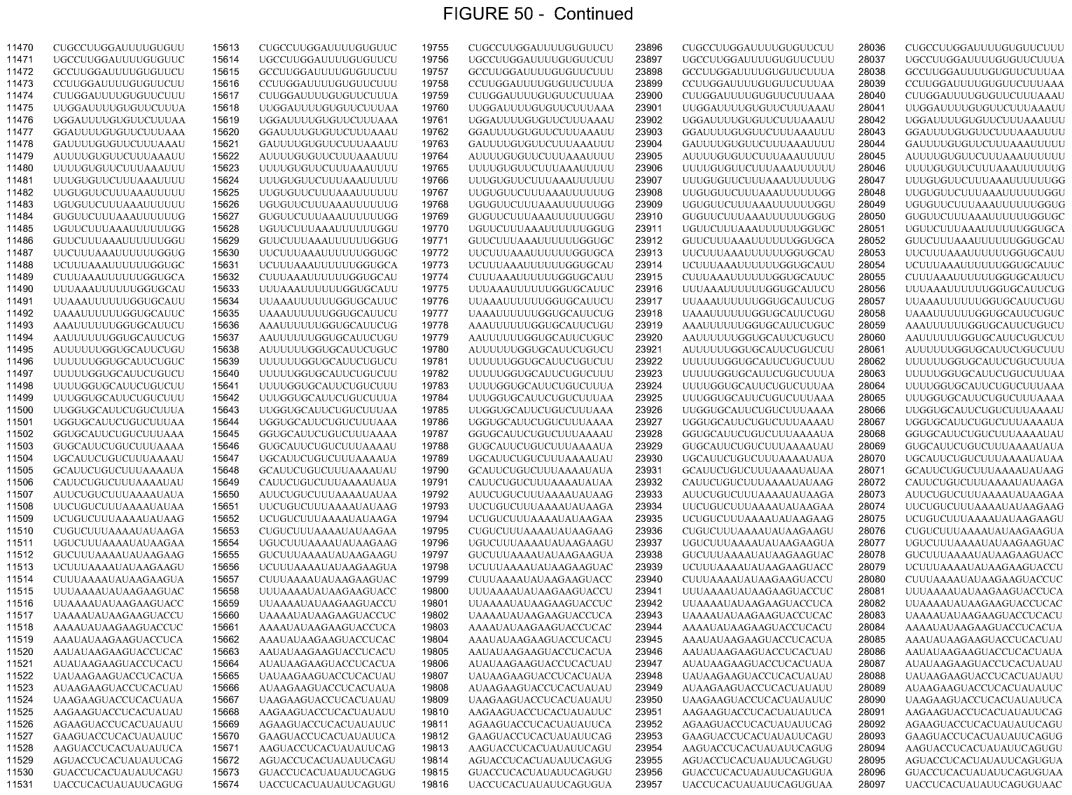

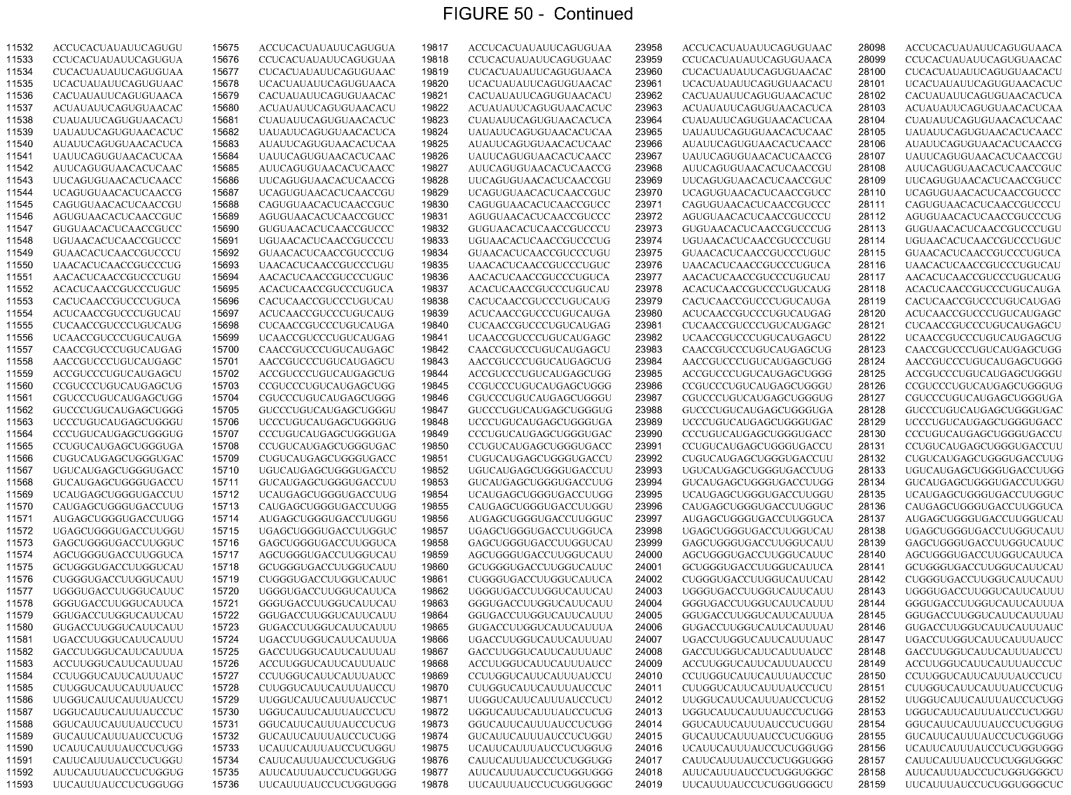

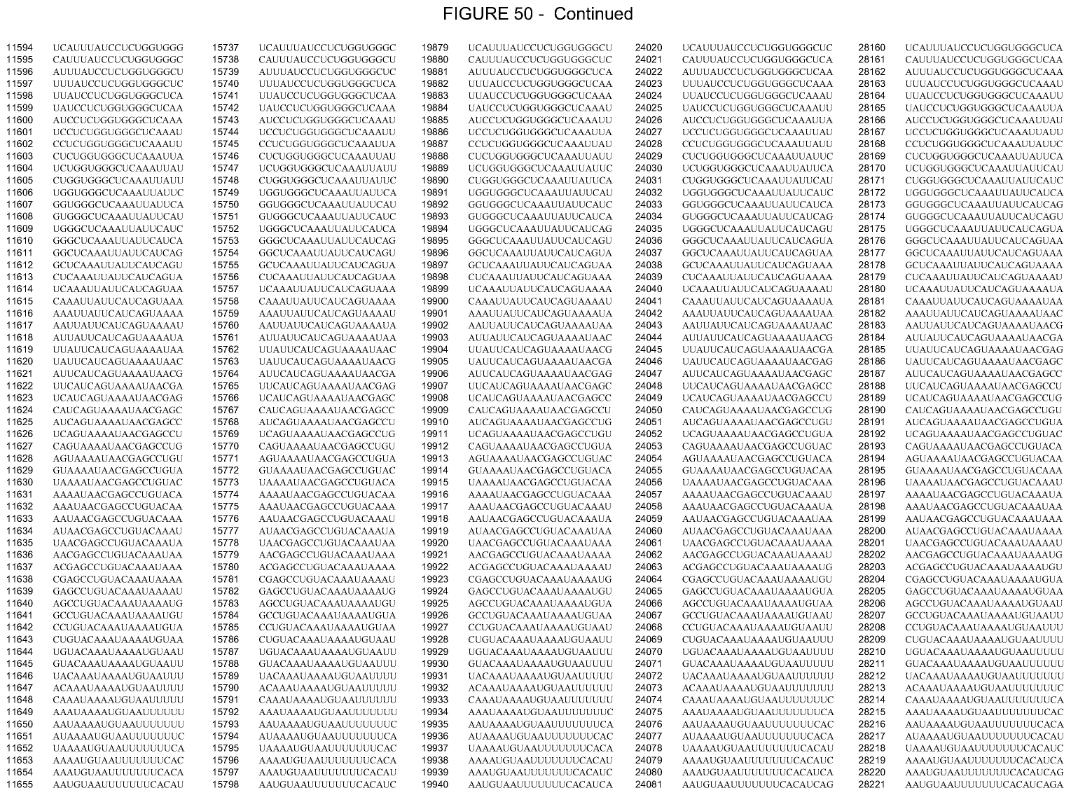

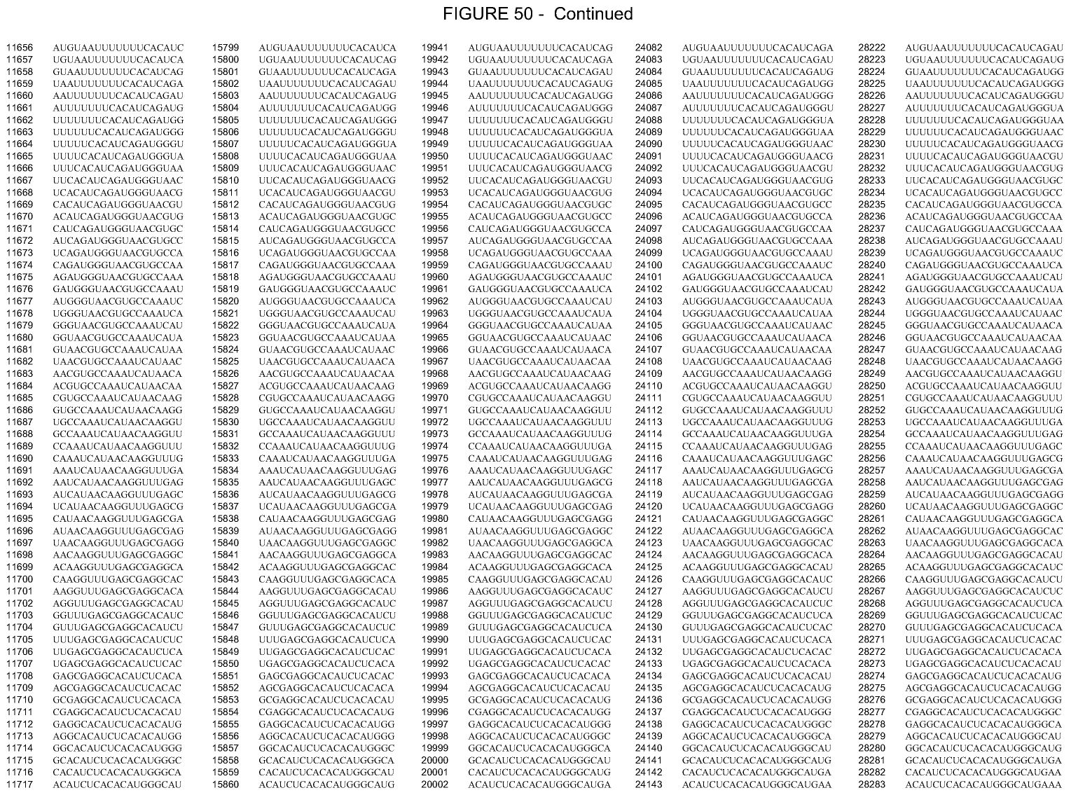

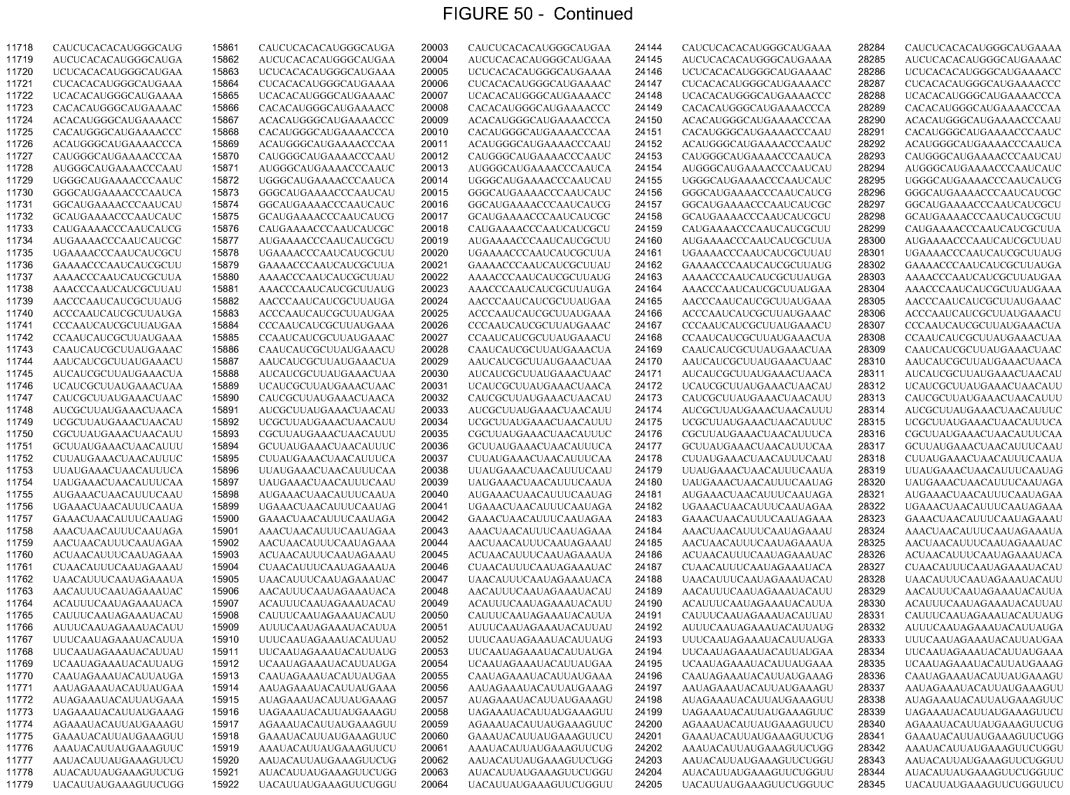

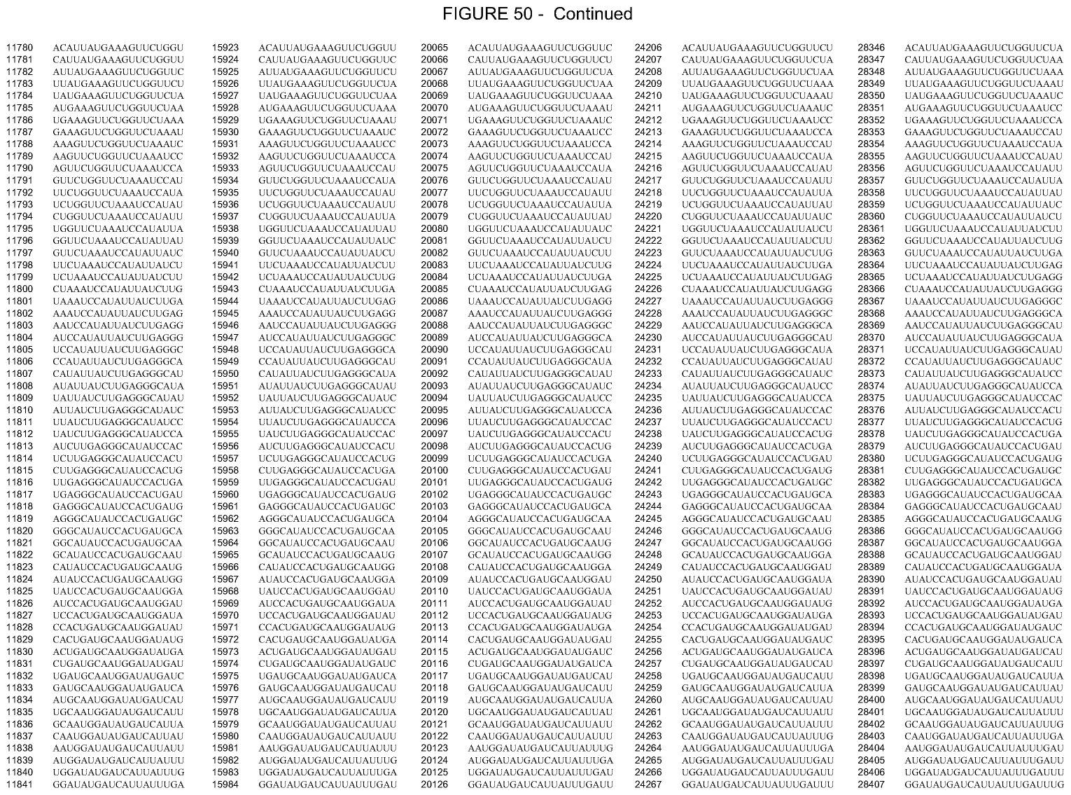

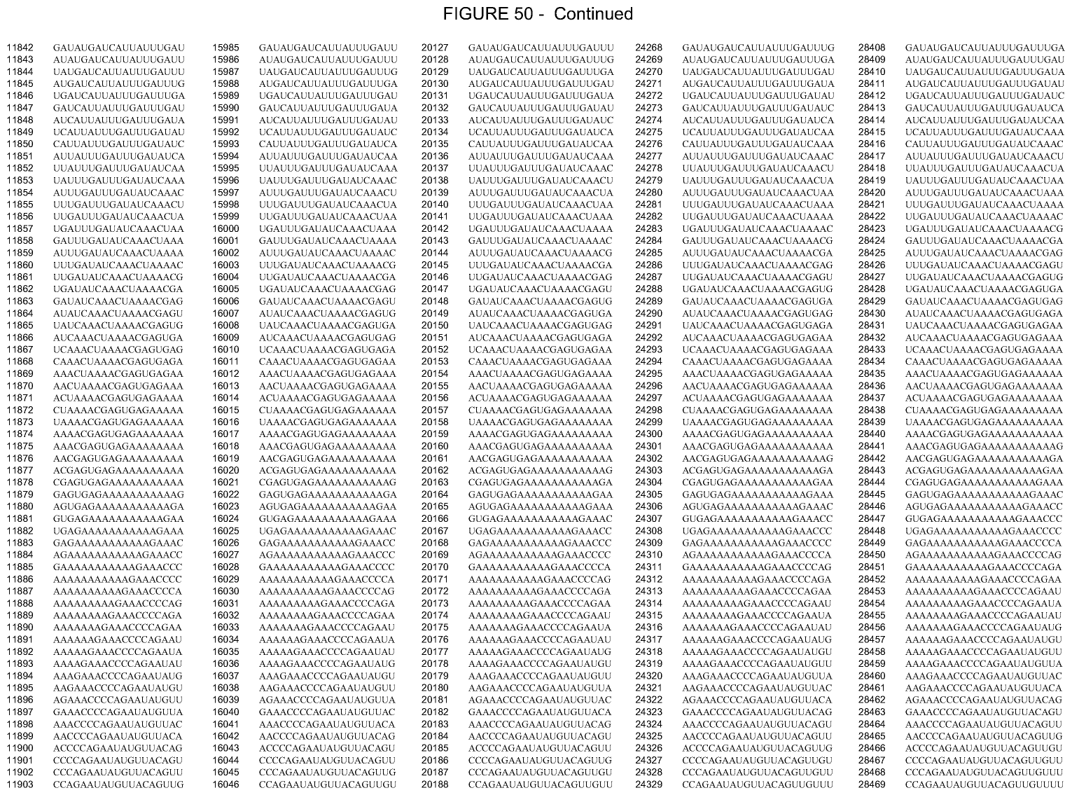

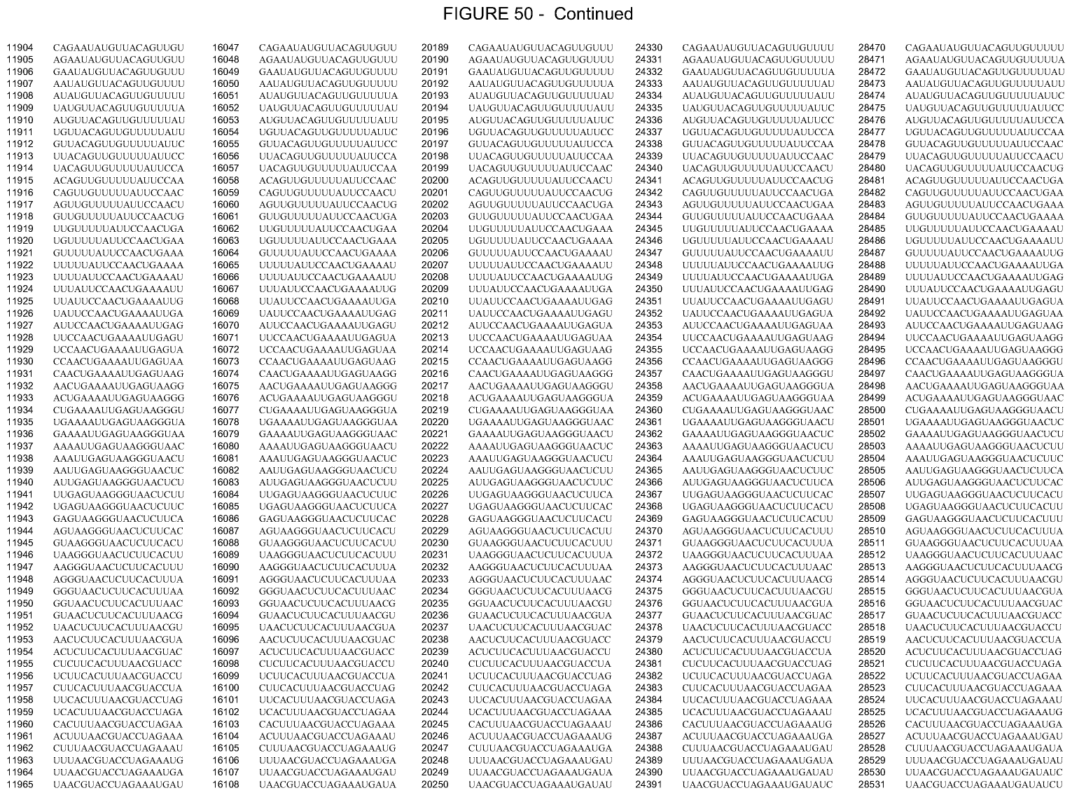

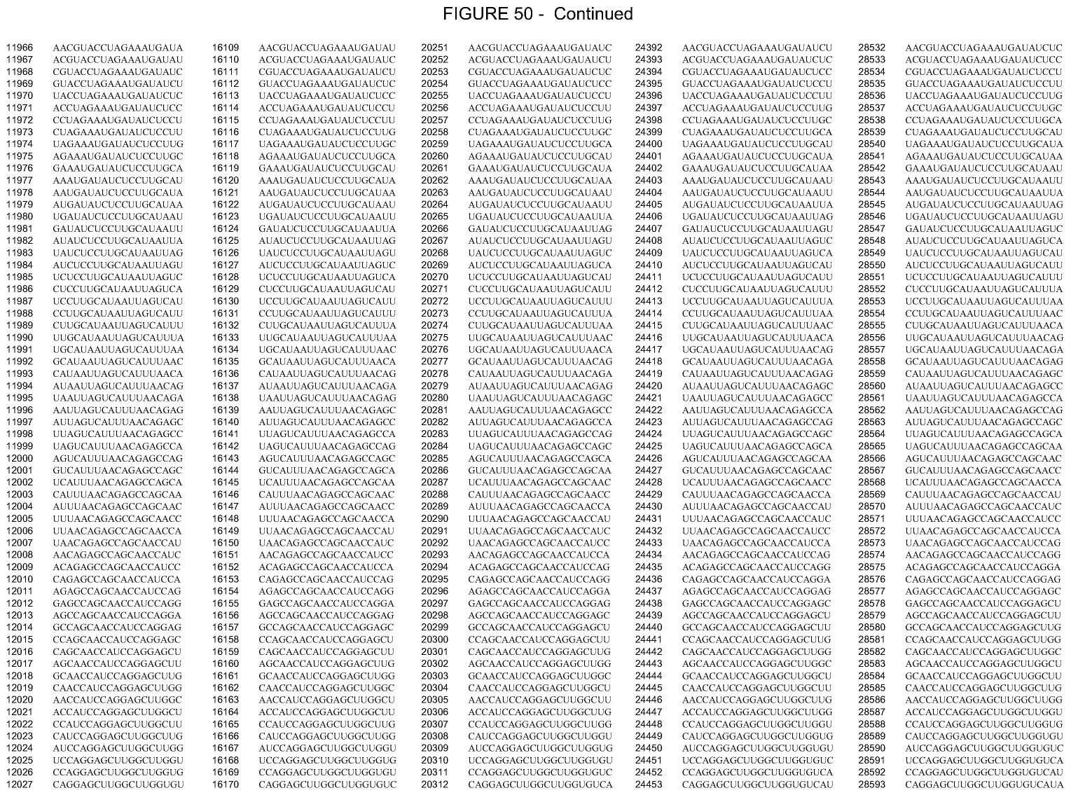

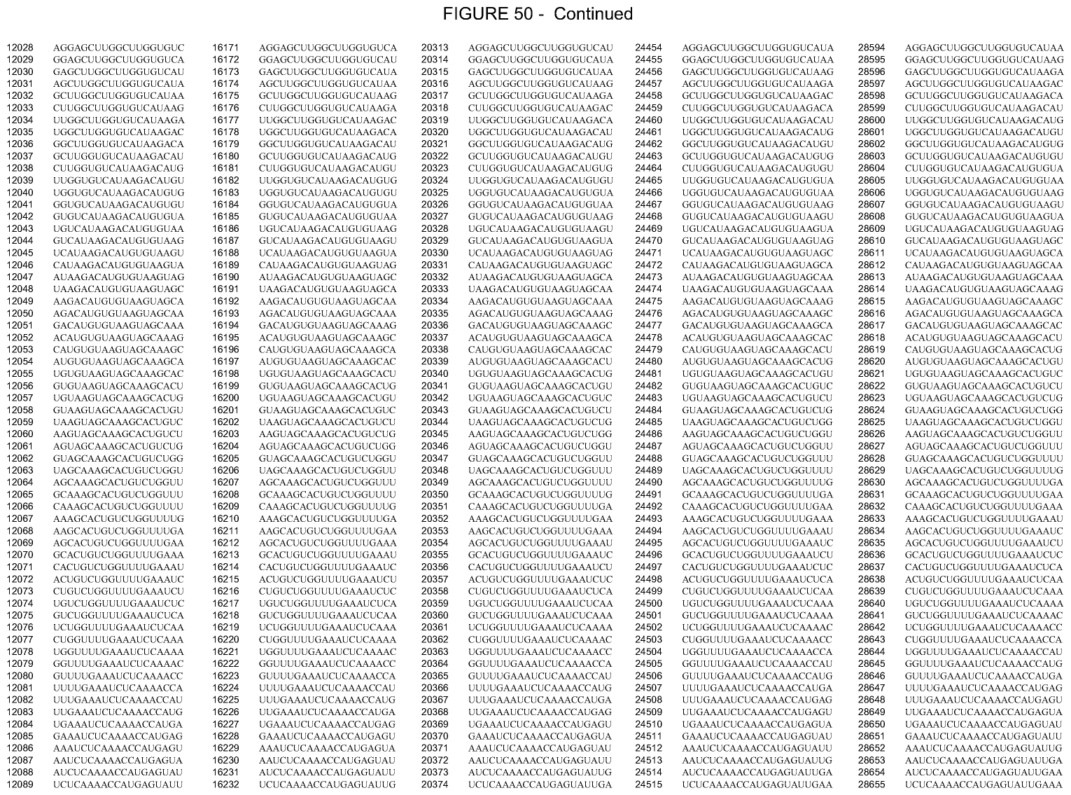

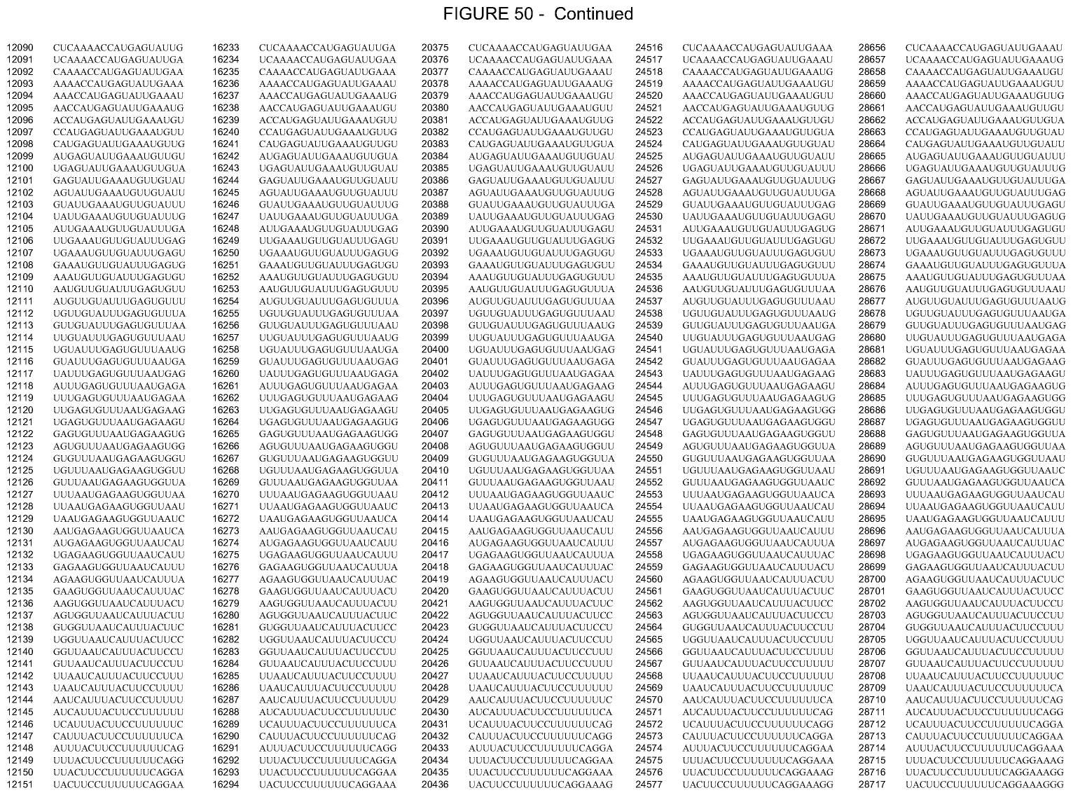

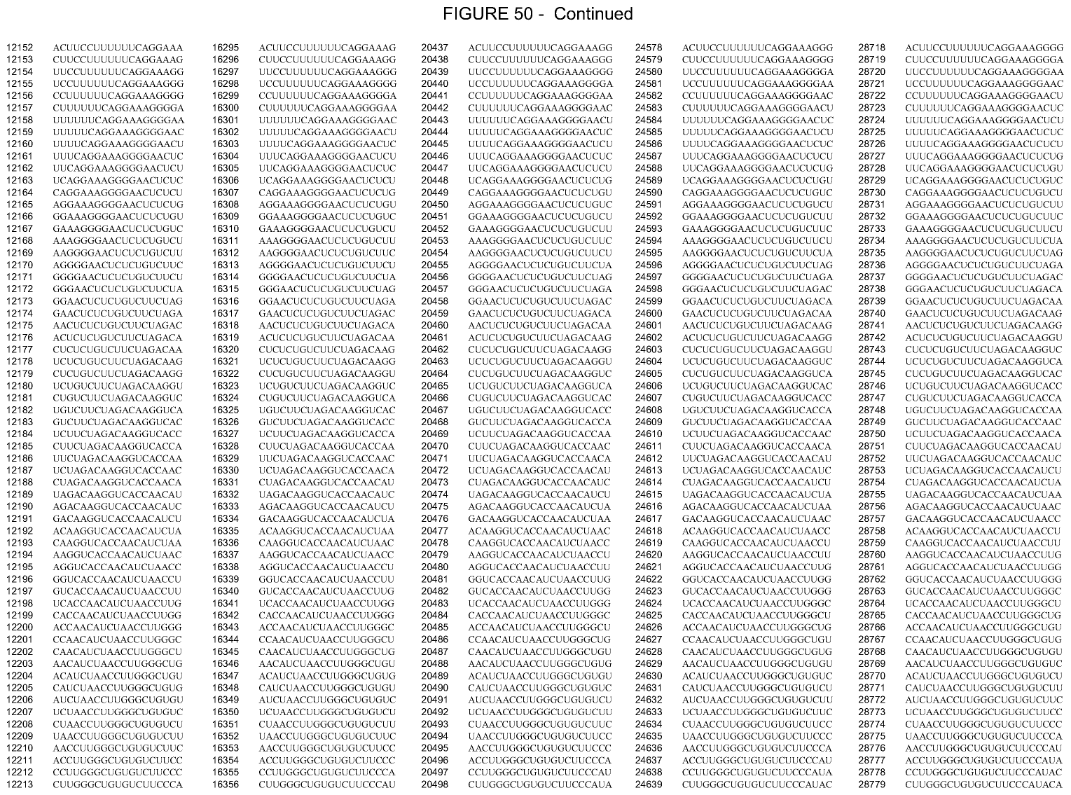

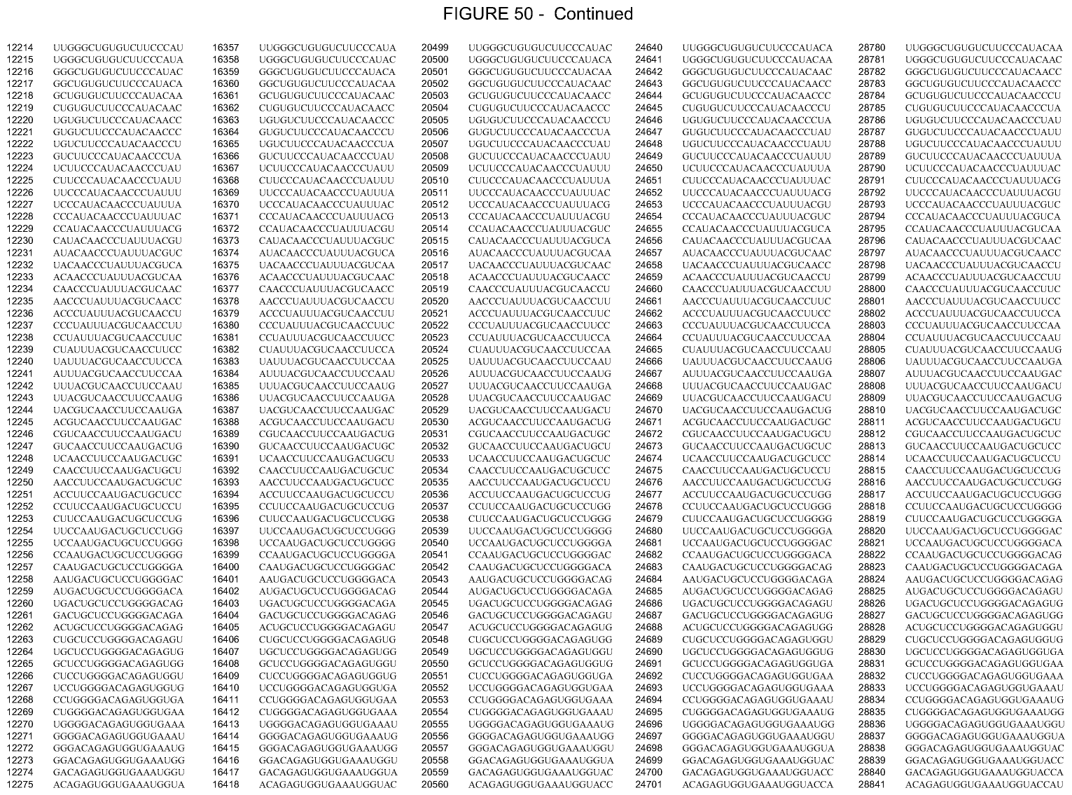

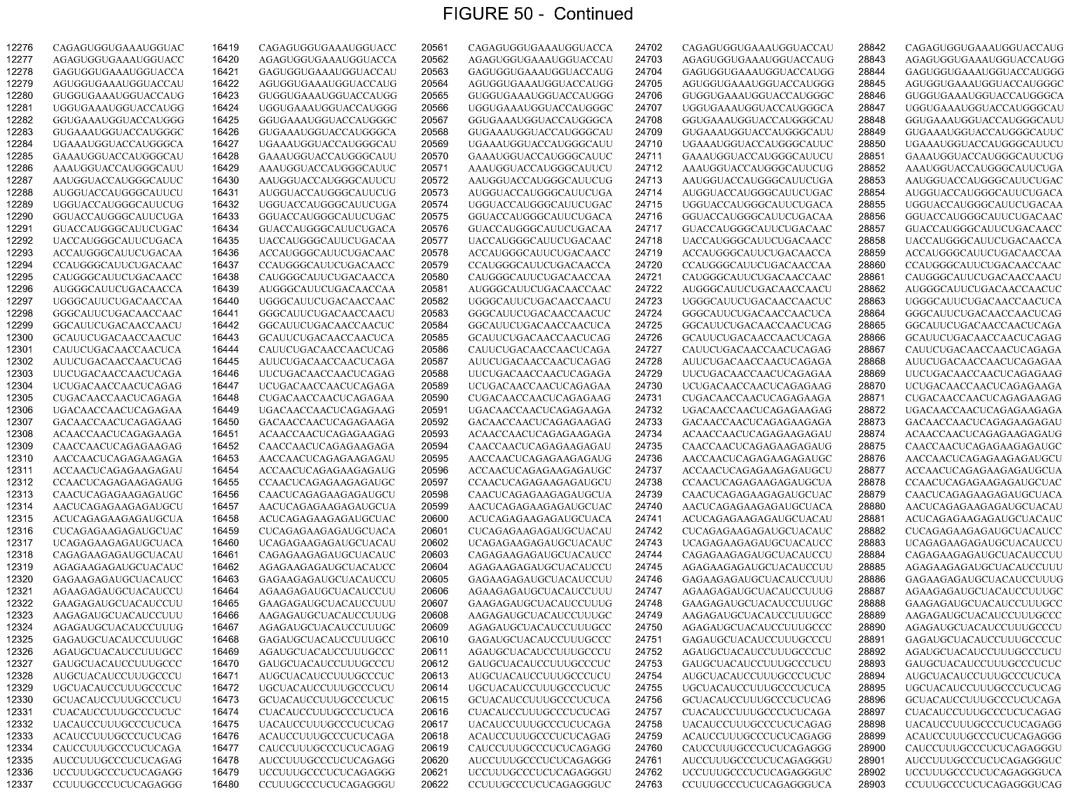

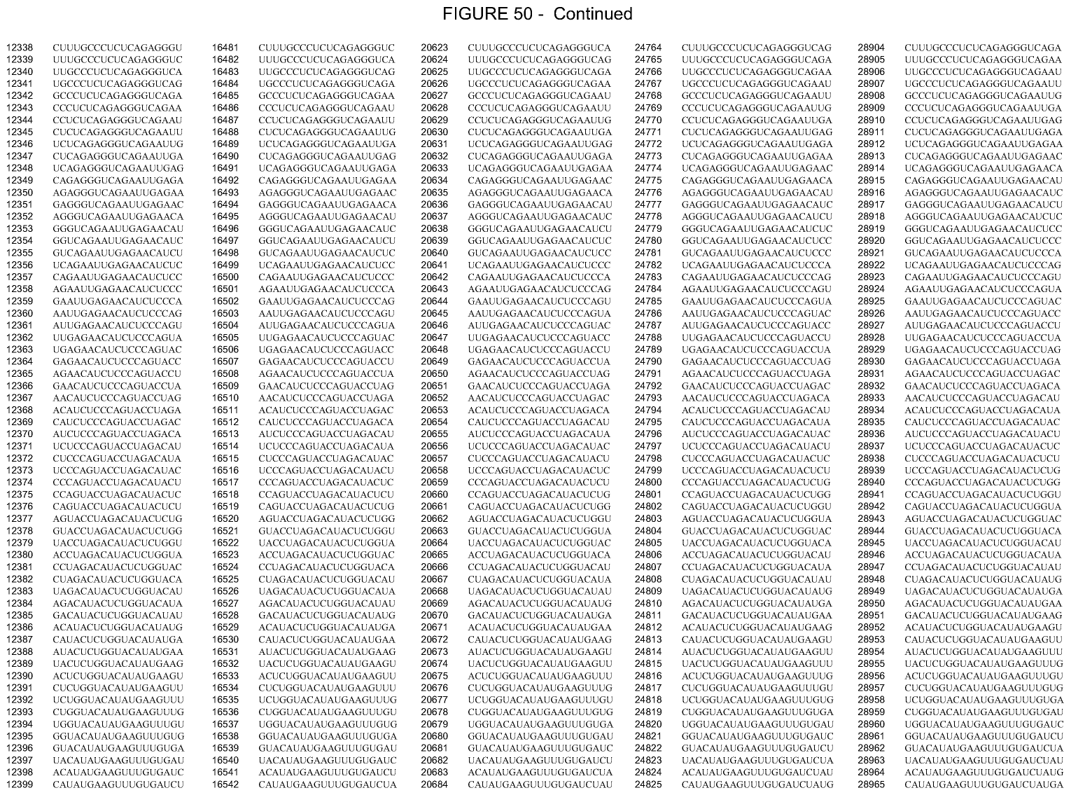

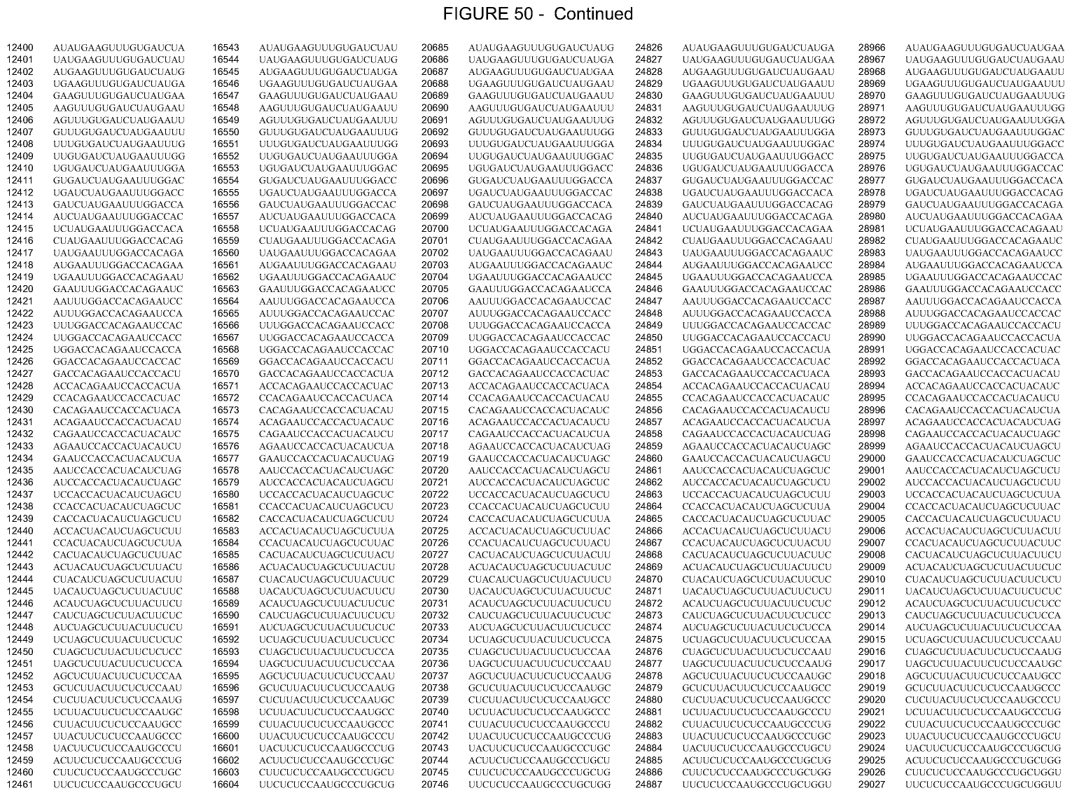

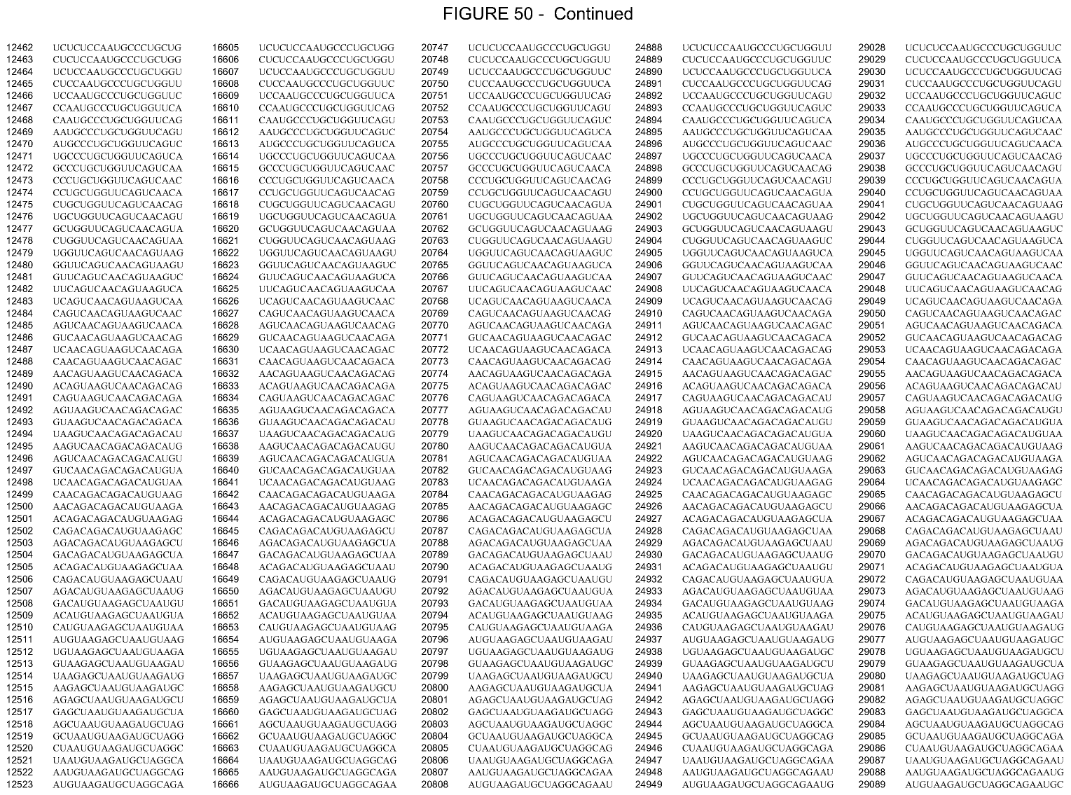

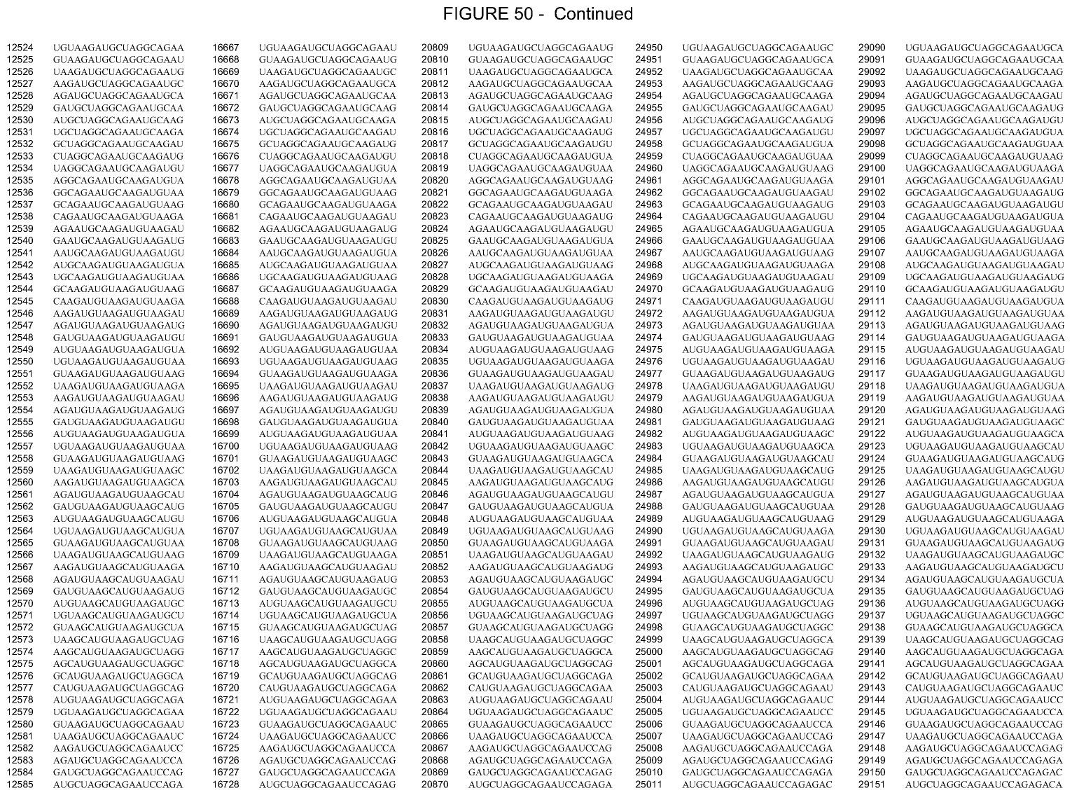

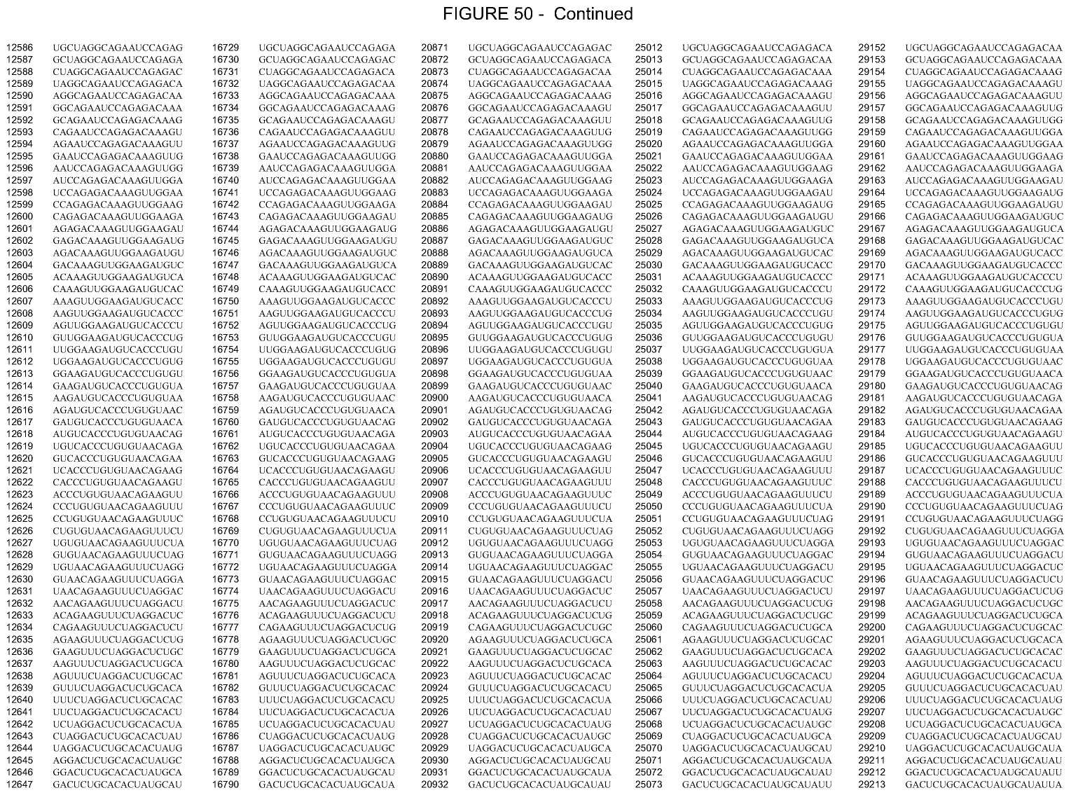

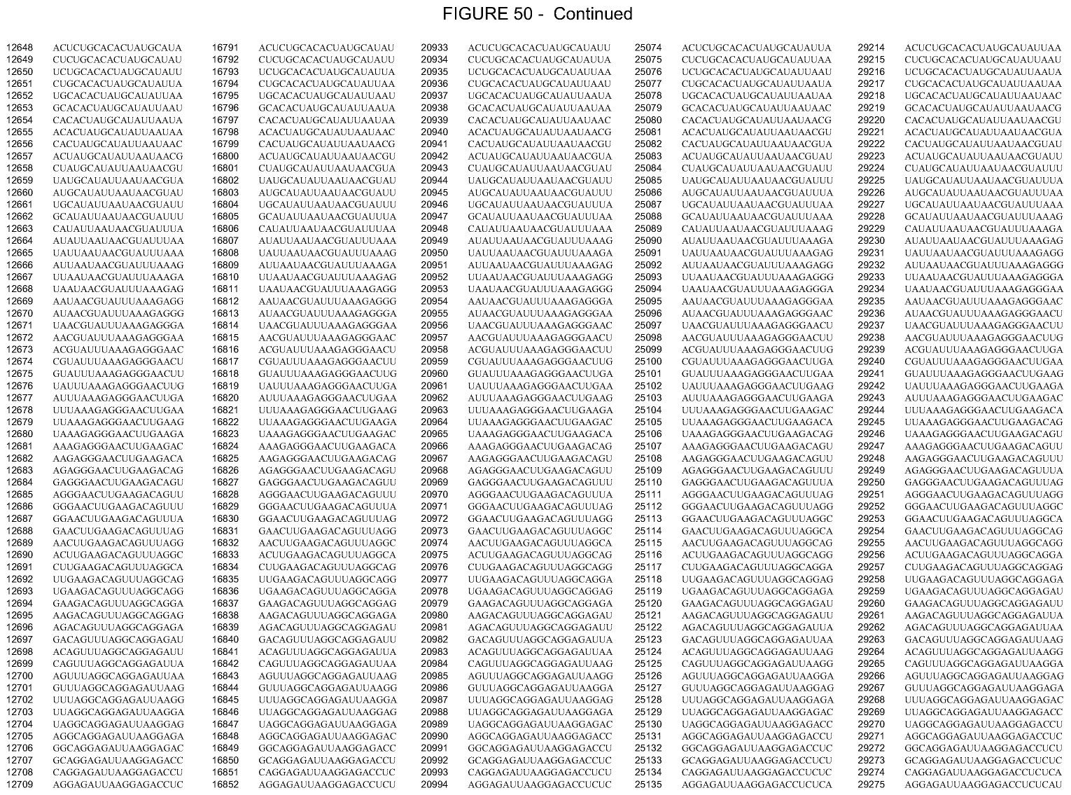

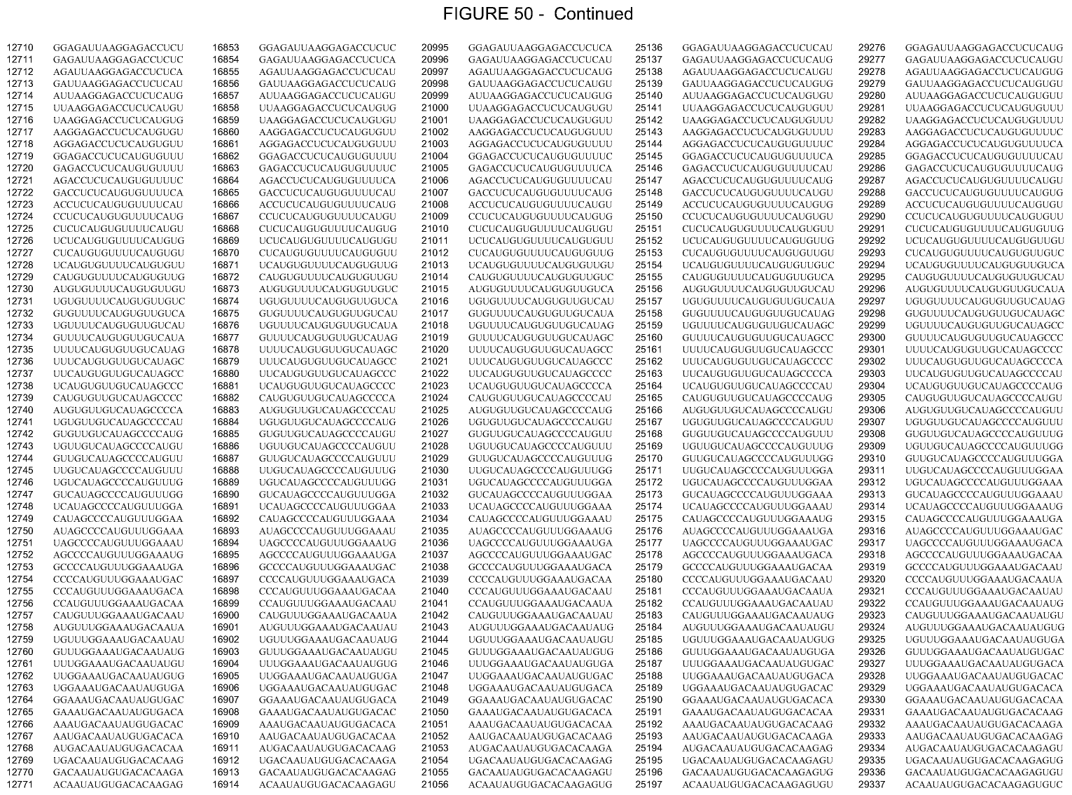

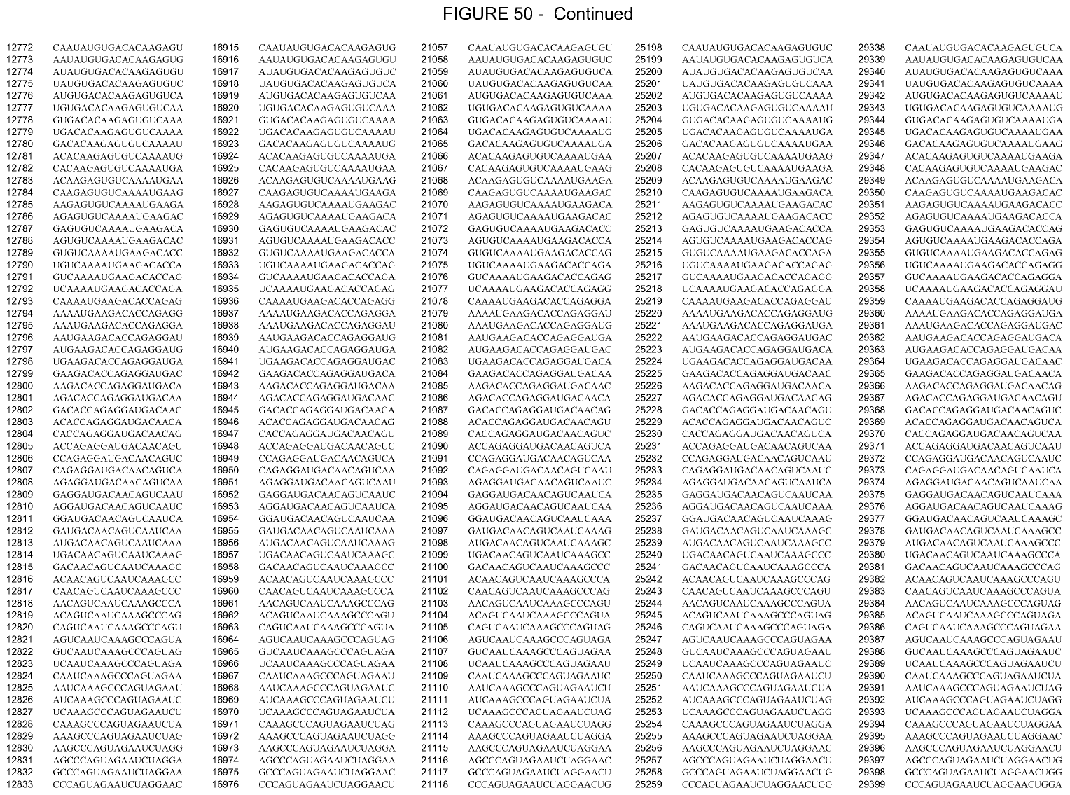

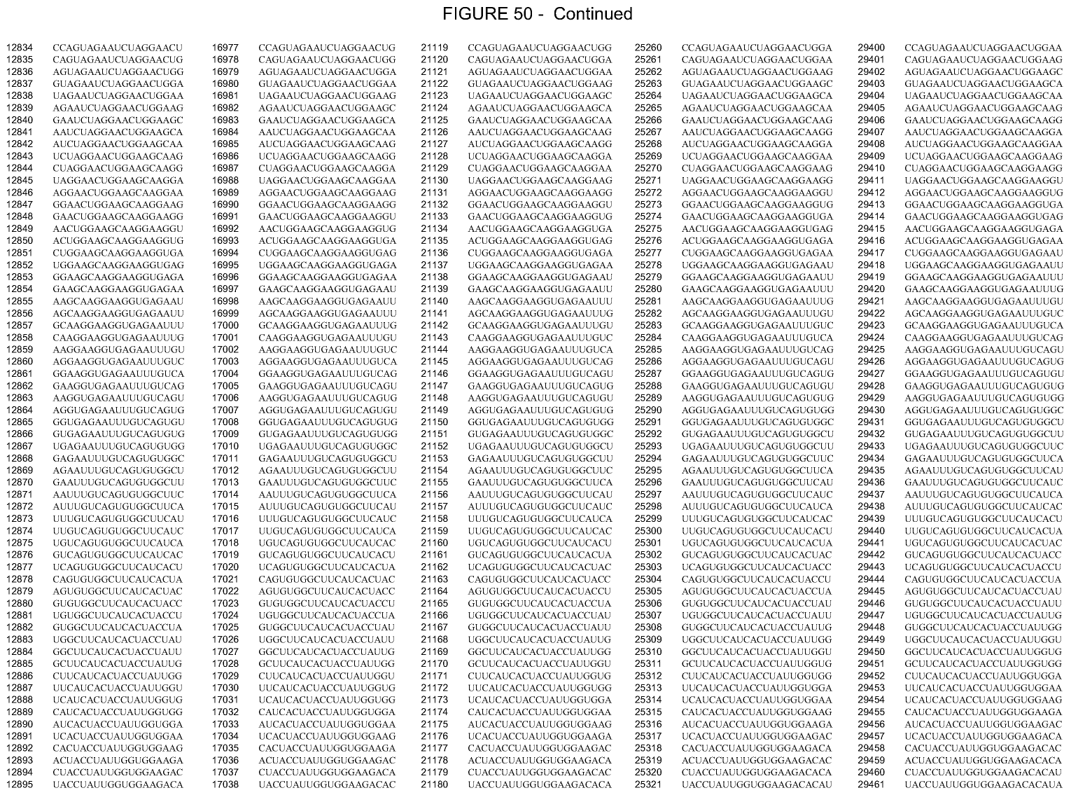

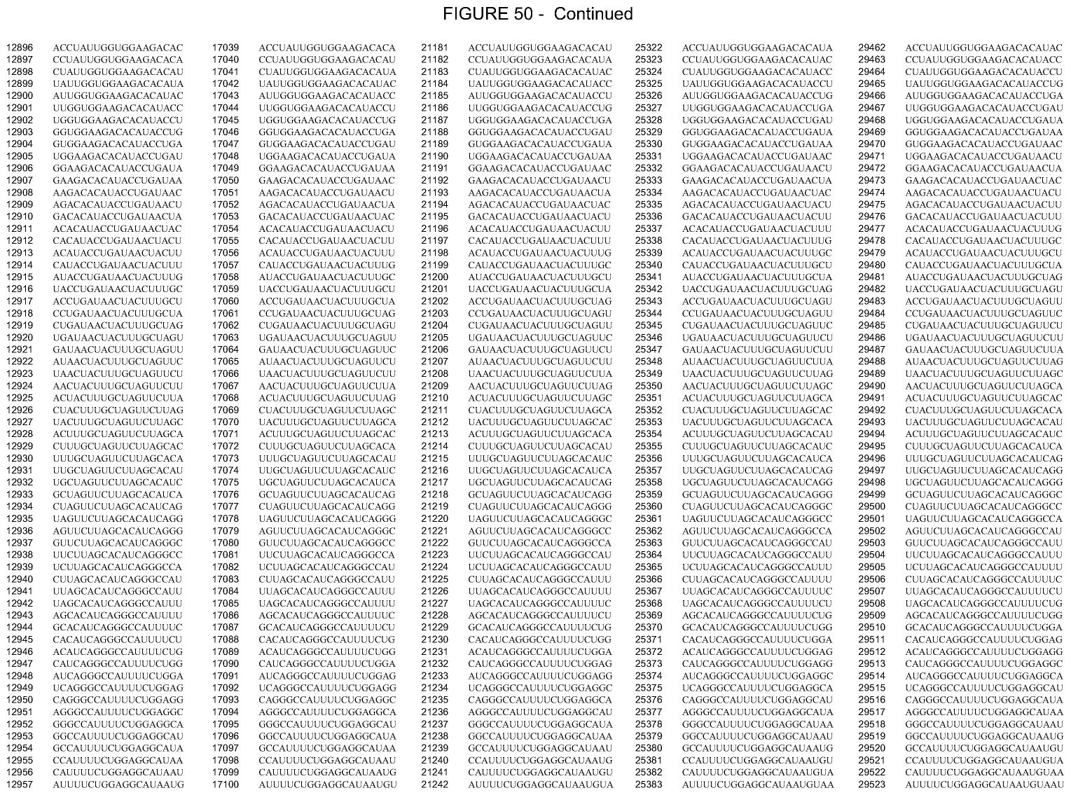

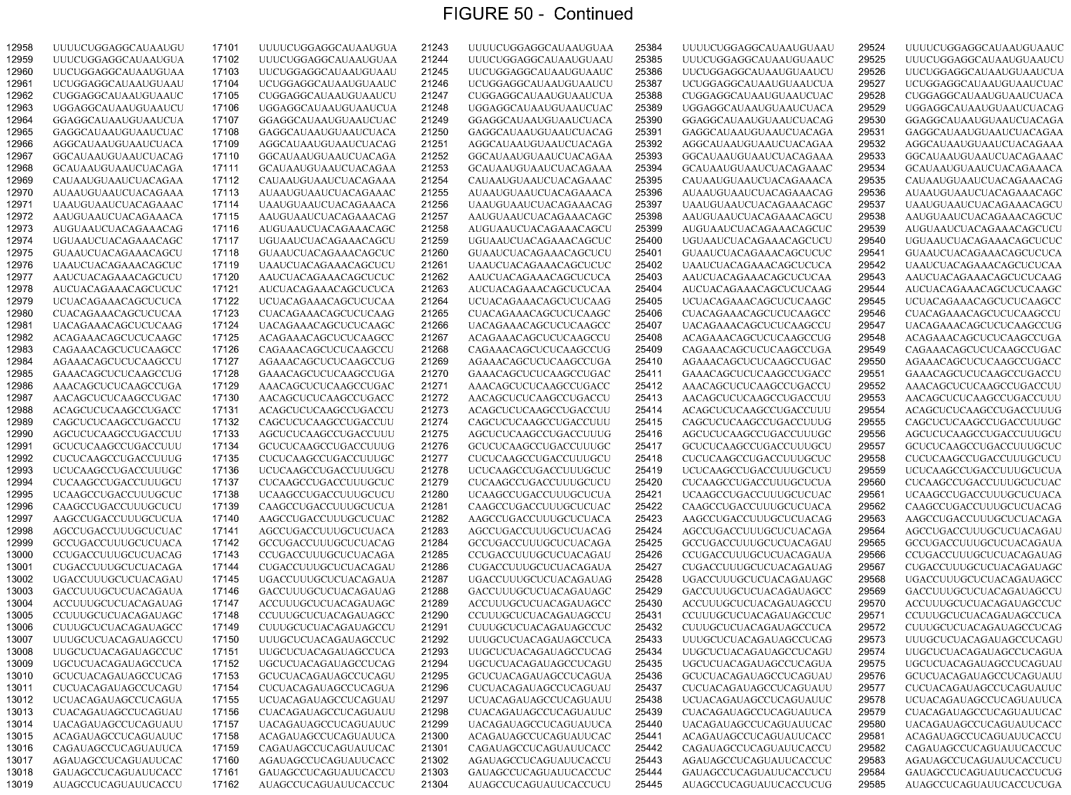

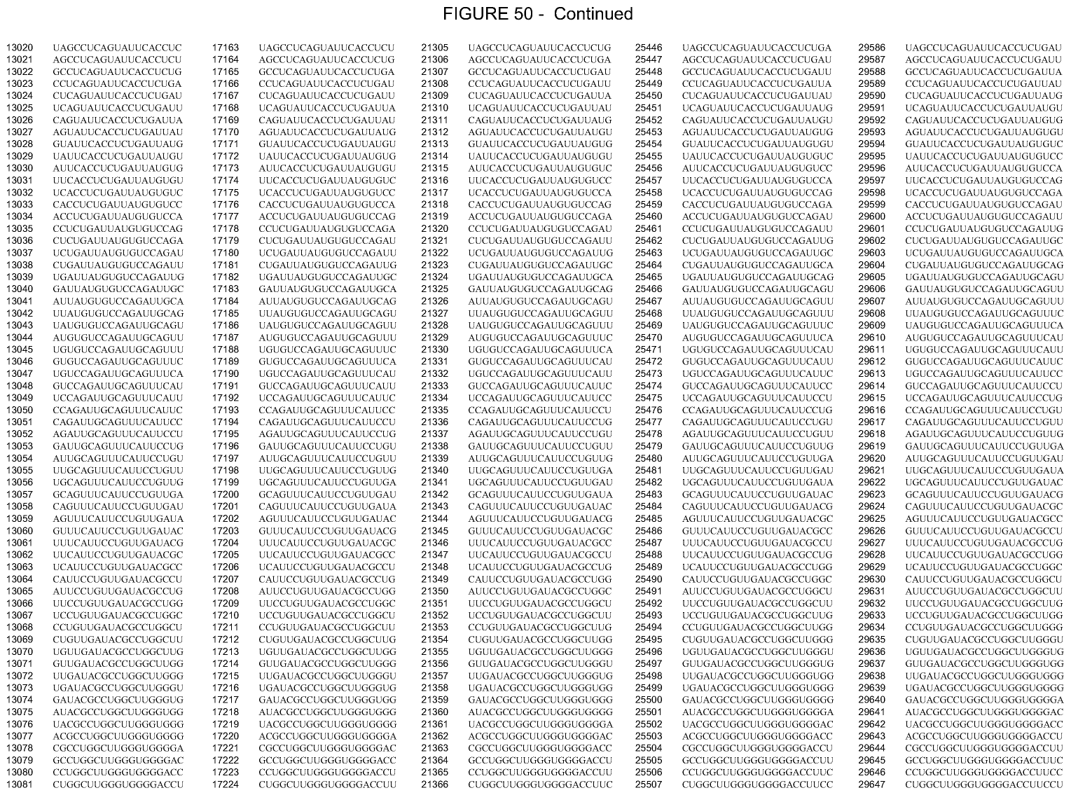

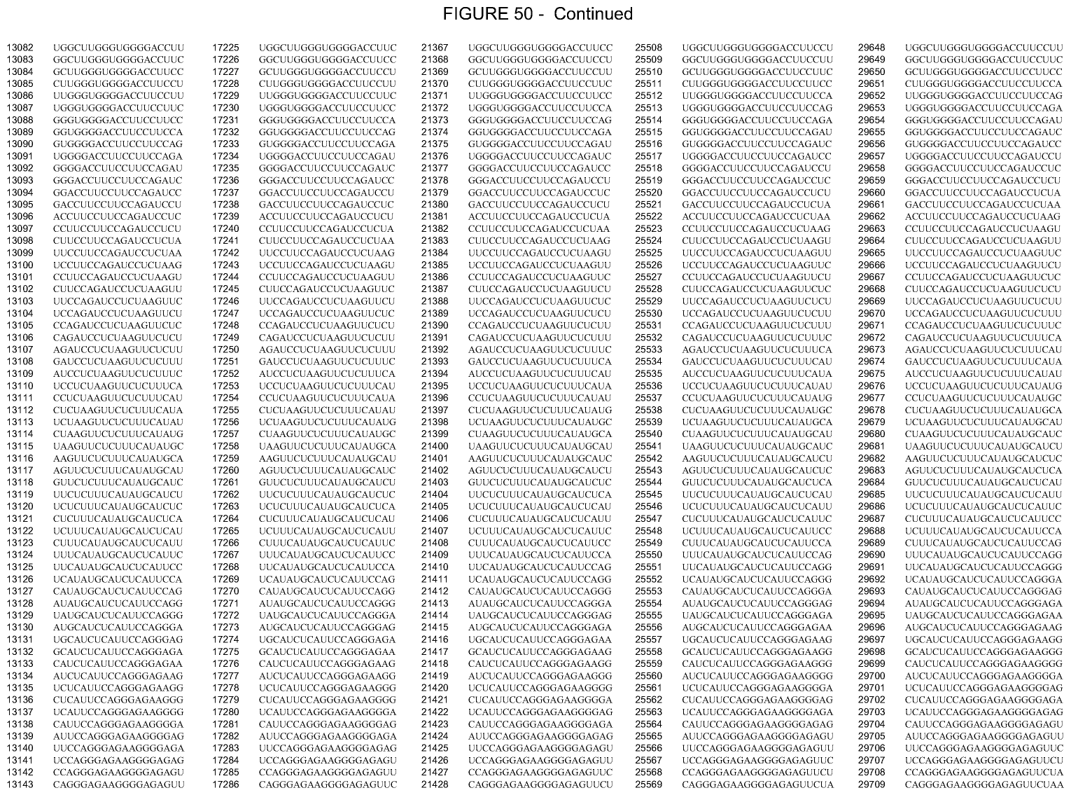

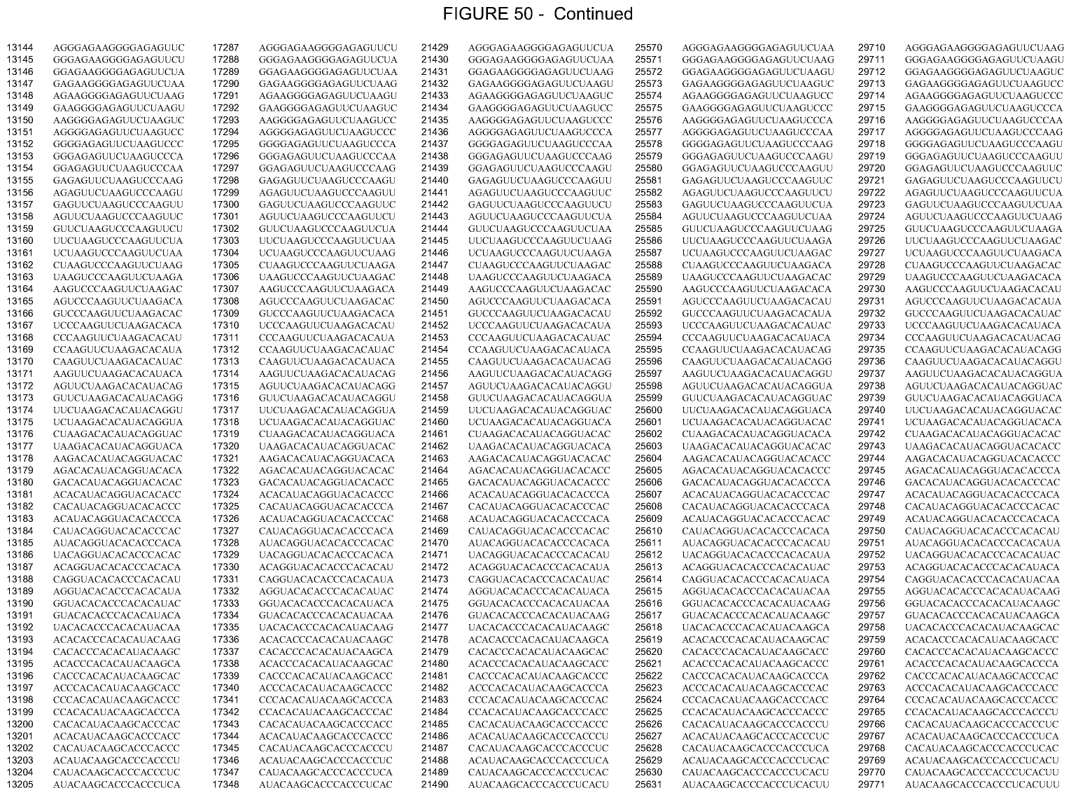

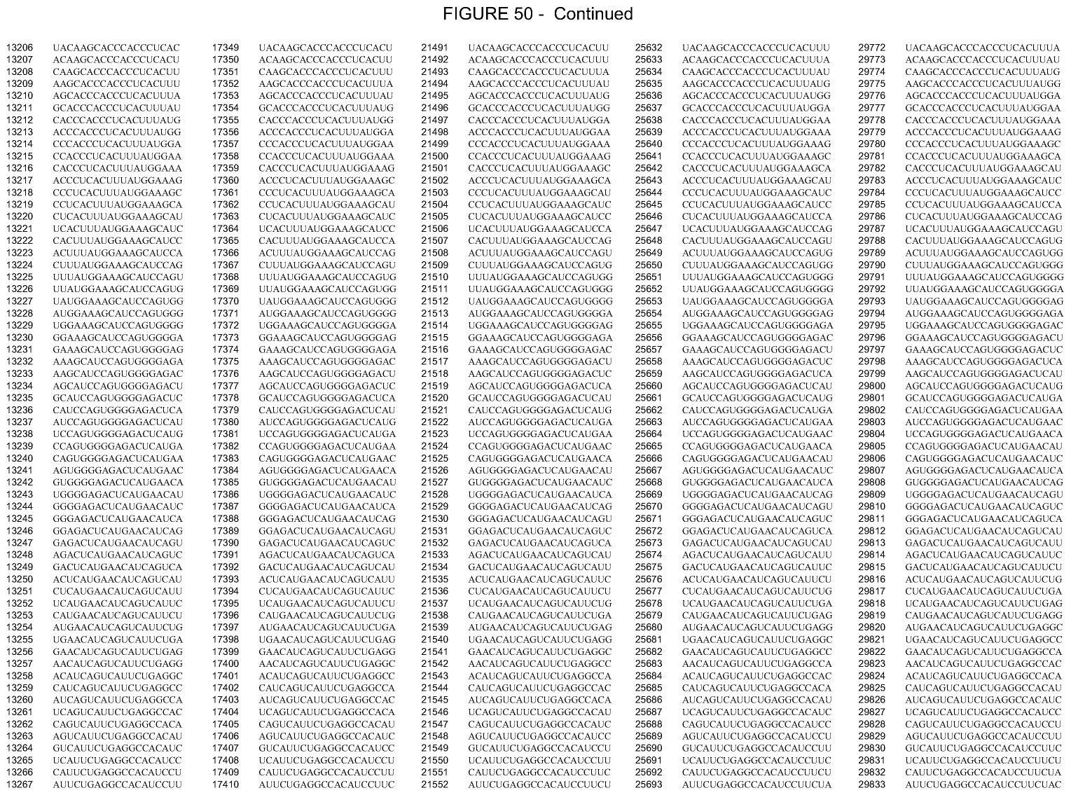

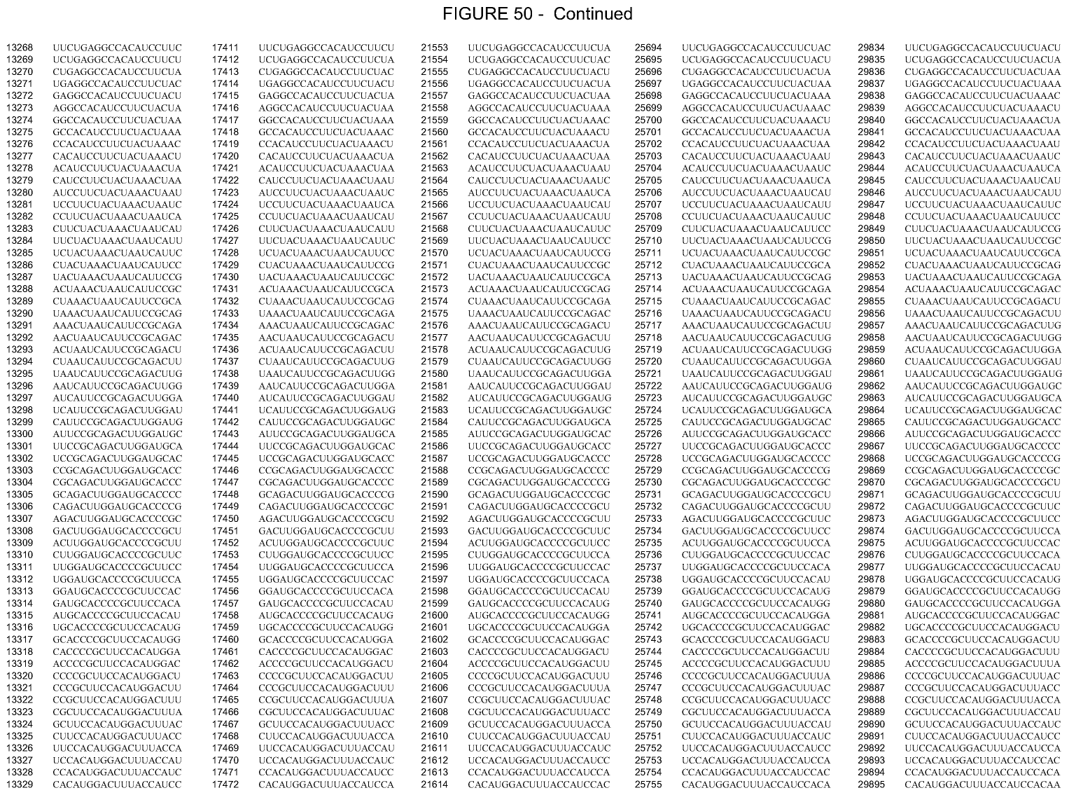

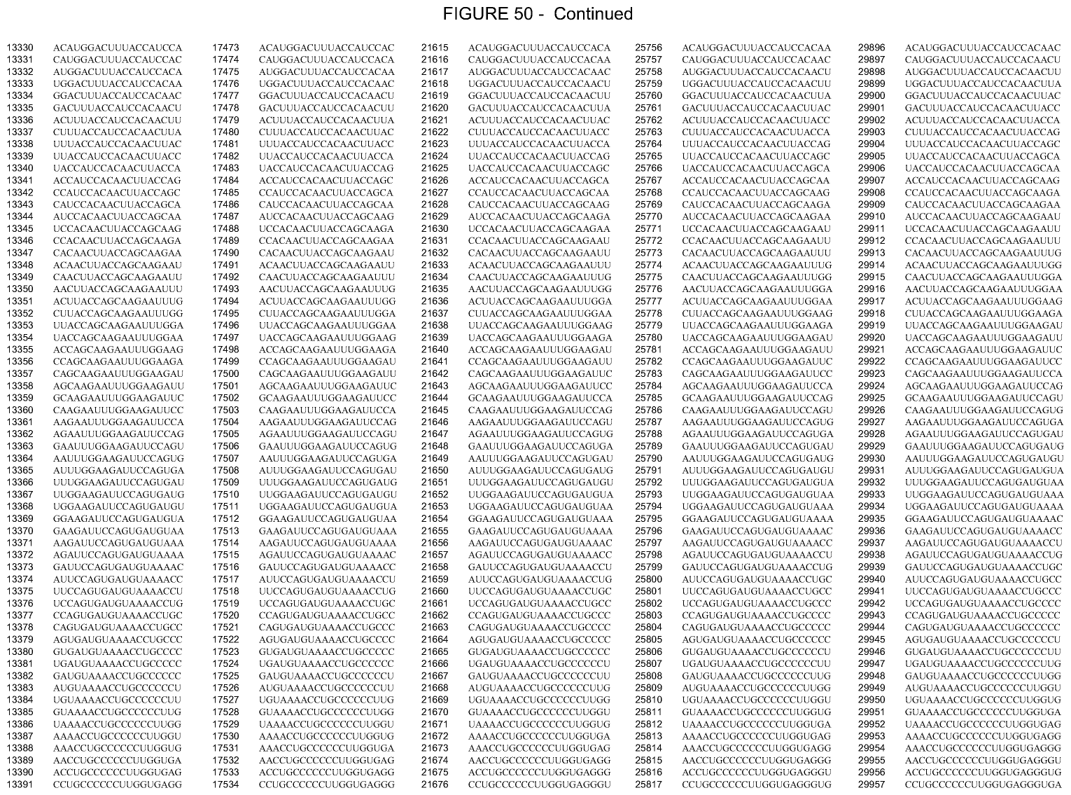

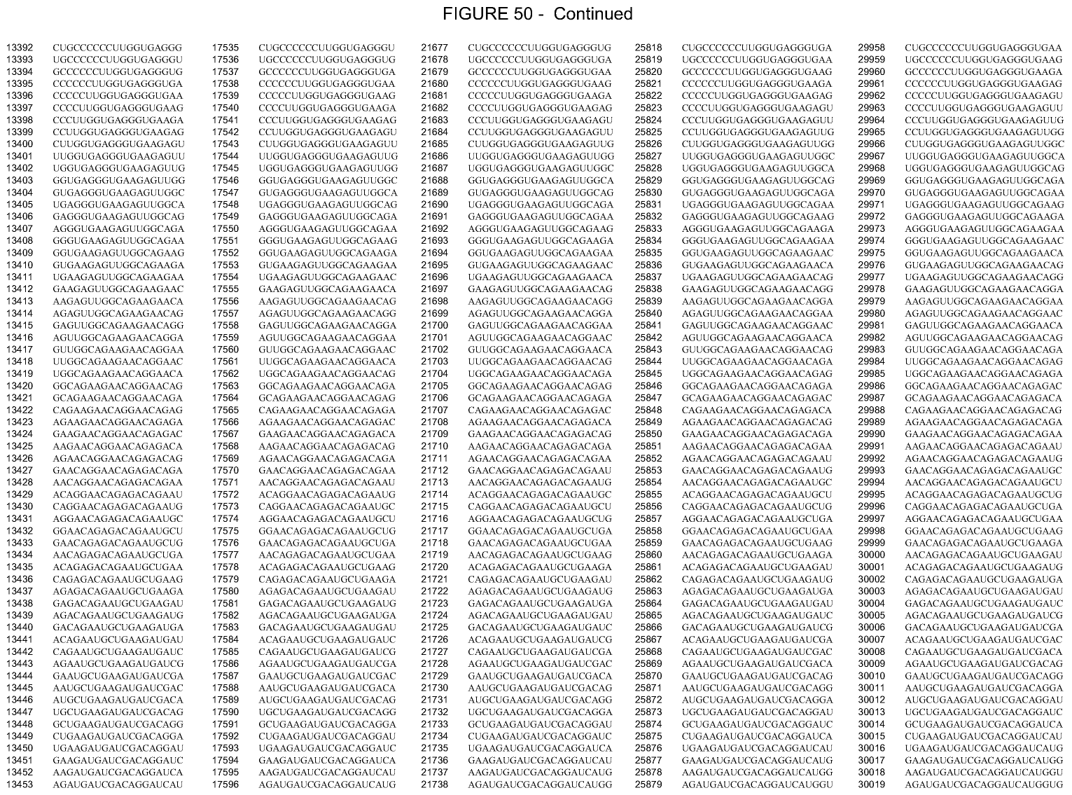

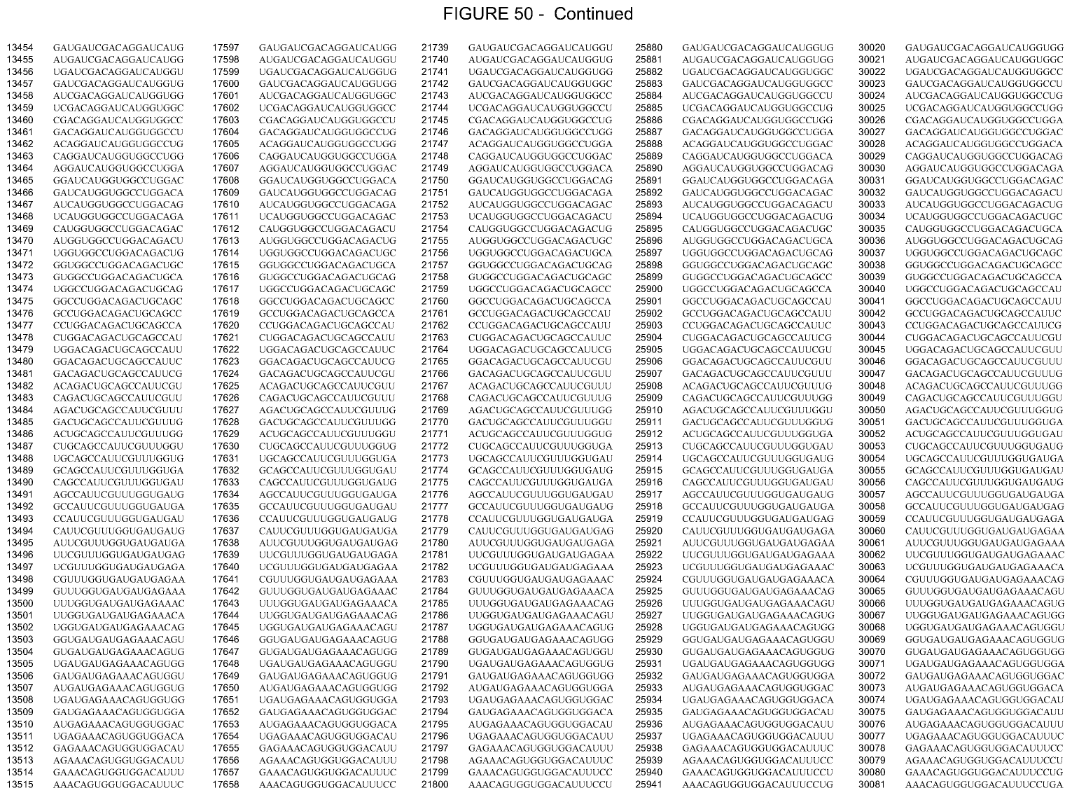

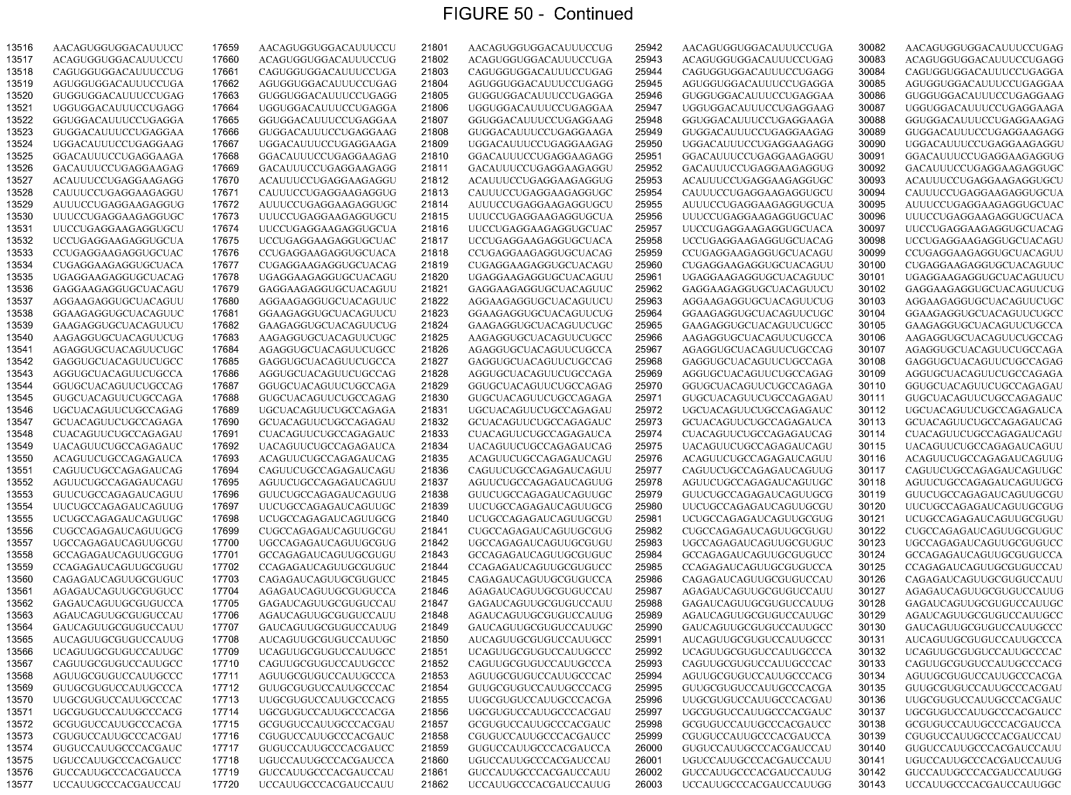

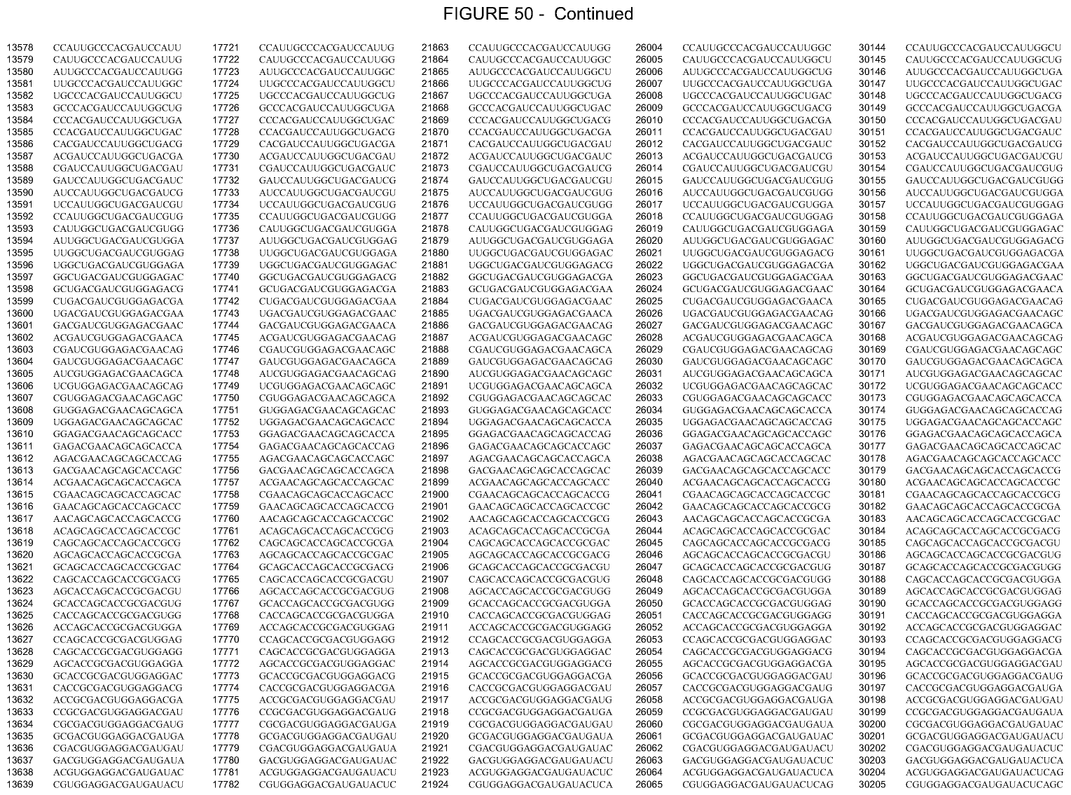

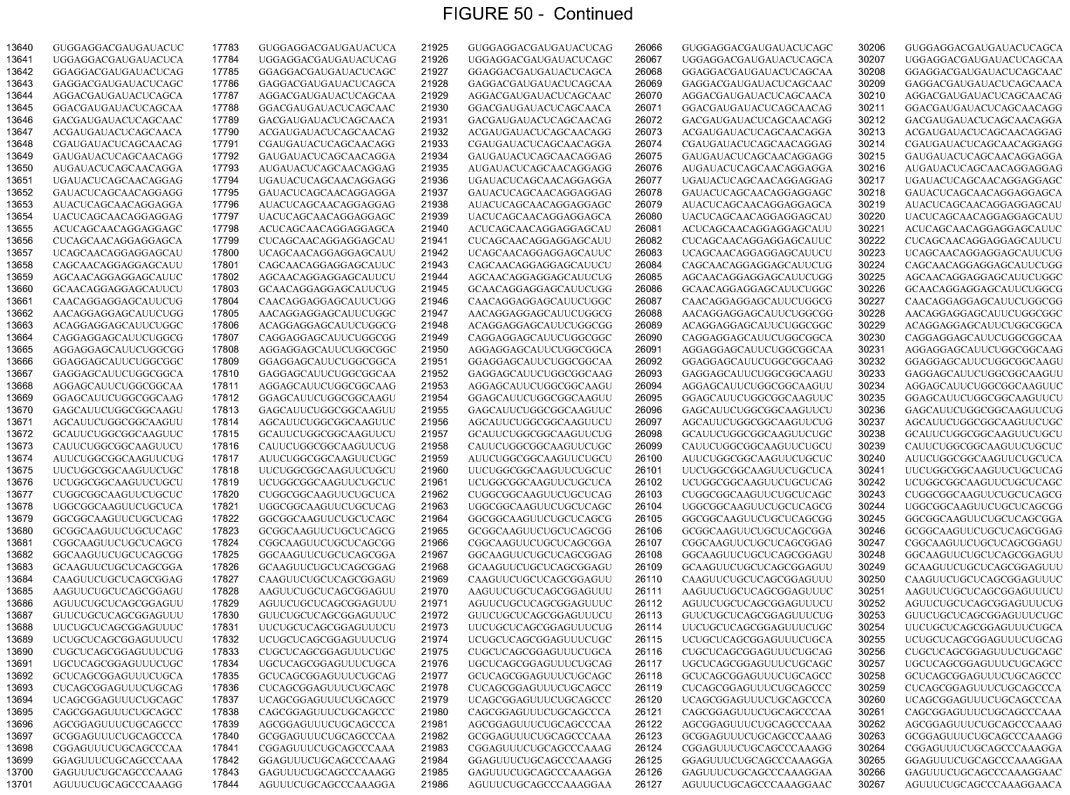

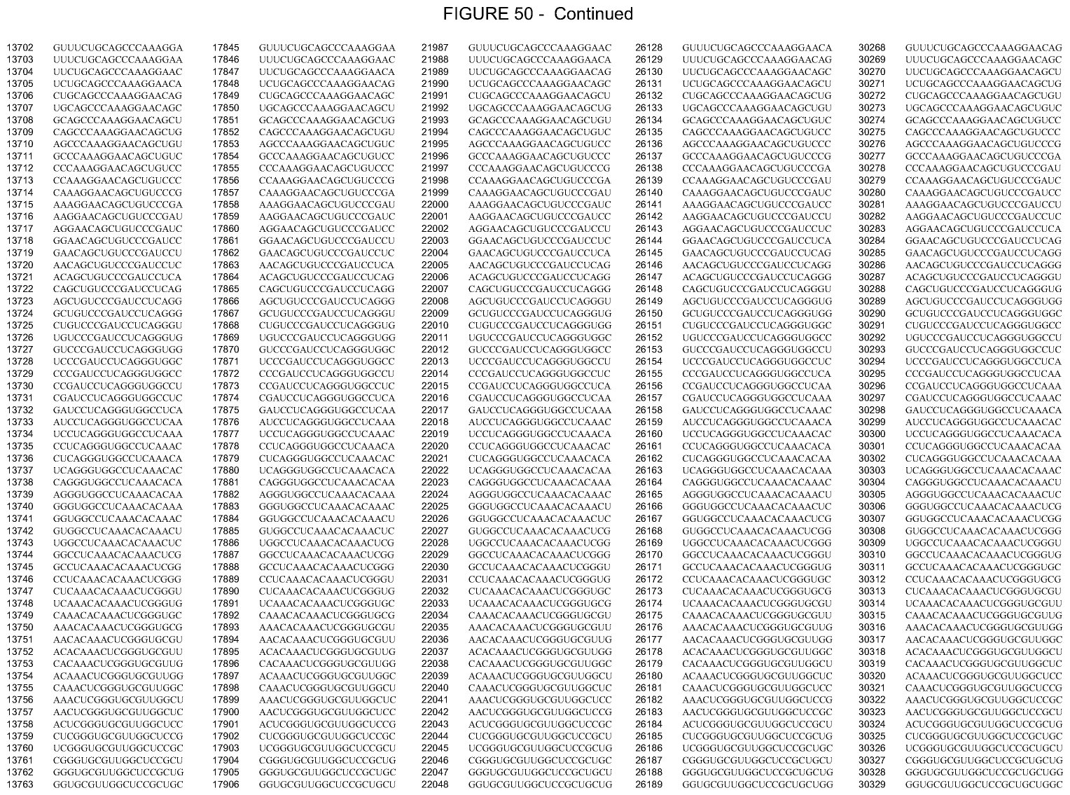

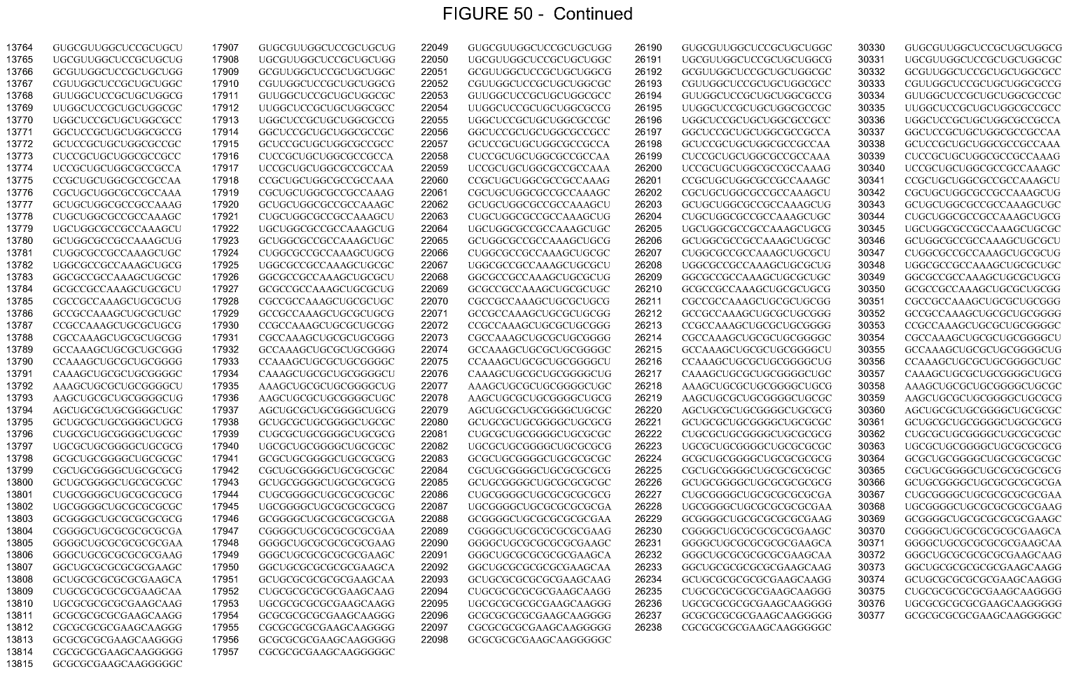

Exemplary miPMP22s comprise the RNA sequence set out in any one or more of SEQ ID NOs: 9-16, or a variant thereof comprising at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs 9-16. Final processed guide strand sequences corresponding to SEQ ID NOs: 9-16 are respectively set out in SEQ ID NOs: 17-24. The disclosure additionally provides the antisense guide strands set out in FIG. 48 and contemplates variants of each of those antisense guide strands that are at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identical. The disclosure additionally provides the antisense guide strands set out in FIG. 50 and contemplates variants of each of those antisense guide strands that are at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% identical.

The disclosure contemplates polynucleotides encoding one or more copies of these sequences are combined into a single delivery vehicle, such as a vector. Thus, the disclosure includes vectors comprising a nucleic acid of the disclosure or a combination of nucleic acids of the disclosure. Provided are viral vectors (such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein. AAV vectors are exemplified. Non-viral delivery vehicles are also contemplated

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24(7): 1247-1257 (2016). Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus (56 to 65° C. for several hours), making cold preservation of AAV less critical. AAV may even be lyophilized. Finally, AAV-infected cells are not resistant to superinfection.

As exemplified herein, the AAV vector lacks rep and cap genes. The AAV can be a recombinant AAV (rAAV) or a self-complementary recombinant AAV (scAAV). The AAV has a capsid serotype can be from, for example, AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, or AAVrh.10.

Viral vectors provided include, for example, AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), AAVrh.10 (i.e., an AAV containing AAVrh.10 ITRs and AAVrh.10 capsid proteins), AAV11 (i.e., an AAV containing AAV11 ITRs and AAV11 capsid proteins), AAV12 (i.e., an AAV containing AAV12 ITRs and AAV12 capsid proteins), or AAV13 (i.e., an AAV containing AAV13 ITRs and AAV13 capsid proteins).

DNA plasmids of the disclosure comprise recombinant AAV (rAAV) genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74. IAAV DNA in the rAAV genomes can be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

The AAV vector can be a pseudotyped AAV, containing ITRs from one AAV serotype and capsid proteins from a different AAV serotype. The pseudo-typed AAV can be AAV2/9 (i.e., an AAV containing AAV2 ITRs and AAV9 capsid proteins). The pseudotyped AAV can be AAV2/8 (i.e., an AAV containing AAV2 ITRs and AAV8 capsid proteins). The pseudotyped AAV can be AAV2/1 (i.e., an AAV containing AAV2 ITRs and AAV1 capsid proteins).

The AAV vector can contain a recombinant capsid protein, such as a capsid protein containing a chimera of one or more of capsid proteins from AAV1, AAV2, AAV3, AAV4, AAV5, AAV6, AAV7, AAV8, AAV9, AAVrh74, AAVrh.8, or AAVrh.10, AAV10, AAV11, AAV12, or AAV13. Other types of rAAV variants, for example rAAV with capsid mutations, are also contemplated. See, for example, Marsic et al., Molecular Therapy, 22(11): 1900-1909 (2014). As noted above, the nucleotide sequences of the genomes of various AAV serotypes are known in the art.

The disclosure provides AAV to deliver miPMP22s which target PMP22 mRNA to inhibit PMP22 expression. AAV can be used to deliver miPMP22s under the control of an RNA polymerase III (Pol III)-based promoter. AAV is used to deliver miPMP22s under the control of a U6 promoter. AAV is used to deliver miPMP22s under the control of a H1 promoter. AAV is used to deliver miPMP22s under the control of an RNA polymerase II (Pol II)-based promoter. AAV is used to deliver miPMP22s under the control of an U7 promoter. AAV is used to deliver MiPMP22s under the control of a Schwann cell-specific promoter. AAV is used to deliver miPMP22s under the control of an MPZ promoter. AAV is used to deliver miPMP22s under the control of a PMP22 promoter.

In nature, the U6 promoter controls expression of the U6 RNA, a small nuclear RNA (snRNA) involved in splicing, and which has been well-characterized [Kunkel et al., Nature, 322(6074):73-77 (1986); Kunkel et al., Genes Dev. 2(2):196-204 (1988); Paule et al., Nuc. Acids Res., 28(6):1283-1298 (2000)]. The U6 promoter is used to control vector-based expression in mammalian cells [Paddison et al., Proc. Natl. Acad. Sci. USA, 99(3):1443-1448 (2002); Paul et al., Nat. Biotechnol.,20(5):505-518 (2002)] because (1) the promoter is recognized by RNA polymerase III (poly III) and controls high-level, constitutive expression of RNA; and (2) the promoter is active in most mammalian cell types. The disclosure includes use of both murine and human U6 promoters.

AAV vectors herein lack rep and cap genes. The AAV can be a recombinant AAV, a recombinant single-stranded AAV (ssAAV), or a recombinant self-complementary AAV (scAAV).

rAAV genomes of the disclosure comprise one or more AAV ITRs flanking a polynucleotide encoding, for example, one or more miPMP22s. Commercial providers such as Ambion Inc. (Austin, TX), Darmacon Inc. (Lafayette, CO), InvivoGen (San Diego, CA), and Molecular Research Laboratories, LLC (Herndon, VA) generate custom inhibitory RNA molecules. In addition, commercial kits are available to produce custom siRNA molecules, such as SILENCER™ siRNA Construction Kit (Ambion Inc., Austin, TX) or psiRNA System (InvivoGen, San Diego, CA).

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing [Samulski et al., Proc. Natl. Acad. S6. USA, 79:2077-2081 (1982)], addition of synthetic linkers containing restriction endonuclease cleavage sites [Laughlin et al., Gene, 23:65-73 (1983)] or by direct, blunt-end ligation [Senapathy & Carter, J. Biol. Chem., 259:4661-4666 (1984)]. The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, Current Opinions in Biotechnology, 1533-1539 (1992); and Muzyczka, Curr. Topics in Microbiol. and Immunol., 158: 97-129 (1992). Various approaches are described in Ratschin et al., Mol. Cell. Biol. 4:2072 (1984); Hermonat et al., Proc. Natl. Acad. Sci. USA, 81: 6466 (1984); Tratschin et al., Mol. Cell. Biol. 5: 3251 (1985); McLaughlin et al., J. Virol., 62:1963 (1988); and Lebkowski et al., Mol. Cell. Biol., 7: 349 (1988). Samulski et al., J. Virol., 63: 3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Hum. Gene Ther., 4:609-615 (1993); Clark et al., Gene Ther., 3:1124-1132 (1996); U.S. Pat. Nos. 5,786,211; 5,871,982; 6,258,595; and McCarty, Mol. Ther., 16(10): 1648-1656 (2008). The foregoing documents are hereby incorporated by reference in their entirety herein, with particular emphasis on those sections of the documents relating to rAAV production. The production and use of self-complementary (sc) rAAV are specifically contemplated and exemplified.

The disclosure further provides packaging cells that produce AAV vectors. Packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (rAAV) (i.e., infectious encapsidated rAAV particles) are thus provided herein. The genomes of the rAAV lack AAV rep and cap DNA, that is, there is no AAV rep or cap DNA between the ITRs of the genomes of the rAAV.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

Compositions comprising the nucleic acids and viral vectors of the disclosure are provided. Compositions comprising delivery vehicles (such as rAAV) described herein are provided. Such compositions also comprise a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁶, or more DNase resistant particles (DRP) [or viral genomes (vg)] per ml.

Methods of transducing a target cell with a delivery vehicle (such as rAAV), in vivo or in vitro, are contemplated. The in vivo methods comprise the step of administering an effective dose, or effective multiple doses, of a composition comprising a delivery vehicle (such as rAAV) to an subject (including a human patient) in need thereof. If the dose is administered prior to development of a disorder/disease, the administration is prophylactic. If the dose is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the invention is CMT1A. In families known to carry pathological PMP22 gene duplications or mutations, the methods can be carried out before the onset of disease. In other patients, the methods are carried out after diagnosis.

Molecular, biochemical, histological, and functional outcome measures demonstrate the therapeutic efficacy of the methods. Outcome measures are described, for example, in Chapters 32, 35 and 43 of Dyck and Thomas, Peripheral Neuropathy, Elsevier Saunders, Philadelphia, PA, 4^{th} Edition, Volume 1 (2005) and in Burgess et al., Methods MoL. Biol., 602: 347-393 (2010). Outcome measures include, but are not limited to, one or more of the reduction or elimination of mutant PMP22 mRNA or protein in affected tissues, PMP22 gene knockdown, increased body weight and improved muscle strength. Others include, but are not limited to, nerve histology (axon number, axon size and myelination), neuromuscular junction analysis, and muscle weights and/or muscle histology. Others include, but are not limited to, nerve conduction velocity-ncv, electromyography-emg, and synaptic physiology.

In the methods of the disclosure, expression of PMP22 in a subject is inhibited by at least 10, at least 20, at least 30, at least 40, at least 50, at least 60, at least 70, at least 80, at least 90, at least 95, at least 98 percent, at least 99 percent, or 100 percent compared to expression in the subject before treatment.

Combination therapies are also contemplated by the invention. Combination as used herein includes both simultaneous treatment and sequential treatments. Combinations of methods described herein with standard medical treatments and supportive care are specifically contemplated.

Administration of an effective dose of a nucleic acid, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. An effective dose can be delivered by a combination of routes. For example, an effective dose is delivered intravenously and intramuscularly, or intravenously and intracerebroventricularly, and the like. An effective dose can be delivered in sequence or sequentially. An effective dose can be delivered simultaneously. Route(s) of administration and serotype(s) of AAV components of the rAAV (in particular, the AAV ITRs and capsid protein) of the invention are chosen and/or matched by those skilled in the art taking into account the infection and/or disease state being treated and the target cells/tissue(s) that are to express the miRNAs.

In particular, actual administration of delivery vehicle (such as rAAV) may be accomplished by using any physical method that will transport the delivery vehicle (such as rAAV) into a target cell of an subject. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as glial cells (e.g., Schwann cells). See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction of cells such as Schwann cells with rAAV provided herein results in sustained expression of PMP22 miRNAs. The present invention thus provides methods of administering/delivering rAAV which express PMP22 miRNAs to a subject, preferably a human being. These methods include transducing cells and tissues (including, but not limited to, glial cells such as Schwann cells, peripheral motor neurons, sensory motor neurons, tissues such as muscle, and organs such as liver and brain) with one or more rAAV described herein. Transduction may be carried out with gene cassettes comprising cell-specific control elements.

The term “transduction” is used to refer to, as an example, the administration/delivery of miPMP22s to a target cell either in vivo or in vitro, via a replication-deficient rAAV described herein resulting in the expression of miPMP22s by the target cell (e.g., Schwann cells).

Thus, methods are provided of administering an effective dose (or doses, administered essentially simultaneously or doses given at intervals) of rAAV described herein to subject in need thereof.

### Other Terminology

As used herein, singular forms “a,” “and,” and “the” include plural referents unless the context clearly indicates otherwise. Thus, for example, reference to “an antibody” includes multiple antibodies.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1-, 2-, 3-, 4-, 5-, 6-, 7-, 8-, 9-, 10-, 11-, 12-, 13-, 14-, 15-, 16-, 17-, 18-, 19-, or 20-fold, etc., as well as 1.1-, 1.2-, 1.3-, 1.4-, or 1.5-fold, etc., 2.1-, 2.2-, 2.3-, 2.4-, or 2.5-fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, “can” or “can be” indicates something contemplated by the inventors that is functional and available as part of the subject matter provided.

### EXAMPLES

Aspects and exemplary embodiments of the invention are illustrated by the following examples.

### Example 1

Design and in vitro Testing of miPMP22 Targeting PMP22

Artificial miRNAs are based on the natural mir-30, maintaining important structural and sequence elements required for normal miRNA biogenesis but replacing the mature mir-30 sequences with 22-nt of complementarity with the PMP22 gene. See FIG. 1 which shows an exemplary general miRNA shuttle structure.

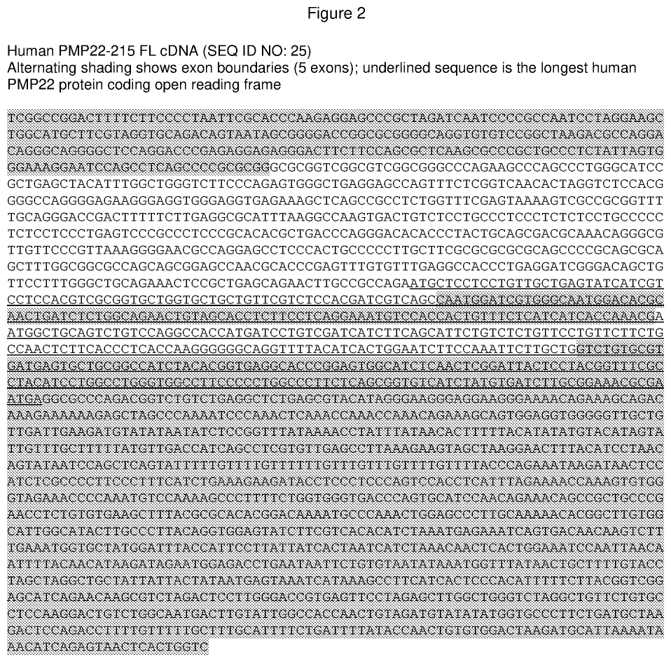

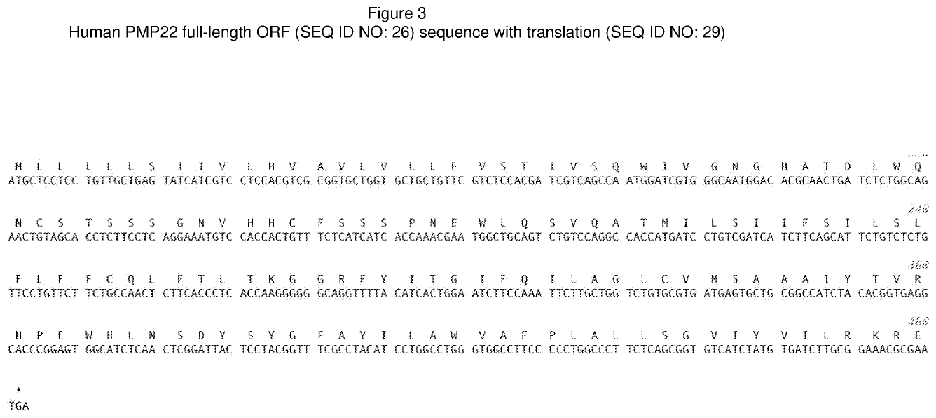

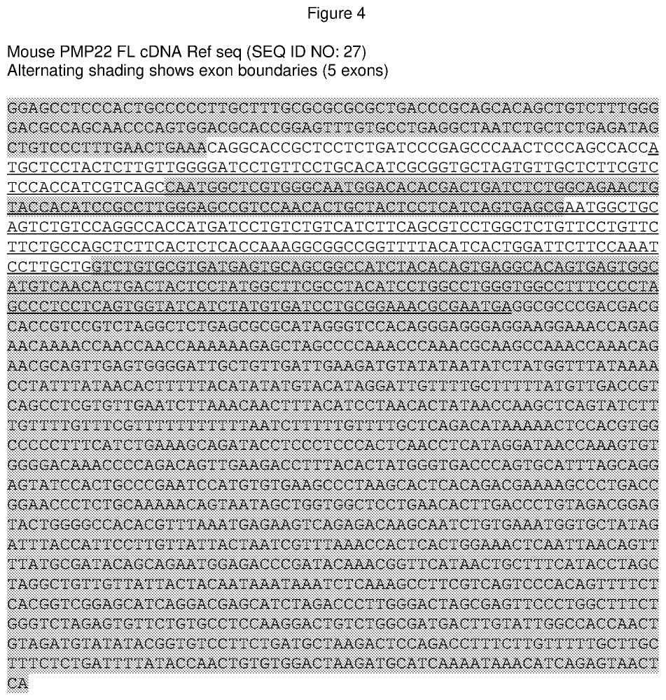

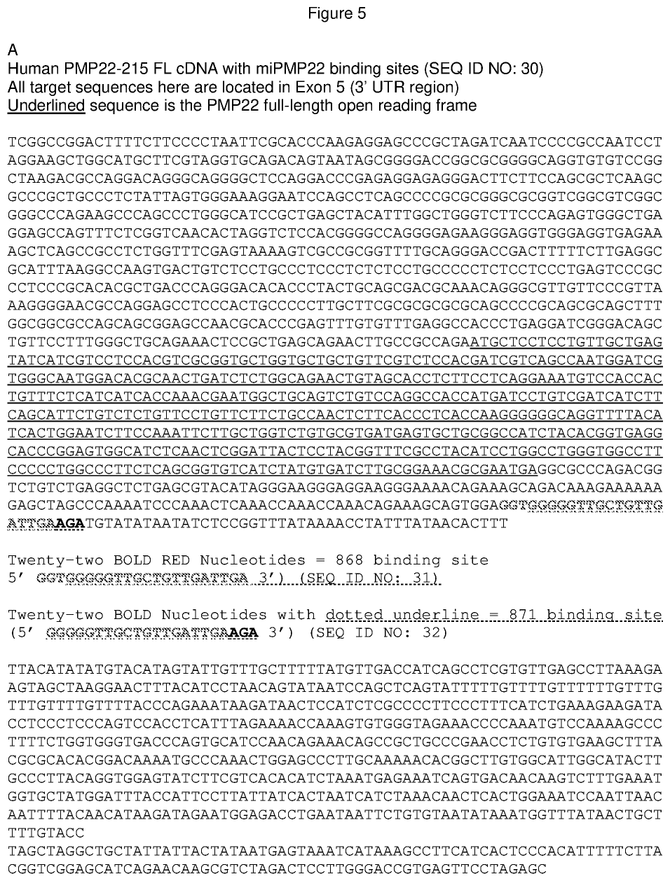

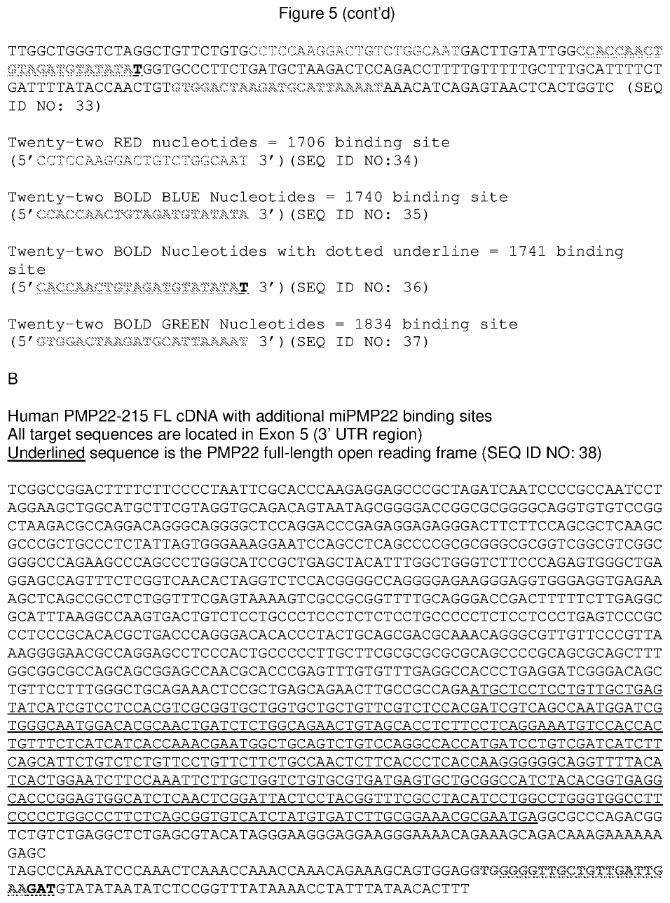

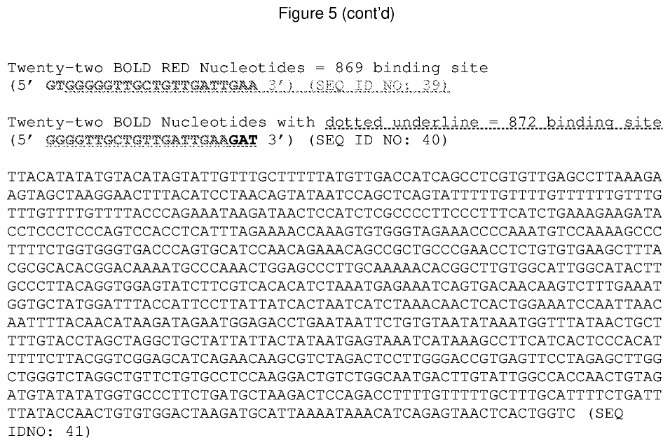

The PMP22 artificial miRNAs (miPMP22s) were designed to target conserved regions between the mouse PMP22 gene and the human PMP22 gene. A full length human PMP22 cDNA sequence is shown in FIG. 2 and with a translation in FIG. 3, while a mouse PMP22 cDNA is shown in FIG. 4. All the miPMP22s bind to conserved regions of the 3′ UTR in Exon 5. FIG. 5 shows the binding sites of six miRNAs referred to herein as miPMP22-868, miPMP22-871, miPMP22-1706, miPMP22-1740, miPMP22-1741 and miPMP22-1834 within a human PMP22 cDNA.

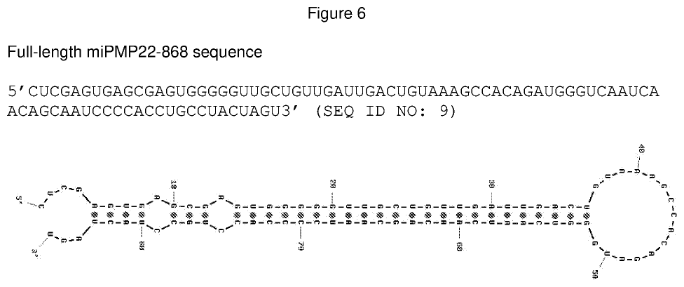

The miPMP22-868 DNA template sequence is
5′CTCGAGTGAGCGAGTGGGGGTTGCTGTTGATTGACTGTAAAGCCACAGATGGGTCAATCAACAGCA ATCCCCACCTGCCTACTAGT3′ (SEQ ID NO: 1), which encodes the full length RNA sequence
5′CUCGAGUGAGCGAGUGGGGGUUGCUGUUGAUUGACUGUAAAGCCACAGAUGGGUCAAUCAACAGCA AUCCCCACCUGCCUACUAGU3′ (SEQ ID NO: 9). FIG. 6 shows the folded full length RNA sequence generated using Unafold. The processed mature double-stranded miPMP22-868 is

```
    5′ UGGGGGUUGCUGUUGAUUGACU 3′ Sense strand (passenger strand) (SEQ ID NO: 43)
          ||||||||||||||||||||
3′ CCACCCCUAACGACAACUAACU 5′ Antisense strand (guide strand) (SEQ ID NO: 17)
```

while the processed antisense guide strand is 5′ UCAAUCAACAGCAA. CCCCACC 3′ (SEQ ID NO: 17).

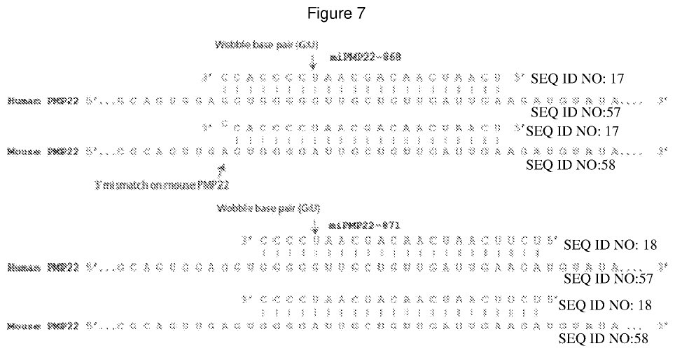

The fifteenth nucleotide in the antisense guide strand was changed to a “U” so that instead of a traditional Watson-Crick base pair (C:G) at that position in the duplex, the base pair is a wobble U:G base pair for two main reasons. See FIG. 7. First, the mouse and human PMP22 genes have a sequence polymorphism at this binding site. In humans, the nucleotide is a G, while in mice it is an A. RNA molecules can form G:U base pairs (2 hydrogen bonds) as well as G:C base pairs (3 hydrogen bonds). Like DNA, RNA cannot form G:A base-pairs. Thus, the nucleotide is changed to a U, so that it can base pair with both the mouse and human PMP22 transcripts at this position. Second, changing this base also reduced the G:C content of the miRNA duplex from about 55% to 50%. A long stretch of five consecutive G:C base pairs at the 3′ end of the antisense molecule could possibly inhibit unwinding of the duplex and reduce silencing. Because G:U base-pairs have only two hydrogen bonds, while GC base-pairs have three hydrogen bonds, inserting the U at this position still allows base-pairing of the antisense and sense strands of the miRNA but with a slightly weaker interaction.

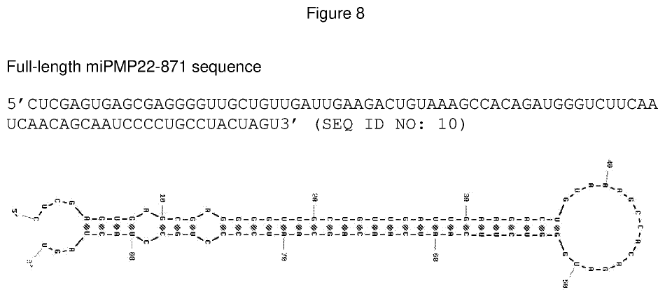

The miPMP22-871 DNA template sequence is
5′CTCGAGTGAGCGAGGGGTTGCTGTTGATTGAAGACTGTAAAGCCACAGATGGGTCTTCAATCAACA GCAATCCCCTGCCTACTAGT3′ (SEQ ID NO: 2), which encodes the full length RNA sequence
5′CUCGAGUGAGCGAGGGGUUGCUGUUGAUUGAAGACUGUAAAGCCACAGAUGGGUCUUCAAUCAACA GCAAUCCCCUGCCUACUAGU3′ (SEQ ID NO: 10). FIG. 8 shows the folded full length RNA sequence generated using Unafold. The processed mature double-stranded miPMP22-871 is

```
    5′ GGGUUGCUGUUGAUUGAAGACU 3′ Sense strand (passenger strand) (SEQ ID NO: 44)
          ||||||||||||||||||||
3′ CCCCUAACGACAACUAACUUCU 5′ Antisense strand (guide strand) (SEQ ID NO: 18)
```

while the processed antisense guide strand is 5′ UCUUCAAUCAACAGCAA CCCC 3′ (SEQ ID NO: 18).

In a similar manner to miPMP22-868, the eighteenth nucleotide in the miPMP22-871 antisense guide strand was changed to a “U”. See FIG. 7.

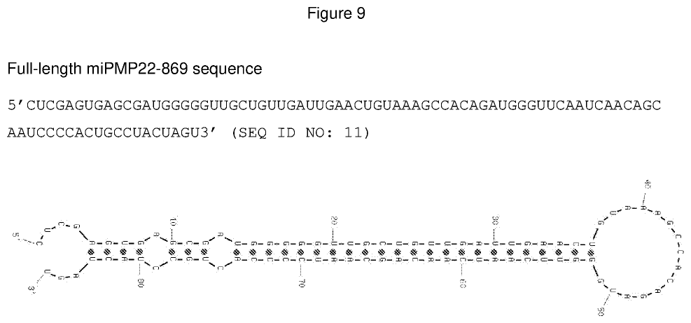

The miPMP22-869 DNA template sequence is
5′CTCGAGTGAGCGATGGGGGTTGCTGTTGATTGAACTGTAAAGCCACAGATGGGTTCAATC AACAGCAATCCCCACTGCCTACTAGT3′ (SEQ ID NO: 3), which encodes the full length RNA sequence
5′CUCGAGUGAGCGAUGGGGGUUGCUGUUGAUUGAACUGUAAAGCCACAGAUGGGUUCAAUC AACAGCAAUCCCCACUGCCUACUAGU3′ (SEQ ID NO: 11). FIG. 9 shows the folded full length RNA sequence generated using Unafold. The processed mature double-stranded miPMP22-869 is

5′ GGGGGUUGCUGUUGAUUGAACU 3′ Sense strand (passenger strand) (SEQ ID NO: 45)
||||||||||||||||||||
3′ CACCCCUAACGACAACUAACUU 5′ Antisense strand (guide strand) (SEQ ID NO: 19)

while the processed antisense guide strand is 5′ UUCAAUCAACAGCAA CCCCAC 3′ (SEQ ID NO: 19). In a similar manner to miPMP22-868, the sixteenth nucleotide in the miPMP22-869 antisense guide strand was changed to a “U”.

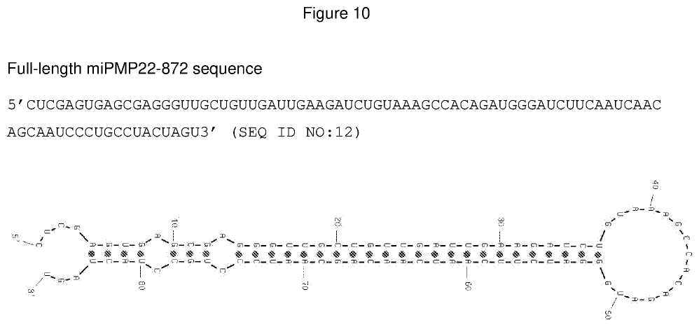

The miPMP22-872 DNA template sequence is
5′CTCGAGTGAGCGAGGGTTGCTGTTGATTGAAGATCTGTAAAGCCACAGATGGGATCTTCA ATCAACAGCAATCCCTGCCTACTAGT3′ (SEQ ID NO: 4), which encodes the full length RNA sequence
5′CUCGAGUGAGCGAGGGUUGCUGUUGAUUGAAGAUCUGUAAAGCCACAGAUGGGAUCUUCA AUCAACAGCAAUCCCUGCCUACUAGU3′ (SEQ ID NO: 12). FIG. 10 shows the folded full length RNA sequence generated using Unafold. The processed mature double-stranded miPMP22-872 is

5′ GGUUGCUGUUGAUUGAAGAUCU 3′ Sense strand (passenger strand) (SEQ ID NO: 46)
||||||||||||||||||||
3′ CCCCAACGACAACUAACUUCUA 5′ Antisense strand (guide strand) (SEQ ID NO: 20)

while the processed antisense guide strand is 5′ AUCUUCAAUCAACAGCAA CCC 3′ (SEQ ID NO: 20). In a similar manner to miPMP22-868, the nineteenth nucleotide in the miPMP22-869 antisense guide strand was changed to a “U”.

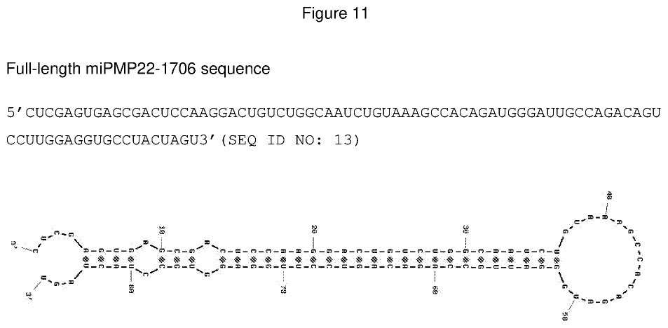

The miPMP22-1706 DNA template sequence is
5′CTCGAGTGAGCGACTCCAAGGACTGTCTGGCAATCTGTAAAGCCACAGATGGGATTGCCAGACAGT CCTTGGAGGTGCCTACTAGT3′ (SEQ ID NO: 5), which encodes the full length RNA sequence
5′CUCGAGUGAGCGACUCCAAGGACUGUCUGGCAAUCUGUAAAGCCACAGAUGGGAUUGCCAGACAGU CCUUGGAGGUGCCUACUAGU3′ (SEQ ID NO: 13). FIG. 11 shows the folded full length RNA sequence generated using Unafold. The processed mature double-stranded miPMP22-1706 is

5′ UCCAAGGACUGUCUGGCAAUCU 3′ Sense strand (passenger strand) (SEQ ID NO: 47)
||||||||||||||||||||
3′ GGAGGUUCCUGACAGACCGUUA 5′ Antisense strand (guide strand) (SEQ ID NO: 21)

while the processed antisense guide strand is 5′ AUUGCCAGACAGUCCUUGGAGG 3′ (SEQ ID NO: 21). The binding of miPMP22-1706 to mouse PMP22 includes a G:U base-pair (which has two hydrogen bonds) as shown below.

```
5′ GUUCUGUGCCUCCAAGGACUGUCUGGCAAUGACUUGUA 3′ HUMAN PMP22 (SEQ ID NO: 48)
                      ||||||||||||||||||||||
3′ GGAGGUUCCUGACAGACCGUUA 5′ miPMP22-1706 (SEQ ID NO: 21)
                      ||||||||||||||||||||||
5′ GUUCUGUGCCUCCAAGGACUGUCUGGCGAUGACUUGUA 3′ MOUSE PMP22 (SEQ ID NO: 49)
```

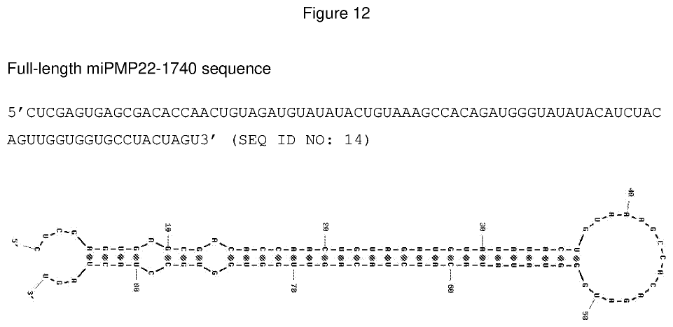

The miPMP22-1740 DNA template sequence is
5′CTCGAGTGAGCGACACCAACTGTAGATGTATATACTGTAAAGCCACAGATGGGTATATACATCTAC AGTTGGTGGTGCCTACTAGT3′ (SEQ ID NO: 6), which encodes the full length RNA sequence
5′CUCGAGUGAGCGACACCAACUGUAGAUGUAUAUACUGUAAAGCCACAGAUGGGUAUAUACAUCUAC AGUUGGUGGUGCCUACUAGU3′ (SEQ ID NO: 14). FIG. 12 shows the folded full length RNA sequence generated using Unafold. The processed mature double-stranded miPMP22-1740 is

5′ ACCAACUGUAGAUGUAUAUACU 3′ Sense strand (passenger strand) (SEQ ID NO: 50)
||||||||||||||||||||
3′ GGUGGUUGACAUCUACAUAUAU 5′ Antisense strand (guide strand) (SEQ ID NO: 22)

while the processed antisense guide strand is 5′ UAUAUACAUCUACAGUUGGUGG 3′ (SEQ ID NO: 22). The binding of miPMP22-1740 to human and mouse PMP22 is shown below.

```
5′ UUGGCCACCAACUGUAGAUGUAUAUAUGGU 3′ HUMAN PMP22 (SEQ ID NO: 51)
              ||||||||||||||||||||||
3′ GGUGGUUGACAUCUACAUAUAU 5′ miPMP22-1740 (SEQ ID NO: 22)
              ||||||||||||||||||||||
5′ UUGGCCACCAACUGUAGAUGUAUAUACGGU 3′ MOUSE PMP22 (SEQ ID NO: 52)
```

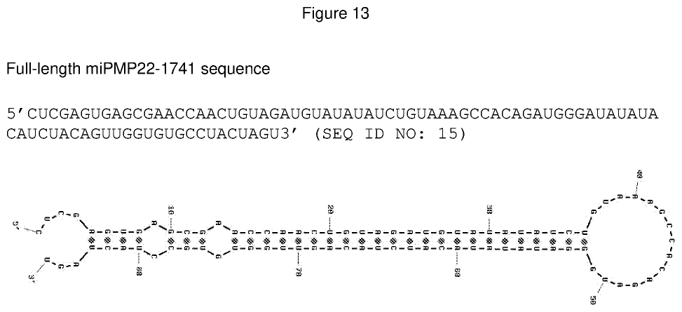

The miPMP22-1741 DNA template sequence is
5′ CTCGAGTGAGCGAACCAACTGTAGATGTATATATCTGTAAAGCCACAGATGGGATATATA CATCTACAGTTGGTGTGCCTACTAGT3′ (SEQ ID NO: 7), which encodes the full length RNA sequence
5′CUCGAGUGAGCGAACCAACUGUAGAUGUAUAUAUCUGUAAAGCCACAGAUGGGAUAUAUA CAUCUACAGUUGGUGUGCCUACUAGU3′ (SEQ ID NO: 15). FIG. 13 shows the folded full length RNA sequence generated using Unafold. The processed mature double-stranded miPMP22-1741 is

5′ CCAACUGUAGAUGUAUAUAUCU 3′ Sense strand (passenger strand) (SEQ ID NO: 53)
||||||||||||||||||||
3′ GUGGUUGACAUCUACAUAUAUA 5′ Antisense strand (guide strand) (SEQ ID NO: 23)

while the processed antisense guide strand is 5′ AUAUAUACAUCUACAGUUGGUG 3′ (SEQ ID NO: 23). The binding of miPMP22-1741 to human and mouse PMP22 is shown below.

```
5′ UUGGCCACCAACUGUAGAUGUAUAUAUGGU 3′ HUMAN PMP22 (SEQ ID NO: 51)
                ||||||||||||||||||||||
3′ GUGGUUGACAUCUACAUAUAUA 5′ miPMP22-1741 (SEQ ID NO: 23)
                |||||||||||||||||||||
5′ UUGGCCACCAACUGUAGAUGUAUAUACGGU 3′ MOUSE PMP22 (SEQ ID NO: 52)
```

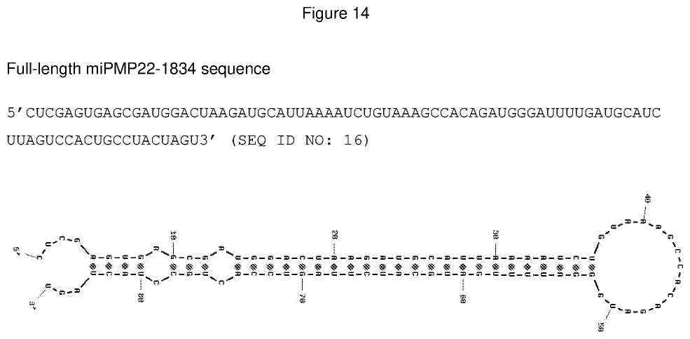

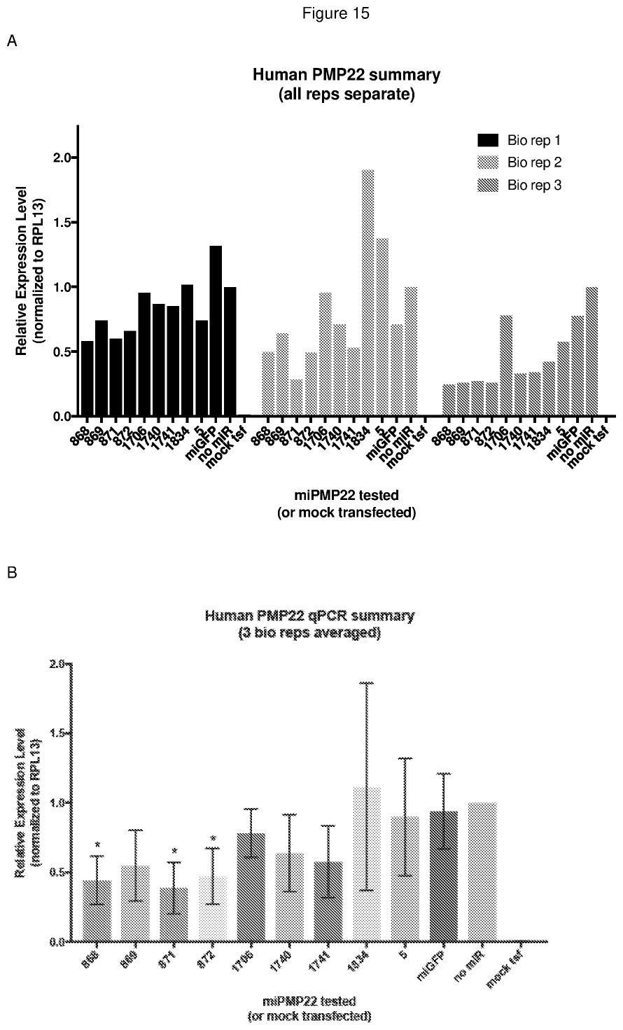

The miPMP22-1834 DNA template sequence is
5′CTCGAGTGAGCGATGGACTAAGATGCATTAAAATCTGTAAAGCCACAGATGGGATTTTGATGCATC TTAGTCCACTGCCTACTAGT 3′ (SEQ ID NO: 8), which encodes the full length RNA sequence
5′CUCGAGUGAGCGAUGGACUAAGAUGCAUUAAAAUCUGUAAAGCCACAGAUGGGAUUUUGAUGCAUC UUAGUCCACUGCCUACUAGU3′ (SEQ ID NO: 16). FIG. 14 shows the folded full length RNA sequence generated using Unafold. The processed mature double-stranded miPMP22-1834 is

5′ GGACUAAGAUGCAUUAAAAUCU 3′ Sense strand (passenger strand) (SEQ ID NO: 54)
||||||||||||||||||||
3′ CACCUGAUUCUACGUAGUUUUA 5′ Antisense strand (guide strand) (SEQ ID NO: 24)

while the processed antisense guide strand is 5′ AUUUUGAUGCAUCUUAGUCCAC 3′ (SEQ ID NO: 24). The binding of miPMP22-1834 to human and mouse PMP22 is shown below.

```
5′ CUGUGUGGACUAAGAUGCAUUAAAAUAAAC 3′ HUMAN PMP22 (SEQ ID NO: 55)
                  ||||||||||||||||||||||||
3′ CACCUGAUUCUACGUAGUUUUA 5′ miPMP22-1834 (SEQ ID NO: 24)
                  ||||||||||||||||||||||||
5′ CUGUGUGGACUAAGAUGCAUCAAAAUAAAC 3′ MOUSE PMP22 (SEQ ID NO: 56)
```

Otherwise, the miPMP22 sequences were generally designed according to Boudreau et al., Chapter 2 of Harper (Ed.), RNA Interference Techniques, Neuromethods, Vol. 58, Springer Science+Business Media, LLC (2011).

This design strategy provides two major advantages: (1) non-allele specific PMP22 gene silencing and (2) testing for efficacy in mice with direct translatability in humans.

### Example 2

In Vitro Testing of miPMP22s

The miPMP22 template sequences were cloned into the U6T6 expression vector [Boudreau et al., pages 19-37 in RNA Interference Methods, Harper (Ed.), Humana Springer Press (2011). The miRNA expression cassette is ˜500 bp in size. The miPMP22s were then co-expressed with Human PMP22 (synthesized by Genscript in pCDNA3.1 expression vector) in HEK293T cells using a 4:1 miR:target molar ratio. Cells were transfected using Lipofectamine2000 and incubated for 24 h. Total RNA was collected using Trizol (Invitrogen), random primed cDNA was synthesized (High Capacity cDNA RT Kit, ThermoFisher), and PMP22 knock-down was assessed by qRT-PCR using a Taqman probe against human and murine PMP22 (Hs00165556_m1, Mm01333393_m1, ThermoFisher).

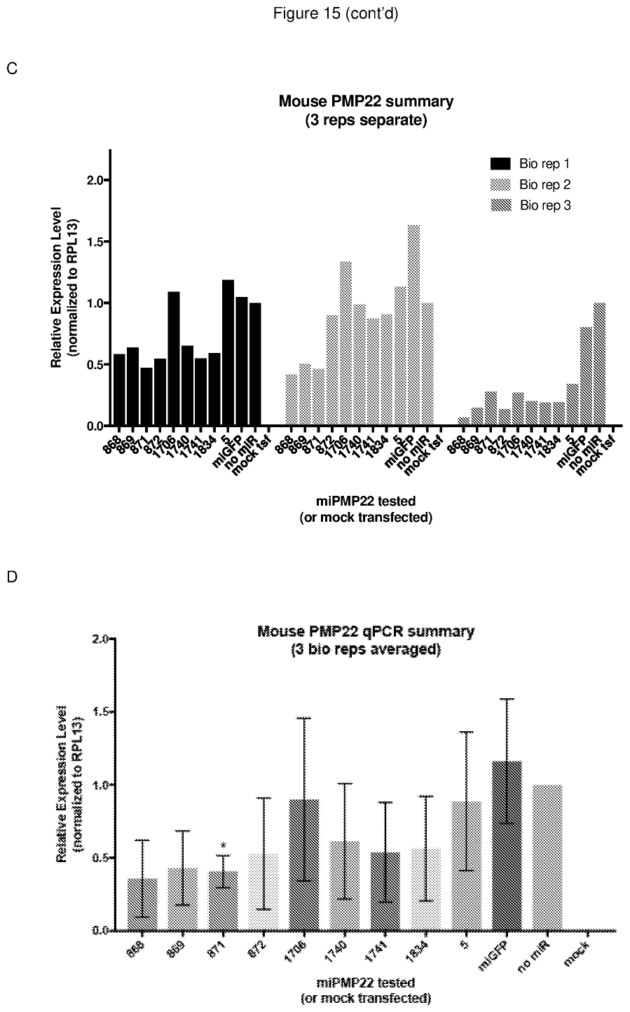

The qRT-PCR knock-down testing identified three lead candidates: miPMP22-868, miMPM22-871, and miPMP22-872. These three miPMP22s were able to significantly reduce Human PMP22 transcript level compared to the untreated (“no miR”) condition (FIG. 15). Results are the average of three independent experiments.

Template sequences encoding the two strongest miPMP22s (868 and 871) were cloned into a scAAV9 for in vivo delivery as described below.

### Example 3

Production of scAAV9 Encoding miPMP22s

The miPMP22-868 and -871 template sequences were cloned into the scAAV9 construct generically named “scAAV-NP.U6.miPMP22.CMV.eGFP” for in vivo delivery. The scAAV9 also contained a CMV promoter-driven eGFP reporter gene. The scAAV9 comprised a mutant AAV2 inverted terminal repeat (ITR) and a wild type AAV2 ITR that enable packaging of self-complementary AAV genomes. The resulting scAAV9 are referred to as “AAV9-miR868” (short for the scAAV9 construct scAAV-NP.U6.miPMP22-868.CMV.eGFP) and “AAV9-miR871” (short for the scAAV9 construct scAAV-NP.U6.miPMP22-871.CMV.eGFP). A non-targeting scAAV referred to as “AAV9-miRLacZ” (short for a scAAV construct scAAV-NP.U6.miRLacZ.CMV.eGFP).

The scAAV9 were produced by transient transfection procedures using a double-stranded AAV2-ITR-based vector, with a plasmid encoding Rep2Cap9 sequence as previously described [Gao et al., J. Virol., 78: 6381-6388 (2004)] along with an adenoviral helper plasmid pHelper (Stratagene, Santa Clara, CA) in 293 cells. The scAAV9 were produced in three separate batches for the experiments and purified by two cesium chloride density gradient purification steps, dialyzed against PBS and formulated with 0.001% Pluronic-F68 to prevent virus aggregation and stored at 4° C. All vector preparations were titered by quantitative PCR using Taq-Man technology. Purity of vectors was assessed by 4-12% sodium dodecyl sulfate-acrylamide gel electrophoresis and silver staining (Invitrogen, Carlsbad, CA). scAAV9 viruses were generated and titered by the Viral Vector Core at The Research Institute at Nationwide Children's Hospital.

### Example 4

### Animal Model

A C61-Het mice colony was established starting from two breeding pairs gifted by Prof. R. Martini (University Hospital of Wurzburg). This model is known to express four copies of the human PMP22 gene in addition to the endogenous mouse gene resulting to a two-fold overexpression of human PMP22 transgene when compared to endogenous wild type murine PMP22 [Huxley et al., (1996) supra; Huxley et al., (1998) supra; and Sereda et al., NeuroMolecular Med., 8(1-2): 205-216 (2006)]. All experimental procedures were conducted in accordance with animal care protocols approved by the Cyprus Government's Chief Veterinary Officer (project license CY/EXP/PR.L2/2012) according to national law, which is harmonized with EU guidelines (EC Directive 86/609/EEC).

### Example 5

### Intrathecal Vector Delivery

AAV9-miR871 (targeting), AAV9-miR868 (targeting) and AAV9-miRLacZ viruses (non-targeting control) were injected intrathecally into 2-month or 2-month old C61 Het mice to examine their effect on human/mouse PMP22 and other myelin-related proteins. For intrathecal delivery, a 50-μL Hamilton syringe connected to a 26-gauge needle was used to inject 20 μl of AAV9 stock containing an estimated amount of AAV9-miRLacZ: 1.66×10¹³ DRP/ml, AAV9-miR871: 3.0×10¹³ DRP/ml, or AAV9-miR868: 2.7×10¹³ DRP/ml vectors into anesthetized mice in the L5-L6 intrathecal space at a slow rate of 5 μl/min. A correct injection was verified by flick of the tail as previously described [Kagiava et al., P.N.A.S. USA, 113(17): E2421-E2429 (2016); Kagiava et al., Hum. Mol. Genet., 27(8):1460-1473 (2018); Kagiava et al., Methods in Molecular Biology, 1791: 277-285 (2018); and Schiza et al., Brain, 142(5): 1227-1241 (2019)].

### Example 6

### Biodistribution and Expression

The AAV9 miR871 (targeting) and miRLacZ viruses (non-targeting control) were injected intrathecally as described above into the 2-month old C61 Het mice to examine their effect on human/mouse PMP22 and other myelin-related proteins. Mice were analyzed for immunostaining after 4 and 8 weeks post-injection for the eGFP reporter gene expression as well as by Real-Time PCR or immunoblot analysis of the PMP22 expression 6 weeks post-injection. AAV9 vector biodistribution in PNS tissues was also assessed by Vector Genome Copy Number (VGCN) analysis.

For immunohistochemistry, mice were anaesthetized and transcardially perfused with phosphate-buffered saline (PBS) followed by fresh 4% paraformaldehyde. The lumbar spinal cord with spinal roots attached as well as sciatic and femoral nerves were dissected and frozen for cryosections. Part of the sciatic and femoral nerves were also teased into fibers under a stereoscope. Sections and fibers were permeabilized in cold acetone and incubated at room temperature with a blocking solution of 5% BSA containing 0.5% Triton-X for 1 h. The slides were incubated overnight at 4° C. with the primary antibody which was rabbit antisera against eGFP (1:2,000; Invitrogen) diluted in blocking solution. Slides were then washed in PBS and incubated with rabbit cross-affinity purified secondary antibody (Jackson ImmunoResearch, diluted 1:500) for 1 h at room temperature. Cell nuclei were visualized with DAPI. Slides were mounted with fluorescent mounting medium and images were photographed under a fluorescence microscope with a digital camera using NIS-elements software (Nikon). eGFP was also visible as an auto-fluorescent signal without employing an anti-eGFP antibody. The percentage of eGFP+SCs of lumbar spinal roots, sciatic nerves and femoral nerves was counted as recently described [Kagiava et al, Hum. Mol. Genet., 28(21): 3528-3616 (2019)].

For Immunoblot analysis, fresh lumbar roots, sciatic and femoral nerves were collected from groups of C61 Het mice that were intrathecally injected at 2 months of age with AAV9-miR871 or AAV9-miRLacZ vectors and sacrificed either at 4 and 8 weeks post-injected (biodistribution experiments) or at six weeks post-injection (silencing experiments), lysed in ice-cold RIPA buffer (10 mM sodium phosphate, pH 7.0, 150 mM NaCl, 2 mM EDTA, 50 mM sodium fluoride, 1% Nonidet P-40, 1% sodium deoxycholate, and 0.1% SDS) containing a mixture of protease inhibitors (Roche). Proteins (150 μg) from the lysates were fractionated by 12% SDS/PAGE and then transferred to a PVDF membrane (GE Healthcare Life Sciences) using a semidry transfer unit. Nonspecific sites on the membrane were blocked with 5% nonfat milk in PBS with Tween 20 (PBST) for 1 h at room temperature. Immunoblots were incubated with anti-huPMP22 (1:500 Abcam), anti-eGFP (1:1000 Abcom) and anti-msTubulin (1:3000; Developmental Studies Hybridoma Bank, for loading control) antibodies at 4° C. overnight. After washing with PBST, the immunoblots were incubated with HRP-conjugated secondary antiserum (Jackson ImmunoResearch, diluted 1:3000) in 5% milk-PBST for 1 h. Blots were again washed with PBST and the bound antibody was visualized by an enhanced chemiluminescence system (GE Healthcare Life Sciences).

For Real-Time PCR analysis, RNA was isolated from snap-frozen lumbar roots, sciatic and femoral nerves from groups of C61 Het mice that were intrathecally injected at 2 months of age with AAV9-miR871, AAV9-miR868 or AAV9-miRLacZ vectors and sacrificed six weeks post-injection using Qiagen RNeasy® Lipid Tissue Mini Kit following the manufacturer's protocol. After DNase treatment, RNA was quantified by spectrophotometry and 0.3 μg of RNA was used to synthesize cDNA employing TaqMan™ reverse-transcription reagents. Then the levels of huPMP22, muPMP22, muMPZ, muCNP, muGldn and muGJB1 mRNA were quantified using Taqman gene expression assays (Applied Biosystems) and muGAPDH assay as an endogenous control. huPMP22, muPMP22, muMPZ, muCNP muGldn and muGJB1 expression levels in AAV9-miR871 and AAV9-miR868 treated mice were compared to analogous expression levels of AAV9-miRLacZ treated littermates.

For Vector Genome Copy Number (VGCN) analysis, DNA was extracted from lumbar root and sciatic nerves using Meslo MagPurix Tissue DNA extraction kit following manufacturer instructions. For detection and quantification of vector genomes in extracted DNA, Droplet Digital PCR analysis was conducted using probes against on eGFP (reporter gene) and TRFC (loading control). The average VGCN per cell was calculated as the total VGCN divided by the total cell number.

Immunostaining (percentage of eGFP-expressing cells) and immunoblot (normalized ratio of optic densities) data were compared using unpaired Student t-test GraphPad Prism5 software. Significance level for all comparisons, P<0.05. Further details of each statistical analysis are indicated with each result.

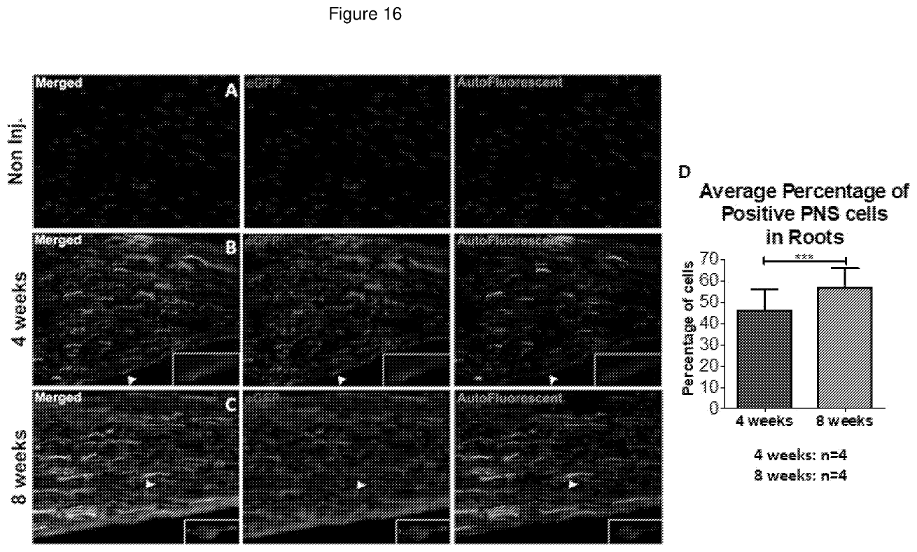

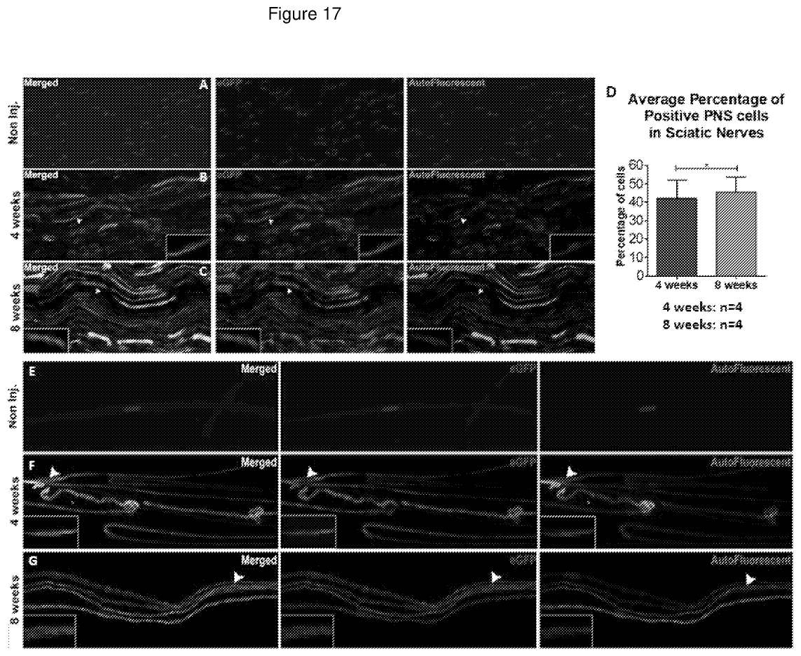

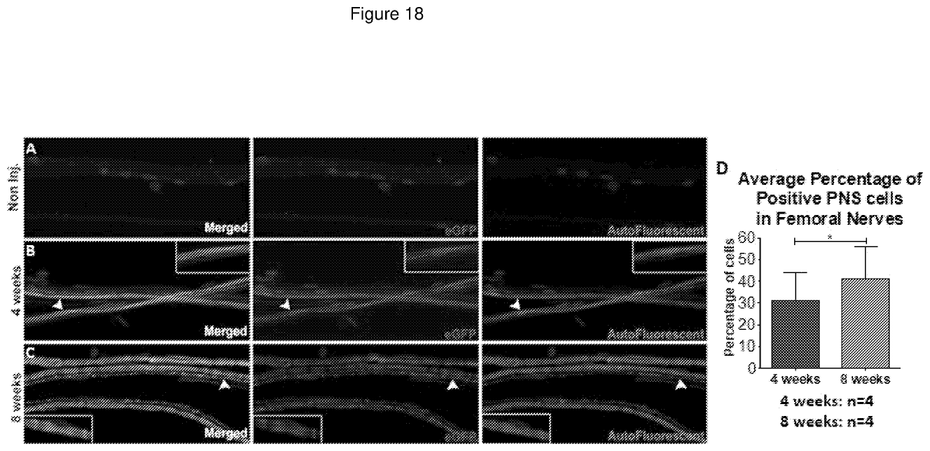

In lumbar roots of adult mice, expression rates analysis showed that 45.96% and 56.82% of all PNS cells were positive for eGFP, 4 and 8 weeks post injection, respectively (FIG. 16). In sciatic nerves, expression rates analysis showed that 42.06% and 45.74% of all cells were positive for eGFP, 4 and 8 weeks post injection, respectively (FIG. 17). In femoral nerves, expression rates analysis showed that 31.06% and 41.09% of cells were positive for eGFP, 4 and 8 weeks post injection, respectively (FIG. 18). As expected from the fact that expression was driven by ubiquitous promoters, we also detected eGFP signal along axons both in the spinal cord as well as in lumbar roots, sciatic and femoral nerves.

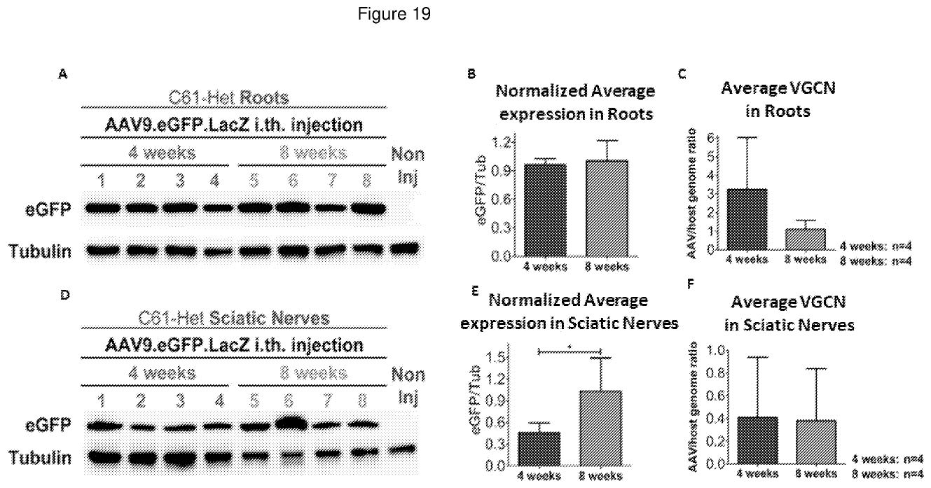

The expression of eGFP in adult mice was also demonstrated by immunoblot analysis using the anti-eGFP antibody in lumbar root and sciatic nerve lysates obtained from two groups of 2-month old mice which were intrathecally injected with AAV9-U6-miRLacZ-CMV-eGFP. For this purpose, fresh lumbar roots and sciatic nerves were collected at 4 and 8 weeks post-injection and lysed in ice-cold RIPA buffer. The predicted eGFP protein band at 30 kDa was detectable in lumbar roots (Average roots eGFP/Tub ratio: 4 weeks: 0.96, 8 weeks: 1.01) as well as in sciatic nerves (Average sciatic nerve eGFP/Tub ratio: 4 weeks: 0.46, 8 weeks: 1.03) of examined AAV9-miLacZ treated mice compared to the negative control, but not at the same level (FIG. 19 A, B, D, E).

Furthermore, VGCN analysis performed on the extracted genomic DNA from lumbar roots (Average roots VGCN: 4 weeks: 3.25, 8 weeks: 1.9) and sciatic nerves (Average sciatic nerve VGCN: 4 weeks: 0.41, 8 weeks: 0.38) of mice 4 and 8 weeks post injection showed overall an adequate and stable over time biodistribution although with variation among animals (FIG. 19 C, F).

### Example 7

### In Vivo AAV-Mediated Gene Silencing of PMP22

After confirming adequate biodistribution and expression of the control AAV9-miRLacZ vector in PNS tissues, the effects of AAV9-miR871 and AAV9-miR868 at selected transcripts of C61-Het mice were assessed.

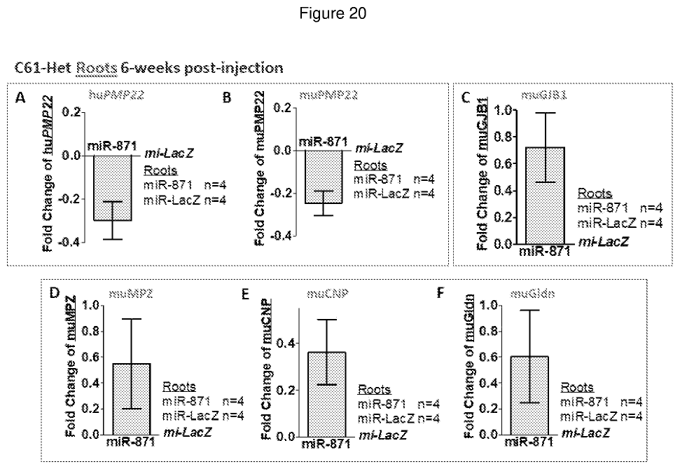

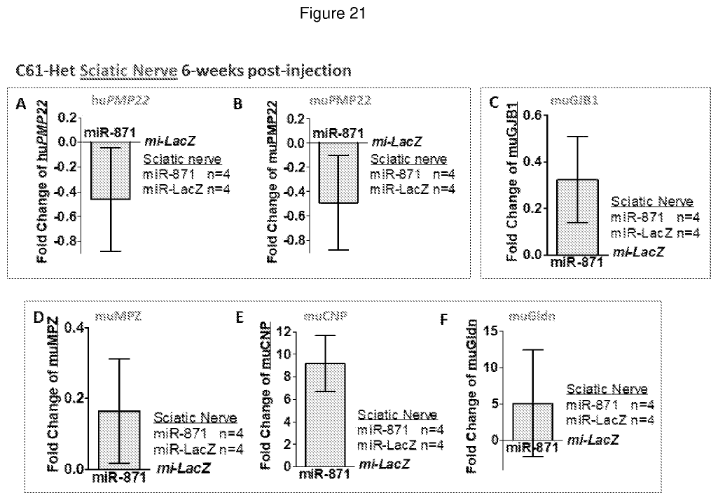

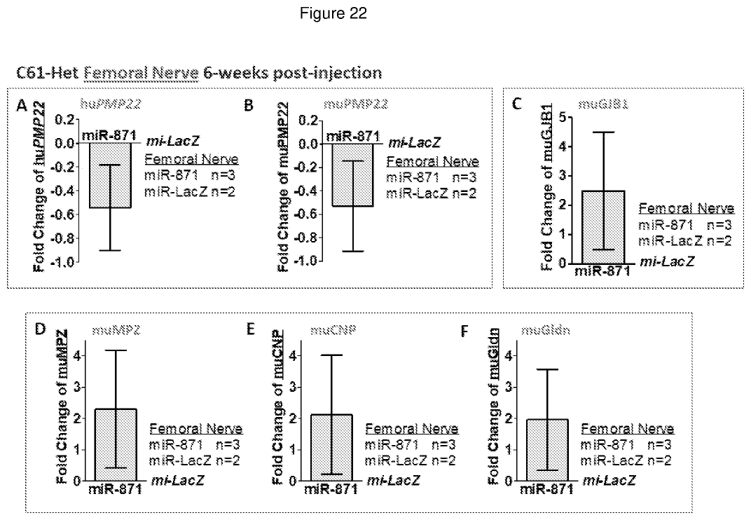

The lumbar roots of the C61-Het mice treated with AAV9-miR871 showed reduced human and mouse PMP22 mRNA levels by 0.29 and 0.25 fold, respectively, while mRNA levels of other myelin related proteins including GJB1, MPZ, CNP and Gldn were increased by 0.72, 0.54, 0.36 and 0.61 fold, respectively (FIG. 20). In accordance, the sciatic nerves of the mice treated with AAV9-miR871 showed reduced levels of human and mouse PMP22 mRNA levels by 0.46 and 0.49 fold, respectively, while mRNA levels of other myelin related proteins including GJB1, MPZ, CNP and Gldn were increased by 0.32, 0.16, 9.16 and 5.06 fold, respectively (FIG. 21). The femoral nerves of the AAV9-miR871 treated mice showed reduced levels of human and mouse PMP22 mRNA levels by 0.54 and 0.53 fold, respectively, while mRNA levels of other myelin related proteins including GJB1, MPZ, CNP and Gldn were increased by 2.49, 2.3, 2.12 and 1.96 fold, respectively (FIG. 22).

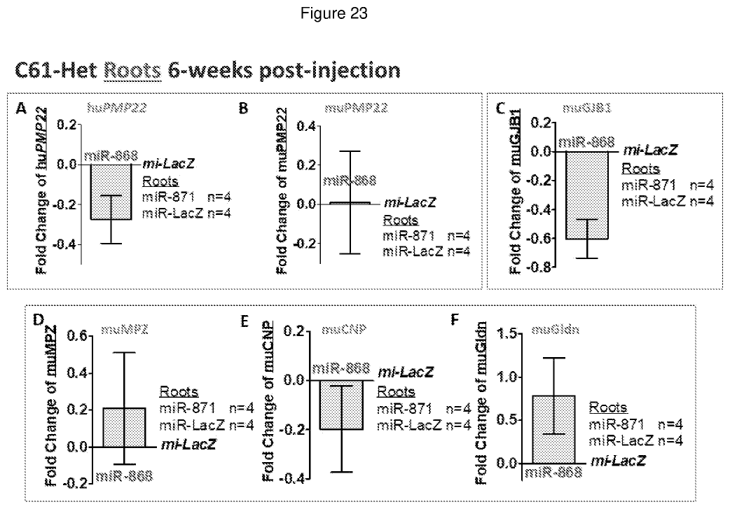

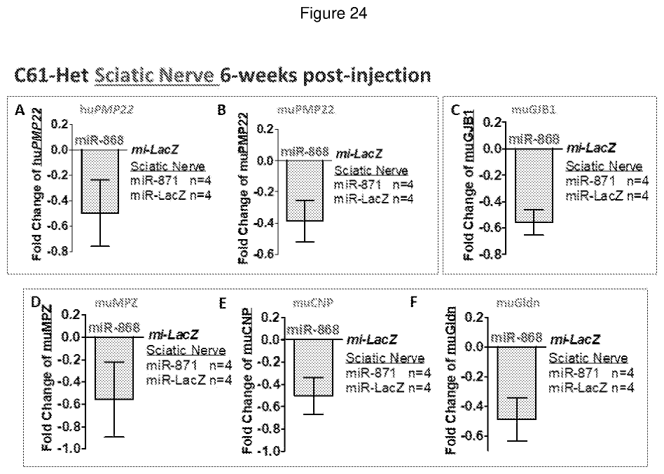

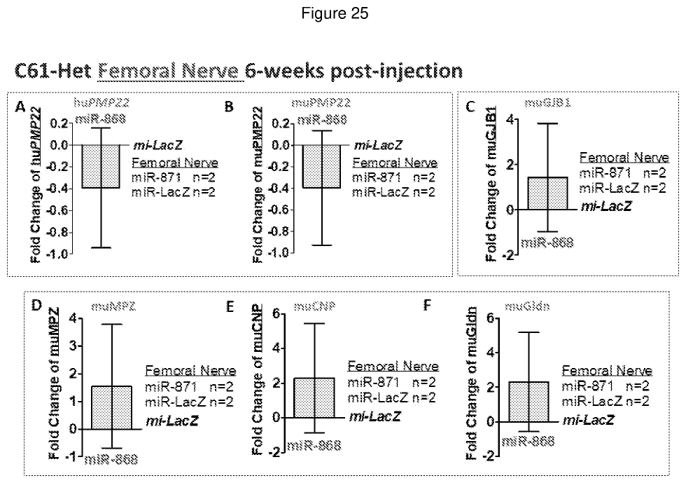

The lumbar roots of the C61-Het mice treated with AAV9-miR868 showed reduced human and mouse PMP22 mRNA levels by 0.27 and 0.01 fold, respectively, while mRNA levels of other myelin related proteins including GJB1, MPZ, CNP and Gldn were altered by −0.6, 0.21, −0.24 and 0.78 fold, respectively (FIG. 23). The sciatic nerves of the mice treated with AAV9-miR868 showed reduced levels of human and mouse PMP22 mRNA levels by 0.49 and 0.38 fold, respectively, while mRNA levels of other myelin related proteins including GJB1, MPZ, CNP and Gldn was decreased by 0.56, 0.55, 0.50 and 0.49 fold, respectively (FIG. 24). The femoral nerves of the AAV9-miR868 treated mice showed reduced levels of human and mouse PMP22 mRNA levels by 0.39 and 0.40 fold, respectively, while mRNA levels of other myelin related proteins including GJB1, MPZ, CNP and Gldn were increased by 1.44, 1.54, 2.28 and 2.30 fold, respectively (FIG. 25).

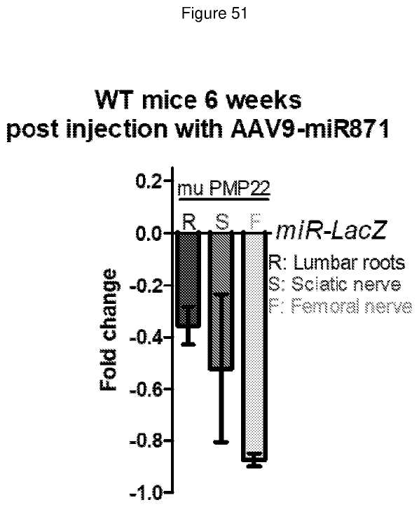

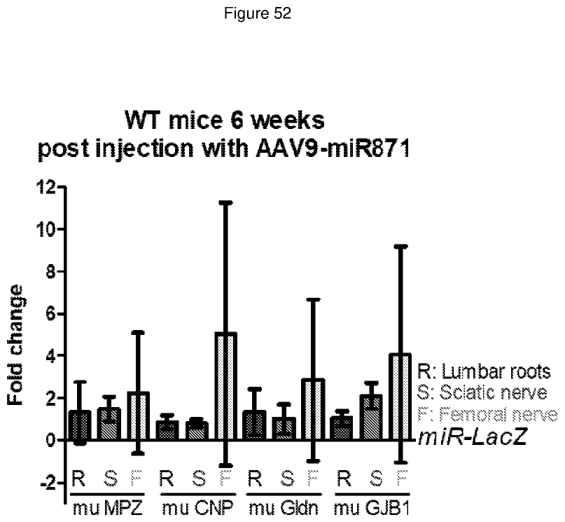

The lumbar roots of WT mice treated with AAV9-miR871 showed reduced mouse PMP22 mRNA levels by 0.36 fold, while mRNA levels of other myelin related proteins including GJB1, MPZ, CNP and Gldn were increased by 1.02, 1.31, 0.85 and 1.31 fold, respectively (FIGS. 51 and 52). In accordance, the sciatic nerves of WT mice treated with AAV9-miR871 showed reduced levels of mouse PMP22 mRNA levels by 0.52 fold while mRNA levels of other myelin related proteins including GJB1, MPZ, CNP and Gldn were increased by 2.09, 1.47, 0.79 and 1.01 fold, respectively (FIGS. 51 and 52). The femoral nerves of WT AAV9-miR871 treated mice showed reduced levels mouse PMP22 mRNA levels by 0.87 while mRNA levels of other myelin related proteins including GJB1, MPZ, CNP and Gldn were increased by 4.06, 2.29, 5.03 and 2.86 fold, respectively (FIGS. 51 and 52).

Based on the results, AAV9-miR871 was chosen as the most promising in silencing the hu/ms PMP22 mRNA while also resulting in the enhanced transcription of other myelin proteins. Therefore, its effect on human PMP22 protein levels of lumbar roots, sciatic and femoral nerves was assessed using immunoblot analysis.

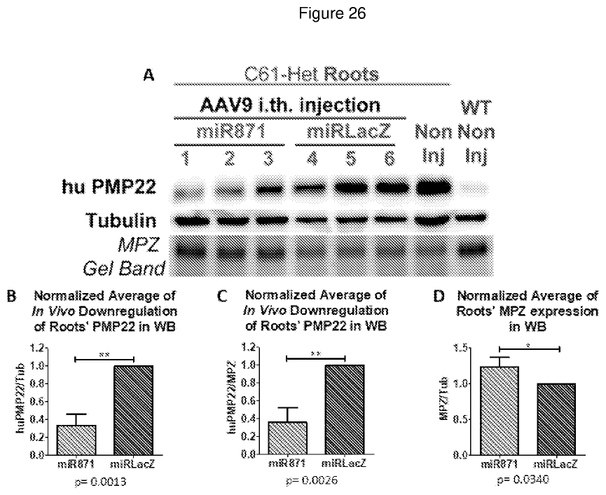

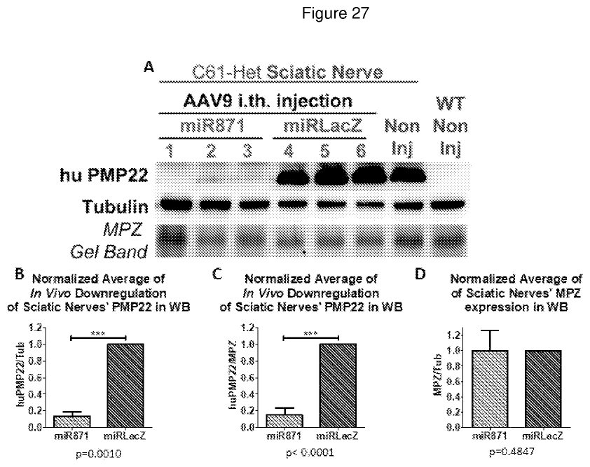

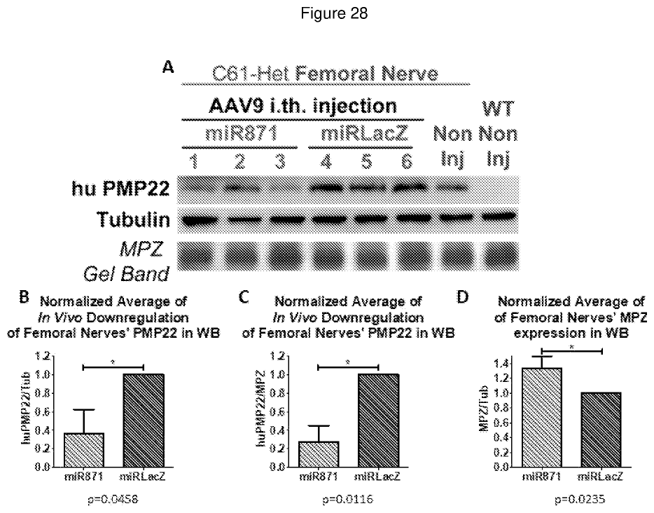

PMP22 protein band was normalized using either tubulin immunoblot band or Myelin protein zero (MPZ) SDS gel band (FIG. 26), in both scenarios root HuPMP22 protein levels were shown to be significantly reduced after miR871 treatment by more than 60% (HuPMP22/Tub: 67% silencing, HuPMP22/MPZ: 64% silencing) when compared to miRLacZ treated mice. When normalize to tubulin MPZ protein expression was shown to be increased by 24% when compared to miRLacZ treated mice. Similarly, HuPMP22 protein of sciatic nerves was significantly reduced by more than 85% (HuPMP22/Tub: 87% silencing, HuPMP22/MPZ: 85% silencing) while MPZ protein expression remained unchanged (MPZ/Tub: 0.008% silencing) when compared to miRLacZ treated mice (FIG. 27). In femoral nerves HuPMP22 protein levels were significantly reduced by more than 60% (HuPMP22/Tub: 64% silencing, HuPMP22/MPZ: 72% silencing) while MPZ protein expression was significantly increased by 34% when compared to miRLacZ treated mice (FIG. 28).

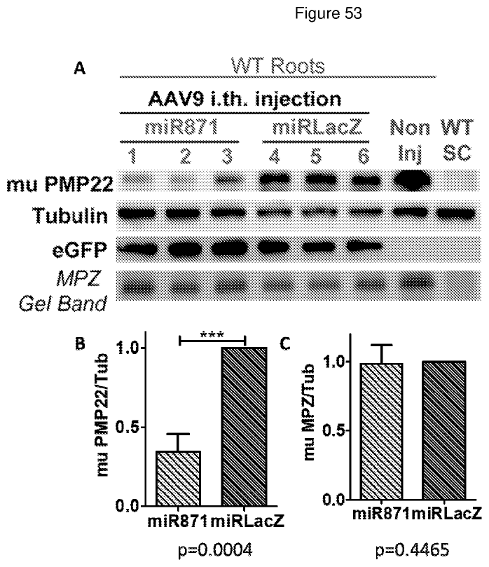

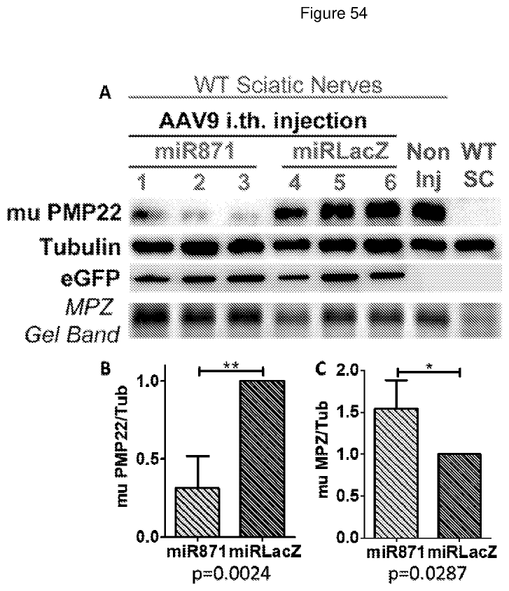

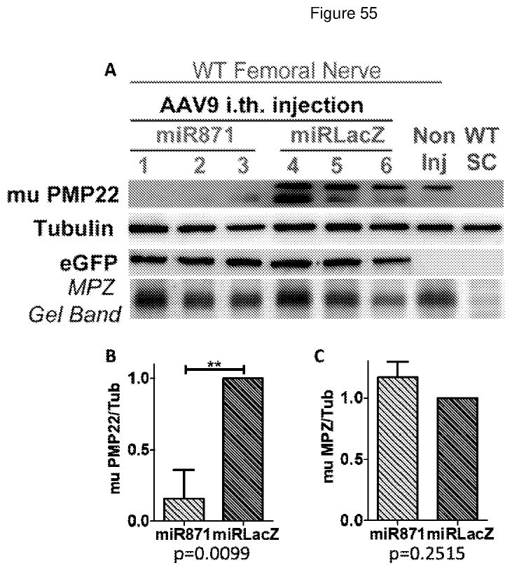

Similarly, AAV9-miR871 injection in WT mice reduced the levels of murine PMP22 in lumbar roots by 66% while leaving MPZ levels unchanged (FIG. 53). In WT sciatic nerves, AAV9-miR871 injection resulted in 69% reduction of murine PMP22 while increasing MPZ protein levels by 54% (FIG. 54). WT femoral nerves responded similarly to WT lumbar roots as AAV9-miR871 injection reduced murine PMP22 protein levels by 99% while leaving MPZ protein levels unchanged (FIG. 55). Statistics: Immunoblot (ratio of optic densities) data were compared using Student t-test GraphPad Prism5 software. Significance level for all comparisons, P<0.05.

### Example 8

Therapeutic Trials with AAV9-miR871 Vector to Rescue the Mouse Model of CMT1A

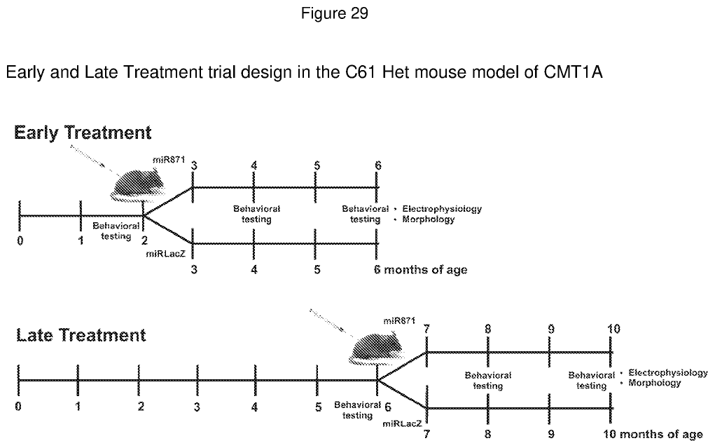

After confirming adequate silencing effects of AAV9-miR871 vector in adult C61 Het mice with already advanced peripheral nerve pathology, early- and late-onset treatment trials were conducted according to the trial design shown in FIG. 29. Randomized, non-targeting vector-controlled treatment trials were conducted in the C61 Het mouse model of CMT1A using groups injected either with AAV9-miR871 or AAV9-miRLacZ at the age of 2 months (early treatment) or at the age of 6 months (late treatment).

C61-Het mice were randomized into four groups according to their age and the treatment received, AAV9-miR871 or AAV9-miRLacZ. Outcome analysis was performed four months post-injection for both treatment time points and included motor behavioral performance, motor nerve conduction studies as well as morphometric analysis to determine the degree of demyelination by measuring the percentage of thinly myelinated and demyelinated axons, along with the total number of onion bulb formations. Outcome analysis also included plasma quantification of Nfl levels as well as CD20, CD45, CD3 and CD68 marker immune response of C61 Het-AAV9-miR871 early and late treatment mice groups.

Littermate mice in each age group were randomized to either receiving the targeting vector (AAV9-miR871; treatment group) or non-targeting vector (AAV9-miRLacZ; control group). Randomization was based on animal numbering after tailing (mice with odd numbers will be randomized to treated and mice with even numbers to control treatment). Mice were evaluated by clinical testing, at the age of 6 months for early treatment and at the age of 10 months for late treatment, followed by electrophysiological study, plasma collection for Nfl analysis, as well as perfusion and quantitative morphometric and immune response analyses. Primary endpoint was considered the rescue of pathological changes in lumbar roots and femoral motor nerve. Secondary endpoints were the significant improvement in sciatic nerve motor conduction velocities, improvement in clinical motor behavioral performance, improved immunological reaction in lumbar roots, femoral motor nerve and liver as well as plasma Nfl levels.

Statistics: Behavioral, electrophysiological and morphological analysis data of the miRLacZ-, miR871-, early or late, treated mice were compared using the Mann-Whitney test GraphPad Prism5 software. Significance level for all comparisons, P<0.05. Further details of each statistical analysis are indicated in each result.

### Example 9

### Behavioral Testing

Strength and coordination were compared in treated and control-treated animals as described in [Kagiava (2016), supra; Schiza, supra]. Examiners were blinded to the treatment status (treated or control vector-treated) of the animals. Motor performance was assessed before the injection at the age of 2 (early treatment) or 6 (late treatment) months and again at 2 and 4 months post-injection by rotarod (at 5 and 17.5 rpm), foot grip and hang tests.

Rotarod testing: Motor balance and coordination was determined according to described protocols [Kagiava, Hum. MoL. Genet. (2018), supra; Savvaki, supra] using an accelerating rotarod apparatus (Ugo Basile, Italy). Training of animals was consisted of three trials per day with 15 min intervals for resting between trials for three consecutive days. Mice were placed on the rod with the speed gradually increasing from 2.5 to 25 rpm. The trial ends when the mouse falls from the rod, missteps, or when it remains on the rod for 600 s. Testing was performed on the 4^{th} day using two different speeds, 5 and 17.5 rpm. Latency (s) to fall is calculated for each speed.

Grip strength testing: To measure grip strength mice were held by the tail and lowered towards the apparatus (Ugo Basile) until they grab the grid with their hindlimbs, then gently pulled back until they release the grid. Each session consists of six consecutive trials. Measurements of the force in g were indicated on the equipment.

Wire hang testing: The wire hang test seeks to evaluate motor function and grip strength. The test begins with the animal hanging from an elevated wire. The animal was placed on the wire top, which was then inverted; the latency to when the animal falls was recorded. This test was performed once a day for three days consecutive and then the average performance was calculated.

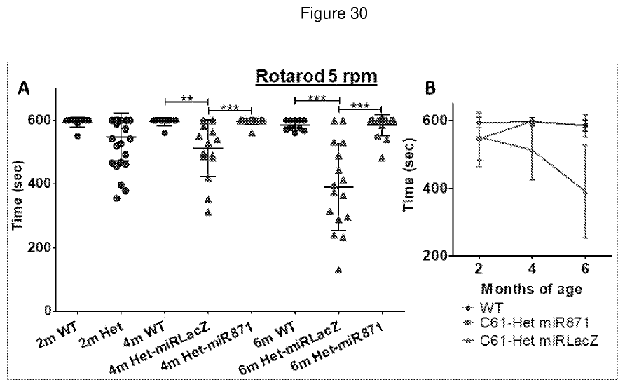

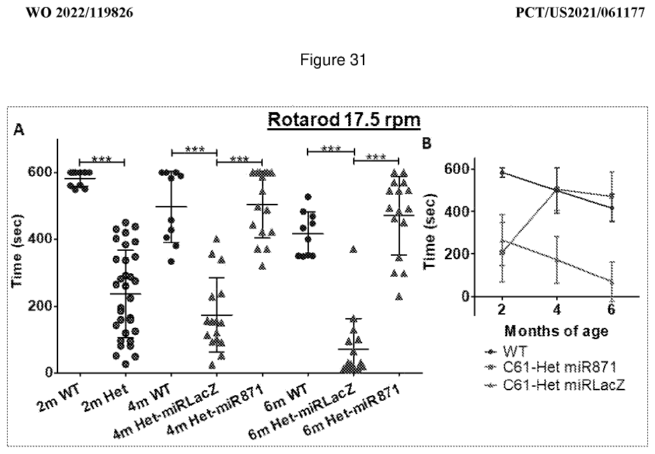

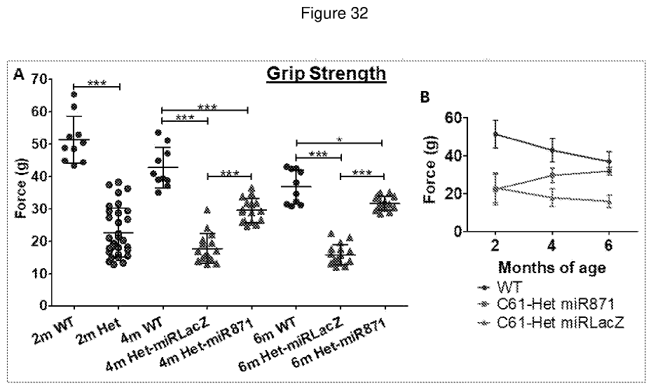

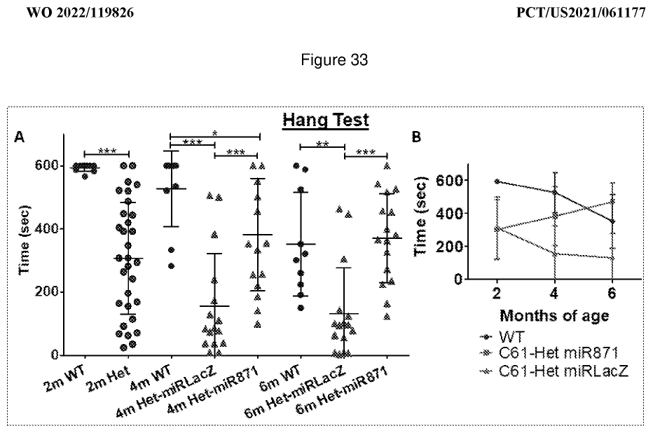

For the early treatment group, motor performances were assessed before the injection at the age of 2 months and again 2 months post-injection and 4 months post-injection by rotarod, foot grip and hang tests (FIGS. 30-33). Comparing 2-month-old WT and non-injected C61 Het mice with the above tests proves the significantly impaired motor performance of the CMT1A model. The only test that did not show a significant difference among these two groups was rotarod at 5 rpm at 2 months of age, but it did show progressive deterioration in the CMT1A model over time that was rescued by treatment (FIG. 30). All other tests employed to assess motor performance showed that 4- and 6-month-old WT mice differ significantly to their age-matched AAV9-miRLacZ treated mice (FIGS. 30-33).

For the early treatment trial groups, 2-month-old C61 Het mice were randomly allocated to each treatment (miR871 or miRLacZ). As expected, 2 months old C61 Het mice that were either treated with AAV9-miR871 or AAV9-miRLacZ, showed no significant differences at baseline before initiating the treatment. Importantly, at 4 and 6 months of age, 2 and 4 months after treatment, all motor performance tests (rotarod 5 & 17.5 rpm, grip strength, wire hang test) showed a significant improvement of AAV9-miR871 treated mice when compared to AAV9-miLacZ treated control littermates (FIGS. 30-33).

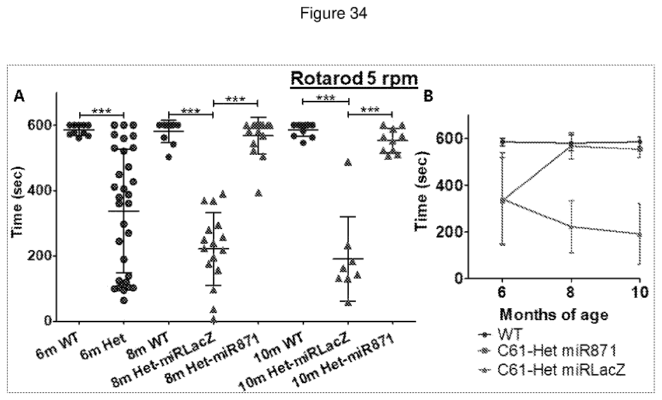

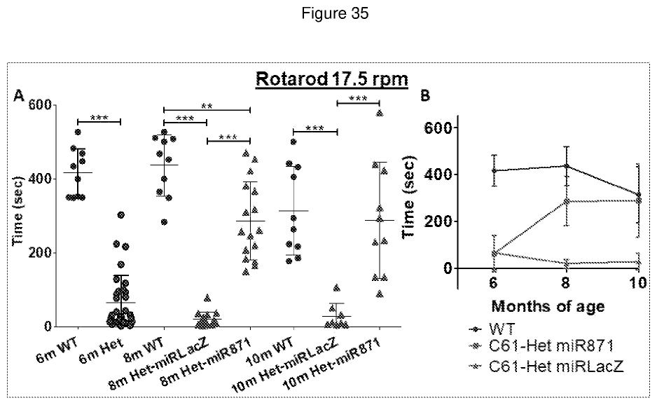

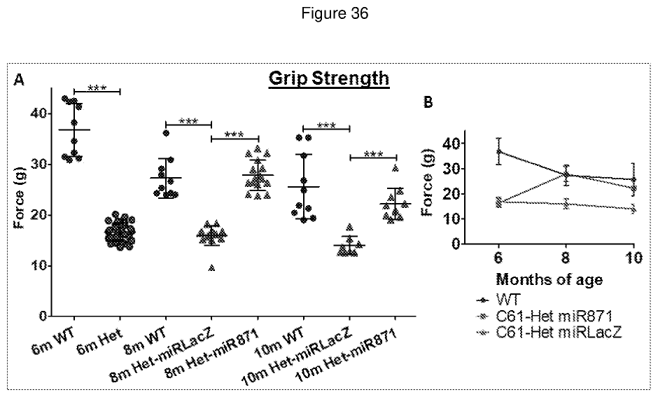

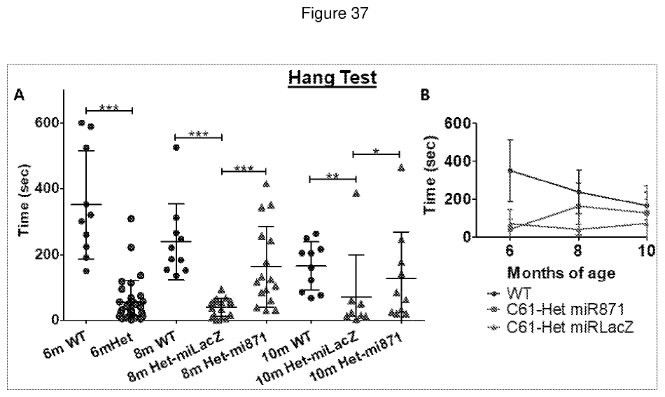

For the late treatment group, motor performances were assessed before the injection at the age of 6 months and again 2 months post-injection and 4 months post-injection by rotarod, foot grip and hang tests (FIGS. 34-37). Comparing 6-month-old WT and non-injected C61 Het mice with the above tests proves the significantly impaired motor performance of the model. All other tests employed to assess motor performance showed that 6- and 8-month-old WT mice differ significantly to their age-matched AAV9-miRLacZ treated mice (FIGS. 34-37).

For the late treatment trial groups, 6-month-old C61 Het mice were randomly allocated to each treatment (miR871 or miRLacZ). As expected, 6 months old C61 Het mice that were either treated with AAV9-miR871 or AAV9-miRLacZ, showed no significant differences at baseline before initiating the treatment. Importantly, at 6 and 8 months of age, 2 and 4 months after treatment, all motor performance tests (rotarod 5 & 17.5 rpm, grip strength, wire hang test) showed a significant improvement of AAV9-miR871 treated mice when compared to AAV9-miLacZ treated control littermates (FIGS. 34-37).

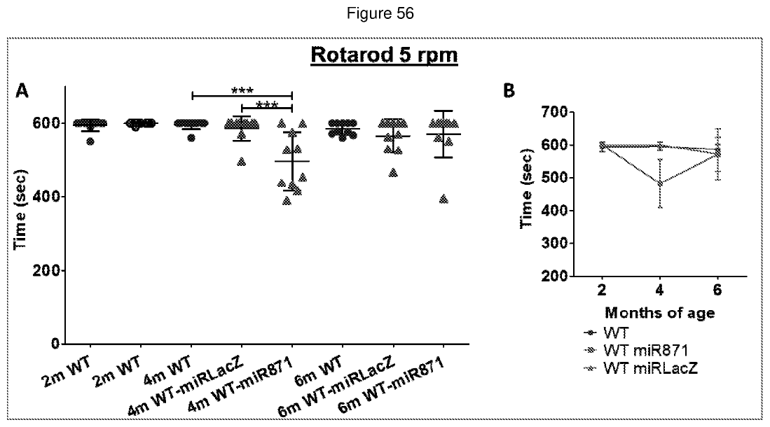

For the WT injected groups, 2-month-WT mice were randomly allocated to each treatment (miR871 or miRLacZ). As expected, for all motor performance tests 2 months old WT mice that were either treated with AAV9-miR871 or AAV9-miRLacZ, showed no significant differences at baseline before injection. For the WT injected groups, motor performances were assessed before the injection at the age of 2 months and again 2 months post-injection and 4 months post-injection by rotarod, foot grip and hang tests (FIGS. 56-59).

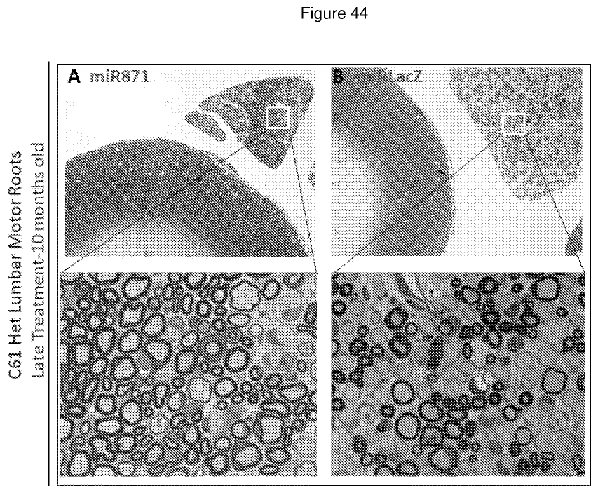

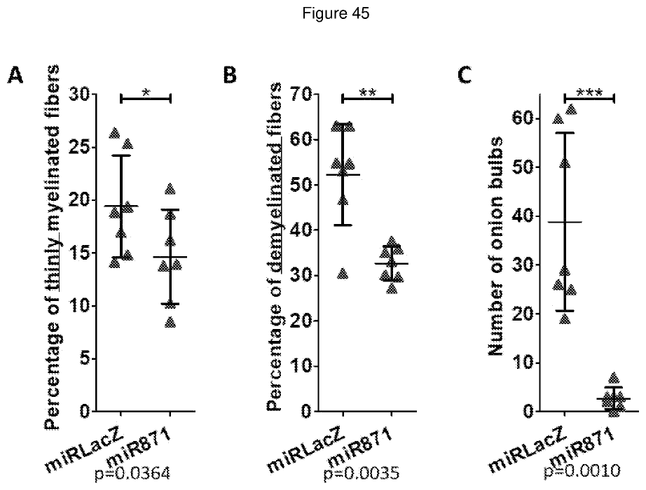

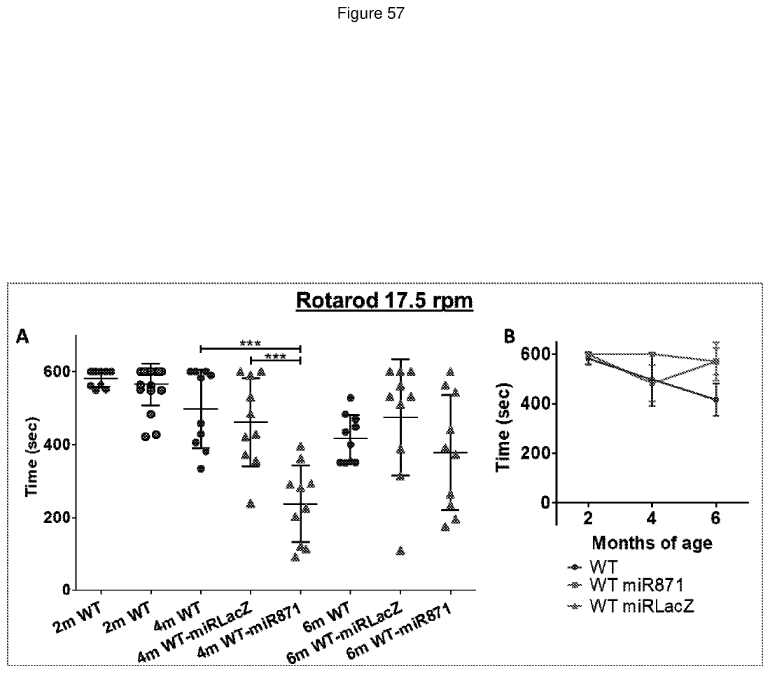

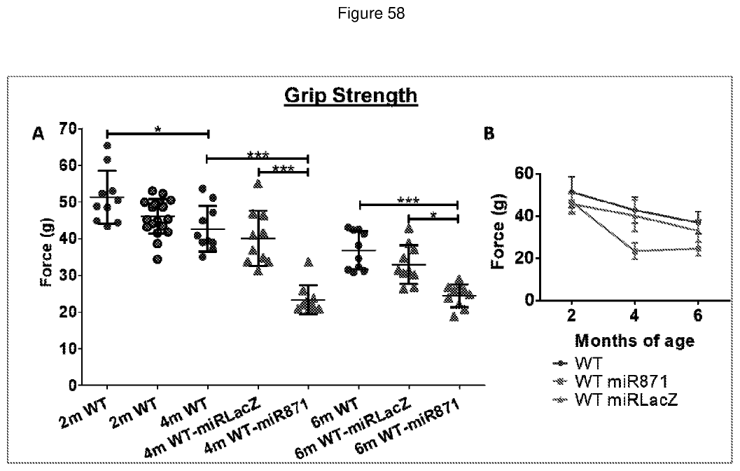

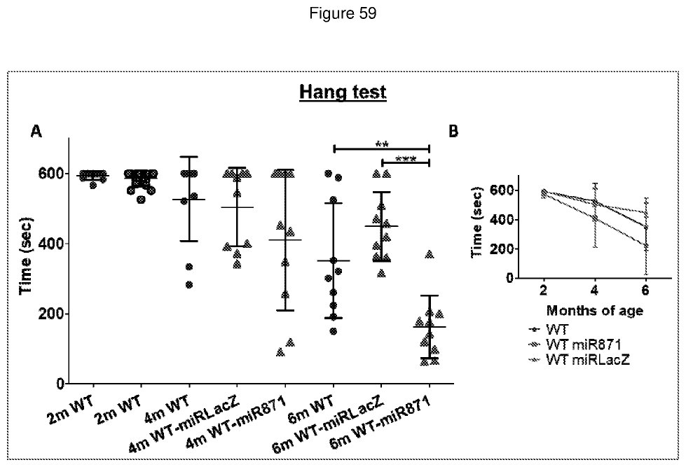

Rotarod analysis of WT injected groups at 5 and 17.5 rpm showed that AAV9-miR871 injection at WT mice negatively affected their motor performance at 2 months post injection (FIGS. 56-57). This phenotype was not observed at 4 months post injection as injected and non-injected WT mice presented similar performances (FIG. 44-45). Grip strength analysis, 2 and 4 months post injection, showed significant impairment of WT mice injected with AAV9-miR871 when compared to aged-matched non-injected and mock injected WT mice (FIG. 58). Hang test analysis showed that AAV9-miR871 injection at WT mice negatively affected mice performance only at 4 months post injection time point. At all the other time points hang test performances of baseline and injected WT mice did not present any statistical significant differences (FIG. 59).

### Example 10

### Motor Nerve Conduction Velocity (MNCV), Sciatic Nerve Amplitude of the Compound Muscle Action Potential (CMAP) and Hindlimb Clasp Observation

C61 Het mice show MNCVs of sciatic nerve around 28 m/s at 2 months of age, 22 m/s at 6 and 10 months of age [Huxley (1998), supra; Kohl et al., American Journal of Pathology, 176(3): 1390-1399 (2010)]. MNCVs properties of the sciatic nerves were compared in treatment groups as described previously [Huxley (1998), supra; Kohl, supra; Zielasek et al., Muscle & Nerve, 19(8): 946-952 (1996)]. For MNCV and CMAP, the left and right sciatic nerves were stimulated in anesthetized animals at the sciatic notch and distally at the ankle via bipolar electrodes with supramaximal square-wave pulses (5 V) of 0.05 ms. MNCV was calculated by dividing the distance between the stimulating and recording electrodes by the result of subtracting the distal latency from the proximal latency. The latencies of CMAP were recorded by a bipolar electrode inserted between digits 2 and 3 of the hind paw and measured from the stimulus artifact to the onset of the negative M-wave deflection. A fixed distance was used between distal stimulation and recording sites for calculating distal latency to avoid errors arising from variations in ankle-paw distance in each mouse. MNCV and CMAP of sciatic nerve was measured for early treated group at 6 months of age, late treated group at 10 months of age and WT-injected groups at 6 months of age, with all time points being 4 months after treatment, in order to assess functional properties in treated and control mice groups.

Statistics: MNCV and CMAP were compared using One way ANOVA with Tukey's Multiple Comparison Test GraphPad Prism5 software. Significance level for all comparisons, P<0.05.

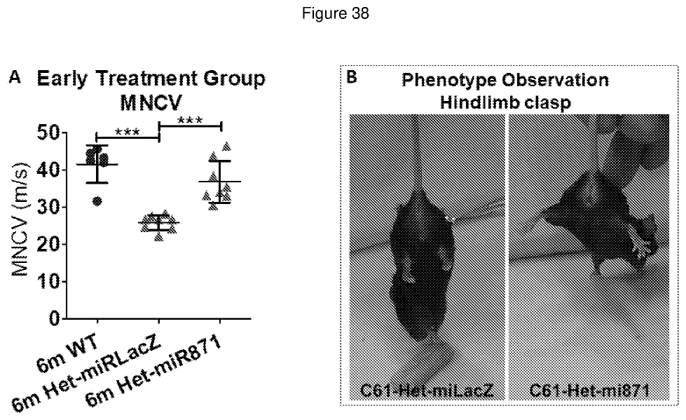

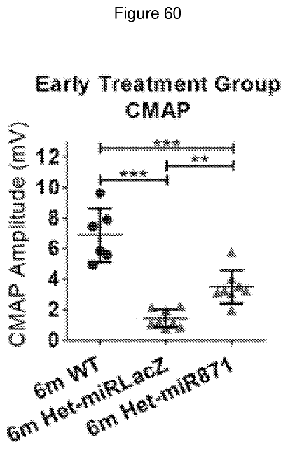

MNCV of early-treated mice sciatic nerves were measured 4 months after treatment, at the age of 6 months, in order to assess functional properties in miR871- and miRLacZ treated groups. MNCV and CMAP values were significantly improved in the miR871-treated group, reaching the average of 36.88 m/s and 3.51 mV, respectively (n=8) while MNCV and CMAP values in the miRLacZ group (n=8) were on average 25.9 m/s and 1.44 mV, respectively (FIG. 38A; p<0.0001 and FIG. 60). MNCV values of the miR871 treated group were close to those of the WT mice which the average of 41.61 m/s (n=6; p>0.05). However, CMAP values of miR871 group did not reach WT levels (WT CMAP score: 6.9 mV). Improved motor performance in early-treated C61-Het mice correlated with improvement in electrophysiological properties.

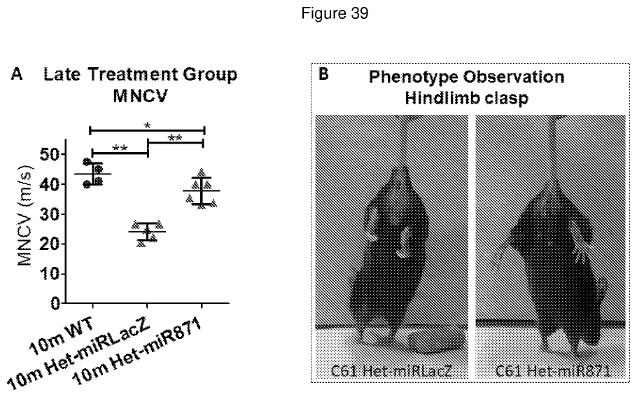

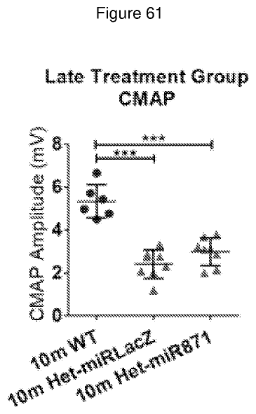

Similarly to early-treatment group, MNCV and CMAP of late-treated sciatic nerve were measured four months after treatment, at the age of 10 months, in order to assess functional properties in miR871 and miRLacZ groups. MNCV values were significantly improved in the miR871 treated group, reaching the average of 37.69 m/s (n=6) while velocities in the miRLacZ group (n=5) were on average 24.12 m/s (FIG. 39A; p=0.0040 and FIG. 61). However, MNCV values of the miR871 late-treated group did not manage to reach the values of the WT mice which had an average of 43.38 m/s (n=4; p=0.0333). CMAP values of 10-month old WT and miRLacZ mice differ significantly, with their averages being 5.33 and 2.4 mV, respectively. This phenotype was not improved after late treatment with AAV9-miR871 as the miR871 mice group had an average CMAP score of 2.98 mV. As in early-treated group, improved motor performance in early-treated C61-Het mice correlated with improvement in electrophysiological properties.

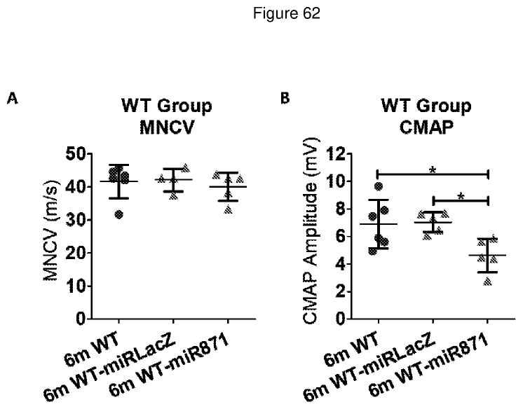

MNCV and CMAP of WT mice injected with AAV9-miRLacZ or AAV9-miR871 were measured four months after treatment, at the age of 6 months, in order to assess functional properties in miR871 and miRLacZ groups. MNCV values of WT, miRLacZ and miR871 treated groups did not differ, scoring the averages of 41.61, 42.09 and 40.07 m/s, respectively (FIG. 62A-B). CMAP values of 6 months old WT and miRLacZ mice did not differ significantly, with their averages being 6.89 and 7.03 m/V, respectively. miR871 group presented decreased CMAP score, with the average being 4.62 m/V.

Hindlimb clasping is a marker of disease progression in a number of mouse models of peripheral neuropathy [Arnaud et al., P.N.A.S. USA, 106(41): 17528-17533 (2009)]. The hindlimb clasping phenotype was observed in C61 Het mice starting from the first months of age and progressing until 10 months of age. During this observation, mice were suspended by the tail and abnormal clenching of toes and clasping was monitored as an indication of a peripheral nervous system defect.

Statistics: Hindlimbs opening angle data were compared using One way ANOVA with Tukey's Multiple Comparison Test GraphPad Prism5 software. Significance level for all comparisons, P<0.05.

Six-month-old C61 Het mice that were injected with AAV9-miRLacZ (mock) when 2 months old presented abnormal clenching of toes and clasping of hind limb phenotype upon suspension by the tail, suggestive of the presence of a peripheral nervous system defect. This phenotype was completely rescued in 6-months-old C61 Het mice that were injected with AAV9-miR871 at 2 months of age as they presented normal clenching without clasping of hind limbs (FIG. 38B).

10-month-old C61 Het mice that were injected with AAV9-miRLacZ (mock) when 6 months old presented abnormal clenching of toes and clasping of hind limb phenotype upon suspension by the tail, suggestive of the presence of a peripheral nervous system PNS defect. Similarly to the early treatment group, this phenotype was completely rescued in 10-months-old C61 Het mice that were injected with AAV9-miR871 at 6 months of age as they presented normal clenching without clasping of hind limbs (FIG. 39B).

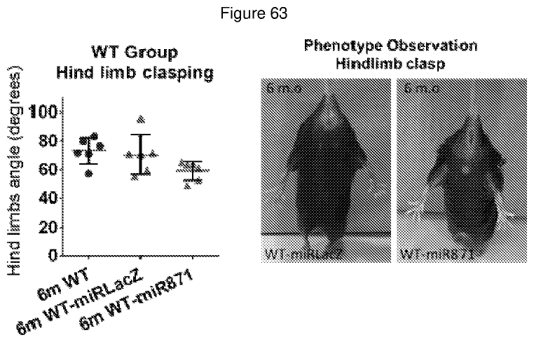

Non-injected WT and injected WT with either AAV9-miRLacZ or AAV9-miR871 did not presented any statistically significant difference in their hindlimb clasping phenotype (FIG. 63). Their hindlimb opening with the averages were 73.19, 70.54 and 59.09 degrees, respectively.

### Example 11

### Morphometric Analysis

Lumbar motor roots and femoral motor nerves of 6-month-old or 10-month-old AAV9-miR871 and AAV9-miRLacZ treated C61 Het mice were obtained for quantitative analysis of myelination following perfusion with 2.5% glutaraldehyde, osmication, dehydration, and embedding in araldite resin (all purchased from Agar Scientific, Essex, UK), as previously described [Kagiava (2019), supra]. Transverse semi-thin sections (1 μm) of the lumbar spinal cord with roots attached and the middle portion of the femoral motor nerves were obtained and stained with alkaline toluidine blue (Sigma-Aldrich, Munich, Germany). Sections were used to examine the degree of abnormal myelination in both groups. In brief, all demyelinated, thinly myelinated and normally myelinated axons were counted using the following criteria: axons larger than 1 μm without a myelin sheath were considered demyelinated; axons with myelin sheaths<10% of the axonal diameter were considered thinly myelinated; axons surrounded by circumferentially arranged Schwann cell processes and extracellular matrix were considered as “onion bulbs”; all other myelinated axons were considered normally myelinated. All pathological analyses were performed blinded to the treatment condition of each mouse. Morphological analysis was performed in multiple motor roots, as well as bilateral femoral motor nerves, and results were averaged per mouse. The number of abnormally myelinated fibers, including demyelinated and thinly myelinated fibers, were counted and percentage of fibers in each category was calculated. For onion bulb formations, the total number of bulbs per mouse was counted and presented as such.

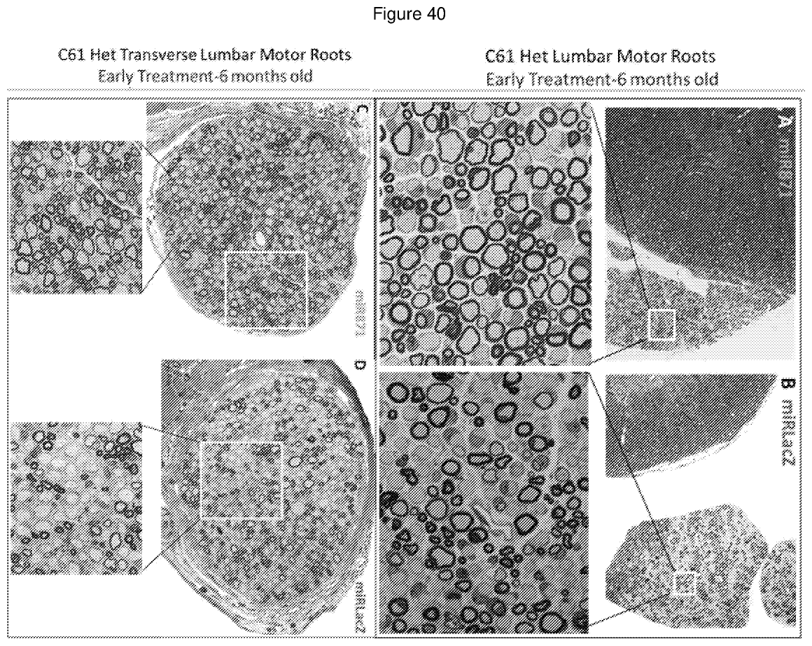

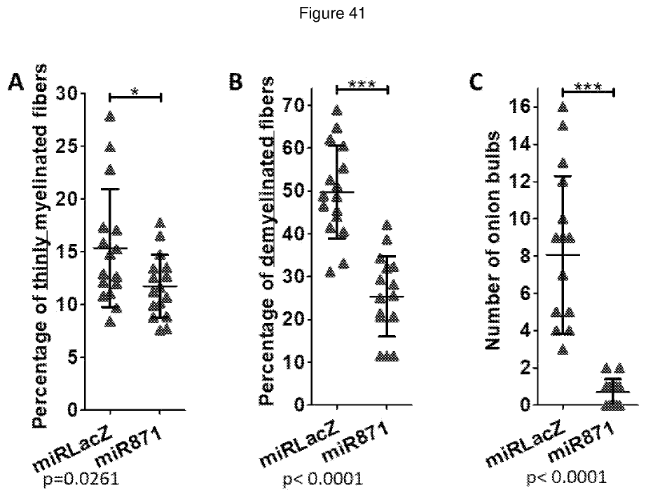

Morphological analysis showed reduction in the abnormally myelinated fibers in all PNS tissues examined (roots and femoral nerve) of early treated C61-Het mice with AAV9-miR871 (FIGS. 40-43). In the anterior lumbar motor roots of the AAV9-miR871 treated mice the percentage of abnormally myelinated fibers was reduced compared to the AAV9-miRLacZ group (FIGS. 40-41). In detail, the percentage of thinly myelinated fibers was 11.74% in the AAV9-miR871 treated mice (n=16), compared with 15.35% in the AAV9-miRLacZ treated mice (n=16; p=0.0261, Mann-Whitney test). Likewise, the percentage of demyelinated fibers was 25.36% in the AAV9-miR871 treated mice (n=16), compared with 49.74% in the AAV9-miRLacZ treated mice (n=16; p<0.0001, Mann-Whitney test). Onion bulb formation was also reduced after treatment with AAV9-miR871 (average of 0.69 onion bulb formations per section; n=16) compared to AAV9-miRLacZ treated (average of 8.06 onion bulb formations/section, n=16; p<0.0001, Mann-Whitney test).

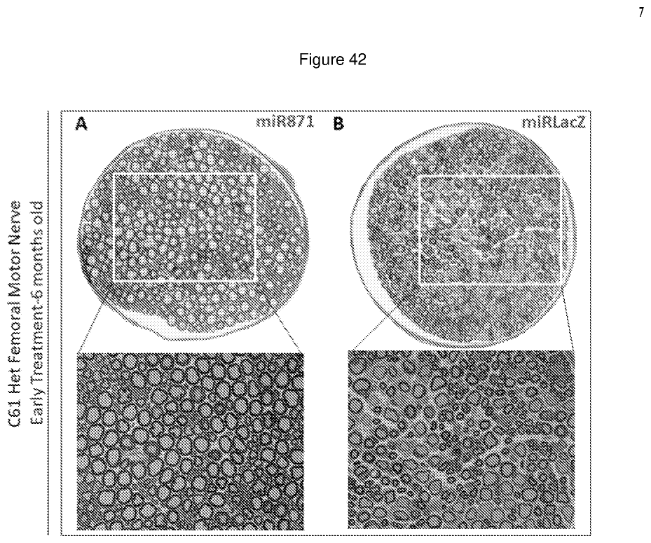

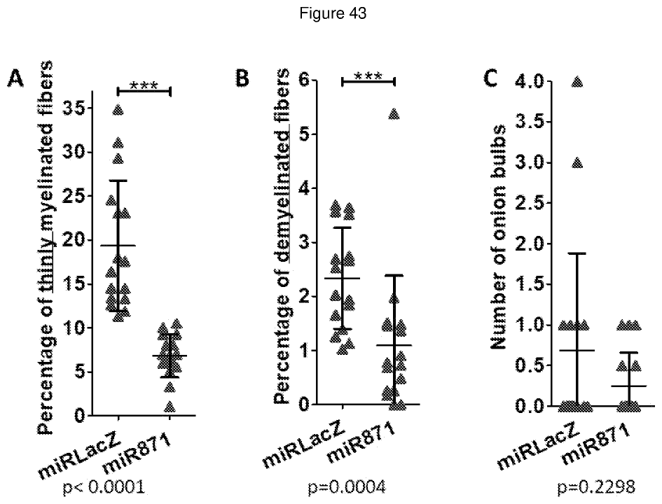

Likewise, in the femoral motor nerves of the AAV9-miR871 early-treated mice the percentage of abnormally myelinated fibers was reduced compared to the AAV9-miRLacZ group (FIGS. 42-43). The percentage of thinly myelinated fibers was 6.83% in the AAV9-miR871 treated mice (n=16), compared with 19.36% in the AAV9-miRLacZ treated mice (n=16; p<0.0001, Mann-Whitney test), while the percentage of demyelinated fibers was 1.09% in the AAV9-miR871 treated mice (n=16), compared with 2.33% in the AAV9-miRLacZ treated mice (n=16; p=0.0004, Mann-Whitney test). Not enough onion bulb formations were observed in miRLacZ femoral motor nerves of the early treatment group, resulting in no significant alternation in this formations after miR871 treatment (miRLacZ: 0.69%, miR871: 0.25%).

As in early treatment group, lumbar motor roots and femoral motor nerves of 10-month-old AAV9-miR871 or AAV9-miRLacZ treated C61 Het mice were obtained for quantitative analysis of myelination from groups of mice. Morphological analysis showed reduction in the abnormally myelinated fibers in all PNS tissues examined (roots and femoral nerve) of late treated C61-Het mice with AAV9-miR871 (FIG. 44-47). In the anterior lumbar motor roots of the AAV9-miR871 treated mice the percentage of abnormally myelinated fibers was reduced compared to the AAV9-miRLacZ group (FIG. 44-45). In detail, the percentage of thinly myelinated fibers was 14.62% in the AAV9-miR871 treated mice (n=7), compared with 19.39% in the AAV9-miRLacZ treated mice (n=7; p=0.0364, Mann-Whitney test). Likewise, the percentage of demyelinated fibers was 32.65% in the AAV9-miR871 treated mice (n=7), compared with 52.25% in the AAV9-miRLacZ treated mice (n=7; p=0.0035, Mann-Whitney test). Onion bulb formation was also reduced after treatment with AAV9-miR871 (average of 38.86 onion bulb formations per section; n=7) compared to AAV9-miRLacZ treated (average of 2.71 onion bulb formations/section, n=7; p=0.0010, Mann-Whitney test).

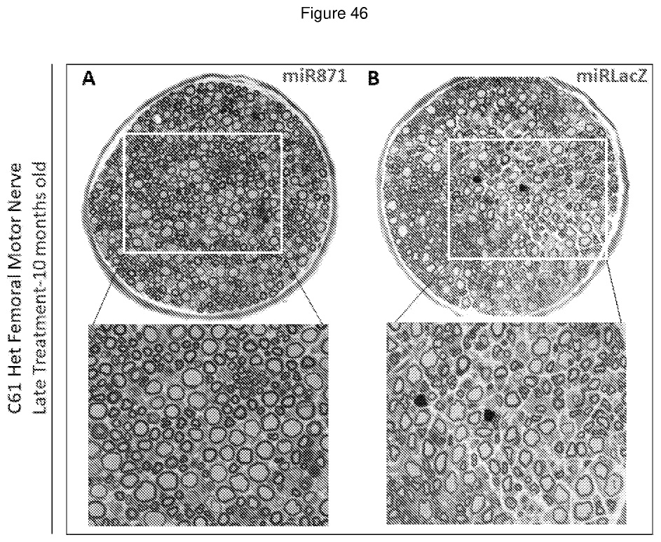

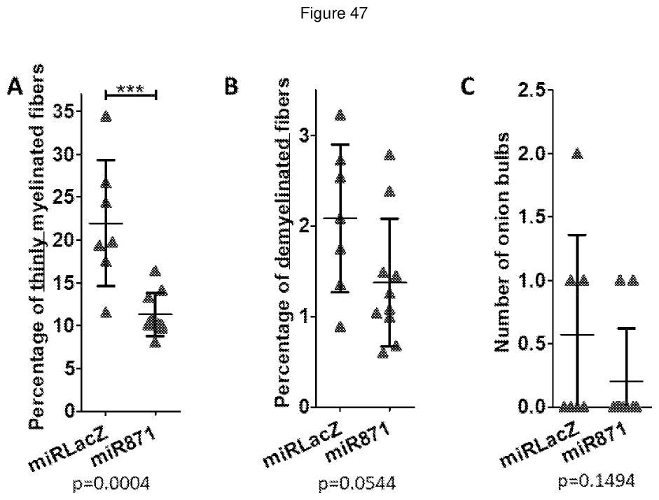

Likewise, in the femoral motor nerves of the AAV9-miR871 late-treated mice the percentage of abnormally myelinated fibers was reduced compared to the AAV9-miRLacZ group (FIG. 46-47). The percentage of thinly myelinated fibers was 11.31% in the AAV9-miR871 treated mice (n=10), compared with 21.95% in the AAV9-miRLacZ treated mice (n=7; p=0.0004, Mann-Whitney test). However, late treatment did not manage to reduce significantly the percentage of demyelinated fibers in AAV9-miR871 (n=10) treated mice when compared to AAV9-miRLacZ (n=7) treated mice (AAV9-miR871: 1.37%, miRLacZ: 2.08%; p=0.0544, Mann-Whitney test). Not enough onion bulb formations were observed in miRLacZ femoral motor nerves of the early treatment group, resulting in no significant alternation in this formations after AAV9-miR871 treatment (miRLacZ: 0.57%, miR871: 0.20%).

Statistics: Morphometric analysis data were compared using Mann-Whitney test GraphPad Prism5 software. Significance level for all comparisons, P<0.05.

### Example 12

### PMP22 Splice Forms

PMP22 has several splice variants. Six alternative splice forms were identified empirically in three different studies. [Visigalli et al., supra; Suter et al., supra; and Huehne and Rautenstrauss, Int. J. Mol. Med., 7(6): 669-675 (2001). In addition, Ensembl.org shows twenty-six different human PMP22 splice forms, most of which are predicted in silico. Three of these splice forms likely undergo nonsense-mediated decay (NMD) and therefore do not encode proteins. In addition, there are four other truncated, non-coding processed transcripts that could arise from this locus (Ensembl transcripts 205, 206, 227, 229). Thus, Ensembl predicts twenty-three possible protein-coding transcripts arising from the PMP22 locus, producing eight possible protein isoforms.

### Human PMP22 Splice Forms EMSEMBL.org

| Name | Transcript ID | bp | Protein | Biotype | CCDs |
|---|---|---|---|---|---|
| PMP22-215 | ENST00000674673.1 | 2423 | 160aa | Protein coding | CCDS11168 |
| PMP22-223 | ENST00000675808.1 | 2047 | 160aa | Protein coding | CCDS11168 |
| PMP22-213 | ENST00000646419.2 | 1855 | 118aa | Protein coding | CCDS82078 |
| PMP22-201 | ENST00000312280.9 | 1828 | 160aa | Protein coding | CCDS11168 |
| PMP22-210 | ENST00000612492.5 | 1828 | 160aa | Protein coding | CCDS11168 |
| PMP22-230 | ENST00000676221.1 | 1819 | 160aa | Protein coding | CCDS11168 |
| PMP22-202 | ENST00000395936.7 | 1796 | 118aa | Protein coding | CCDS82078 |
| PMP22-226 | ENST00000675950.1 | 1789 | 160aa | Protein coding | CCDS11168 |
| PMP22-224 | ENST00000675819.1 | 1774 | 160aa | Protein coding | CCDS11168 |
| PMP22-214 | ENST00000674651.1 | 1755 | 160aa | Protein coding | CCDS11168 |
| PMP22-212 | ENST00000644020.1 | 1738 | 118aa | Protein coding | CCDS82078 |
| PMP22-217 | ENST00000674868.1 | 1312 | 160aa | Protein coding | CCDS11168 |
| PMP22-221 | ENST00000675350.1 | 1058 | 160aa | Protein coding | CCDS11168 |
| PMP22-204 | ENST00000426385.4 | 4161 | 125aa | Protein coding | — |
| PMP22-222 | ENST00000675551.1 | 1822 | 165aa | Protein coding | — |
| PMP22-203 | ENST00000395938.7 | 1803 | 217aa | Protein coding | — |
| PMP22-231 | ENST00000676329.1 | 1757 | 194aa | Protein coding | — |
| PMP22-209 | ENST00000580584.3 | 1716 | 92aa | Protein coding | — |
| PMP22-225 | ENST00000675854.1 | 1667 | 92aa | Protein coding | — |
| PMP22-219 | ENST00000674947.1 | 1662 | 217aa | Protein coding | — |
| PMP22-207 | ENST00000494511.7 | 1616 | 92aa | Protein coding | — |
| PMP22-228 | ENST00000676161.1 | 1575 | 113aa | Protein coding | — |
| PMP22-216 | ENST00000674707.1 | 1543 | 92aa | Protein coding | — |

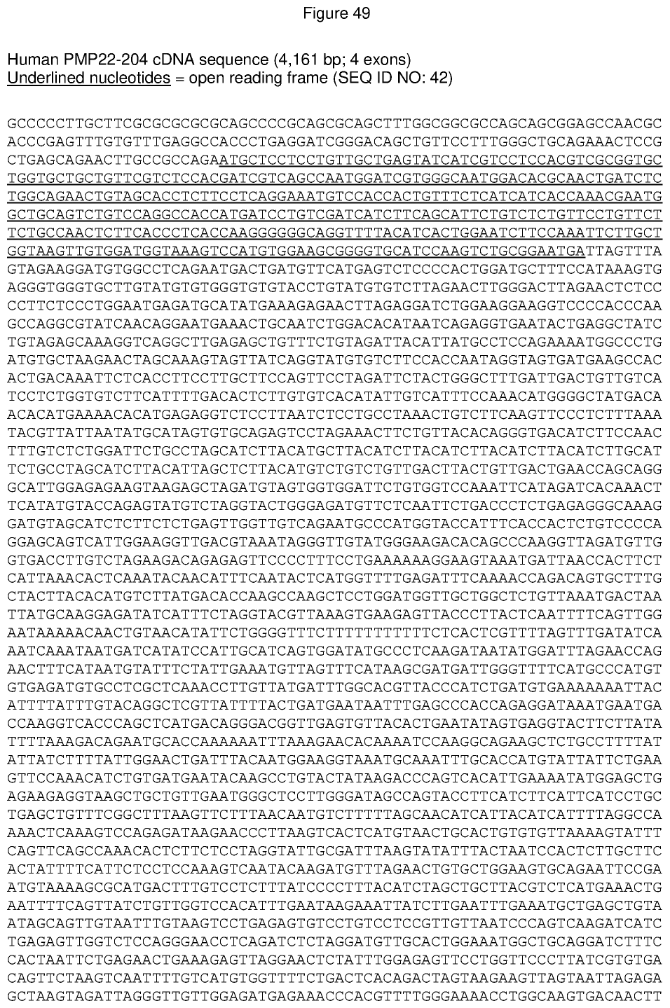

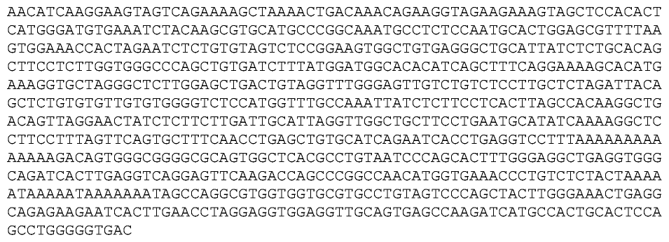

The two longest Ensembl transcripts are PMP22-215 (2,423 bp) and PMP22-204 (4,161 bp). PMP22-215 encodes the full-length, 160-amino acid PMP22 protein, while PMP22-204 produces a shorter, 125-amino acid isoform. FIG. 49 shows the PMP22-204 cDNA sequence.

The miPMP22-868 and miPMP22-871 described above target binding sites in twenty-two of the twenty-three possible protein-coding PMP22 transcripts (FIG. 50). The one transcript exception is PMP22-204, which contains a retained intron at the end of exon 4, thereby producing an alternative 3′ untranslated region (3′ UTR).

### Example 13

Immune Response Analysis 6 Weeks after AAV9-miRLacZ Intrathecal Injection in C61 Het Mice

Two-month old C61 Het mice were intrathecally injected with AAV9-miRLacZ and were then sacrificed either at 6 weeks post injection (3.5 months old) or at 4 months post injection (6 months old). Tissues were collected for immunohistochemistry analysis as described before above in Example 6. Lumbar roots, sciatic nerves and liver sections were stained against CD20 (1:100 Santa Cruz), CD45 (1:100 Abcam), CD68 (1:50 Biorad) and CD3 (1:100 Abcam). Cell nuclei were visualized with DAP. The percentage of CD20, CD45, CD68 and CD3 positive cells was calculated in relation to the total cell number.

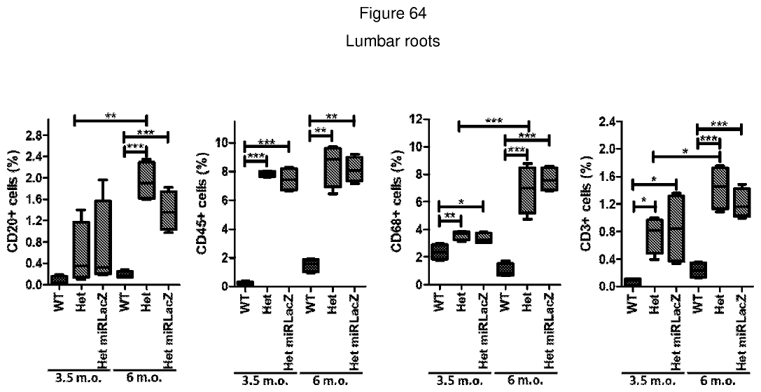

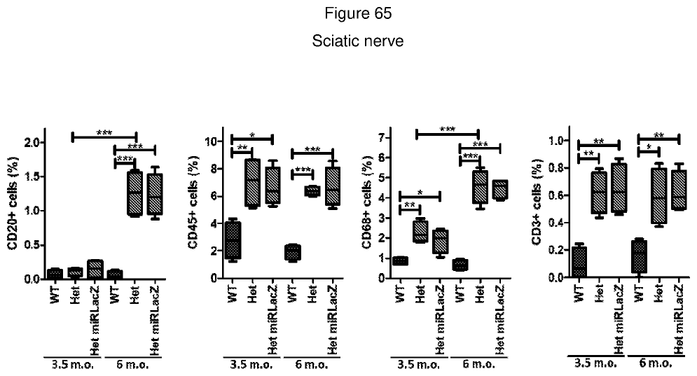

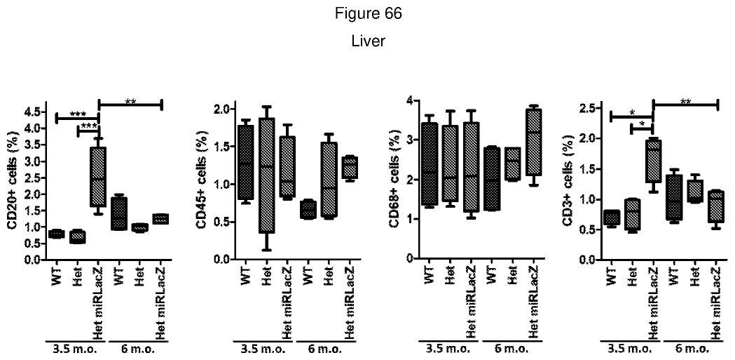

Immunological response to the intrathecal delivery of AAV9-miRLacZ to 2-month old C61 Het mice was analyzed either at 6 weeks or 4 months post-injection by quantifying B-cell marker CD20, leukocyte marker CD45, macrophage marker CD68 and T-cell marker CD3 at lumbar roots (FIG. 64), sciatic nerve (FIG. 65) and liver (FIG. 66). AAV9-miRLacz injected C61 Het mice were compared to age matched non-injected C61 Het and WT (expressing only normal levels of murine PMP22) mice.

CD20, CD45, CD3 and CD68 markers immune response analysis of lumbar roots (FIG. 64) showed that CD20 levels of WT, C61 Het and C61 Het-AAV9-miRLacZ did not differ significantly at the 6-week time point. Non-injected C61 Het CD20 levels were significantly increased when comparing 3.5 with 6 months old values. Increased levels of CD20 were presented in non-injected and AAV9-miRLacZ injected C61 Het mice at 4 months post injection, mice were 6 months old, when compared to aged matched WT mice. AAV9-miRLacZ injection in C61 Het mice did not affect CD20 levels when compared to non-injected C61 Het mice. CD45, CD68 and CD3 levels of lumbar roots at both time points were shown to be elevated in non-injected and AAV9-miRLacZ C61 Het mice when compared to aged matched non-injected WT mice. CD68 and CD3 levels of non-injected C61 Het mice were increased as the animals aged. AAV9-miRLacZ injection in C61 Het mice did not affected CD markers levels when compared to non-injected C61 Het mice.

CD20, CD45, CD3 and CD68 markers immune response analysis of sciatic nerves (FIG. 65) showed that CD20 levels at the 6-week time point of WT, C61 Het and C61 Het-AAV9-miRLacZ did not differ significantly. Non-injected C61 Het CD20 levels were significantly increased when comparing 3.5- with 6-month old values. Increased levels of CD20 were presented in non-injected and AAV9-miRLacZ injected C61 Het mice at 4 months post injection, mice were 6 months old, when compared to aged matched WT mice. AAV9-miRLacZ injection in C61 Het mice did not affect CD20 levels when compared to non-injected C61 Het mice. CD45, CD68 and CD3 levels of sciatic nerves at both time points were shown to be elevated in non-injected and AAV9-miRLacZ C61 Het mice when compared to aged matched non-injected WT mice. CD68 levels of baseline C61 Het mice were increased as the animals aged. AAV9-miRLacZ injection in C61 Het mice did not affect CD markers levels when compared to non-injected C61 Het mice.

CD20, CD45, CD3 and CD68 marker immune response analysis of liver (FIG. 66) showed that non-injected WT and C61 Het express similar numbers of immune response markers. CD20 and CD3 levels of C61 Het-AAV9-miRLacZ mice at the 6-week time point were increased when compared to non-injected WT and C61 Het. This increase was balanced back to baseline levels at the 4-month time point.

According to these data, non-injected C61 Het mice present elevated immune response markers in lumbar roots and sciatic nerve sections that increased with age when compared to age-matched WT controls. This phenotype was not affected by AAV9-miRLacZ injection. Both 3.5- and 6-month old non-injected C61 Het mice presented normal levels of immune response markers in their liver when compared to aged matched control. At 4 weeks post injection time point, C61 Het-AAV9-miRLacZ injected mice presented increased the levels of CD20 and CD3 positive cells of the liver, indicating a systemically immune response to AAV9 injection. This phenotype was ameliorated as the post injection time was progressing as no inflammatory response was detected in C61 Het mice injected with AAV9-miRLacZ after 4 months of the injection.

Statistics: Immunostaining (percentage of CD positive cells) data were compared using One way ANOVA with Tukey's Multiple Comparison Test GraphPad Prism5 software. Significance level for all comparisons, P<0.05.

### Example 14

### Plasma Neurofilament Light (Nfl) Levels

In order to further evaluate the effectiveness of our treatment, we performed Nfl biomarker analysis from blood samples on baseline WT, C61 Het, C61 Het injected with AAV9-miRLacZ and C61 Het injected with AAV9-miR871, at early and late treatment end time points. Nfl concentrations are a dynamic measure of axonal damage and serve as a biomarker for CMT disease severity. Blood was collected prior to sacrificing the animals using standard methods [Parasuraman, et al., Journal of pharmacology & pharmacotherapeutics, 1, (2): 87-93 (2010)].

Blood samples were collected as previously described and processed within one hour [Kagiava et al., Gene therapy, Online ahead of print (2021)]. Blood samples were collected in EDTA-containing tubes and centrifuged at 20° C. at 3500 rpm for 10 min. Centrifugation separated blood samples in two phases and the top plasma phase was collected and stored at −80° C. until testing. Plasma Nfl concentration was measured at University College London (UCL) using a commercially available NF-Light kit on a Single molecule array (Simoa) HD-1 instrument (Quanterix, Billerica, MA) [Rohrer et al., Neurology, 87 (13): 1329-1336 (2016); Sandelius et al., Neurology, 90 (6): e518-e524 (2018)].

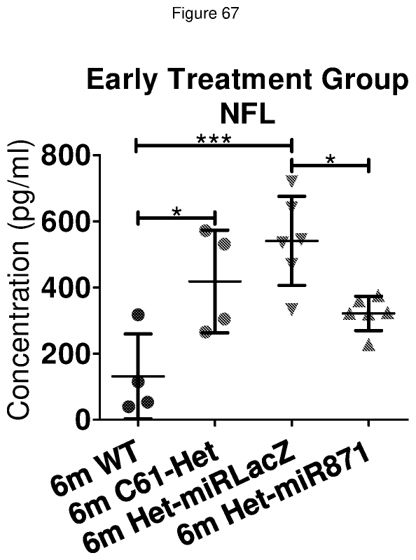

Nfl concentration of non-injected 6 months old C61 Het mice was elevated (n=4, 418.07 μg/ml) compared to age matched non-injected WT control mice (n=4, 131.10 μg/ml) (FIG. 67). Early treated C61 Het mice with AAV9-miR971 (n=6) presented lower concentration (321.37 μg/ml) of Nfl compared to AAV9-miRLacZ group (n=6, 540.65 μg/ml), with AAV9-miR971 scores being close to WT levels (131.10 μg/ml) (FIG. 62). Injection with AAV9-miRLacZ did not resulted in any alternations to plasma Nfl levels when compared to non-injected C61 Het mice (FIG. 67).

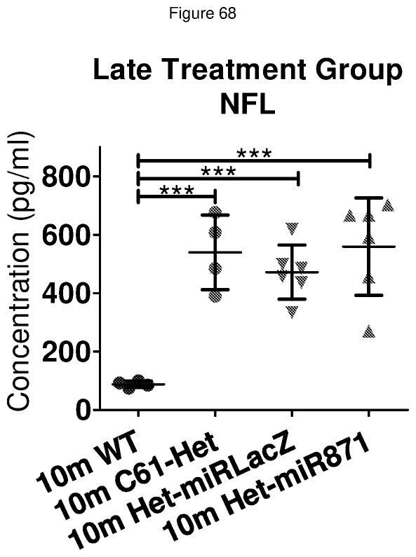

Nfl concentration of non-injected 10 months old C61 Het mice was elevated (n=4, 539.66 μg/ml) compared to age matched non-injected WT control mice (n=4, 88.07 μg/ml) (FIG. 68). Late treated C61 Het mice with AAV9-miR971 did not presented improved plasma Nfl levels when compared to aged matched non-injected C61 Het or C61 Het injected with AAV9-miRLacZ (FIG. 68). Non injected as well as AAV9-miRLacZ and AAV9-miR871 injected C61 Het mice presented similar levels of Nfl (C61 Het AAV9-miRLacZ: 471.99 μg/ml, C61 Het AAV9-miR871: 559.28 μg/ml) (FIG. 68).

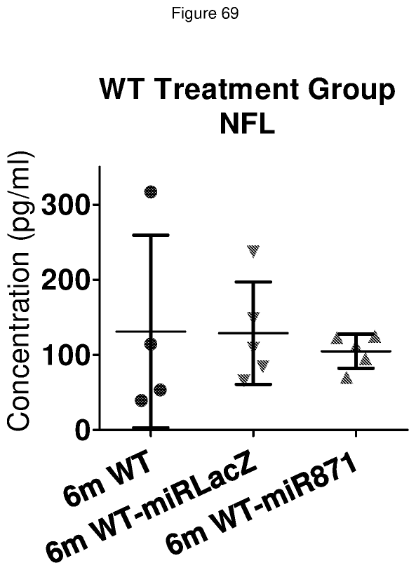

Nfl concentration of WT-injected (miRLacZ: n=5, miR871: n=5) and non-injected (n=4) 6 months old mice was similar score with no statistically significant difference among them (6 m WT: 131.10 μg/ml, WT AAV9-miRLacZ: 128.93 μg/ml, WT AAV9-miR871: 104.92 μg/ml) (FIG. 69).

Statistics: Nfl concentration data were compared using One way ANOVA with Tukey's Multiple Comparison Test GraphPad Prism5 software. Significance level for all comparisons, P<0.05.

### Example 15

Immune Response Analysis 4 Months after AAV9-miR871 Intrathecal Injection in C61 Het Mice

C61 Het mice were intrathecally injected with AAV9-miR871 at either 2 months (early treatment) or 6 months (late treatment) of age and were then sacrificed at 4 months post injection (6 or 10 months of age, respectively). Tissues were collected for immunohistochemistry analysis as described before at “Example 6”. Lumbar roots, sciatic nerves and liver sections were stained against CD20 (1:100 Santa Cruz), CD45 (1:100 Abcam), CD68 (1:50 Biorad) and CD3 (1:100 Abcam). Cell nuclei were visualized with DAP. The percentage of CD20, CD45, CD68 and CD3 positive cells was calculated in relation to the total cell number.

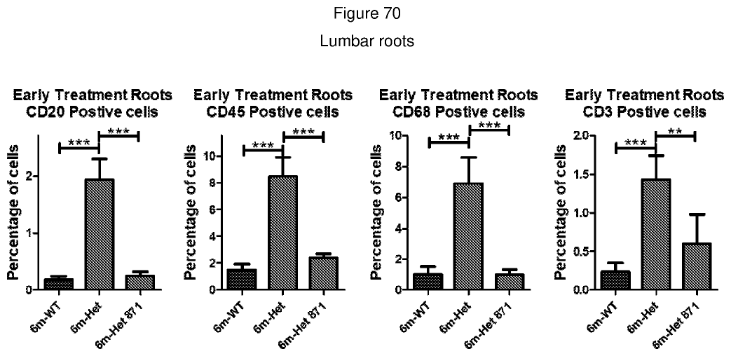

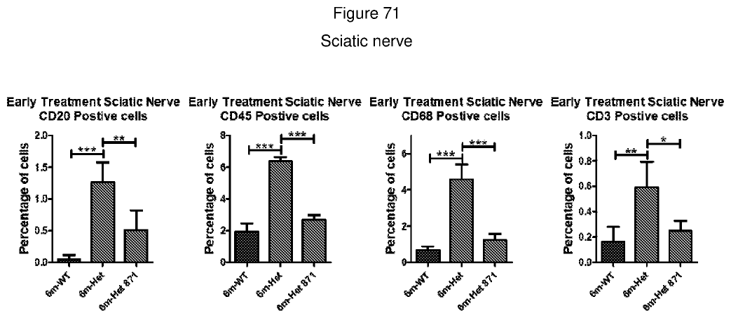

Lumbar roots and sciatic nerves sections of 6 months old non-injected C61 Het mice presented higher levels of CD20, CD45, CD68 and CD3 positive cells compared to aged matched non-injected WT mice (FIGS. 70-71). Lumbar roots and sciatic nerves of early treated C61 Het mice injected with AAV9-miR871 showed reduced levels of CD20, CD45, CD68 and CD3 positive cells, with these scores reaching WT levels (FIGS. 70-71). According to these data, lumbar roots and sciatic nerves of 6 months old C61 Het mice present elevated scores of immune response markers that were decreased back to WT levels after early treatment with AAV9-miR871.

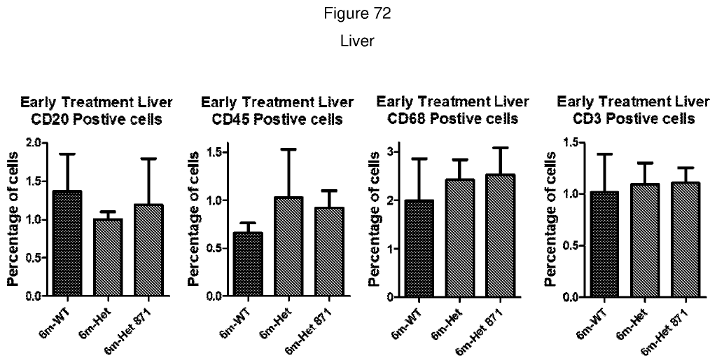

Liver sections of 6 months non-injected WT and C61 Het as well as C61 Het mice injected with AAV9-miR871 showed similar scores of CD20, CD45, CD68 and CD3 positive cells (FIG. 72). According to these data, livers of 6 months old C61 Het mice do not express extra inflammatory response at baseline or 4 months post-injection with AAV9-miR871.

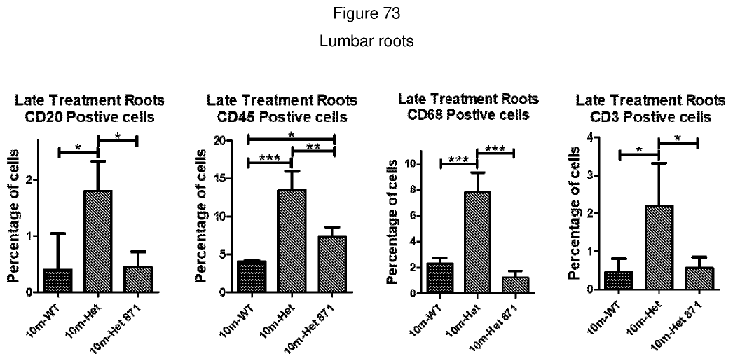

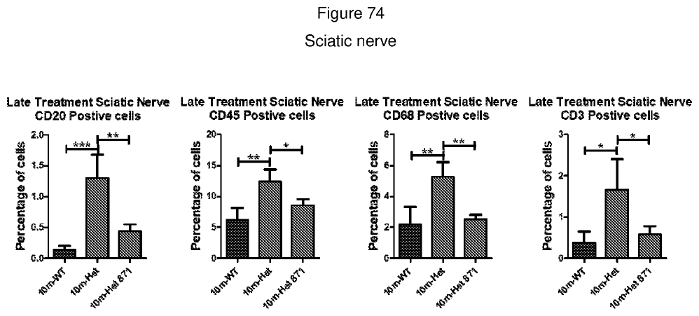

Lumbar roots and sciatic nerves sections of 10 months old non-injected C61 Het mice presented higher levels of CD20, CD45, CD68 and CD3 positive cells compared to aged matched non-injected WT mice (FIGS. 73-74). Lumbar roots and sciatic nerves of late treated C61 Het mice injected with AAV9-miR871 showed reduced levels of CD20, CD45, CD68 and CD3 positive cells (FIGS. 73-74). C61 Het-AA9-miR871 scores reached WT levels, with only exception being CD45 score of lumbar roots (FIGS. 73-74). According to these data, lumbar roots and sciatic nerves of 10 months old C61 Het mice present elevated scores of immune response markers that were decreased to WT levels after early treatment with AAV9-miR871, with only exception being CD45 marker of lumbar roots.

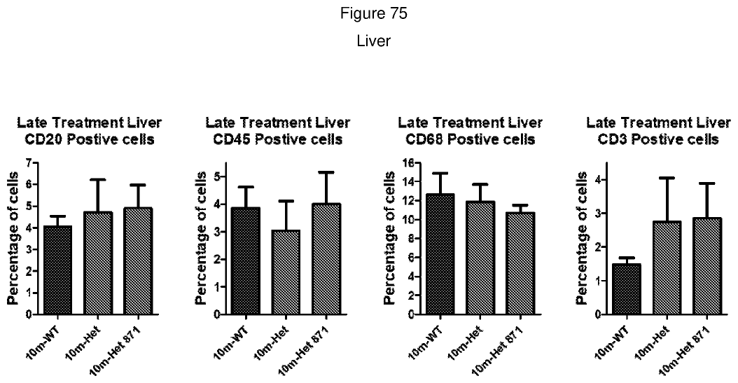

Liver sections of 10 months non-injected WT and C61 Het as well as C61 Het mice injected with AAV9-miR871 showed similar scores of CD20, CD45, CD68 and CD3 positive cells (FIG. 75). According to these data, livers of 6 months old C61 Het mice do not express extra inflammatory response at baseline or 4 months post-injection with AAV9-miR871.

### Example 16

### VGCN Analysis of PNS and Non-PNS Tissues of Early and Late Treatment Groups, 4 Months Post Injection

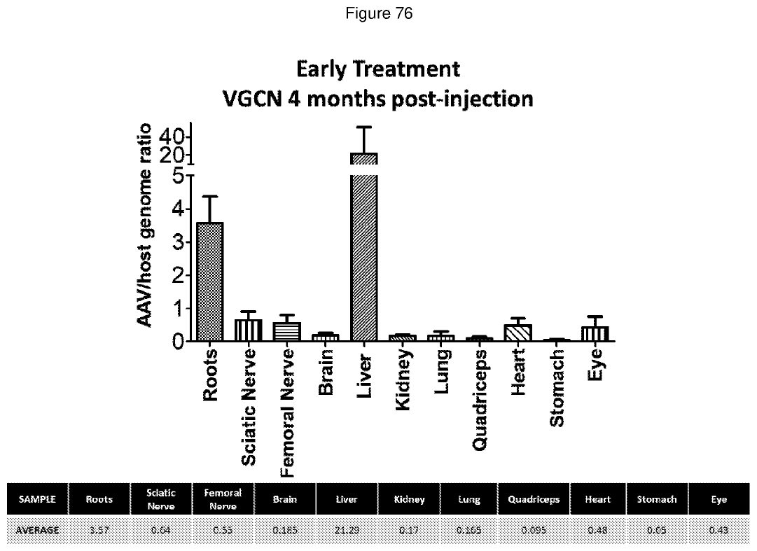

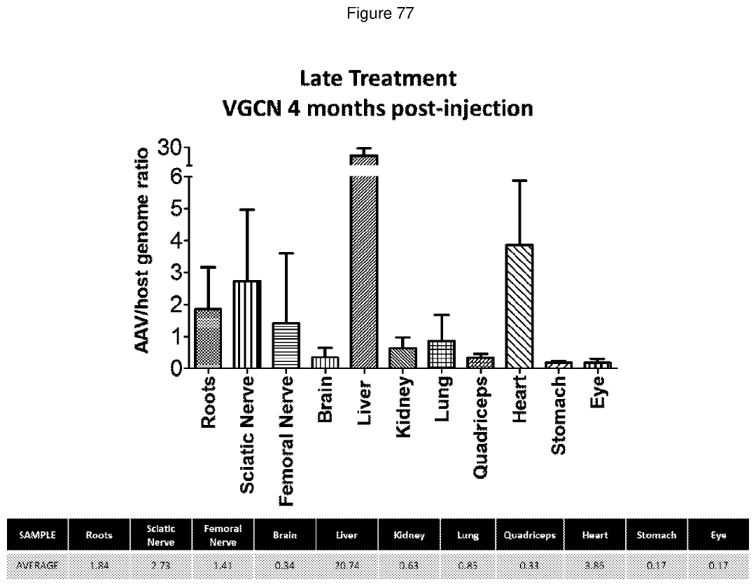

C61 Het mice were injected with AAV9-miR871 either at 2 months of age (early treatment) or 6 months of age (late treatment) and were sacrificed 4 months post injection when mice were 6 or 10 months old, respectively. PNS (lumbar roots, sciatic and femoral nerve) as well as non-PNS (brain, liver, kidney, lung, quadriceps, heart, stomach and eye) samples were collected and processed for VGCN analysis as described in Example 6”. AAV9 viral vector particles were detectable in all examined tissues at significantly high amounts 4 months post injection of both treatment groups (FIGS. 76-77). For both treatment groups, liver was the tissues with the highest VGCN score and stomach was the tissue with the lowest VCGN score (FIGS. 76-77).

While the present invention has been described in terms of specific embodiments, it is understood that variations and modifications will occur to those skilled in the art. Accordingly, only such limitations as appear in the claims should be placed on the invention.

All documents referred to in this application are hereby incorporated by reference in their entirety.

1. A nucleic acid comprising:
(a) a template nucleic acid set forth in any one of SEQ ID NOs: 1-8;
(b) a nucleic acid encoding a PMP22 artificial inhibitory RNA at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the polynucleotide sequence set forth in any one of SEQ ID NOs: 9-16,
(c) a nucleic acid encoding a PMP22 artificial inhibitory RNA set forth in any one of SEQ ID NOs: 9-16;
(d) a nucleic acid encoding a PMP22 antisense guide strand at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the polynucleotide sequence set forth in any one of SEQ ID NOs: 17-24; or
(e) a nucleic acid encoding a PMP22 antisense guide strand set forth in any one of SEQ ID NOs: 17-24.

2. A viral vector comprising the nucleic acid of claim 1 or a combination of any one or more thereof.

3. The viral vector of claim 2, wherein the viral vector is an adeno-associated virus (AAV), adenovirus, lentivirus, retrovirus, poxvirus, baculovirus, herpes simplex virus, vaccinia virus, or a synthetic virus.

4. The viral vector of claim 3, wherein the viral vector is an AAV.

5. The viral vector of claim 4, wherein the AAV lacks rep and cap genes.

6. The viral vector of claim 4, wherein the AAV is a recombinant AAV (rAAV) or a self-complementary recombinant AAV (scAAV).

7. The viral vector of claim 4, wherein the AAV has a capsid serotype of: AAV-1, AAV-2, AAV-3, AAV-4, AAV-5, AAV-6, AAV-7, AAV-8, AAV-9, AAV-10, AAV-11, AAV-12, AAV-13, AAV-anc80, orAAV rh.74.

8. The viral vector of claim 4, wherein the AAV has a capsid serotype of AAV-9.

9. The viral vector of claim 4, wherein the AAV is a pseudotyped AAV.

10. The viral vector of claim 9, wherein the AAV is AAV2/8 or AAV2/9.

11. The viral vector of claim 4, wherein expression of the nucleic acid encoding the PMP22 artificial inhibitory RNA is under the control of a U6 promoter.

12. A composition comprising the nucleic acid of claim 1 and a pharmaceutically acceptable carrier.

13. A composition comprising the viral vector of claim 4 and a pharmaceutically acceptable carrier.

14. A composition comprising a delivery vehicle capable of delivering agents to a Schwann cell and a nucleic acid encoding an artificial inhibitory RNA, wherein the artificial inhibitory RNA binds a segment of a messenger RNA (mRNA) encoded by a human peripheral myelin protein-22 (PMP22) gene, and, optionally, a pharmaceutically acceptable carrier.

15. The composition of claim 14, wherein the human PMP22 gene comprises the sequence of SEQ ID NO: 25, or a variant thereof at least 70%, 75%, 80%, 81%, 82%, 83%, 84%, 85%, 86%, 87%, 88%, 89%, 90%, 91%, 92%, 93%, 94%, 95%, 96%, 97%, 98%, or 99%, identical to the sequence of SEQ ID NO: 25.

16. The composition of claim 14, wherein the mRNA segment is complementary to a sequence within nucleotides 1 to 2423 of SEQ ID NO: 25.

17. The composition of claim 16, wherein the mRNA segment is complementary to a sequence within nucleotides 1412-1433 or 1415-1436 of SEQ ID NO: 25.

18. The composition of claim 14, wherein the delivery vehicle is a viral vector.

19. The composition of claim 18, wherein the viral vector is an adeno-associated virus (AAV), adenovirus, lentivirus, retrovirus, poxvirus, baculovirus, herpes simplex virus, vaccinia virus, or a synthetic virus.

20. The composition of claim 19, wherein the viral vector is an AAV.

21. The composition of claim 20, wherein the AAV lacks rep and cap genes.

22. The composition of claim 20, wherein the AAV is a recombinant AAV (rAAV), a recombinant single-stranded AAV (ssAAV), or a self-complementary recombinant AAV (scAAV).

23. The composition of claim 20, wherein the AAV has a capsid serotype selected from the group consisting of: AAV-1, AAV-2, AAV-3, AAV-4, AAV-5, AAV-6, AAV-7, AAV-8, AAV-9, AAV-10, AAV-11, AAV-12, AAV-13, AAV-anc80, and AAV rh.74.

24. The composition of claim 20, wherein the AAV has a capsid serotype of AAV-9.

25. The composition of claim 20, wherein the AAV is a pseudotyped AAV.

26. The composition of claim 25, wherein the AAV is AAV2/8 or AAV2/9.

27. The composition of claim 14, wherein expression of the nucleic acid encoding the PMP22 artificial inhibitory RNA is under the control of a U6 promoter.

28. A method of delivery to a Schwann cell with a duplicated peripheral myelin protein-22 (PMP22) gene, the method comprising administering to a subject with the Schwann cell
the nucleic acid of claim 1.

29. A method of treating a subject suffering from overexpression of a peripheral myelin protein-22 (PMP22) gene, the method comprising administering to the subject
the nucleic acid of claim 1.

30. The method of claim 29 wherein the subject suffers from Charcot-Marie-Tooth Disease Type 1A (CMT1A).

31. The method of claim 30, wherein the subject is a human subject.
